(19) United States
(12) Patent Application Publication
(10) Pub. No.: US 2023/0321228 A1
(43) Pub. Date: Oct. 12, 2023

(54) VACCINE ADJUVANTS AND METHODS OF SYNTHESIZING AND USING THE SAME

(71) Applicant: Amyris, Inc., Emeryville, CA (US)

(72) Inventors: Christopher John PADDON, Emeryville, CA (US); Karl Joseph FISHER, Emeryville, CA (US)

(21) Appl. No.: 18/014,881

(22) PCT Filed: Jul. 7, 2021
(86) PCT No.: PCT/US2021/040714

§ 371 Date: Jan. 6, 2023

Related U.S. Application Data
(60) Provisional application No. 63/048,910, filed on Jul. 7, 2020.

(51) Int. Cl. A61K 39/39; C07C 43/15; C07C 43/215; C07C 49/258

(52) U.S. Cl. CPC A61K 39/39; C07C 43/15; C07C 43/215; C07C 49/258; A61K 2039/55511

(57) ABSTRACT

The disclosure provides compounds useful as adjuvants in vaccines, as well as methods of synthesizing such compounds and methods of using such compounds in the formulation of a vaccine. The disclosure also features methods of administering such vaccines to a subject (e.g., a mammalian subject, such as a human) in order to treat or prevent one or more diseases, such as a disease caused by a viral or bacterial infection.

### FIELD OF THE INVENTION

The disclosure relates to the field of vaccine adjuvant formulations and therapeutic and prophylactic uses of the same.

### BACKGROUND OF THE INVENTION

Vaccine adjuvants are compounds that have intrinsic immunomodulatory properties and, when administered in conjunction with an antigen, potentiate host antigen-specific immune responses compared to responses raised when antigen is given alone. Despite recent progress in vaccine adjuvant development, there remains a need for sustainable alternatives to adjuvants that are isolated from natural sources. Moreover, there remains a need for improved adjuvants that more potently enhance the therapeutic or prophylactic strength of a vaccine.

### SUMMARY OF THE INVENTION

The present disclosure relates to compounds useful as adjuvants in vaccines, as well as methods of synthesizing such compounds and using the same in the formulation of a vaccine. The disclosure also features methods of administering such vaccines to a subject (e.g., a mammalian subject, such as a human) in order to treat or prevent one or more diseases, such as a disease caused by a viral or bacterial infection.

In a first aspect (“A1”), the disclosure features a compound represented by formula (I-a)

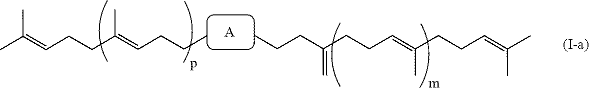

wherein A is

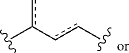

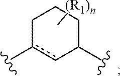

each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
each

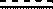

is, independently, a single bond or a double bond;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

In another aspect (“A2”), the disclosure features a compound represented by formula (I-b)

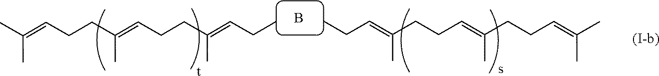

wherein B is —O—, —S—, -O(C(R₁₀)₂)ₙO-, -(C(R₁₀)₂)ₙ-, -N(R₁₁)₂-,

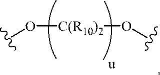

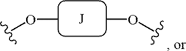

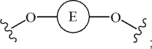

J is optionally substituted alkylene, optionally substituted heteroalkylene, optionally substituted alkenylene, optionally substituted heteroalkenylene, optionally substituted alkynylene, or optionally substituted heteroalkynylene;
E is optionally substituted carbocyclyl or optionally substituted heterocyclyl;
each of R₁₀ and R₁₁ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, halogen, or mono-, di- or trisaccharyl;
each n is, independently, an integer from 1 to 6 (e.g., 1, 2, 3, 4, 5, or 6);
s is an integer from 0 to 3 (e.g., 0, 1, 2, or 3);
t is an integer from 0 to 3 (e.g., 0, 1, 2, or 3); and
u is an integer from 1 to 8 (e.g., 1, 2, 3, 4, 5, 6, 7, or 8);
or a pharmaceutically acceptable salt thereof.

In some embodiments of A2, each of R₁₀ and R₁₁ is, independently, hydrogen, optionally substituted alky, optionally substituted aminoalkyl, or mono-, di- or trisaccharyl.

In some embodiments of A1, the compound is represented by formula (II)

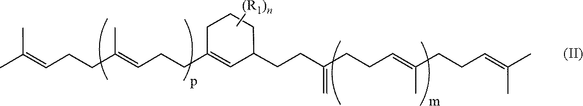

wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (II-a)

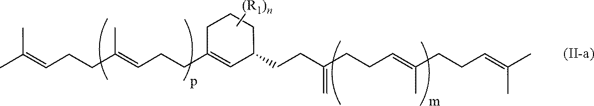

wherein each R₁ is, independently, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (II-b)

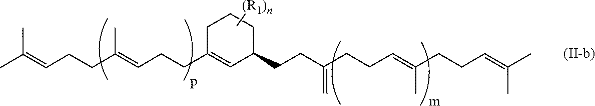

wherein each R₁ is, independently, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (I-a), (II), (II-a), and (II-b), each R₁ is, independently, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of any one of compounds (I-a), (II), (II-a), and (II-b), each R₁ is, independently, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of any one of compounds (I-a), (II), (II-a), and (II-b), each R₁ is, independently,

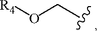

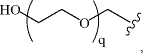

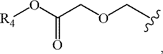

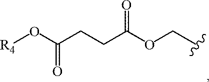

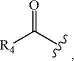

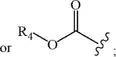

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of any one of compounds (I-a), (II), (II-a), and (II-b), each R₁ is, independently,

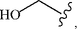

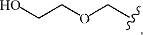

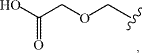

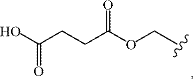

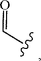

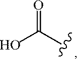

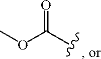

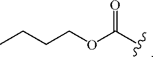

In some embodiments of any one of compounds (I-a), (II), (II-a), and (II-b), each R₁ is,
independently,

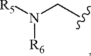

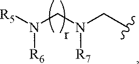

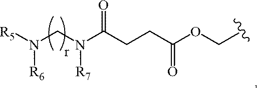

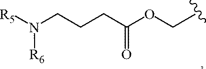

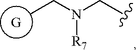

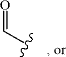

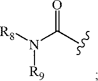

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of any one of compounds (I-a), (II), (II-a), and (II-b), n is 1 or 2.

In some embodiments of any one of compounds (I-a), (II), (II-a), and (II-b), m is 1.

In some embodiments of any one of compounds (I-a), (II), (II-a), and (II-b), p is 1.

In some embodiments of A1, the compound is represented by formula (III)

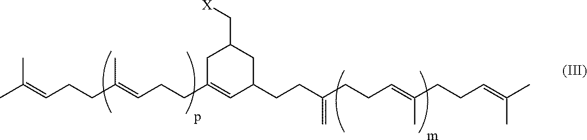

wherein X is optionally substituted aminoalkyl, optionally substituted alkoxy, optionally substituted acyloxy, hydroxyl, optionally substituted amino, mono-, di- or trisaccharyl, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (III-a)

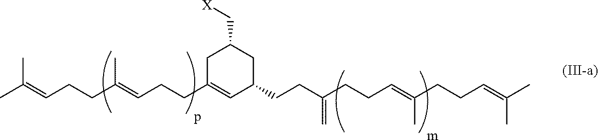

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (III-b)

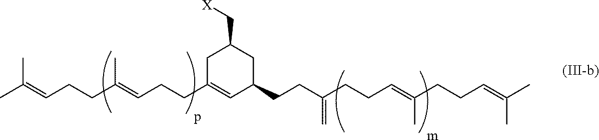

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (III-c)

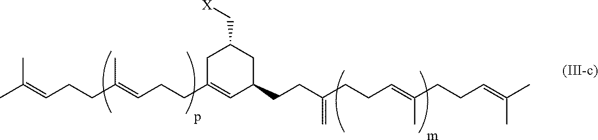

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (III-d)

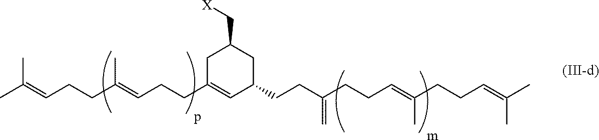

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of A1, the compound is represented by formula (IV)

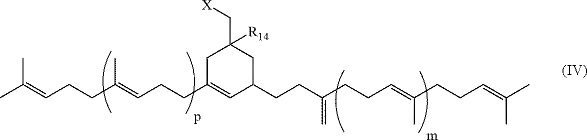

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IV-a)

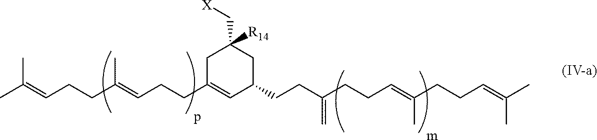

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IV-b)

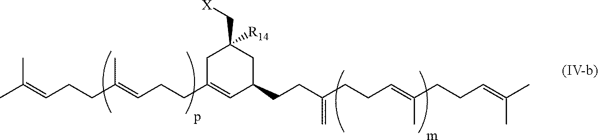

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IV-c)

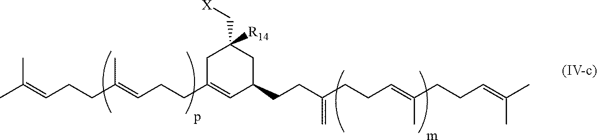

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IV-d)

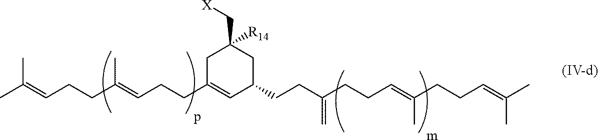

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of A1, the compound is represented by formula (V)

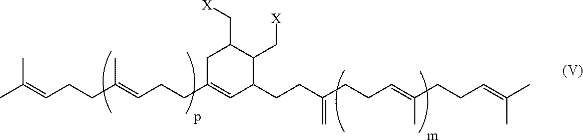

wherein each X is, independently, optionally substituted aminoalkyl, optionally substituted alkoxy, optionally substituted acyloxy, hydroxyl, optionally substituted amino, mono-, di- or trisaccharyl, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (V-a)

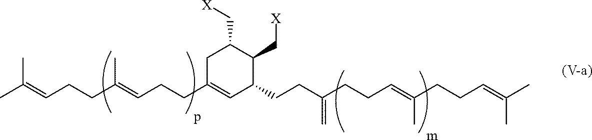

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (V-b)

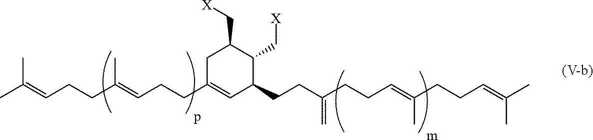

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (V-c)

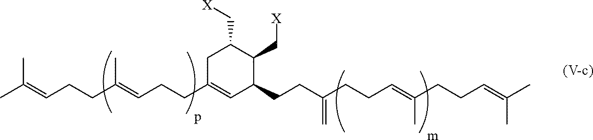

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (V-d)

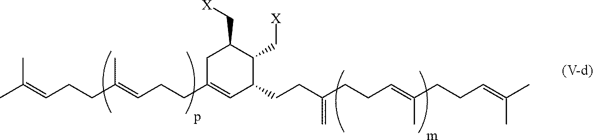

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (III), (III-a), (III-b), (III-c), (III-d), (IV), (IV-a), (IV-b), (IV-c), (IV-d), (V), (V-a), (V-b), (V-c), and (V-d),each X is, independently,

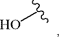

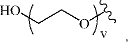

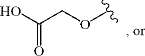

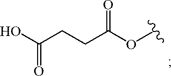

wherein v is an integer from 1 to 15 (e.g., 1, 2, 3, 4, 5, 6, 7, 8, 9, 10, 11, 12, 13, 14, or 15).

In some embodiments of any one of compounds (III), (III-a), (III-b), (III-c), (III-d), (IV), (IV-a), (IV-b), (IV-c), (IV-d), (V), (V-a), (V-b), (V-c), and (V-d), each X is, independently,

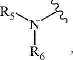

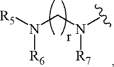

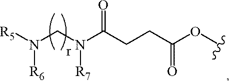

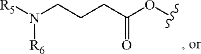

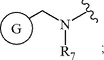

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments of any one of compounds (III), (III-a), (III-b), (III-c), (III-d), (IV), (IV-a), (IV-b), (IV-c), (IV-d), (V), (V-a), (V-b), (V-c), and (V-d), m is 1.

In some embodiments of any one of compounds (III), (III-a), (III-b), (III-c), (III-d), (IV), (IV-a), (IV-b), (IV-c), (IV-d), (V), (V-a), (V-b), (V-c), and (V-d), p is 1.

In some embodiments of A1, the compound is represented by formula (VI)

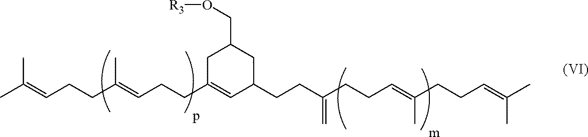

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VI-a)

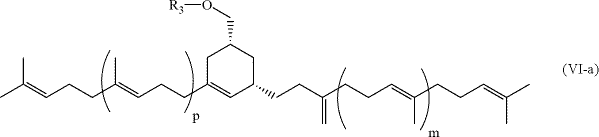

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VI-b)

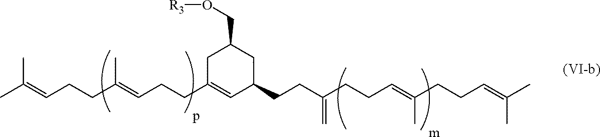

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VI-c)

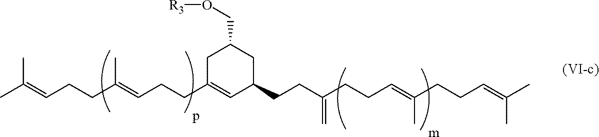

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VI-d)

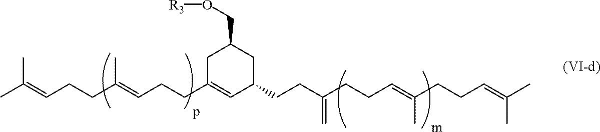

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of A1, the compound is represented by formula (VII)

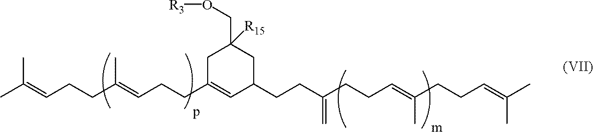

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VII-a)

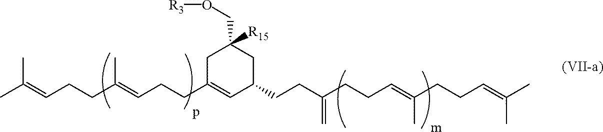

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VII-b)

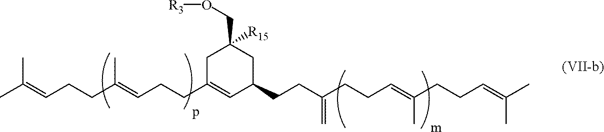

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VII-c)

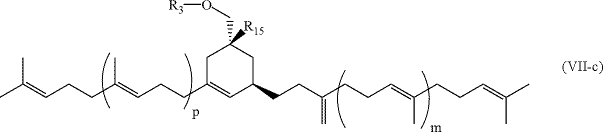

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VII-d)

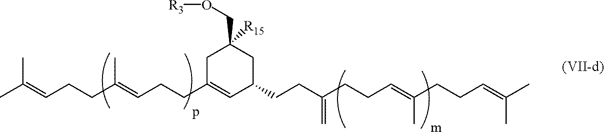

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of A1, the compound is represented by formula (VIII)

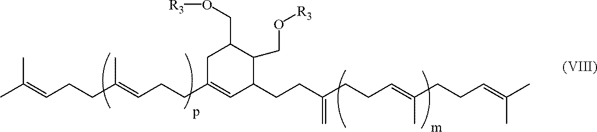

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VIII-a)

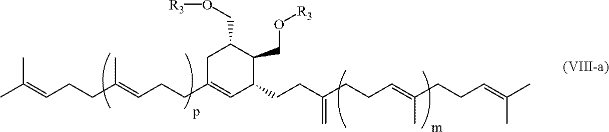

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VIII-b)

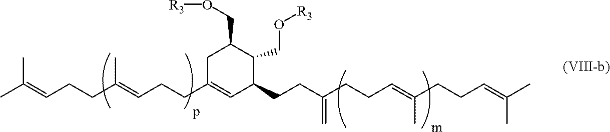

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VIII-c)

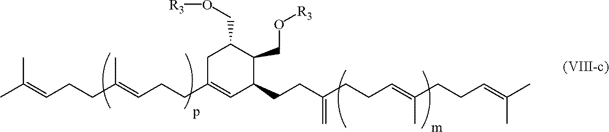

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (VIII-d)

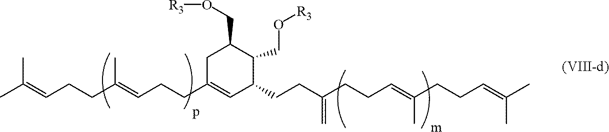

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (VI), (VI-a), (VI-b), (VI-c), (VI-d), (VII), (VII-a), (VII-b), (VII-c), (VIII-d), (VIII), (VIII-a), (VIII-b), (VIII-c), and (VIII-d), each R₃ is, independently, hydrogen,

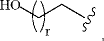

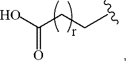

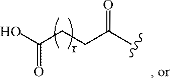

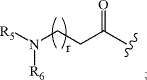

wherein each of R₅ and R₆ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 0 to 6 (e.g., 0, 1, 2, 3, 4, 5, or 6).

In some embodiments of any one of compounds (VI), (VI-a), (VI-b), (VI-c), (VI-d), (VII), (VII-a), (VII-b), (VII-c), (VIII-d), (VIII), (VIII-a), (VIII-b), (VIII-c), and (VIII-d), m is 1.

In some embodiments of any one of compounds (VI), (VI-a), (VI-b), (VI-c), (VI-d), (VII), (VII-a), (VII-b), (VII-c), (VIII-d), (VIII), (VIII-a), (VIII-b), (VIII-c), and (VIII-d), p is 1.

In some embodiments of A1, the compound is represented by formula (IX)

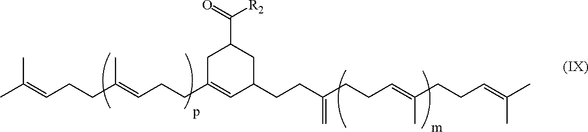

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IX-a)

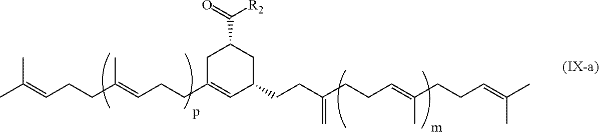

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IX-b)

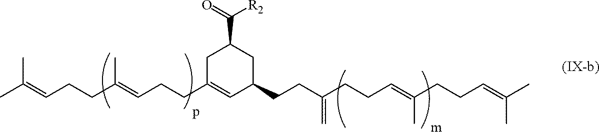

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IX-c)

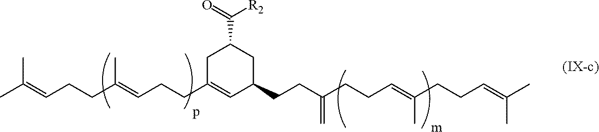

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (IX-d)

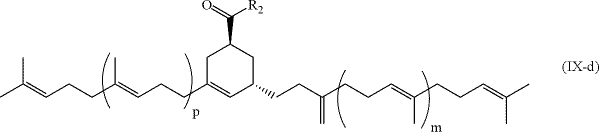

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of A1, the compound is represented by formula (X)

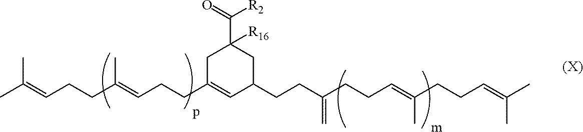

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (X-a)

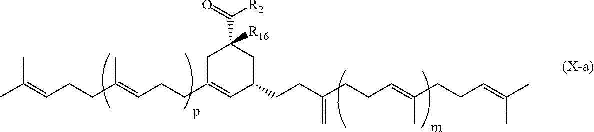

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (X-b)

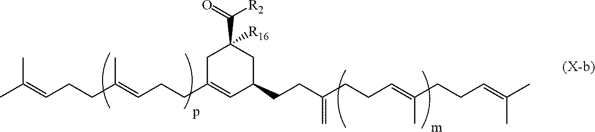

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (X-c)

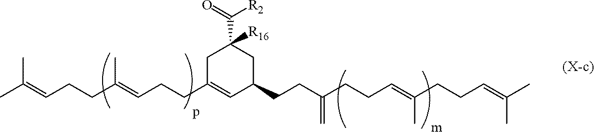

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (X-d)

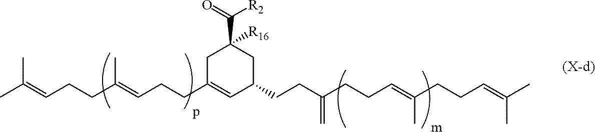

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of A1, the compound is represented by formula (XI)

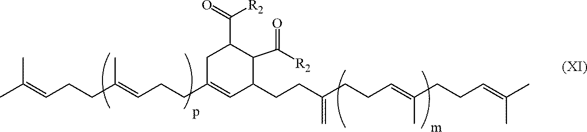

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (XI-a)

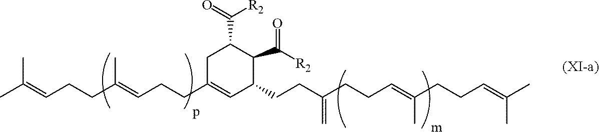

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (XI-b)

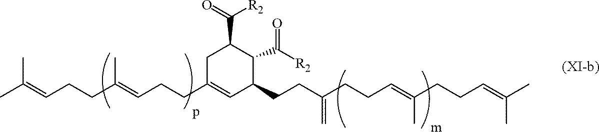

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (XI-c)

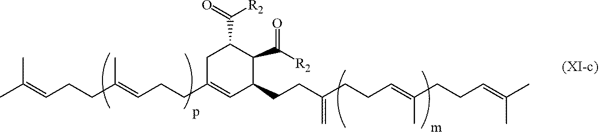

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, the compound is represented by formula (XI-d)

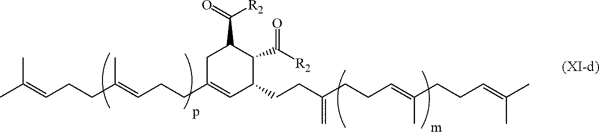

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d),each R₂ is, independently, hydrogen, hydroxyl,

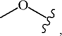

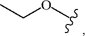

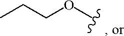

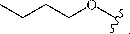

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d), each R₂ is, independently,

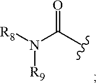

wherein each of R₈ and R₉ is, independently, hydrogen, optionally substituted alkyl, optionally substituted alkenyl, optionally substituted alkynyl, or optionally substituted aminoalkyl.

In some embodiments, each of R₈ and R₉ is, independently, alkylaminoalkyl or heterocyclyl alkyl.

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d), m is 1.

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d), p is 1.

In some embodiments of A1, the compound is represented by formula (XII)

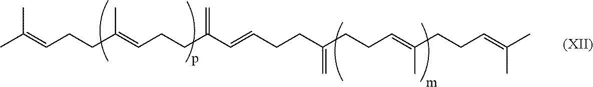

wherein m is an integer from 0 to 3 (e.g., 0, 1, 2, or 3); and
p is an integer from 0 to 3 (e.g., 0, 1, 2, or 3).

In some embodiments of compound (XII), m is 1.

In some embodiments of compound (XII), p is 1.

In some embodiments of A2, the compound is represented by formula (XIII)

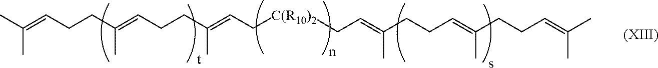

wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
n is an integer from 1 to 6 (e.g., 1, 2, 3, 4, 5, or 6);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XIII), each R₁₀ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of compound (XIII), each R₁₀ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of compound (XIII), each R₁₀ is, independently, hydrogen,

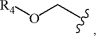

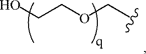

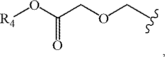

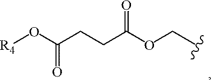

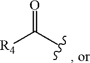

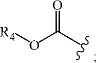

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of compound (XIII), each R₁₀ is, independently, hydrogen,

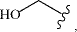

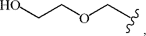

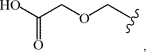

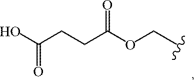

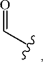

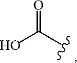

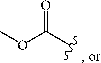

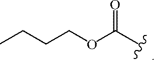

In some embodiments of compound (XIII), each R₁₀ is, independently, hydrogen,

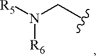

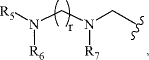

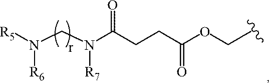

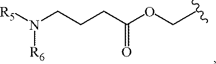

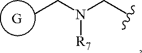

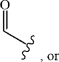

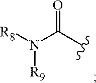

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of compound (XIII), n is 1 or 2.

In some embodiments of compound (XIII), s is 1.

In some embodiments of compound (XIII), t is 1.

In some embodiments of A2, the compound is represented by formula (XIV)

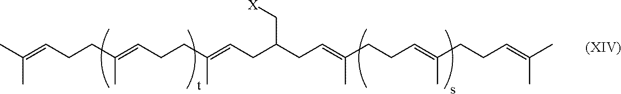

wherein X is optionally substituted alkoxy, optionally substituted acyloxy, hydroxyl, optionally substituted amino, optionally substituted sulfonyloxy, optionally substituted siloxy, carbonyl, or halogen;
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XIV), X is

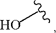

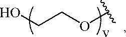

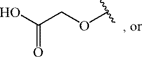

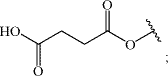

wherein v is an integer from 1 to 15 (e.g., 1, 2, 3, 4, 5, 6, 7, 8, 9, 10, 11, 12, 13, 14, or 15).

In some embodiments of compound (XIV), X is

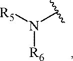

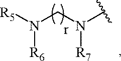

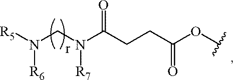

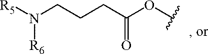

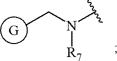

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments of compound (XIV), s is 1.

In some embodiments of compound (XIV), t is 1.

In some embodiments of A2, the compound is represented by formula (XV)

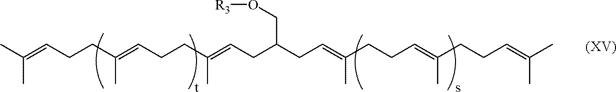

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XV), R₃ is hydrogen,

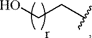

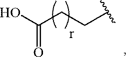

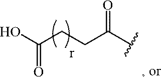

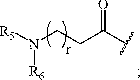

wherein each of R₅ and R₆ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 0 to 6 (e.g., 0, 1, 2, 3, 4, 5, or 6).

In some embodiments of compound (XV), s is 1.

In some embodiments of compound (XV), t is 1.

In some embodiments of A2, the compound is represented by formula (XVI)

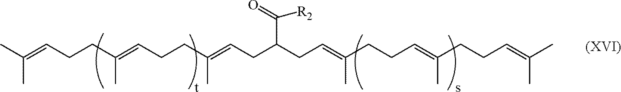

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of A2, the compound is represented by formula (XVII)

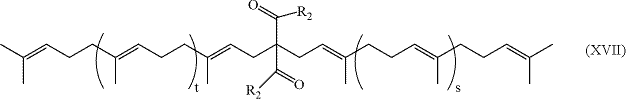

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XVI) or (XVII), each R₂ is, independently, hydrogen, hydroxyl,

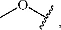

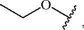

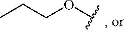

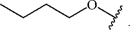

In some embodiments of compound (XVI) or (XVII), s is 1.

In some embodiments of compound (XVI) or (XVII), t is 1.

In some embodiments of A2, the compound is represented by formula (XVIII)

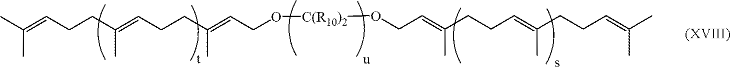

wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
u is an integer from 1 to 8 (e.g., 1, 2, 3, 4, 5, 6, 7, or 8);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XVIII), each R₁₀ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of compound (XVIII), each R₁₀ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of compound (XVIII), each R₁₀ is, independently, hydrogen,

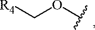

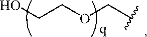

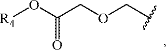

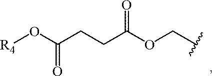

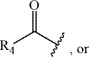

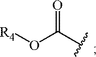

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of compound (XVIII), each R₁₀ is, independently, hydrogen,

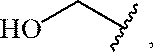

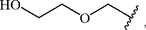

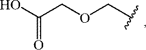

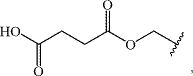

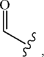

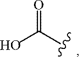

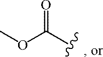

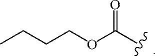

In some embodiments of compound (XVIII), each R₁₀ is, independently, hydrogen,

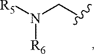

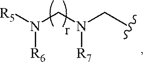

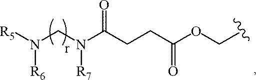

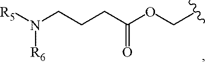

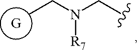

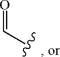

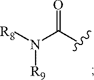

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of compound (XVIII), u is 1 or 2.

In some embodiments of compound (XVIII), s is 1.

In some embodiments of compound (XVIII), t is 1.

In some embodiments of A2, the compound is represented by formula (XIX)

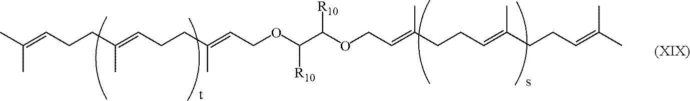

wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, or optionally substituted heteroalkynyl;
s is an integer from 0 to 3 (e.g., 0, 1, 2, or 3); and
t is an integer from 0 to 3 (e.g., 0, 1, 2, or 3).

In some embodiments of A2, the compound is represented by formula (XX)

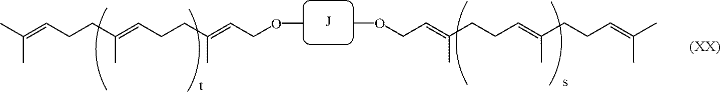

wherein J is optionally substituted alkylene, optionally substituted heteroalkylene, optionally substituted alkenylene, optionally substituted heteroalkenylene, optionally substituted alkynylene, or optionally substituted heteroalkynylene.

In some embodiments of compound (XX), J is optionally substituted alkenylene or optionally substituted heteroalkenylene.

In some embodiments of A2, the compound is represented by formula (XXI)

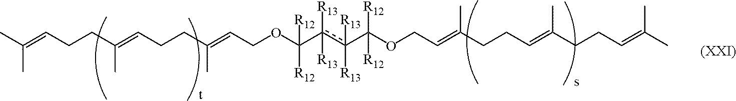

wherein each of R₁₂ and R₁₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
wherein each

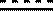

is, independently, a single bond or a double bond;
wherein if

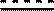

is a double bond, only one R₁₃ is present at each carbon of the double bond;
s is an integer from 0 to 3 (e.g., 0, 1, 2, or 3); and
t is an integer from 0 to 3 (e.g., 0, 1, 2, or 3).

In some embodiments of A2, the compound is represented by formula (XXII)

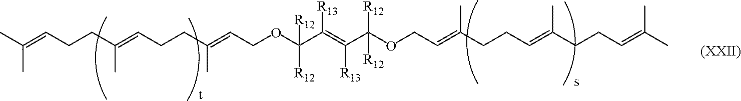

wherein each of R₁₂ and R₁₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
wherein s is an integer from 0 to 3 (e.g., 0, 1, 2, or 3); and
t is an integer from 0 to 3 (e.g., 0, 1, 2, or 3).

In some embodiments of A2, the compound is represented by formula (XXIII)

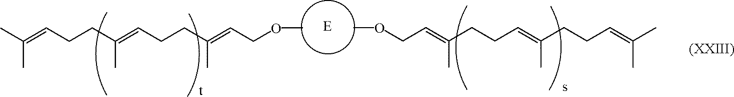

wherein E is optionally substituted cycloalkyl, optionally substituted heterocyclyl, optionally substituted aryl, or optionally substituted heteroaryl.

In some embodiments of compound (XXIII), E is a ring selected from

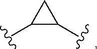

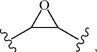

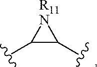

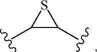

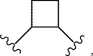

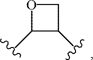

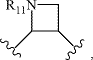

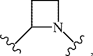

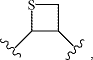

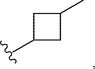

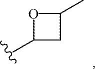

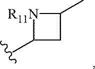

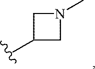

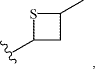

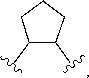

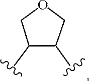

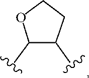

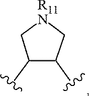

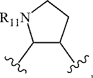

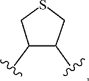

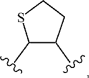

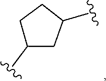

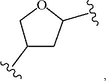

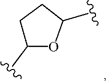

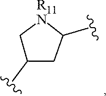

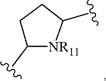

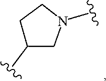

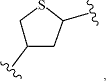

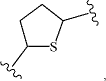

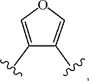

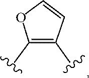

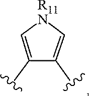

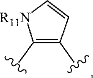

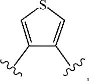

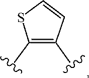

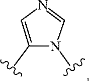

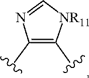

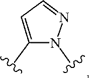

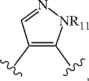

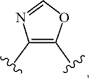

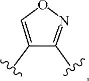

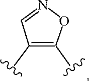

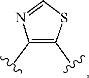

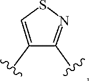

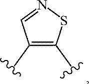

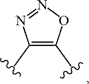

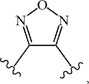

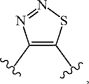

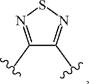

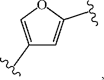

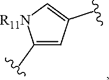

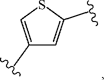

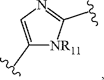

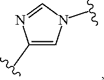

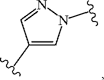

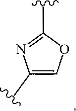

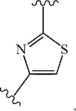

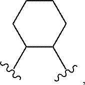

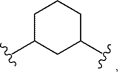

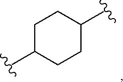

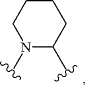

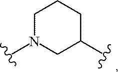

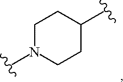

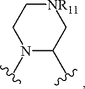

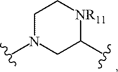

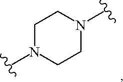

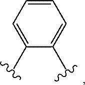

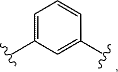

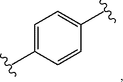

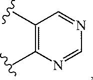

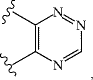

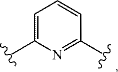

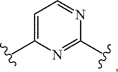

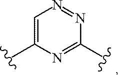

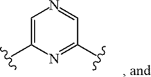

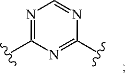

wherein each R₁₁ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, or carbonyl;
and wherein each ring of E may be optionally substituted, as the valency of each ring permits.

In some embodiments of compound (XXIII), E is

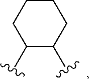

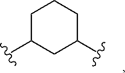

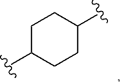

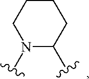

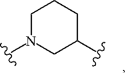

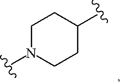

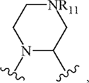

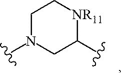

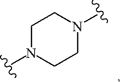

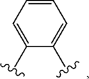

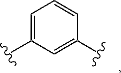

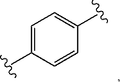

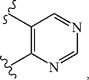

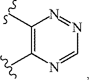

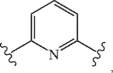

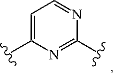

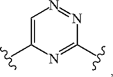

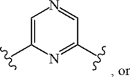

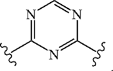

In some embodiments of compound (XXIII), s is 1.

In some embodiments of compound (XXIII), t is 1.

In some embodiments of A2, the compound is represented by formula (XXIV)

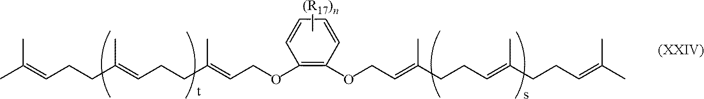

wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4 (e.g., 0, 1, 2, 3, or 4);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of A2, the compound is represented by formula (XXV)

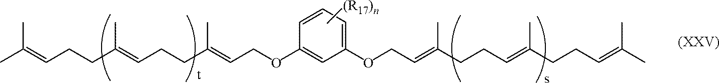

wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4 (e.g., 0, 1, 2, 3, or 4);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of A2, the compound is represented by formula (XXVI)

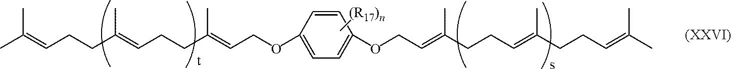

wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4 (e.g., 0, 1, 2, 3, or 4);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of any one of compounds (XXIV), (XXV), or (XXVI), each R₁₇ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of any one of compounds (XXIV), (XXV), or (XXVI), each R₁₇ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of any one of compounds (XXIV), (XXV), or (XXVI), each R₁₇ is, independently, hydrogen,

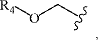

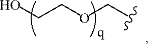

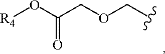

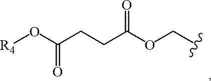

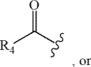

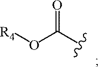

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of any one of compounds (XXIV), (XXV), or (XXVI), each R₁₇ is, independently, hydrogen,

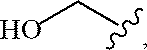

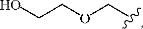

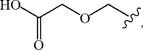

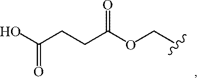

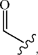

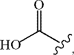

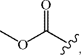

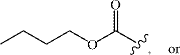

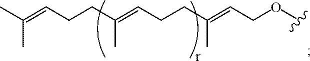

and wherein each r is an integer from 0 to 3 (e.g., 0, 1, 2, or 3).

In some embodiments of any one of compounds (XXIV), (XXV), or (XXVI), each R₁₇ is, independently, hydrogen,

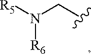

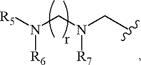

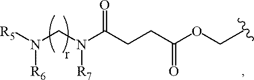

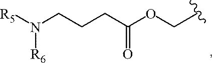

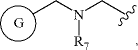

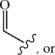

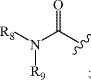

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of any one of compounds (XXIV), (XXV), or (XXVI), n is 1 or 2.

In some embodiments of any one of compounds (XXIV), (XXV), or (XXVI), s is 1.

In some embodiments of any one of compounds (XXIV), (XXV), or (XXVI), t is 1.

In some embodiments, the disclosure features a compound represented by formula (1)

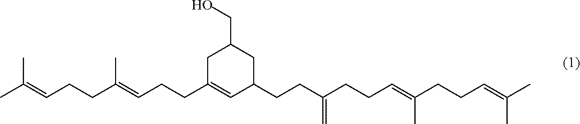 [SMILES: C=C(CC/C=C(\C)CCC=C(C)C)CCC1C=C(CC/C=C(\C)CCC=C(C)C)CC(CO)C1]

In some embodiments, the disclosure features a compound represented by formula (2)

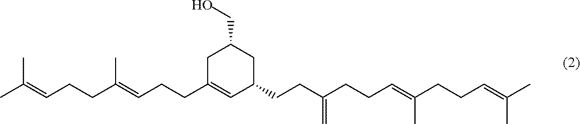 [SMILES: C=C(CC/C=C(\C)CCC=C(C)C)CC[C@@H]1C=C(CC/C=C(\C)CCC=C(C)C)C[C@H](CO)C1]

In some embodiments, the disclosure features a compound represented by formula (3)

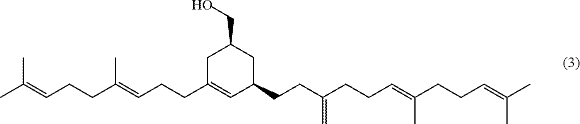 [SMILES: C=C(CC/C=C(\C)CCC=C(C)C)CC[C@H]1C=C(CC/C=C(\C)CCC=C(C)C)C[C@@H](CO)C1]

In some embodiments, the disclosure features a compound represented by formula (4)

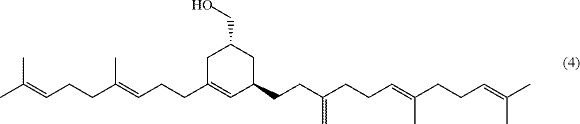 [SMILES: C=C(CC/C=C(\C)CCC=C(C)C)CC[C@H]1C=C(CC/C=C(\C)CCC=C(C)C)C[C@H](CO)C1]

In some embodiments, the disclosure features a compound represented by formula (5)

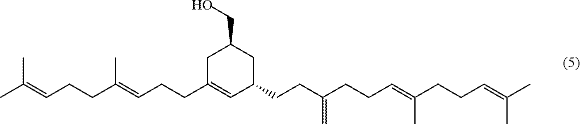 [SMILES: C=C(CC/C=C(\C)CCC=C(C)C)CC[C@@H]1C=C(CC/C=C(\C)CCC=C(C)C)C[C@@H](CO)C1]

In some embodiments, the disclosure features a compound represented by formula (6)

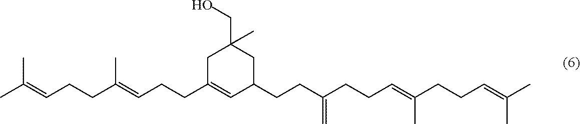 [SMILES: C=C(CC/C=C(\C)CCC=C(C)C)CCC1C=C(CC/C=C(\C)CCC=C(C)C)CC(C)(CO)C1]

In some embodiments, the disclosure features a compound represented by formula (7)

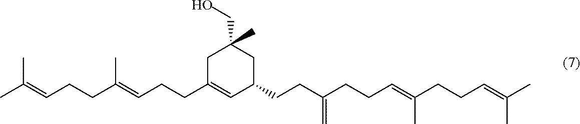 [SMILES: C=C(CC/C=C(\C)CCC=C(C)C)CC[C@@H]1C=C(CC/C=C(\C)CCC=C(C)C)C[C@](C)(CO)C1]

In some embodiments, the disclosure features a compound represented by formula (8)

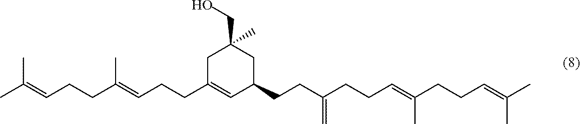 [SMILES: C=C(CC/C=C(\C)CCC=C(C)C)CC[C@H]1C=C(CC/C=C(\C)CCC=C(C)C)C[C@@](C)(CO)C1]

In some embodiments, the disclosure features a compound represented by formula (9)

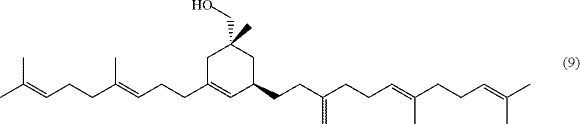 [SMILES: C=C(CC/C=C(\C)CCC=C(C)C)CC[C@H]1C=C(CC/C=C(\C)CCC=C(C)C)C[C@](C)(CO)C1]

In some embodiments, the disclosure features a compound represented by formula (10)

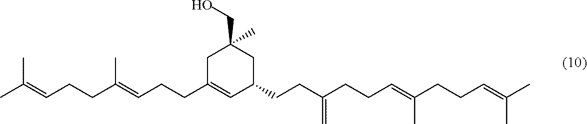 [SMILES: C=C(CC/C=C(\C)CCC=C(C)C)CC[C@@H]1C=C(CC/C=C(\C)CCC=C(C)C)C[C@@](C)(CO)C1]

In some embodiments, the disclosure features a compound represented by formula (11)

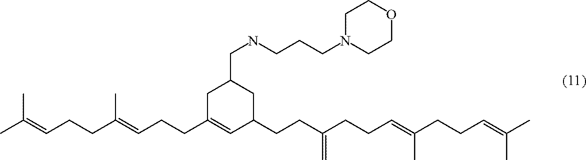 [SMILES: C=C(CC/C=C(\C)CCC=C(C)C)CCC1C=C(CC/C=C(\C)CCC=C(C)C)CC(CNCCCN2CCOCC2)C1]

In some embodiments, the disclosure features a compound represented by formula (12)

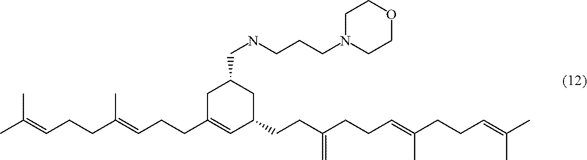 [SMILES: C=C(CC/C=C(\C)CCC=C(C)C)CC[C@@H]1C=C(CC/C=C(\C)CCC=C(C)C)C[C@H](CNCCCN2CCOCC2)C1]

In some embodiments, the disclosure features a compound represented by formula (13)

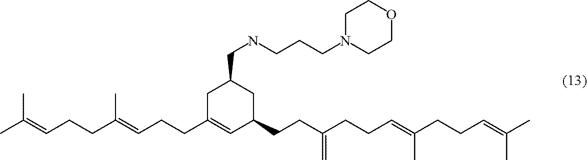 [SMILES: C=C(CC/C=C(\C)CCC=C(C)C)CC[C@H]1C=C(CC/C=C(\C)CCC=C(C)C)C[C@@H](CNCCCN2CCOCC2)C1]

In some embodiments, the disclosure features a compound represented by formula (14)

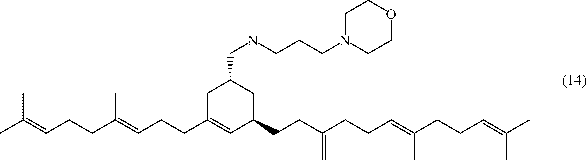 [SMILES: C=C(CC/C=C(\C)CCC=C(C)C)CC[C@H]1C=C(CC/C=C(\C)CCC=C(C)C)C[C@H](CNCCCN2CCOCC2)C1]

In some embodiments, the disclosure features a compound is represented by formula (15)

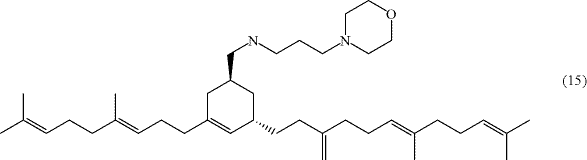 [SMILES: C=C(CC/C=C(\C)CCC=C(C)C)CC[C@@H]1C=C(CC/C=C(\C)CCC=C(C)C)C[C@@H](CNCCCN2CCOCC2)C1]

In some embodiments, the disclosure features a compound represented by formula (16)

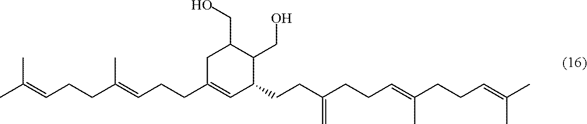 [SMILES: C=C(CC/C=C(\C)CCC=C(C)C)CC[C@@H]1C=C(CC/C=C(\C)CCC=C(C)C)CC(CO)C1CO]

In some embodiments, the disclosure features a compound represented by formula (17)

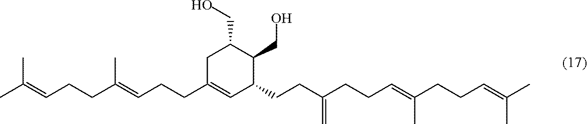 [SMILES: C=C(CC/C=C(\C)CCC=C(C)C)CC[C@@H]1C=C(CC/C=C(\C)CCC=C(C)C)C[C@H](CO)[C@H]1CO]

In some embodiments, the disclosure features a compound represented by formula (18)

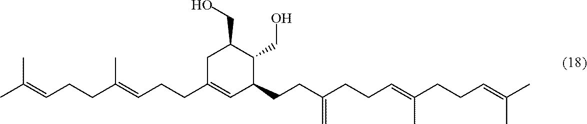 [SMILES: C=C(CC/C=C(\C)CCC=C(C)C)CC[C@H]1C=C(CC/C=C(\C)CCC=C(C)C)C[C@@H](CO)[C@@H]1CO]

In some embodiments, the disclosure features a compound represented by formula (19)

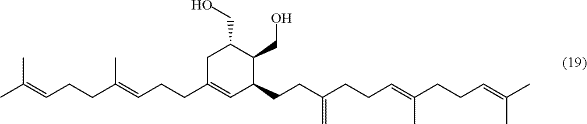 [SMILES: C=C(CC/C=C(\C)CCC=C(C)C)CC[C@H]1C=C(CC/C=C(\C)CCC=C(C)C)C[C@H](CO)[C@H]1CO]

In some embodiments, the disclosure features a compound represented by formula (20)

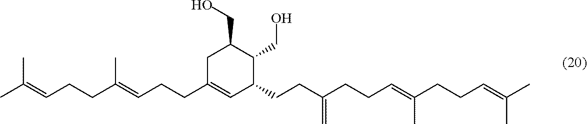 [SMILES: C=C(CC/C=C(\C)CCC=C(C)C)CC[C@@H]1C=C(CC/C=C(\C)CCC=C(C)C)C[C@@H](CO)[C@@H]1CO]

In some embodiments, the disclosure features a compound represented by formula (21)

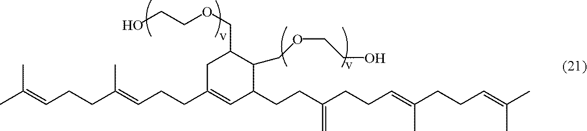

wherein each v is an integer from 1 to 15 (e.g., 0, 1, 2, 3, 4, 5, 6, 7, 8, 9, 10, 11, 12, 13, 14, or 15).

In some embodiments, the disclosure features a compound represented by formula (22)

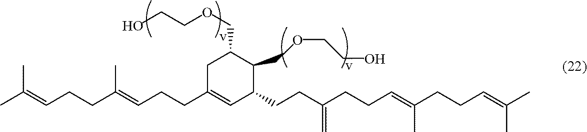

wherein each v is an integer from 1 to 15 (e.g., 0, 1, 2, 3, 4, 5, 6, 7, 8, 9, 10, 11, 12, 13, 14, or 15).

In some embodiments, the disclosure features a compound represented by formula (23)

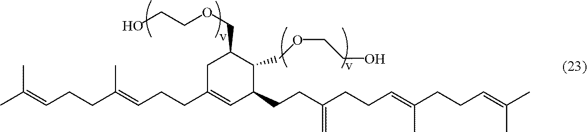

wherein each v is an integer from 1 to 15 (e.g., 0, 1, 2, 3, 4, 5, 6, 7, 8, 9, 10, 11, 12, 13, 14, or 15).

In some embodiments, the disclosure features a compound represented by formula (24)

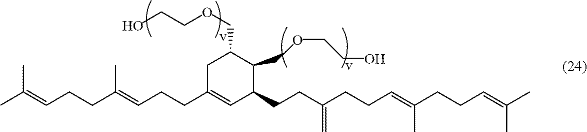

wherein each v is an integer from 1 to 15 (e.g., 0, 1, 2, 3, 4, 5, 6, 7, 8, 9, 10, 11, 12, 13, 14, or 15).

In some embodiments, the disclosure features a compound represented by formula (25)

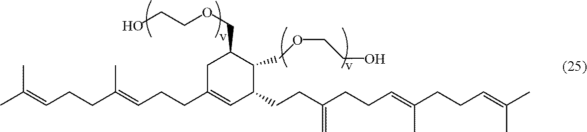

wherein each v is an integer from 1 to 15 (e.g., 0, 1, 2, 3, 4, 5, 6, 7, 8, 9, 10, 11, 12, 13, 14, or 15).

In some embodiments, the disclosure features a compound represented by formula (26)

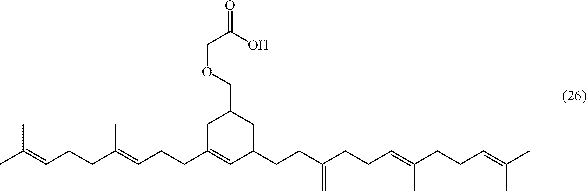 [SMILES: C=C(CC/C=C(\C)CCC=C(C)C)CCC1C=C(CC/C=C(\C)CCC=C(C)C)CC(COCC(=O)O)C1]

or a pharmaceutically acceptable salt thereof.

In some embodiments, the disclosure features a compound represented by formula (27)

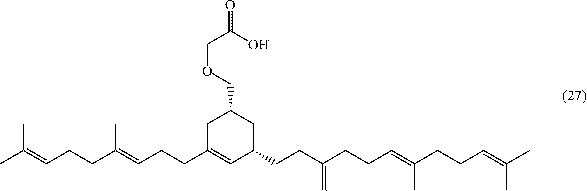 [SMILES: C=C(CC/C=C(\C)CCC=C(C)C)CC[C@@H]1C=C(CC/C=C(\C)CCC=C(C)C)C[C@H](COCC(=O)O)C1]

or a pharmaceutically acceptable salt thereof.

In some embodiments, the disclosure features a compound represented by formula (28)

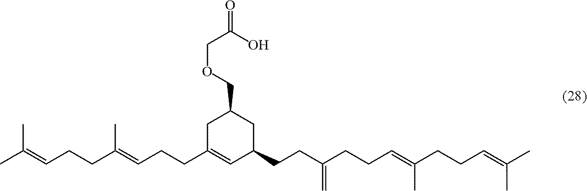 [SMILES: C=C(CC/C=C(\C)CCC=C(C)C)CC[C@H]1C=C(CC/C=C(\C)CCC=C(C)C)C[C@@H](COCC(=O)O)C1]

or a pharmaceutically acceptable salt thereof.

In some embodiments, the disclosure features a compound represented by formula (29)

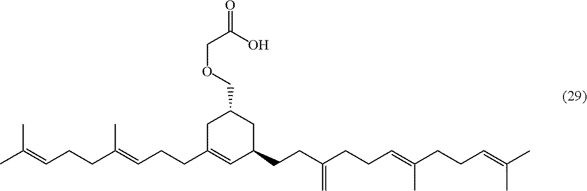 [SMILES: C=C(CC/C=C(\C)CCC=C(C)C)CC[C@H]1C=C(CC/C=C(\C)CCC=C(C)C)C[C@H](COCC(=O)O)C1]

or a pharmaceutically acceptable salt thereof.

In some embodiments, the disclosure features a compound represented by formula (30)

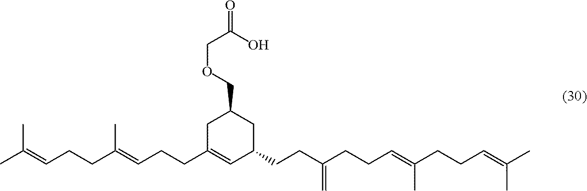 [SMILES: C=C(CC/C=C(\C)CCC=C(C)C)CC[C@@H]1C=C(CC/C=C(\C)CCC=C(C)C)C[C@@H](COCC(=O)O)C1]

or a pharmaceutically acceptable salt thereof.

In some embodiments, the disclosure features a compound represented by formula (31)

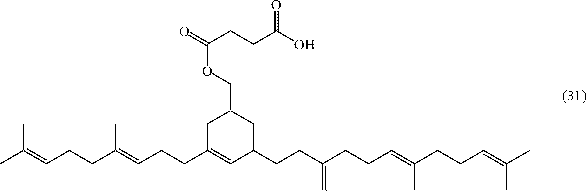 [SMILES: C=C(CC/C=C(\C)CCC=C(C)C)CCC1C=C(CC/C=C(\C)CCC=C(C)C)CC(COC(=O)CCC(=O)O)C1]

or a pharmaceutically acceptable salt thereof.

In some embodiments, the disclosure features a compound represented by formula (32)

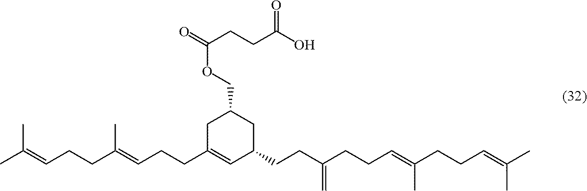 [SMILES: C=C(CC/C=C(\C)CCC=C(C)C)CC[C@@H]1C=C(CC/C=C(\C)CCC=C(C)C)C[C@H](COC(=O)CCC(=O)O)C1]

or a pharmaceutically acceptable salt thereof.

In some embodiments, the disclosure features a compound represented by formula (33)

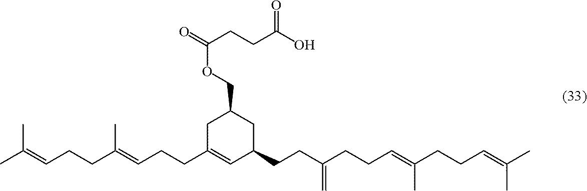 [SMILES: C=C(CC/C=C(\C)CCC=C(C)C)CC[C@H]1C=C(CC/C=C(\C)CCC=C(C)C)C[C@@H](COC(=O)CCC(=O)O)C1]

or a pharmaceutically acceptable salt thereof.

In some embodiments, the disclosure features a compound represented by formula (34)

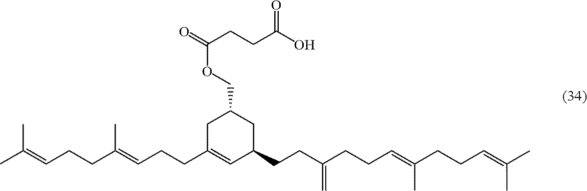 [SMILES: C=C(CC/C=C(\C)CCC=C(C)C)CC[C@H]1C=C(CC/C=C(\C)CCC=C(C)C)C[C@H](COC(=O)CCC(=O)O)C1]

or a pharmaceutically acceptable salt thereof.

In some embodiments, the disclosure features a compound represented by formula (35)

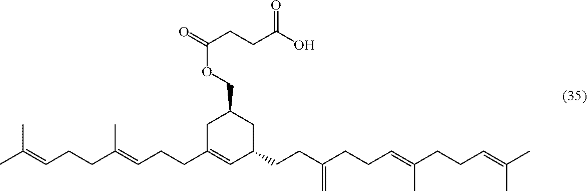 [SMILES: C=C(CC/C=C(\C)CCC=C(C)C)CC[C@@H]1C=C(CC/C=C(\C)CCC=C(C)C)C[C@@H](COC(=O)CCC(=O)O)C1]

or a pharmaceutically acceptable salt thereof.

In some embodiments, the disclosure features a compound represented by formula (36)

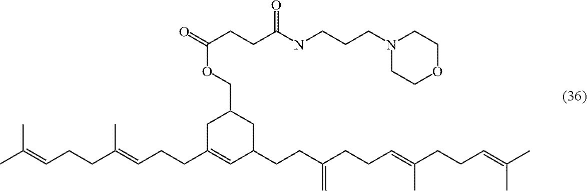 [SMILES: C=C(CC/C=C(\C)CCC=C(C)C)CCC1C=C(CC/C=C(\C)CCC=C(C)C)CC(COC(=O)CCC(=O)NCCCN2CCOCC2)C1]

In some embodiments, the disclosure features a compound represented by formula (37)

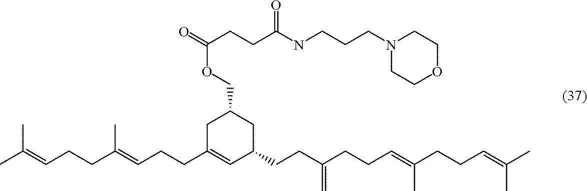 [SMILES: C=C(CC/C=C(\C)CCC=C(C)C)CC[C@@H]1C=C(CC/C=C(\C)CCC=C(C)C)C[C@H](COC(=O)CCC(=O)NCCCN2CCOCC2)C1]

In some embodiments, the disclosure features a compound represented by formula (38)

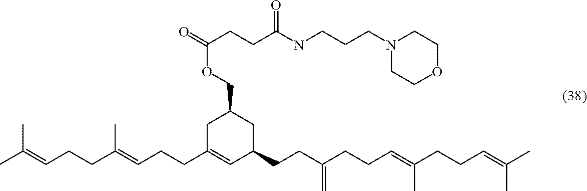 [SMILES: C=C(CC/C=C(\C)CCC=C(C)C)CC[C@H]1C=C(CC/C=C(\C)CCC=C(C)C)C[C@@H](COC(=O)CCC(=O)NCCCN2CCOCC2)C1]

In some embodiments, the disclosure features a compound represented by formula (39)

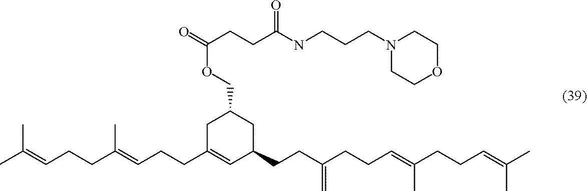 [SMILES: C=C(CC/C=C(\C)CCC=C(C)C)CC[C@H]1C=C(CC/C=C(\C)CCC=C(C)C)C[C@H](COC(=O)CCC(=O)NCCCN2CCOCC2)C1]

In some embodiments, the disclosure features a compound represented by formula (40)

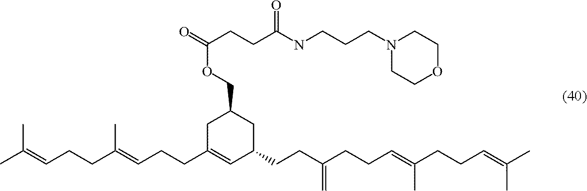 [SMILES: C=C(CC/C=C(\C)CCC=C(C)C)CC[C@@H]1C=C(CC/C=C(\C)CCC=C(C)C)C[C@@H](COC(=O)CCC(=O)NCCCN2CCOCC2)C1]

In some embodiments, the disclosure features a compound represented by formula (41)

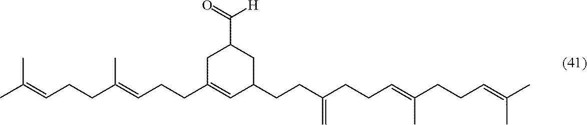 [SMILES: C=C(CC/C=C(\C)CCC=C(C)C)CCC1C=C(CC/C=C(\C)CCC=C(C)C)CC(C=O)C1]

In some embodiments, the disclosure features a compound represented by formula (42)

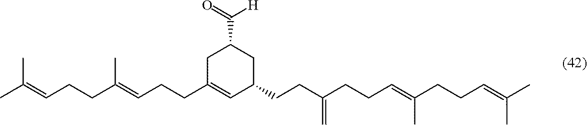 [SMILES: C=C(CC/C=C(\C)CCC=C(C)C)CC[C@@H]1C=C(CC/C=C(\C)CCC=C(C)C)C[C@H](C=O)C1]

In some embodiments, the disclosure features a compound represented by formula (43)

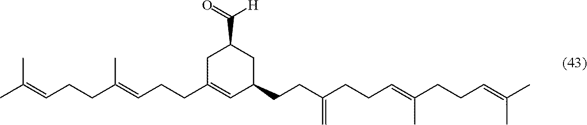 [SMILES: C=C(CC/C=C(\C)CCC=C(C)C)CC[C@H]1C=C(CC/C=C(\C)CCC=C(C)C)C[C@@H](C=O)C1]

In some embodiments, the disclosure features a compound represented by formula (44)

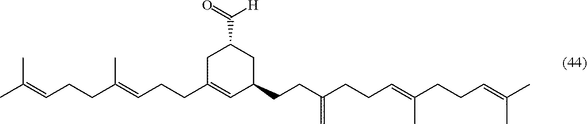 [SMILES: C=C(CC/C=C(\C)CCC=C(C)C)CC[C@H]1C=C(CC/C=C(\C)CCC=C(C)C)C[C@H](C=O)C1]

In some embodiments, the disclosure features a compound represented by formula (45)

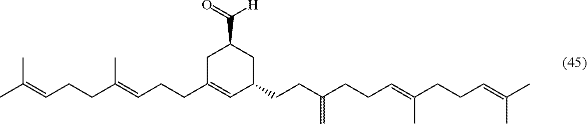 [SMILES: C=C(CC/C=C(\C)CCC=C(C)C)CC[C@@H]1C=C(CC/C=C(\C)CCC=C(C)C)C[C@@H](C=O)C1]

In some embodiments, the disclosure features a compound represented by formula (46)

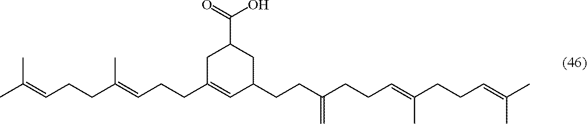 [SMILES: C=C(CC/C=C(\C)CCC=C(C)C)CCC1C=C(CC/C=C(\C)CCC=C(C)C)CC(C(=O)O)C1]

or a pharmaceutically acceptable salt thereof.

In some embodiments, the disclosure features a compound represented by formula (47)

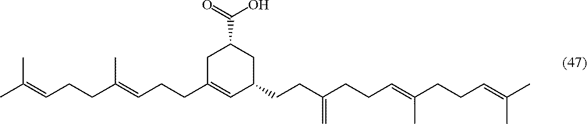 [SMILES: C=C(CC/C=C(\C)CCC=C(C)C)CC[C@@H]1C=C(CC/C=C(\C)CCC=C(C)C)C[C@H](C(=O)O)C1]

or a pharmaceutically acceptable salt thereof.

In some embodiments, the disclosure features a compound represented by formula (48)

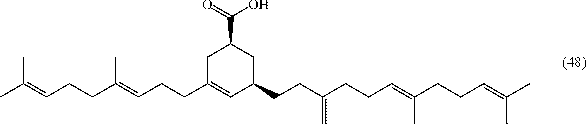 [SMILES: C=C(CC/C=C(\C)CCC=C(C)C)CC[C@H]1C=C(CC/C=C(\C)CCC=C(C)C)C[C@@H](C(=O)O)C1]

or a pharmaceutically acceptable salt thereof.

In some embodiments, the disclosure features a compound represented by formula (49)

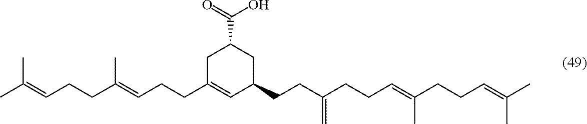 [SMILES: C=C(CC/C=C(\C)CCC=C(C)C)CC[C@H]1C=C(CC/C=C(\C)CCC=C(C)C)C[C@H](C(=O)O)C1]

or a pharmaceutically acceptable salt thereof.

In some embodiments, the disclosure features a compound represented by formula (50)

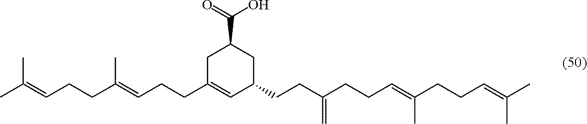 [SMILES: C=C(CC/C=C(\C)CCC=C(C)C)CC[C@@H]1C=C(CC/C=C(\C)CCC=C(C)C)C[C@@H](C(=O)O)C1]

or a pharmaceutically acceptable salt thereof.

In some embodiments, the disclosure features a compound represented by formula (51)

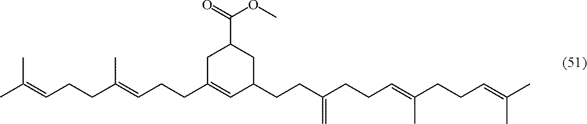 [SMILES: C=C(CC/C=C(\C)CCC=C(C)C)CCC1C=C(CC/C=C(\C)CCC=C(C)C)CC(C(=O)OC)C1]

In some embodiments, the disclosure features a compound represented by formula (52)

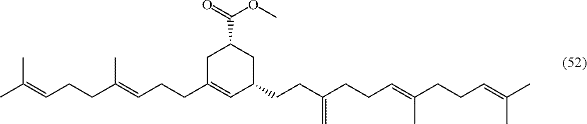 [SMILES: C=C(CC/C=C(\C)CCC=C(C)C)CC[C@@H]1C=C(CC/C=C(\C)CCC=C(C)C)C[C@H](C(=O)OC)C1]

In some embodiments, the disclosure features a compound represented by formula (53)

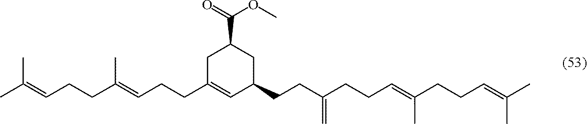 [SMILES: C=C(CC/C=C(\C)CCC=C(C)C)CC[C@H]1C=C(CC/C=C(\C)CCC=C(C)C)C[C@@H](C(=O)OC)C1]

In some embodiments, the disclosure features a compound represented by formula (54)

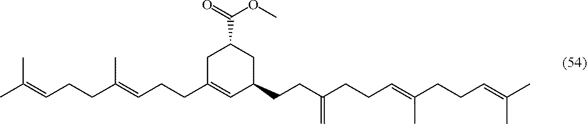 [SMILES: C=C(CC/C=C(\C)CCC=C(C)C)CC[C@H]1C=C(CC/C=C(\C)CCC=C(C)C)C[C@H](C(=O)OC)C1]

In some embodiments, the disclosure features a compound represented by formula (55)

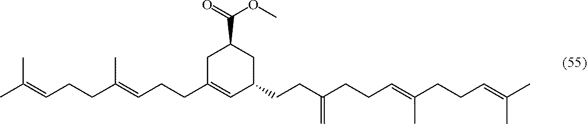 [SMILES: C=C(CC/C=C(\C)CCC=C(C)C)CC[C@@H]1C=C(CC/C=C(\C)CCC=C(C)C)C[C@@H](C(=O)OC)C1]

In some embodiments, the disclosure features a compound represented by formula (56)

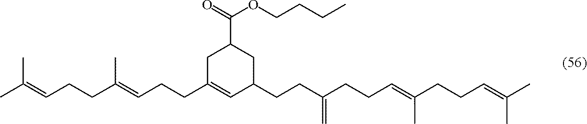 [SMILES: C=C(CC/C=C(\C)CCC=C(C)C)CCC1C=C(CC/C=C(\C)CCC=C(C)C)CC(C(=O)OCCCC)C1]

In some embodiments, the disclosure features a compound represented by formula (57)

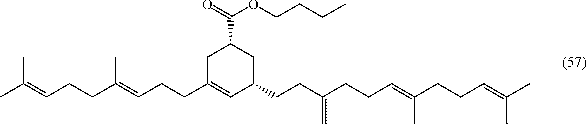 [SMILES: C=C(CC/C=C(\C)CCC=C(C)C)CC[C@@H]1C=C(CC/C=C(\C)CCC=C(C)C)C[C@H](C(=O)OCCCC)C1]

In some embodiments, the disclosure features a compound represented by formula (58)

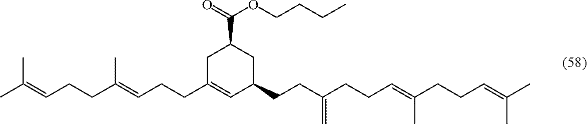 [SMILES: C=C(CC/C=C(\C)CCC=C(C)C)CC[C@H]1C=C(CC/C=C(\C)CCC=C(C)C)C[C@@H](C(=O)OCCCC)C1]

In some embodiments, the disclosure features a compound represented by formula (59)

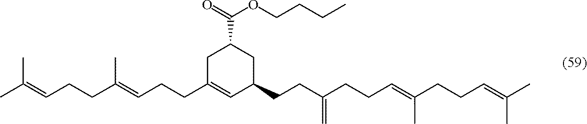 [SMILES: C=C(CC/C=C(\C)CCC=C(C)C)CC[C@H]1C=C(CC/C=C(\C)CCC=C(C)C)C[C@H](C(=O)OCCCC)C1]

In some embodiments, the disclosure features a compound represented by formula (60)

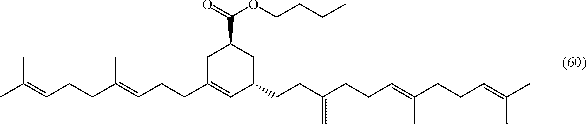 [SMILES: C=C(CC/C=C(\C)CCC=C(C)C)CC[C@@H]1C=C(CC/C=C(\C)CCC=C(C)C)C[C@@H](C(=O)OCCCC)C1]

In some embodiments, the disclosure features a compound represented by formula (61)

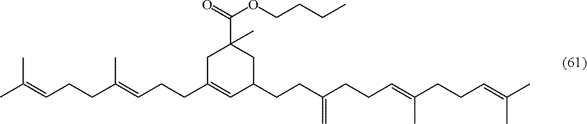 [SMILES: C=C(CC/C=C(\C)CCC=C(C)C)CCC1C=C(CC/C=C(\C)CCC=C(C)C)CC(C)(C(=O)OCCCC)C1]

In some embodiments, the disclosure features a compound represented by formula (62)

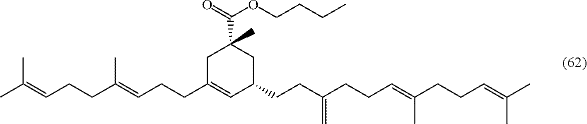 [SMILES: C=C(CC/C=C(\C)CCC=C(C)C)CC[C@@H]1C=C(CC/C=C(\C)CCC=C(C)C)C[C@](C)(C(=O)OCCCC)C1]

In some embodiments, the disclosure features a compound represented by formula (63)

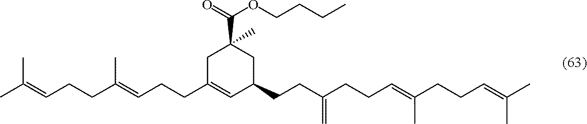 [SMILES: C=C(CC/C=C(\C)CCC=C(C)C)CC[C@H]1C=C(CC/C=C(\C)CCC=C(C)C)C[C@@](C)(C(=O)OCCCC)C1]

In some embodiments, the disclosure features a compound represented by formula (64)

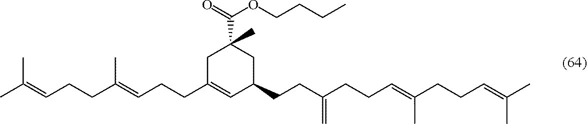 [SMILES: C=C(CC/C=C(\C)CCC=C(C)C)CC[C@H]1C=C(CC/C=C(\C)CCC=C(C)C)C[C@](C)(C(=O)OCCCC)C1]

In some embodiments, the disclosure features a compound represented by formula (65)

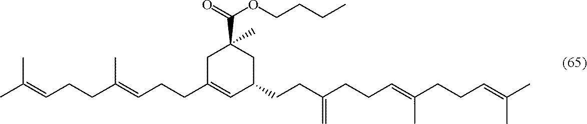 [SMILES: C=C(CC/C=C(\C)CCC=C(C)C)CC[C@@H]1C=C(CC/C=C(\C)CCC=C(C)C)C[C@@](C)(C(=O)OCCCC)C1]

In some embodiments, the disclosure features a compound represented by formula (66)

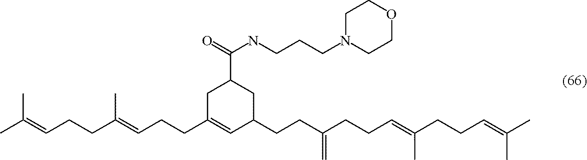 [SMILES: C=C(CC/C=C(\C)CCC=C(C)C)CCC1C=C(CC/C=C(\C)CCC=C(C)C)CC(C(=O)NCCCN2CCOCC2)C1]

In some embodiments, the disclosure features a compound represented by formula (67)

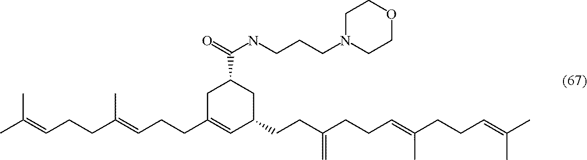 [SMILES: C=C(CC/C=C(\C)CCC=C(C)C)CC[C@@H]1C=C(CC/C=C(\C)CCC=C(C)C)C[C@H](C(=O)NCCCN2CCOCC2)C1]

In some embodiments, the disclosure features a compound represented by formula (68)

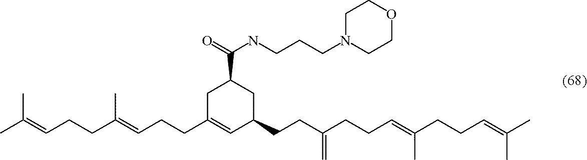 [SMILES: C=C(CC/C=C(\C)CCC=C(C)C)CC[C@H]1C=C(CC/C=C(\C)CCC=C(C)C)C[C@@H](C(=O)NCCCN2CCOCC2)C1]

In some embodiments, the disclosure features a compound represented by formula (69)

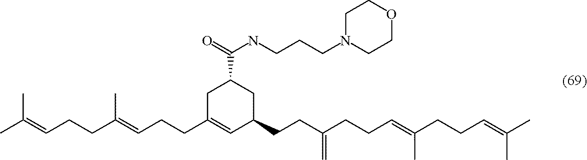 [SMILES: C=C(CC/C=C(\C)CCC=C(C)C)CC[C@H]1C=C(CC/C=C(\C)CCC=C(C)C)C[C@H](C(=O)NCCCN2CCOCC2)C1]

In some embodiments, the disclosure features a compound represented by formula (70)

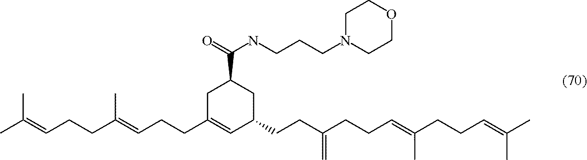 [SMILES: C=C(CC/C=C(\C)CCC=C(C)C)CC[C@@H]1C=C(CC/C=C(\C)CCC=C(C)C)C[C@@H](C(=O)NCCCN2CCOCC2)C1]

In some embodiments, the disclosure features a compound represented by formula (71)

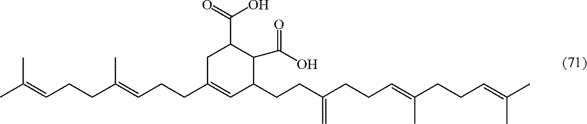 [SMILES: C=C(CC/C=C(\C)CCC=C(C)C)CCC1C=C(CC/C=C(\C)CCC=C(C)C)CC(C(=O)O)C1C(=O)O]

or a pharmaceutically acceptable salt thereof.

In some embodiments, the disclosure features a compound represented by formula (72)

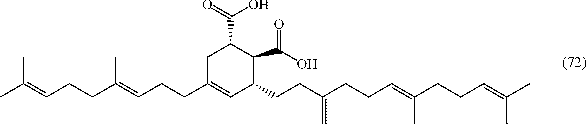 [SMILES: C=C(CC/C=C(\C)CCC=C(C)C)CC[C@@H]1C=C(CC/C=C(\C)CCC=C(C)C)C[C@H](C(=O)O)[C@H]1C(=O)O]

or a pharmaceutically acceptable salt thereof.

In some embodiments, the disclosure features a compound represented by formula (73)

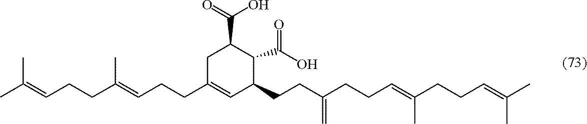 [SMILES: C=C(CC/C=C(\C)CCC=C(C)C)CC[C@H]1C=C(CC/C=C(\C)CCC=C(C)C)C[C@@H](C(=O)O)[C@@H]1C(=O)O]

or a pharmaceutically acceptable salt thereof.

In some embodiments, the disclosure features a compound represented by formula (74)

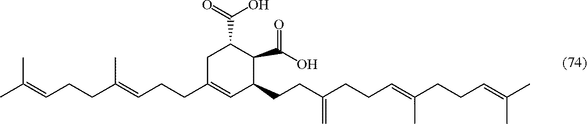 [SMILES: C=C(CC/C=C(\C)CCC=C(C)C)CC[C@H]1C=C(CC/C=C(\C)CCC=C(C)C)C[C@H](C(=O)O)[C@H]1C(=O)O]

or a pharmaceutically acceptable salt thereof.

In some embodiments, the disclosure features a compound represented by formula (75)

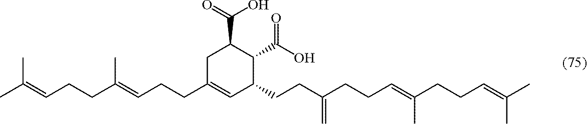 [SMILES: C=C(CC/C=C(\C)CCC=C(C)C)CC[C@@H]1C=C(CC/C=C(\C)CCC=C(C)C)C[C@@H](C(=O)O)[C@@H]1C(=O)O]

or a pharmaceutically acceptable salt thereof.

In some embodiments, the disclosure features a compound represented by formula (76)

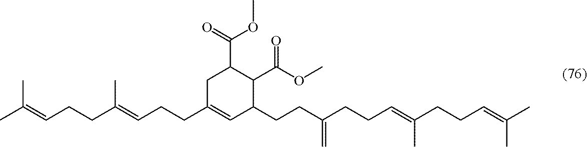 [SMILES: C=C(CC/C=C(\C)CCC=C(C)C)CCC1C=C(CC/C=C(\C)CCC=C(C)C)CC(C(=O)OC)C1C(=O)OC]

In some embodiments, the disclosure features a compound represented by formula (77)

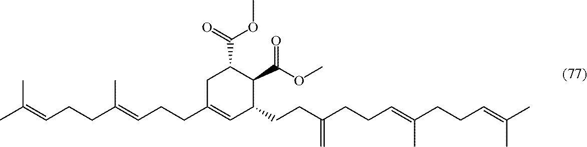 [SMILES: C=C(CC/C=C(\C)CCC=C(C)C)CC[C@@H]1C=C(CC/C=C(\C)CCC=C(C)C)C[C@H](C(=O)OC)[C@H]1C(=O)OC]

In some embodiments, the disclosure features a compound represented by formula (78)

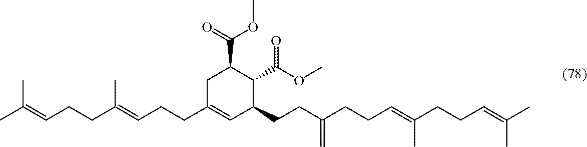 [SMILES: C=C(CC/C=C(\C)CCC=C(C)C)CC[C@H]1C=C(CC/C=C(\C)CCC=C(C)C)C[C@@H](C(=O)OC)[C@@H]1C(=O)OC]

In some embodiments, the disclosure features a compound represented by formula (79)

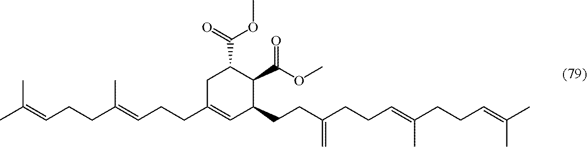 [SMILES: C=C(CC/C=C(\C)CCC=C(C)C)CC[C@H]1C=C(CC/C=C(\C)CCC=C(C)C)C[C@H](C(=O)OC)[C@H]1C(=O)OC]

In some embodiments, the disclosure features a compound represented by formula (80)

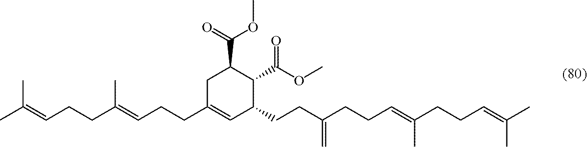 [SMILES: C=C(CC/C=C(\C)CCC=C(C)C)CC[C@@H]1C=C(CC/C=C(\C)CCC=C(C)C)C[C@@H](C(=O)OC)[C@@H]1C(=O)OC]

In some embodiments, the disclosure features a compound represented by formula (81)

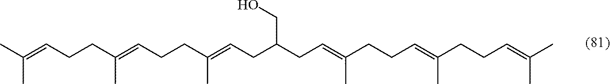 [SMILES: CC(C)=CCC/C(C)=C/CC/C(C)=C/CC(CO)C/C=C(\C)CC/C=C(\C)CCC=C(C)C]

In some embodiments, the disclosure features a compound represented by formula (82)

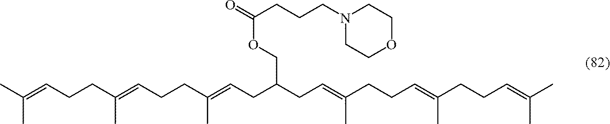 [SMILES: CC(C)=CCC/C(C)=C/CC/C(C)=C/CC(C/C=C(\C)CC/C=C(\C)CCC=C(C)C)COC(=O)CCCN1CCOCC1]

In some embodiments, the disclosure features a compound represented by formula (83)

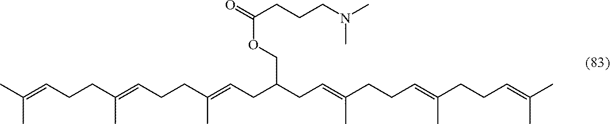 [SMILES: CC(C)=CCC/C(C)=C/CC/C(C)=C/CC(C/C=C(\C)CC/C=C(\C)CCC=C(C)C)COC(=O)CCCN(C)C]

In some embodiments, the disclosure features a compound represented by formula (84)

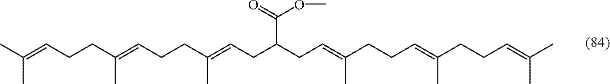 [SMILES: COC(=O)C(C/C=C(\C)CC/C=C(\C)CCC=C(C)C)C/C=C(\C)CC/C=C(\C)CCC=C(C)C]

In some embodiments, the disclosure features a compound represented by formula (85)

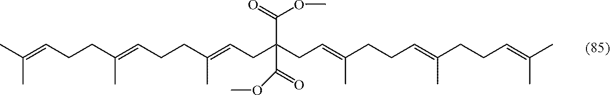 [SMILES: COC(=O)C(C/C=C(\C)CC/C=C(\C)CCC=C(C)C)(C/C=C(\C)CC/C=C(\C)CCC=C(C)C)C(=O)OC]

In some embodiments, the disclosure features a compound represented by formula (86)

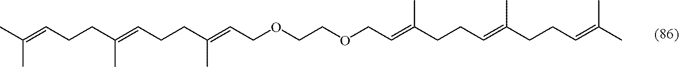 [SMILES: CC(C)=CCC/C(C)=C/CC/C(C)=C/COCCOC/C=C(\C)CC/C=C(\C)CCC=C(C)C]

In some embodiments, the disclosure features a compound represented by formula (87)

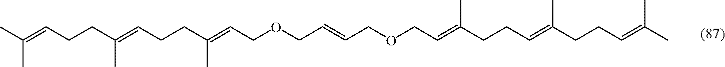 [SMILES: CC(C)=CCC/C(C)=C/CC/C(C)=C/COC/C=C/COC/C=C(\C)CC/C=C(\C)CCC=C(C)C]

In some embodiments, the disclosure features a compound represented by formula (88)

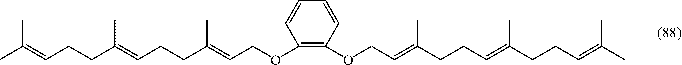 [SMILES: CC(C)=CCC/C(C)=C/CC/C(C)=C/COc1ccccc1OC/C=C(\C)CC/C=C(\C)CCC=C(C)C]

In some embodiments, the disclosure features a compound represented by formula (89)

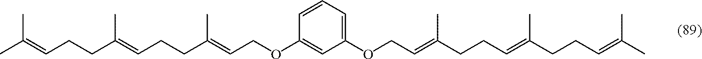 [SMILES: CC(C)=CCC/C(C)=C/CC/C(C)=C/COc1cccc(OC/C=C(\C)CC/C=C(\C)CCC=C(C)C)c1]

In some embodiments, the disclosure features a compound represented by formula (90)

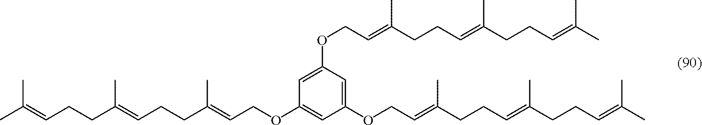 [SMILES: CC(C)=CCC/C(C)=C/CC/C(C)=C/COc1cc(OC/C=C(\C)CC/C=C(\C)CCC=C(C)C)cc(OC/C=C(\C)CC/C=C(\C)CCC=C(C)C)c1]

In some embodiments, the disclosure features a compound represented by formula (91)

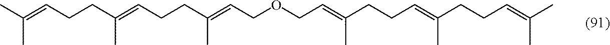 [SMILES: CC(C)=CCC/C(C)=C/CC/C(C)=C/COC/C=C(\C)CC/C=C(\C)CCC=C(C)C]

In another aspect (“A3”), the disclosure features a composition comprising a compound of any one of the foregoing aspects or embodiments of the disclosure. In some embodiments, the compound has a purity of at least 85% by weight (e.g., a purity of 85% by weight, 86% by weight, 87% by weight, 88% by weight, 89% by weight, 90% by weight, 91% by weight, 92% by weight, 93% by weight, 94% by weight, 95% by weight, 96% by weight, 97% by weight, 98% by weight, 99% by weight, 99.1% by weight, 99.2% by weight, 99.3% by weight, 99.4% by weight, 99.5% by weight, 99.6% by weight, 99.7% by weight, 99.8% by weight, 99.9% by weight, 99.99% by weight, or 100% by weight). In some embodiments, the compound has a purity of from about 85% by weight to about 99.9% by weight (e.g., a purity of about 85% by weight, 86% by weight, 87% by weight, 88% by weight, 89% by weight, 90% by weight, 91% by weight, 92% by weight, 93% by weight, 94% by weight, 95% by weight, 96% by weight, 97% by weight, 98% by weight, 99% by weight, 99.1% by weight, 99.2% by weight, 99.3% by weight, 99.4% by weight, 99.5% by weight, 99.6% by weight, 99.7% by weight, 99.8% by weight, or 99.9% by weight).

In some embodiments, the compound has a purity of from about 90% by weight to about 99.9% by weight (e.g., a purity of about 90% by weight, 91% by weight, 92% by weight, 93% by weight, 94% by weight, 95% by weight, 96% by weight, 97% by weight, 98% by weight, 99% by weight, 99.1% by weight, 99.2% by weight, 99.3% by weight, 99.4% by weight, 99.5% by weight, 99.6% by weight, 99.7% by weight, 99.8% by weight, or 99.9% by weight).

In some embodiments, the compound has a purity of from about 95% by weight to about 99.9% by weight (e.g., a purity of about 95.1% by weight, 95.2% by weight, 95.3% by weight, 95.4% by weight, 95.5% by weight, 95.6% by weight, 95.7% by weight, 95.8% by weight, 95.9% by weight, 96% by weight, 96.1% by weight, 96.2% by weight, 96.3% by weight, 96.4% by weight, 96.5% by weight, 96.6% by weight, 96.7% by weight, 96.8% by weight, 96.9% by weight, 97% by weight, 97.1% by weight, 97.2% by weight, 97.3% by weight, 97.4% by weight, 97.5% by weight, 97.6% by weight, 97.7% by weight, 97.8% by weight, 97.9% by weight, 98% by weight, 98.1% by weight, 98.2% by weight, 98.3% by weight, 98.4% by weight, 98.5% by weight, 98.6% by weight, 98.7% by weight, 98.8% by weight, 98.9% by weight, 99% by weight, 99.1% by weight, 99.2% by weight, 99.3% by weight, 99.4% by weight, 99.5% by weight, 99.6% by weight, 99.7% by weight, 99.8% by weight, or 99.9% by weight).

In some embodiments of A3, the compound is present in the composition in an amount of at least 1 kg (e.g., in an amount of 1 kg, 2 kg, 3 kg, 4 kg, 5 kg, 6 kg, 7 kg, 8 kg, 9 kg, 10 kg, 20 kg, 30 kg, 40 kg, 50 kg, 60 kg, 70 kg, 80 kg, 90 kg, or 100 kg, or more). In some embodiments, the compound is present in the composition in an amount of from about 1 kg to about 100 kg (e.g., in an amount of about 1 kg, 2 kg, 3 kg, 4 kg, 5 kg, 6 kg, 7 kg, 8 kg, 9 kg, 10 kg, 11 kg, 12 kg, 13 kg, 14 kg, 15 kg, 16 kg, 17 kg, 18 kg, 19 kg, 20 kg, 21 kg, 22 kg, 23 kg, 24 kg, 25 kg, 26 kg, 27 kg, 28 kg, 29 kg, 30 kg, 31 kg, 32 kg, 33 kg, 34 kg, 35 kg, 36 kg, 37 kg, 38 kg, 39 kg, 40 kg, 41 kg, 42 kg, 43 kg, 44 kg, 45 kg, 46 kg, 47 kg, 48 kg, 49 kg, 50 kg, 51 kg, 52 kg, 53 kg, 54 kg, 55 kg, 56 kg, 57 kg, 58 kg, 59 kg, 60 kg, 61 kg, 62 kg, 63 kg, 64 kg, 65 kg, 66 kg, 67 kg, 68 kg, 69 kg, 70 kg, 71 kg, 72 kg, 73 kg, 74 kg, 75 kg, 76 kg, 77 kg, 78 kg, 79 kg, 80 kg, 81 kg, 82 kg, 83 kg, 84 kg, 85 kg, 86 kg, 87 kg, 88 kg, 89 kg, 90 kg, 91 kg, 92 kg, 93 kg, 94 kg, 95 kg, 96 kg, 97 kg, 98 kg, 99 kg, or 100 kg).

In another aspect (“A4”), the disclosure features an adjuvant formulation containing the compound of any one of aspects A1 and A2, or of any of the embodiments thereof. The adjuvant formulation may further include a pharmaceutically acceptable carrier, diluent, or excipient.

In another aspect (“A5”), the disclosure features a vaccine containing a therapeutically or prophylactically effective amount of the adjuvant formulation of A4 and (i) an antigen and/or (ii) an agent that activates antigen-presenting cells, such as a toll-like receptor 4 (TLR4) agonist (e.g., glucopyranosyl lipid A or lipopolysaccharide). The antigen may be a protein, for example, a protein expressed by a virus, bacterium, protozoan, or cancer cell. In some embodiments, the antigen is encoded by a nucleic acid molecule (e.g., a deoxyribonucleic acid (DNA) or ribonucleic acid (RNA) molecule) encoding such a protein.

In another aspect (“A6”), the disclosure features a vaccine containing the compound of any one of aspects A1 and A2, or of any of the embodiments thereof, covalently bound to (i) an antigen and/or (ii) an agent that activates antigen-presenting cells, such as a TLR4 agonist (e.g., glucopyranosyl lipid A or lipopolysaccharide). The antigen may be a protein, for example, a protein expressed by a virus, bacterium, protozoan, or cancer cell. In some embodiments, the antigen is encoded by a nucleic acid molecule (e.g., a DNA or RNA molecule) encoding such a protein.

In some embodiments of A5 or A6, the antigen is a protein expressed by a virus selected from influenza virus, hepatitis A virus, hepatitis B virus, hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O′nyong′nyong virus, chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi’s sarcoma associated-herpesvirus (KSHV), severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus, rhinovirus, mumps virus, poliovirus, human enterovirus, coxsackievirus, human papilloma virus, adeno-associated virus, astrovirus, JC virus, BK virus, SV40 virus, Norwalk virus, rotavirus, human immunodeficiency virus (HIV), and human T-lymphotropic virus. In some embodiments, the antigen is encoded by a nucleic acid molecule (e.g., a DNA or RNA molecule) encoding a protein expressed by any one or more of the foregoing virions.

In some embodiments of A5 or A6, the antigen is a protein expressed by a coronavirus, such as a beta coronavirus. In some embodiments, the beta coronavirus is SARS-CoV-2, MERS-CoV, SARS-CoV, OC43, or HKU1. In some embodiments, the coronavirus is SARS-CoV-2. In some embodiments, the antigen is encoded by a nucleic acid molecule (e.g., a DNA or RNA molecule) encoding such a protein.

In some embodiments of A5 or A6, the antigen is a protein expressed by an alpha coronavirus, such as 229E or NL63. In some embodiments, the antigen encoded by is a nucleic acid molecule (e.g., a DNA or RNA molecule) encoding such a protein.

In some embodiments of A5 or A6, the antigen is a protein expressed by a bacterium, such as a bacterium belonging to a genus selected from Mycobacterium (e.g., Mycobacterium tuberculosis), Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasturella, Francisella, Brucella, Bartonella, Clostridium, Vibrio, Campylobacter, and Staphylococcus. In some embodiments, the antigen is encoded by a nucleic acid molecule (e.g., a DNA or RNA molecule) encoding such a protein.

In some embodiments of A5 or A6, the antigen is a protein expressed by a protozoan. In some embodiments, the antigen is a protein expressed by a parasite, such as a parasite selected from the group consisting of Plasmodium malariae, Plasmodium vivax, Plasmodium ovale, Plasmodium falciparum, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Trichomonas vaginalis, and Histomonas meleagridis. The parasite may be a helminthic parasite, such as Richuris trichiura, Ascaris lumbricoides, Enterobius vermicularis, Ancylostoma duodenale, Necator americanus, Strongyloides stercoralis, Wuchereria bancrofti, Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, or Echinococcus granulosus. In some embodiments, the antigen is encoded by a nucleic acid molecule (e.g., a DNA or RNA molecule) encoding a protein expressed by any of the above parasites.

In some embodiments of A5 or A6, the antigen is a protein expressed by a cancer cell, or is encoded by a nucleic acid molecule (e.g., a DNA or RNA molecule) encoding such a protein.

For example, in some embodiments, the antigen is a protein expressed by an ovarian cancer cell. Such proteins include Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-16, L1CAM, Mannan-MUC-1, HERV-K-MEL, KK-LC-1, KM-HN-1, LAGE-1, MAGE-A4, SSX-4, TAG-1, and TAG-2, among others. In some embodiments, the antigen is encoded by a nucleic acid molecule (e.g., a DNA or RNA molecule) encoding any of the foregoing proteins.

In some embodiments, the antigen is a protein expressed by a breast cancer cell. Such proteins include ENAH (hMena), mammaglobin-A, NY-BR-1, EpCAM, NY-ESO-1, BAGE-1, HERV-K-MEL, KK-LC-1, KM-HN-1, LAGE-1, MAGE-A1, MAGE-A2, mucink, Sp17, SSX-2, TAG-1, TAG-2, TRAG-3, Her2/Neu, c-myc, cyclin B1, MUC1, p53, p62, and Survivin, among others. In some embodiments, the antigen is encoded by a nucleic acid molecule (e.g., a DNA or RNA molecule) encoding any of the foregoing proteins.

In some embodiments, the antigen is a protein expressed by a pancreatic cancer cell. Such proteins include ENAH (hMena), PBF, K-ras, Mesothelin, and mucink, among others. In some embodiments, the antigen is encoded by a nucleic acid molecule (e.g., a DNA or RNA molecule) encoding any of the foregoing proteins.

In some embodiments, the antigen is a protein expressed by a colorectal cancer cell. Such proteins include ENAH (hMena), Intestinal carboxyl esterase, CASP-5, COA-1, OGT, OS-9, TGF-βRII, NY-ESO-1, CEA, HERV-K-MEL, KK-LC-1, KM-HN-1, LAGE-1, MAGE-A2, Sp17, TAG-1, TAG-2, c-myc, cyclin B1, MUC1, p53, p62, Survivin, and gp70, among others. In some embodiments, the antigen is encoded by a nucleic acid molecule (e.g., a DNA or RNA molecule) encoding any of the foregoing proteins.

In some embodiments, the antigen is a protein expressed by a lung cancer cell. Such proteins include CD274, mdm-2, α-actinin-4, Elongation factor 2, ME1, NFYC, NY-ESO-1, GAGE-1/2/8, HERV-K-MEL, KK-LC-1, KM-HN-1, LAGE-1, MAGE-A2, MAGE-A6, Sp17, TAG-1, TAG-2, TRAG-3, XAGE-1b/GAGED2a, c-myc, cyclin B1, Her2/Neu, MUC1, p53, p62, and Survivin, among others. In some embodiments, the antigen is encoded by a nucleic acid molecule (e.g., a DNA or RNA molecule) encoding any of the foregoing proteins.

In some embodiments, the antigen is a protein expressed by a prostate cancer cell. Such proteins include DKK1, ENAH (hMena), Kallikrein 4, PSMA, STEAP1, PAP, PSA, NY-ESO-1, BAGE-1, GAGE-1/2/8, GAGE-3/4/5/6/7, HERV-K-MEL, KK-LC-1, KM-HN-1, LAGE-1, and Sp17, among others. In some embodiments, the antigen is encoded by a nucleic acid molecule (e.g., a DNA or RNA molecule) encoding any of the foregoing proteins.

In some embodiments, the antigen is a protein expressed by a melanoma cell. Such proteins include gp100, Hepsin, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, NY-ESO-1, BAGE-1, GAGE-1/2/8, GAGE-3/4/5/6/7, GnTVf, HERV-K-MEL, KK-LC-1, KM-HN-1, LAGE-1, LY6K, MAGE-A1, MAGE-A6, MAGE-A10, MAGE-A12, MAGE-C2, NA88-A, Sp17, SSX-2, SSX-4, and TRAG-3, among others. In some embodiments, the antigen is encoded by a nucleic acid molecule (e.g., a DNA or RNA molecule) encoding any of the foregoing proteins.

In some embodiments, the antigen is a protein expressed by a squamous cell carcinoma cell. Such proteins include CASP-8, p53, and SAGE, among others. In some embodiments, the antigen is encoded by a nucleic acid molecule (e.g., a DNA or RNA molecule) encoding any of the foregoing proteins.

In some embodiments, the antigen is a protein expressed by a chronic myeloid leukemia cell. Such proteins include BCR-ABL, dek-can, EFTUD2, and GAGE-3/4/5/6/7, among others. In some embodiments, the antigen is encoded by a nucleic acid molecule (e.g., a DNA or RNA molecule) encoding any of the foregoing proteins.

In some embodiments, the antigen is a protein expressed by an acute lymphoblastic leukemia cell. Such proteins include ETV6-AML1, and GAGE-3/4/5/6/7, among others. In some embodiments, the antigen is encoded by a nucleic acid molecule (e.g., a DNA or RNA molecule) encoding any of the foregoing proteins.

In some embodiments, the antigen is a protein expressed by an acute myelogenous leukemia cell. Such proteins include FLT3-ITD, Cyclin-A1, and GAGE-3/4/5/6/7, among others. In some embodiments, the antigen is encoded by a nucleic acid molecule (e.g., a DNA or RNA molecule) encoding any of the foregoing proteins.

In some embodiments, the antigen is a protein expressed by a chronic lymphocytic leukemia cell. Such proteins include FNDC3B and GAGE-3/4/5/6/7, among others. In some embodiments, the antigen is encoded by a nucleic acid molecule (e.g., a DNA or RNA molecule) encoding any of the foregoing proteins.

In some embodiments, the antigen is a protein expressed by a multiple myeloma cell. Such proteins include MAGE-C1, NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

In some embodiments, the antigen is a protein expressed by a bladder cancer cell. Such proteins include BAGE-1, GAGE-1/2/8, GAGE-3/4/5/6/7, MAGE-A4, MAGE-A6, SAGE, NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

In some embodiments, the antigen is a protein expressed by a neuroblastoma cell. Such proteins include NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

In some embodiments of A5 or A6, the vaccine is formulated as a lipid nanoparticle. The lipid nanoparticle may be structured, for example, such that the core contains the compound of any one of aspects A1 and A2, or any embodiment thereof. A nucleic acid (e.g., DNA or RNA), encoding the antigen may be located on the exterior or interior of the lipid nanoparticle. In some embodiments, the lipid nanoparticle further contains a cationic lipid or a non-cationic lipid.

In another aspect (“A7”), the disclosure features a method of inducing an antigen-specific immune response in a subject (e.g., a mammalian subject, such as a human). The method may include, for example, administering to the subject the vaccine of aspect A5 or A6, or of any of the embodiments thereof.

In another aspect (“A8”), the disclosure features a method of treating or preventing a viral, bacterial, or protozoan infection, or a cancer, in a subject (e.g., a mammalian subject, such as a human) by, for example, administering to the subject the vaccine of aspect A5 or A6, or of any of the embodiments thereof.

In another aspect (“A9”), the disclosure features a method of synthesizing a compound represented by formula (II)

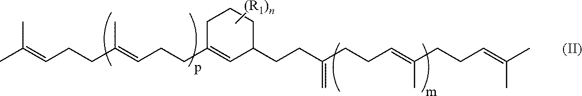

or a pharmaceutically acceptable salt thereof, by reacting a diene represented by formula (XXVII)

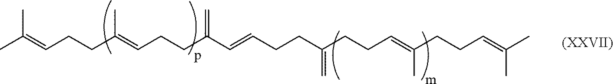

with a dienophile represented by formula (XXVIII)

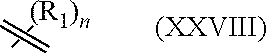

under Diels-Alder reaction conditions,
wherein formula (XXVIII) represents an ethene molecule optionally substituted with up to n instances of R₁;
wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, optionally substituted haloalkyl, or halogen;
n is an integer from 0 to 4 (e.g., 0, 1, 2, 3, or 4);
m is an integer from 0 to 3 (e.g., 0, 1, 2, or 3); and
p is an integer from 0 to 3 (e.g., 0, 1, 2, or 3).

In another aspect (“A10”), the disclosure features a method of synthesizing a compound represented by formula (XI)

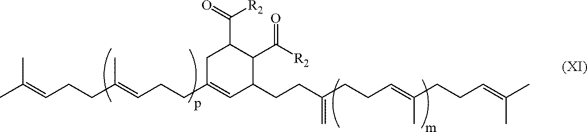

or a pharmaceutically acceptable salt thereof, by reacting a diene represented by formula (XXVII)

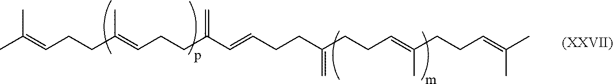

with a dienophile represented by formula (XXIX)

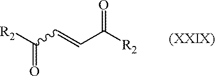

under Diels-Alder reaction conditions,
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In another aspect (“A11”), the disclosure features a method of synthesizing a compound represented by formula (XXX)

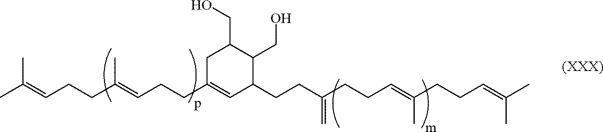

by reacting a diene represented by formula (XXVII)

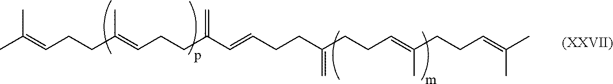

with a dienophile represented by formula (XXIX)

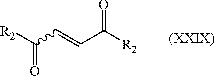

under Diels-Alder reaction conditions, thereby forming an intermediate represented by formula (XXXI)

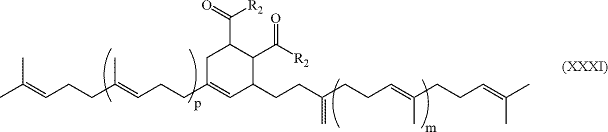

and subsequently reacting the intermediate represented by formula (XXXI) with a reducing agent, thereby producing a compound represented by formula (XXX),
wherein each R₂ is, independently, hydrogen, optionally substituted alkoxy, optionally substituted acyloxy, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

### Definitions

### Chemical Terms

The chemical terminology used herein is for the purpose of describing various aspects and embodiments of the disclosure and is not intended to be limiting.

In the following chemical definitions, a notation in which an integral number immediately follows an atomic symbol indicates the quantity of atoms of that element that are present in a particular chemical moiety. As will be understood, other atoms, such as hydrogen atoms, or substituent groups described herein, may be present, as necessary, to satisfy the valence of a particular atom. For example, an unsubstituted “C₂ alkyl group” has the formula —CH₂CH₃. When used in conjunction with the groups defined herein, a reference to a number of carbon atoms includes the divalent carbon in acetal and ketal groups but does not include the carbonyl carbon in acyl, ester, carbonate, amide, or carbamate groups. A reference to a number of oxygen, nitrogen, or sulfur atoms in a heteroaryl group only includes those atoms that form a part of a heterocyclic ring.

As used herein, a phrase of the form “optionally substituted X” (e.g., optionally substituted alkyl) is intended to be equivalent to “X, wherein X is optionally substituted” (e.g., “alkyl, wherein the alkyl is optionally substituted”). It is not intended to mean that the feature “X” (e.g., alkyl) per se is optional. As described herein, certain compounds may contain one or more “optionally substituted” moieties. In general, the term “substituted”, whether preceded by the term “optionally” or not, means that one or more hydrogens of the designated moiety are replaced with a suitable substituent, such as any of the substituents or groups described herein. When the term “optionally substituted” is used in isolation (i.e., without specifying what the optional substituents are), the substituents may be alkyl, aminoalkyl, heteroalkyl, alkenyl, heteroalkenyl, alkynyl, heteroalkynyl, carbocyclyl, heterocyclyl, aryl, heteroaryl, carbonyl, alkoxy, acyloxy, hydroxyl, oxo, haloalkyl, or halogen. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents that may be used in conjunction with the compounds of the disclosure are preferably those that result in the formation of stable or chemically feasible compounds. The term “stable,” as used herein, refers to compounds that are not substantially altered when subjected to conditions that allow for their production, detection, and, in certain embodiments, recovery, purification, and use for one or more of the purposes disclosed herein.

As used herein, the term “aliphatic” refers to a saturated or unsaturated, straight, branched, or cyclic hydrocarbon. The term “aliphatic” includes, but is not limited to, alkyl, alkenyl, alkynyl, cycloalkyl, cycloalkenyl, and cycloalkynyl moieties, and thus incorporates each of these definitions. In some embodiments, “aliphatic” is used to indicate those aliphatic groups having from 1 to 20 carbon atoms. The aliphatic chain may be, for example, mono-unsaturated, di-unsaturated, tri-unsaturated, or polyunsaturated, or alkynyl. Unsaturated aliphatic groups can be in a cis or trans configuration. In some embodiments, the aliphatic group contains from 1 to about 12 carbon atoms, such as from 1 to about 6 carbon atoms or from 1 to about 4 carbon atoms. In some embodiments, the aliphatic group contains from 1 to about 8 carbon atoms. In some embodiments, the aliphatic group is C₁-C₂, C₁-C₃, C₁-C₄, C₁-C₅, or C₁-C₆. The specified ranges used herein indicate an aliphatic group having each member of the range described as an independent species. For example, the term “C₁-C₆ aliphatic” as used herein indicates a straight or branched alkyl, alkenyl, or alkynyl group having from 1, 2, 3, 4, 5, or 6 carbon atoms and is intended to mean that each of these is described as an independent species. For example, the term “C₁-C₄ aliphatic” as used herein indicates a straight or branched alkyl, alkenyl, or alkynyl group having from 1, 2, 3, or 4 carbon atoms and is intended to mean that each of these is described as an independent species. In some embodiments, the aliphatic group is substituted with one or more functional groups that results in the formation of a stable moiety.

As used herein, the term “heteroaliphatic” refers to an aliphatic moiety that contains at least one heteroatom in its chain, such as an amine, carbonyl, carboxy, oxo, thio, phosphate, phosphonate, nitrogen, phosphorus, silicon, or boron atom in place of a carbon atom. In some embodiments, the heteroatom present is nitrogen. In some embodiments, the heteroatom present is oxygen. In some embodiments, the heteroatom present is sulfur. The term “heteroaliphatic” includes, but is not limited to, heteroalkyl, heteroalkenyl, heteroalkynyl, heterocycloalkyl, heterocycloalkenyl, and heterocycloalkynyl moieties. In some embodiments, “heteroaliphatic” is used to indicate a heteroaliphatic group (cyclic, acyclic, substituted, unsubstituted, branched or unbranched) having from 1 to 20 carbon atoms. In some embodiments, the heteroaliphatic group is optionally substituted in a manner that results in the formation of a stable moiety. Nonlimiting examples of heteroaliphatic moieties are polyethylene glycol, polyalkylene glycol, amide, polyamide, glycolide, polylactide, polyglycolide, thioether, ether, alkyl-heterocycle-alkyl, -O-alkyl-O-alkyl, and alkyl-O-haloalkyl.

As used herein, the term “acyl” refers to a carbonyl substituent, such as a carbonyl substituent in which the carbonyl carbon is bound to an alkyl group, an alkenyl group, an alkynyl group, an optionally substituted oxygen moiety, an optionally substituted nitrogen moiety, and the like. Exemplary acyl groups include, without limitation, formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

As used herein, the term “acyloxy” refers to the chemical moiety -OC(O)R in which R is C₁-C₆, alkyl, aryl, heteroaryl, C₁-C₆ alkyl aryl, or C₁-C₆ alkyl heteroaryl.

As used herein, the term “alkyl” refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms). As used herein, the term “alkylene” refers to a divalent alkyl group.

As used herein, the term “alkenyl,” whether recited alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms). As used herein, the term “alkenylene” refers to a divalent alkenyl group.

As used herein, the term “alkynyl,” whether recited alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms). As used herein, the term “alkynylene” refers to a divalent alkynyl group.

As used herein, the term “amino” represents -N(R^{N1})₂, wherein each R^{N1} is, independently, H, OH, NO₂, N(R^{N2})₂, SO₂OR^{N2,} SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., -N(R^{N1})₂).

As used herein, the term “aryl” refers to an aromatic mono- or polycarbocyclic radical of, e.g., 6 to 12, carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

As used herein, the term “arylalkyl” represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

As used herein, the term “bridged cyclyl” refers to a bridged polycyclic group of 5 to 20 atoms, containing from 1 to 3 bridges. Bridged cyclyl includes bridged carbocyclyl (e.g., norbornyl) and bridged heterocyclyl (e.g., 1,4-diazabicyclo[2.2.2]octane).

As used herein, the term “carbocyclyl” refers to a non-aromatic C₃-C₁₂, monocyclic or polycyclic (e.g., bicyclic or tricyclic) structure in which the rings are formed by carbon atoms. Carbocyclyl structures include cycloalkyl groups (e.g., cyclohexyl) and unsaturated carbocyclyl radicals (e.g., cyclohexenyl). Polycyclic carbocyclyl includes spirocyclic carbocyclyl, bridged carbocyclyl, and fused carbocyclyl. As used herein, the term “carbocyclylene” refers to a divalent carbocyclyl group.

As used herein, the term “cycloalkyl” refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

As used herein, the terms “halo” and “halogen” mean a fluorine (fluoro), chlorine (chloro), bromine (bromo), or iodine (iodo) radical.

As used herein, the term “heteroalkyl” refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers to alkyl-O- (e.g., methoxy and ethoxy), and an “alkylamino” which, as used herein, refers to -N(alkyl)R^{Na}, where R^{Na} is H or alkyl (e.g., methylamino). As used herein, the term “heteroalkylene” refers to a divalent heteroalkyl group.

As used herein, the term “heteroalkenyl” refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers to alkenyl-O-. As used herein, the term “heteroalkenylene” refers to a divalent heteroalkenyl group.

As used herein, the term “heteroalkynyl” refers to an alkynyl group, as defined herein, in which one or more of the constituent carbon atoms have been replaced by nitrogen, oxygen, or sulfur. In some embodiments, the heteroalkynyl group is further substituted with 1, 2, 3, or 4 substituent groups as described herein for alkynyl groups. Examples of heteroalkynyl groups are an “alkynoxy” which, as used herein, refers to alkynyl-O-. As used herein, the term “heteroalkynylene” refers to a divalent heteroalkynyl group.

As used herein, the term “heteroaryl” refers to an aromatic monocyclic or polycyclic structure of 5 to 12 atoms having at least one aromatic ring containing 1, 2, or 3 ring atoms selected from nitrogen, oxygen, and sulfur, with the remaining ring atoms being carbon. In some embodiments, one or two ring carbon atoms of the heteroaryl group are replaced with a carbonyl group. Examples of heteroaryl groups are pyridyl, pyrazoyl, benzooxazolyl, benzoimidazolyl, benzothiazolyl, imidazolyl, oxaxolyl, and thiazolyl. As used herein, the term “heteroarylene” refers to a divalent heteroaryl group.

As used herein, the term “heteroarylalkyl” represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

As used herein, the term “heterocyclyl” refers a monocyclic or polycyclic radical (e.g., bicyclic or tricyclic) having 3 to 12 atoms having at least one non-aromatic ring containing 1, 2, 3, or 4 ring atoms selected from N, O, or S, and no aromatic ring containing any N, O, or S atoms. Polycyclic heterocyclyl includes spirocyclic heterocyclyl, bridged heterocyclyl, and fused heterocyclyl. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. As used herein, the term “heterocyclylene” refers to a divalent heterocyclyl group.

As used herein, the term “heterocyclylalkyl” represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

As used herein, the term “hydroxyalkyl” refers to an alkyl group substituted with an —OH group.

As used herein, the term “hydroxyl” refers to an —OH group.

As used herein, the term “imine” refers to a =NR^{N} group, where R^{N} is, e.g., H or alkyl.

As used herein, the term “N-protecting group” refers to those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4- 20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

As used herein, the term “nitro” refers to an —NO₂ group.

As used herein, the term “oxo” refers to an ═O group.

As used herein, the term “sulfonyl” refers to chemical moiety -SO₂-R in which R is hydrogen, aryl, heteroaryl, C₁-C₆ alkyl, C₁-C₆ alkyl substituted with one or more halogens, such as a —SO₂—CF₃ substituent, C₁-C₆ alkyl aryl, or C₁-C₆ alkyl heteroaryl.

As used herein, the term “sulfonylamino” refers to the chemical moiety -NRSO₂-R′ in which each of R and R′ is independently hydrogen, C₁-C₆ alkyl, aryl, heteroaryl, C₁-C₆ alkyl aryl, or C₁-C₆ alkyl heteroaryl.

As used herein, the term “sulfonyloxy” refers to the chemical moiety -OSO₂-R in which R is hydrogen, C₁-C₆ alkyl, C₁-C₆ alkyl substituted with one or more halogens, such as a -OSO₂-CF₃ substituent, aryl, heteroaryl, C₁-C₆ alkyl aryl, or C₁-C₆ alkyl heteroaryl.

As used herein, the term “thiol” refers to an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups described herein may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, oxo, sulfonyl, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

### Depictions of Chemical Structures

Compounds of the disclosure may have one or more asymmetric carbon atoms and may exist in the form of optically pure enantiomers, mixtures of enantiomers (e.g., racemates), optically pure diastereoisomers, mixtures of diastereoisomers, diastereoisomeric racemates, or mixtures of diastereoisomeric racemates. The optically active forms can be obtained, for example, by resolution of the racemates, by asymmetric synthesis or asymmetric chromatography (chromatography with a chiral adsorbent or eluant). Accordingly, the compounds disclosed herein may exist in various stereoisomeric forms.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. The term “enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms.

Enantiomers of a compound can be prepared, for example, by separating an enantiomer from a racemate using one or more well-known techniques and methods, such as, for example, chiral chromatography and separation methods based thereon. The terms “racemate” and “racemic mixture” refer to a compound containing two enantiomers, wherein such mixtures exhibit no optical activity; i.e., they do not rotate the plane of polarized light. The term “geometric isomer” refers to isomers that differ in the orientation of substituent atoms in relationship to a carbon-carbon double bond, to a cycloalkyl ring, or to a bridged bicyclic system. Atoms (other than H) on each side of a carbon- carbon double bond may be in an E (substituents are on opposite sides of the carbon-carbon double bond) or Z (substituents are oriented on the same side of the carbon-carbon double bond) configuration. “R,” “S,” “S*,” “R*,” “E,” “Z,” “cis,” and “trans,” indicate configurations relative to the core molecule.

When the stereochemistry of a compound disclosed herein is named or depicted by structure, the named or depicted stereoisomer is greater than 50% by weight (e.g., at least 60%, 70%, 80%, 90%, 99%, or 99.9% by weight) relative to its other stereoisomers. For example, when a single enantiomer is named or depicted by structure, the depicted or named enantiomer is greater than 50% by weight (e.g., at least 60%, 70%, 80%, 90%, 99%, or 99.9% by weight) optically pure. Similarly, when a single diastereomer is named or depicted by structure, the depicted or named diastereomer is greater than 50% by weight (e.g., at least 60%, 70%, 80%, 90%, 99%, or 99.9% by weight) pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers.

Additionally, when the stereochemistry of a compound disclosed herein is named or depicted by structure, the named or depicted stereoisomer is greater than 50% by mole fraction (e.g., at least 60%, 70%, 80%, 90%, 99%, or 99.9% by mole fraction) relative to its other stereoisomers. For example, when a single enantiomer is named or depicted by structure, the depicted or named enantiomer is greater than 50% by mole fraction (e.g., at least 60%, 70%, 80%, 90%, 99%, or 99.9% by mole fraction) relative to the other enantiomer. When a single diastereomer is named or depicted by structure, the depicted or named diastereomer is greater than 50% by mole fraction (e.g., at least 60%, 70%, 80%, 90%, 99%, or 99.9% by mole fraction) relative to the other diastereomer(s) of the indicated compound. For enantiomeric compounds, percent purity by mole fraction is calculated as the ratio of the molar quantity of the enantiomer of interest relative to the sum of the molar quantities of (i) the enantiomer of interest and (ii) the optical isomer. Similarly, for diastereomeric compounds, percent purity by moles fraction is calculated as the ratio of the molar quantity of the diastereomer of interest relative to the total molar quantities of all diastereomers present for the indicated compound.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer.

When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The present disclosure embraces all of these forms.

### Isotopic Enrichment or Substitution

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are indented to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful, for example, in tissue distribution assays. Tritiated (i.e., ³H) and carbon-14 (i.e., ¹⁴C) isotopes can be useful, for example, given their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

### Polymorphic Compounds

As will be appreciated by one of skill in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

As used herein, the term “crystalline” or “crystalline form” means having a physical state that is a regular three-dimensional array of atoms, ions, molecules or molecular assemblies. Crystalline forms have lattice arrays of building blocks called asymmetric units that are arranged according to well-defined symmetries into unit cells that are repeated in three-dimensions. In contrast, the term “amorphous” or “amorphous form” refers to an unorganized (no orderly) structure. The physical state of a therapeutic compound may be determined by exemplary techniques such as x-ray diffraction, polarized light microscopy, thermal gravimetric analysis, and/or differential scanning calorimetry.

A compound, salt form, crystal polymorph, therapeutic agent, or other composition described herein may be referred to as being characterized by graphical data “substantially as depicted in” a figure. Such data may include, without limitation, powder X-ray diffractograms, NMR spectra, differential scanning calorimetry curves, and thermogravimetric analysis curves, among others. As is known in the art, such graphical data may provide additional technical information to further define the compound, salt form, crystal polymorph, therapeutic agent, or other composition. As is understood by one of skill in the art, such graphical representations of data may be subject to small variations, e.g., in peak relative intensities and peak positions due to factors such as variations in instrument response and variations in sample concentration and purity. Nonetheless, one of skill in the art will readily be capable of comparing the graphical data in the figures herein with graphical data generated for a compound, salt form, crystal polymorph, therapeutic agent, or other composition and confirm whether the two sets of graphical data are characterizing the same material or two different materials.

### Additional Definitions

Unless otherwise defined, all technical and scientific terms used herein have the same meaning as commonly understood by one of ordinary skill in the art to which the disclosure pertains. Methods and materials are described herein for use in the present disclosure; other, suitable methods and materials known in the art can also be used. The materials, methods, and examples set forth herein are illustrative and not intended to be limiting. All publications mentioned herein are incorporated by reference in their entirety. In case of conflict, the present specification, including definitions, will control.

As used herein, the term “about” refers to a value that is within 10% above or below the value being described. For instance, the phrase “about 50 mg” refers to a value between and including 45 mg and 55 mg.

As used herein, the term “affinity” refers to the strength of a binding interaction between two molecules, such as a ligand and a receptor.

As used herein, the term “derived from” in the context of an antigen (e.g., a pathogen antigen or a cancer cell antigen described herein) refers to a molecular substance (e.g., a protein, carbohydrate, or nucleic acid, such as DNA or RNA) that is characteristic of the antigen. An antigen that is “derived from” a pathogen (e.g., a virus, bacterium, or protozoan) or a cancer cell may be, for example, a protein, peptide fragment, or carbohydrate that is naturally expressed by the pathogen (e.g., the virus, bacterium, or protozoan) or cancer cell. Alternatively, the antigen may be a nucleic acid component of the pathogen (e.g., the virus, bacterium, or protozoan) or cancer cell, such as a DNA or RNA molecule that encodes all or a part of a pathogen protein (e.g., a viral, bacterial, or protozoan protein, such as a coat protein or cell wall protein) or a cancer cell protein. Synthetic antigens derived from or based upon a pathogen or cancer cell nucleic acid or protein sequence (e.g., a viral, bacterial, or protozoan component (for example, coat protein)) are also included in the invention. Additional examples of antigens that are “derived from” a pathogen (e.g., a virus, bacterium, or protozoan) or cancer cell include proteins or peptide fragments thereof that result from the processing of the pathogen by the immune system of a subject (e.g., a mammalian subject, such as a human). Antigens of this type include, without limitation, peptide fragments that associate with major histocompatibility complex (MHC) proteins of an antigen-presenting immune cell upon processing of a pathogen by the immune system of a subject (e.g., a mammalian subject, such as a human). Examples of antigen-presenting immune cells include, without limitation, macrophages and dendritic cells.

As used herein, the term “Diels-Alder reaction conditions” refers to a set of ambient properties that promote the [4 + 2] cycloaddition of a diene and a dienophile. Diels-Alder cycloadditions can be facilitated, for example, by increases in temperature and, in some instances, by the use of polar solvents. Exemplary Diels-Alder reaction conditions are provided in the Examples, below. Additional examples of Diels-Alder reaction conditions are set forth in Kürti and Czakó, Strategic Applications of Named Reactions in Organic Synthesis (Elsevier Science, 2005), the disclosure of which is incorporated herein by reference as it pertains to ambient conditions that facilitate [4 + 2] cycloaddition reactions.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted therefrom.

As used herein, the term “pharmaceutical composition” refers to a mixture containing a therapeutic compound or prophylactic compound to be administered to a subject, such as a mammal, e.g., a human, in order to prevent, treat or control a particular disease or condition affecting or that may affect the mammal.

As used herein, the term “pharmaceutically acceptable” refers to those compounds, materials, compositions and/or dosage forms which are suitable for contact with the tissues of a subject, such as a mammal (e.g., a human) without excessive toxicity, irritation, allergic response, or other deleterious complications commensurate with a reasonable benefit/risk ratio.

As used herein, the term “pharmaceutically acceptable salt” means any pharmaceutically acceptable salt of a compound described herein. For example, pharmaceutically acceptable salts of any of the compounds described herein include those that are within the scope of sound medical judgment, suitable for use in contact with the tissues of humans and animals without undue toxicity, irritation, or allergic response, and are commensurate with a reasonable benefit/risk ratio. Examples of pharmaceutically acceptable salts are described in: Berge et al., J. Pharmaceutical Sciences 66:1-19, 1977 and in Pharmaceutical Salts: Properties, Selection, and Use, (Eds. P.H. Stahl and C.G. Wermuth), Wiley-VCH, 2008. Such salts can be prepared, for example, in situ during the final isolation and purification of a compound described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. The compounds may be prepared or used as pharmaceutically acceptable salts synthesized as addition products of pharmaceutically acceptable acids or bases. Suitable pharmaceutically acceptable salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and/or cells) isolated from a subject.

As used herein, the terms “subject” and “patient” are interchangeable and refer to an organism that receives therapeutic or prophylactic treatment for a particular disease or condition as described herein. Examples of subjects and patients include mammals, such as humans.

As used herein, the term “substantially pure” refers to a compound that has a purity of at least 85%, as assessed, for instance, using nuclear magnetic resonance (NMR) and/or high-performance liquid chromatography (HPLC) techniques described herein or known in the art.

### BRIEF DESCRIPTION OF THE FIGURES

FIG. 1 is a graph comparing the physical stability of an emulsion of squalene to that of emulsions of squalene analogues, DHIS and farnesene thermal dimer. Experimental details for this study are provided in Example 1, below.

FIG. 2 is a graph showing the Mip 1β cytokine stimulation response from human blood exposed to DHIS or farnesene thermal dimer as compared to that induced by squalene. FIG. 2 is plotted using the Min-Max Box and Whisker format. *p<0.05 saline by one-way ANOVA using Dunnet’s multiple comparison correction.

FIG. 3 is a graph showing HAl titers in C57BL/6 mice immunized twice intramuscularly with recombinant H5N1 and DHIS emulsion as compared to immunization using the same antigen with shark squalene emulsion. *p<0.05 vs antigen alone by one-way ANOVA using Dunnet’s multiple comparison correction. Graph is plotted using the Min-Max Box and Whisker format.

FIG. 4 is a graph showing enhancement in antigen specific IgG response in C57BL/6 mice immunized once intramuscularly with a recombinant tuberculosis antigen (ID97) and DHIS emulsion or farnesene thermal dimer emulsion. As a comparator, FIG. 4 also shows the antigen specific IgG response obtained using the same antigen with shark squalene emulsion. *p<0.05 vs antigen alone by one-way ANOVA using Dunnet’s multiple comparison correction. Graph is plotted using the Min-Max Box and Whisker format.

FIG. 5 is a graph showing enhancement in antigen specific CD4+ T cell response indicated by the CD154 marker in mice immunized twice with ID97 and DHIS emulsion or farnesene thermal dimer emulsion. As a comparator, FIG. 5 also shows the antigen specific CD4+ T cell response obtained using the same antigen with shark squalene emulsion. *p<0.05 vs antigen alone by one-way ANOVA using Dunnet’s multiple comparison correction. Graph is plotted using the Min-Max Box and Whisker format.

### DETAILED DESCRIPTION

Described herein are vaccine adjuvant compounds, as well as methods of synthesizing the same. The present disclosure also features methods of formulating vaccines using such adjuvant compounds, as well as methods of administering such a vaccine to a subject (e.g., a mammalian subject, such as a human) for therapeutic or prophylactic treatment. The sections that follow provide a detailed description of the compounds of the disclosure, as well as how these compounds can be prepared and formulated into a vaccine for therapy or prophylaxis.

### Compounds of the Disclosure

Vaccine adjuvants described herein include compounds represented by formula (I-a)

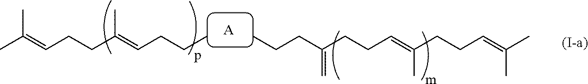

wherein A is

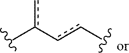

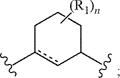

each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
each

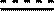

is, independently, a single bond or a double bond;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

Additional examples of vaccine adjuvants described herein are compounds represented by formula (I-b)

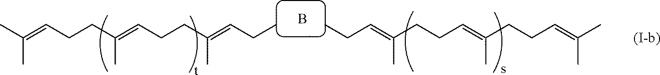

wherein B is —O—, —C(R₁₀)₂—,

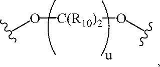

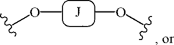

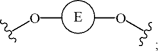

J is optionally substituted alkylene, optionally substituted heteroalkylene, optionally substituted alkenylene, optionally substituted heteroalkenylene, optionally substituted alkynylene, or optionally substituted heteroalkynylene;
E is optionally substituted carbocyclyl or optionally substituted heterocyclyl;
each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
s is an integer from 0 to 3 (e.g., 0, 1, 2, or 3);
t is an integer from 0 to 3 (e.g., 0, 1, 2, or 3); and
u is an integer from 1 to 8 (e.g., 1, 2, 3, 4, 5, 6, 7, or 8);
or a pharmaceutically acceptable salt thereof.

Exemplary vaccine adjuvants of the disclosure include compounds represented by formula (II)

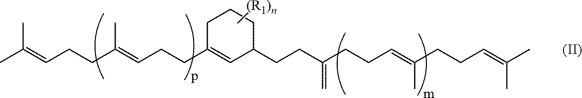

wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (II-a)

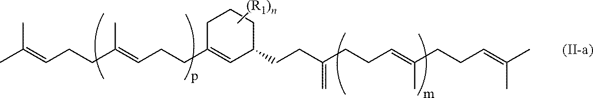

wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (II-b)

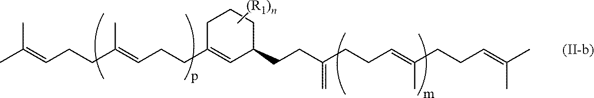

wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6 (e.g., 0, 1, 2, 3, 4, 5, or 6);
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (I-a), (II), (II-a), and (II-b), each R₁ is, independently, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of any one of compounds (I-a), (II), (II-a), and (II-b), each R₁ is, independently, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of any one of compounds (I-a), (II), (II-a), and (II-b), each R₁ is, independently,

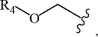

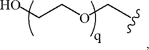

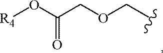

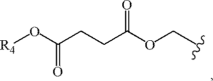

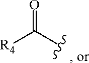

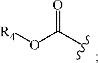

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of any one of compounds (I-a), (II), (II-a), and (II-b), each R₁ is, independently,

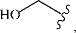

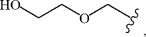

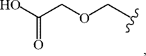

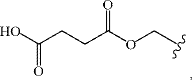

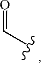

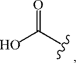

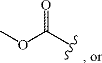

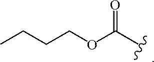

In some embodiments of any one of compounds (I-a), (II), (II-a), and (II-b), each R₁ is, independently,

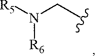

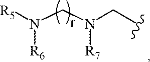

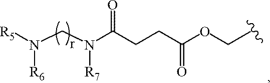

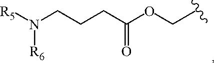

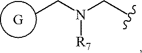

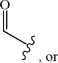

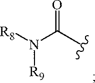

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of any one of compounds (I-a), (II), (II-a), and (II-b), n is 1 or 2.

In some embodiments of any one of compounds (I-a), (II), (II-a), and (II-b), m is 1.

In some embodiments of any one of compounds (I-a), (II), (II-a), and (II-b), p is 1.

Exemplary vaccine adjuvants of the disclosure include compounds represented by formula (III)

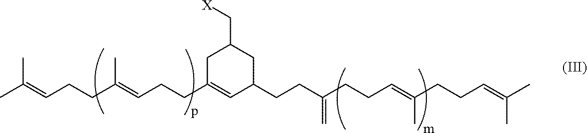

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (III-a)

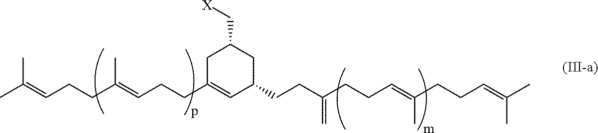

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (III-b)

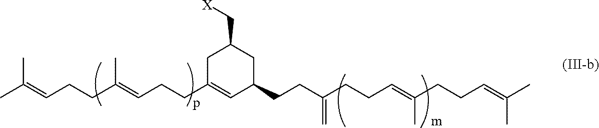

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (III-c)

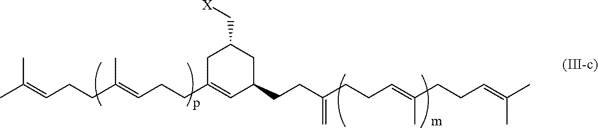

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (III-d)

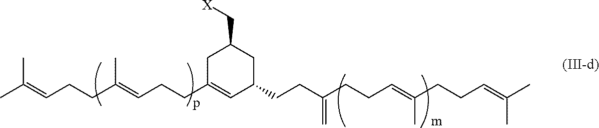

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IV)

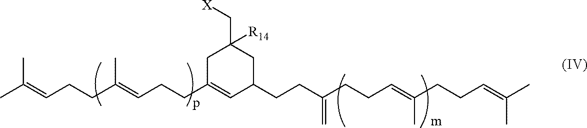

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IV-a)

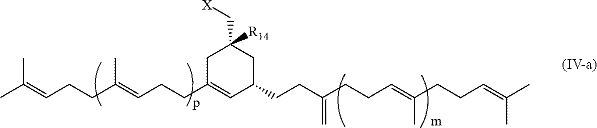

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IV-b)

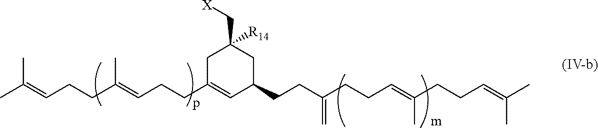

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IV-c)

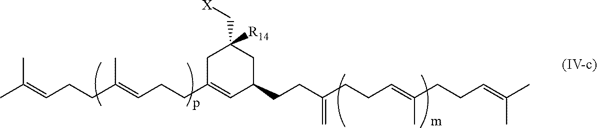

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IV-d)

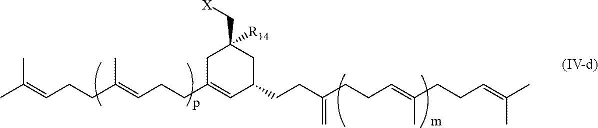

wherein X is optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (V)

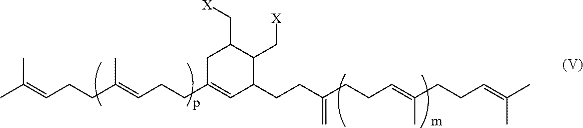

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (V-a)

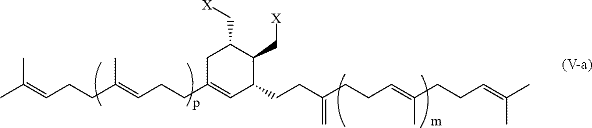

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (V-b)

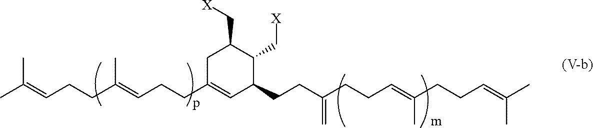

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (V-c)

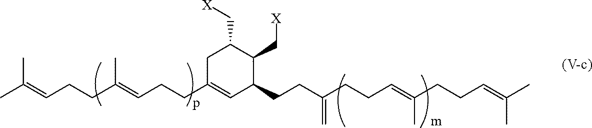

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (V-d)

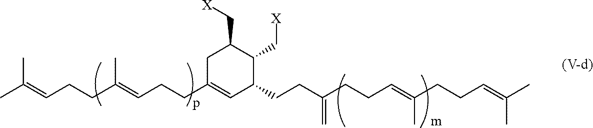

wherein each X is, independently, optionally substituted aminoalkyl, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (III), (III-a), (III-b), (III-c), (III-d), (IV), (IV-a), (IV-b), (IV-c), (IV-d), (V), (V-a), (V-b), (V-c), and (V-d), each X is, independently,

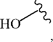

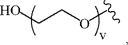

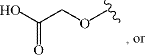

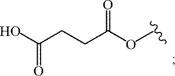

wherein v is an integer from 1 to 15 (e.g., 1, 2, 3, 4, 5, 6, 7, 8, 9, 10, 11, 12, 13, 14, or 15).

In some embodiments of any one of compounds (III), (III-a), (III-b), (III-c), (III-d), (IV), (IV-a), (IV-b), (IV-c), (IV-d), (V), (V-a), (V-b), (V-c), and (V-d), each X is, independently,

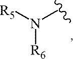

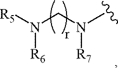

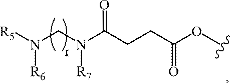

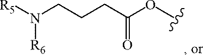

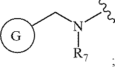

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments of any one of compounds (III), (III-a), (III-b), (III-c), (III-d), (IV), (IV-a), (IV-b), (IV-c), (IV-d), (V), (V-a), (V-b), (V-c), and (V-d), m is 1.

In some embodiments of any one of compounds (III), (III-a), (III-b), (III-c), (III-d), (IV), (IV-a), (IV-b), (IV-c), (IV-d), (V), (V-a), (V-b), (V-c), and (V-d), p is 1.

Exemplary vaccine adjuvants of the disclosure include compounds represented by formula (VI)

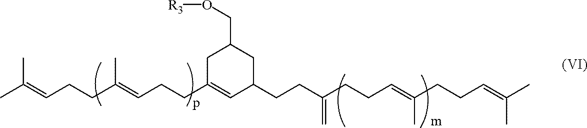

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VI-a)

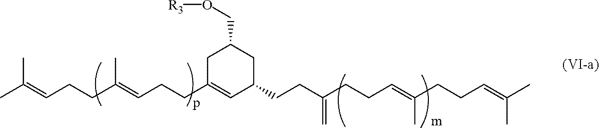

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VI-b)

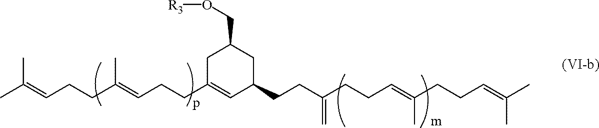

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VI-c)

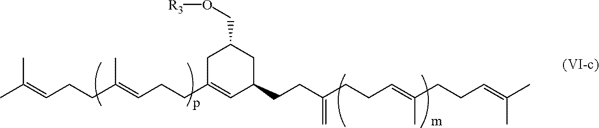

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VI-d)

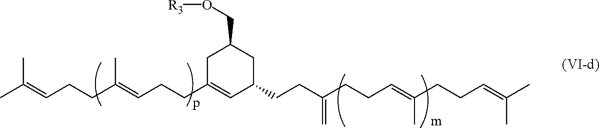

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VII)

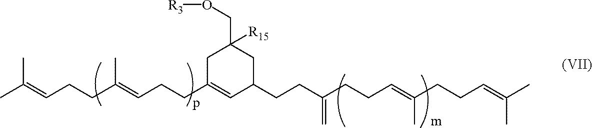

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VII-a)

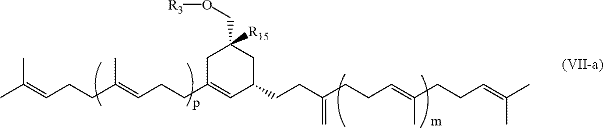

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VII-b)

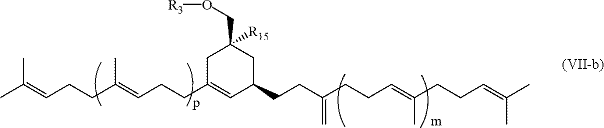

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VII-c)

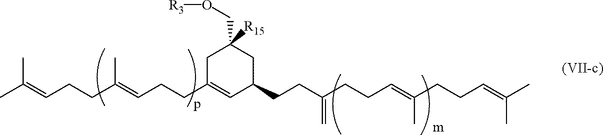

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VII-d)

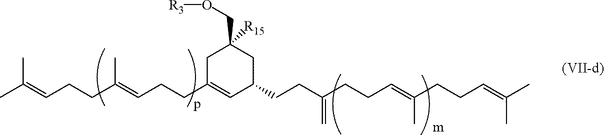

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VIII)

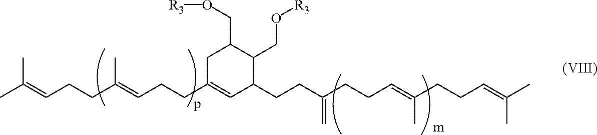

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VIII-a)

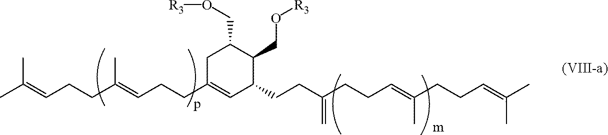

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VIII-b)

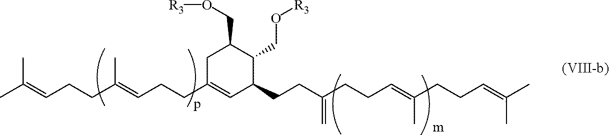

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VIII-c)

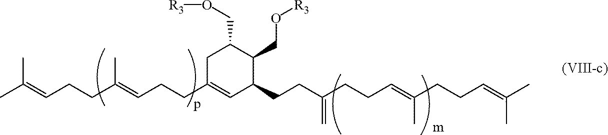

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (VIII-d)

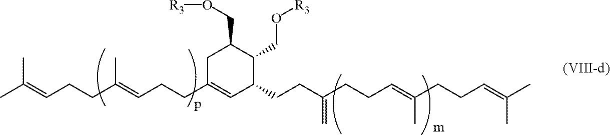

wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (VI), (VI-a), (VI-b), (VI-c), (VI-d), (VII), (VII-a), (VII-b), (VII-c), (VIII-d), (VIII), (VIII-a), (VIII-b), (VIII-c), and (VIII-d), each R₃ is, independently, hydrogen,

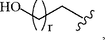

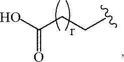

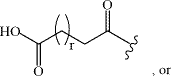

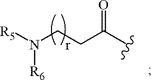

wherein each of R₅ and R₆ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 0 to 6 (e.g., 0, 1, 2, 3, 4, 5, or 6).

In some embodiments of any one of compounds (VI), (VI-a), (VI-b), (VI-c), (VI-d), (VII), (VII-a), (VII-b), (VII-c), (VIII-d), (VIII), (VIII-a), (VIII-b), (VIII-c), and (VIII-d), wherein m is 1.

In some embodiments of any one of compounds (VI), (VI-a), (VI-b), (VI-c), (VI-d), (VII), (VII-a), (VII-b), (VII-c), (VIII-d), (VIII), (VIII-a), (VIII-b), (VIII-c), and (VIII-d), p is 1.

Exemplary vaccine adjuvants of the disclosure include compounds represented by formula (IX)

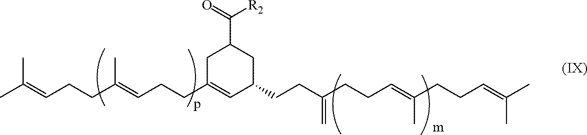

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IX-a)

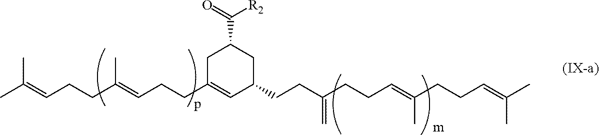

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IX-b)

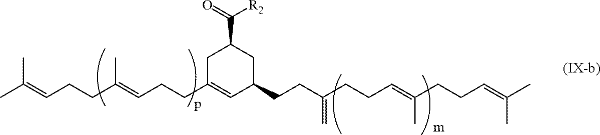

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IX-c)

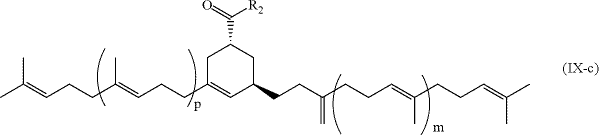

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (IX-d)

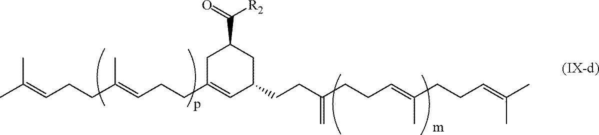

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (X)

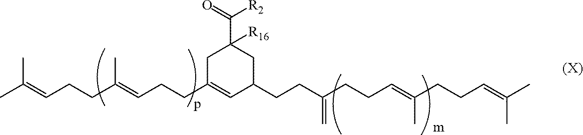

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (X-a)

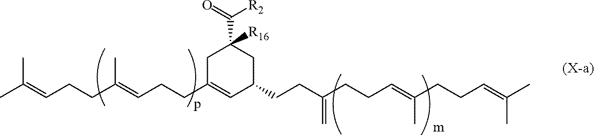

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (X-b)

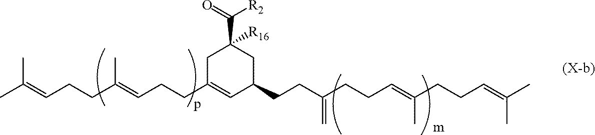

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (X-c)

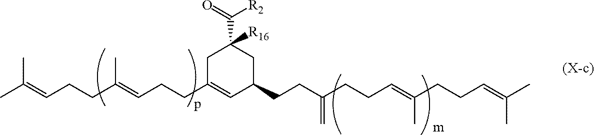

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (X-d)

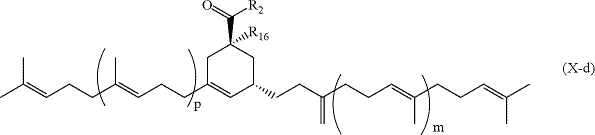

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (XI)

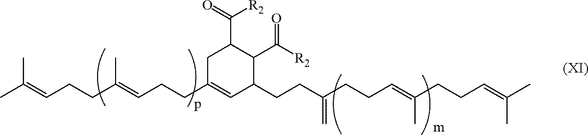

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (XI-a)

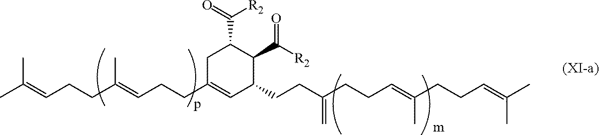

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (XI-b)

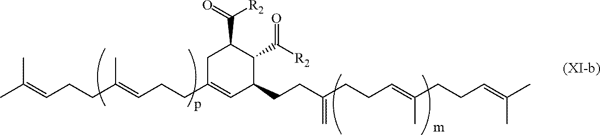

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (XI-c)

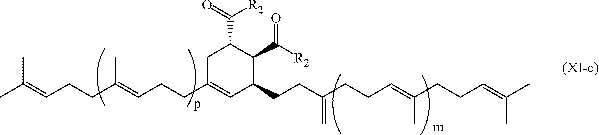

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

Exemplary vaccine adjuvants of the disclosure include compounds represented by formula (XI-d)

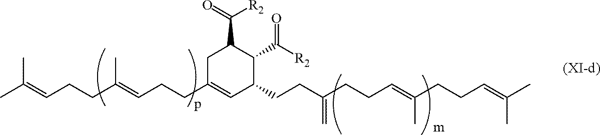

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d), each R₂ is, independently, hydrogen, hydroxyl,

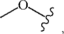

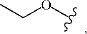

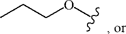

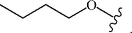

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d), each R₂ is, independently,

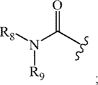

wherein each of R₈ and R₉ is, independently, hydrogen, optionally substituted alkyl, optionally substituted alkenyl, optionally substituted alkynyl, or optionally substituted aminoalkyl.

In some embodiments, each of R₈ and R₉ is, independently, alkylaminoalkyl or heterocyclyl alkyl.

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d), m is 1.

In some embodiments of any one of compounds (IX), (IX-a), (IX-b), (IX-c), (IX-d), (X), (X-a), (X-b), (X-c), (X-d), (XI), (XI-a), (XI-b), (XI-c), and (XI-d), p is 1.

Exemplary vaccine adjuvants of the disclosure include compounds represented by formula (XII)

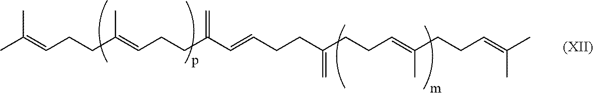

wherein m is an integer from 0 to 3 (e.g., 0, 1, 2, or 3); and
p is an integer from 0 to 3 (e.g., 0, 1, 2, or 3).

In some embodiments of compound (XII), m is 1.

In some embodiments of compound (XII), p is 1.

Exemplary vaccine adjuvants of the disclosure include compounds represented by formula (XIII)

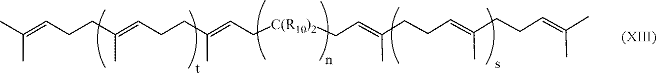

wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
n is an integer from 1 to 6 (e.g., 1, 2, 3, 4, 5, or 6);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XIII), each R₁₀ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of compound (XIII), each R₁₀ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of compound (XIII), each R₁₀ is, independently, hydrogen,

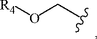

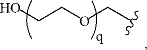

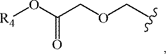

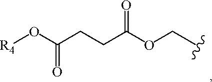

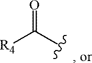

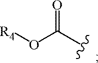

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of compound (XIII), each R₁₀ is, independently, hydrogen,

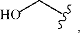

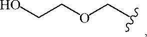

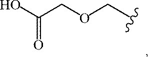

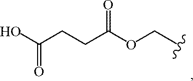

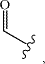

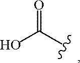

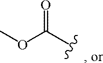

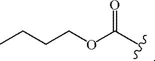

In some embodiments of compound (XIII), each R₁₀ is, independently, hydrogen,

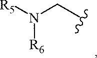

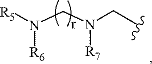

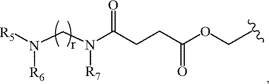

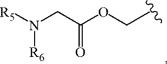

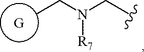

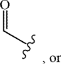

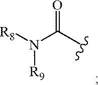

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of compound (XIII), n is 1 or 2.

In some embodiments of compound (XIII), s is 1.

In some embodiments of compound (XIII), t is 1.

Exemplary vaccine adjuvants of the disclosure include compounds represented by formula (XIV)

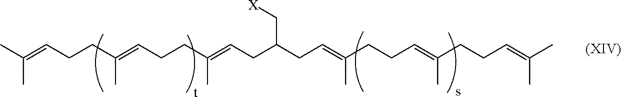

wherein X is optionally substituted alkoxy, optionally substituted acyloxy, hydroxyl, optionally substituted amino, optionally substituted sulfonyloxy, optionally substituted siloxy, carbonyl, or halogen;
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XIV), X is

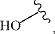

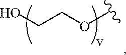

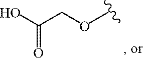

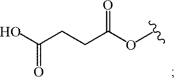

wherein v is an integer from 1 to 15 (e.g., 1, 2, 3, 4, 5, 6, 7, 8, 9, 10, 11, 12, 13, 14, or 15).

In some embodiments of compound (XIV), X is

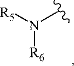

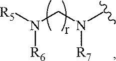

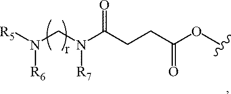

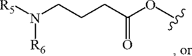

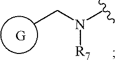

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments of compound (XIV), s is 1.

In some embodiments of compound (XIV), t is 1.

Exemplary vaccine adjuvants of the disclosure include compounds represented by formula (XV)

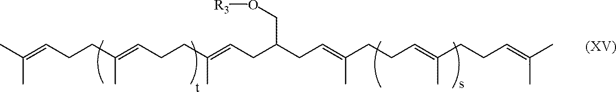

wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XV), R₃ is hydrogen,

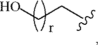

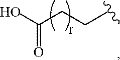

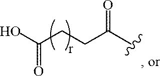

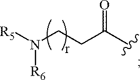

wherein each of R₅ and R₆ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 0 to 6 (e.g., 0, 1, 2, 3, 4, 5, or 6).

In some embodiments of compound (XV), s is 1.

In some embodiments of compound (XV), t is 1.

Exemplary vaccine adjuvants of the disclosure include compounds represented by formula (XVI)

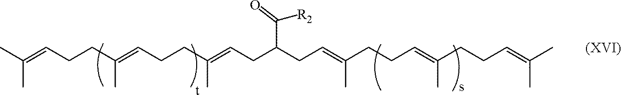

wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
s is an integer from 0 to 3 (e.g., 0, 1, 2, or 3); and
t is an integer from 0 to 3 (e.g., 0, 1, 2, or 3);
or a pharmaceutically acceptable salt thereof.

Exemplary vaccine adjuvants of the disclosure include compounds represented by formula (XVII)

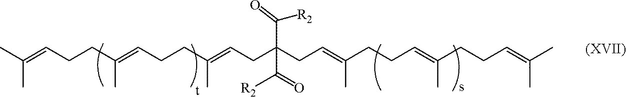

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XVI) or (XVII), each R₂ is, independently, hydrogen, hydroxyl,

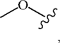

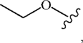

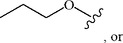

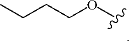

In some embodiments of compound (XVI) or (XVII), s is 1.

In some embodiments of compound (XVI) or (XVII), t is 1.

Exemplary vaccine adjuvants of the disclosure include compounds represented by formula (XVIII)

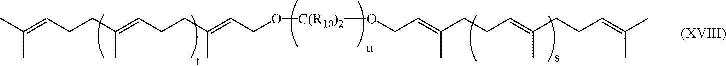

wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
u is an integer from 1 to 8 (e.g., 1, 2, 3, 4, 5, 6, 7, or 8);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of compound (XVIII), each R₁₀ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of compound (XVIII), each R₁₀ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of compound (XVIII), each R₁₀ is, independently, hydrogen,

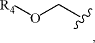

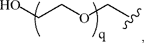

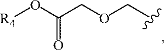

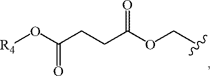

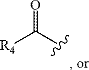

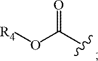

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of compound (XVIII), each R₁₀ is, independently, hydrogen,

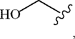

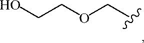

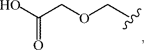

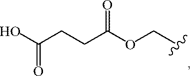

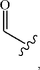

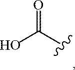

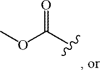

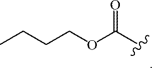

In some embodiments of compound (XVIII), each R₁₀ is, independently, hydrogen,

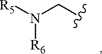

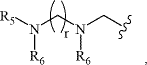

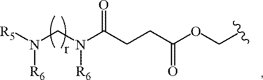

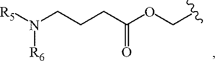

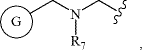

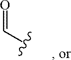

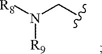

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of compound (XVIII), u is 1 or 2.

In some embodiments of compound (XVIII), s is 1.

In some embodiments of compound (XVIII), t is 1.

Exemplary vaccine adjuvants of the disclosure include compounds represented by formula (XIX)

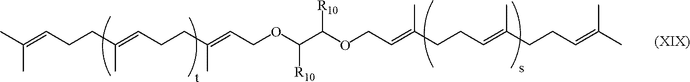

wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, or optionally substituted heteroalkynyl;
s is an integer from 0 to 3 (e.g., 0, 1, 2, or 3); and
t is an integer from 0 to 3 (e.g., 0, 1, 2, or 3).

Exemplary vaccine adjuvants of the disclosure include compounds represented by formula (XX)

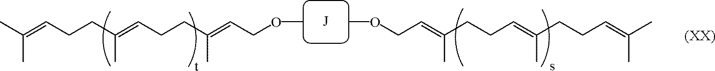

wherein J is optionally substituted alkylene, optionally substituted heteroalkylene, optionally substituted alkenylene, optionally substituted heteroalkenylene, optionally substituted alkynylene, or optionally substituted heteroalkynylene.

In some embodiments of compound (XX), J is optionally substituted alkenylene or optionally substituted heteroalkenylene.

Exemplary vaccine adjuvants of the disclosure include compounds represented by formula (XXI)

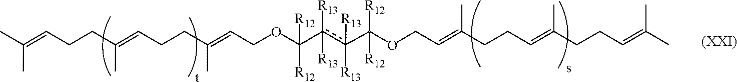

wherein each of R₁₂ and R₁₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
wherein each

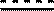

is, independently, a single bond or a double bond;
wherein if

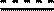

is a double bond, only one R₁₃ is present at each carbon of the double bond;
s is an integer from 0 to 3 (e.g., 0, 1, 2, or 3); and
t is an integer from 0 to 3 (e.g., 0, 1, 2, or 3).

Exemplary vaccine adjuvants of the disclosure include compounds represented by formula (XXII)

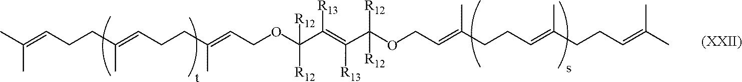

wherein each of R₁₂ and R₁₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
wherein s is an integer from 0 to 3 (e.g., 0, 1, 2, or 3); and
t is an integer from 0 to 3 (e.g., 0, 1, 2, or 3).

Exemplary vaccine adjuvants of the disclosure include compounds represented by formula (XXIII)

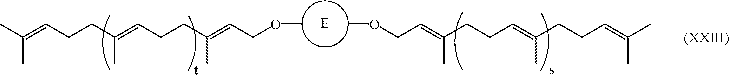

wherein E is optionally substituted cycloalkyl, optionally substituted heterocyclyl, optionally substituted aryl, or optionally substituted heteroaryl.

In some embodiments of compound (XXIII), E is

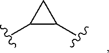

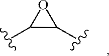

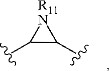

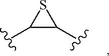

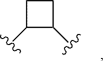

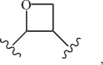

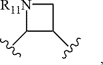

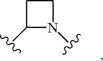

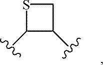

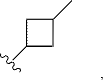

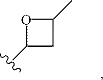

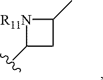

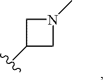

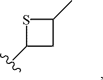

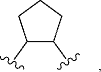

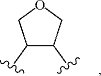

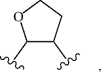

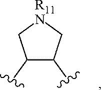

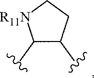

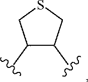

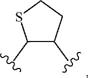

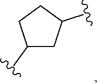

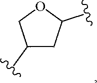

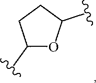

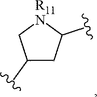

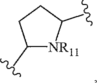

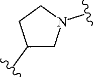

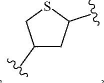

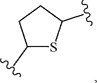

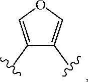

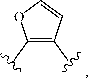

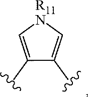

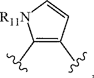

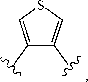

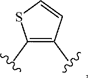

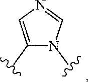

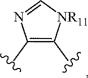

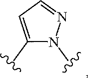

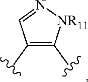

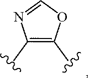

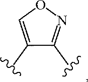

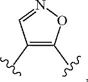

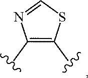

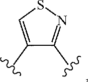

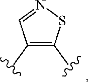

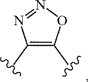

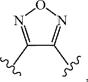

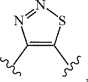

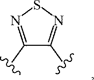

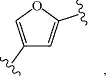

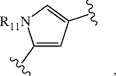

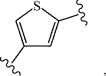

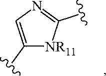

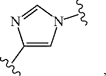

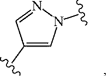

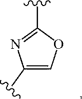

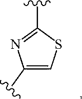

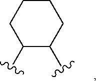

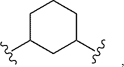

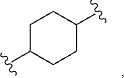

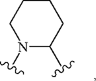

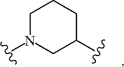

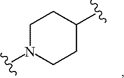

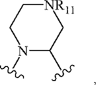

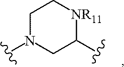

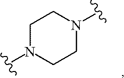

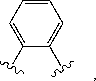

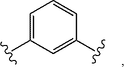

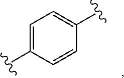

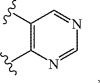

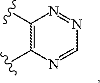

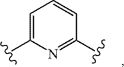

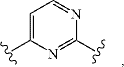

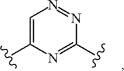

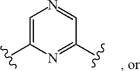

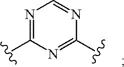

wherein each R₁₁ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, or carbonyl;
and wherein each ring of E may be optionally substituted, as the valency of each ring permits.

In some embodiments of compound (XXIII), E is

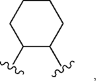

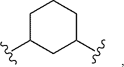

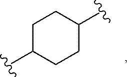

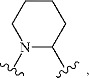

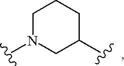

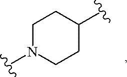

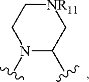

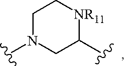

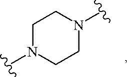

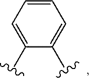

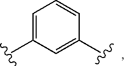

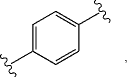

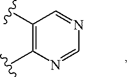

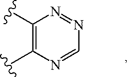

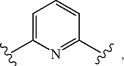

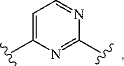

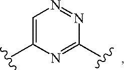

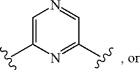

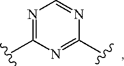

In some embodiments of compound (XXIII), s is 1.

In some embodiments of compound (XXIII), t is 1.

Exemplary vaccine adjuvants of the disclosure include compounds represented by formula (XXIV)

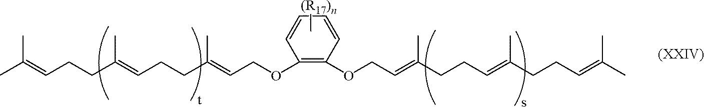

wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4 (e.g., 0, 1, 2, 3, or 4);
s is an integer from 0 to 3 (e.g., 0, 1, 2, or 3); and
t is an integer from 0 to 3 (e.g., 0, 1, 2, or 3);
or a pharmaceutically acceptable salt thereof.

Exemplary vaccine adjuvants of the disclosure include compounds represented by formula (XXV)

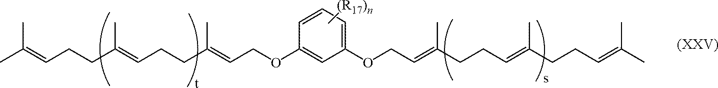

wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4 (e.g., 0, 1, 2, 3, or 4);
s is an integer from 0 to 3 (e.g., 0, 1, 2, or 3); and
t is an integer from 0 to 3 (e.g., 0, 1, 2, or 3);
or a pharmaceutically acceptable salt thereof.

Exemplary vaccine adjuvants of the disclosure include compounds represented by formula (XXVI)

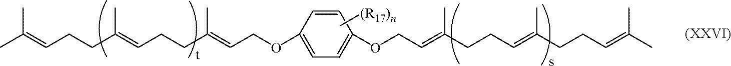

wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4 (e.g., 0, 1, 2, 3, or 4);
s is an integer from 0 to 3 (e.g., 0, 1, 2, or 3); and
t is an integer from 0 to 3 (e.g., 0, 1, 2, or 3);
or a pharmaceutically acceptable salt thereof.

In some embodiments of any one of compounds (XXIV), (XXV), or (XXVI), each R₁₇ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

In some embodiments of any one of compounds (XXIV), (XXV), or (XXVI), each R₁₇ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

In some embodiments of any one of compounds (XXIV), (XXV), or (XXVI), each R₁₇ is, independently, hydrogen,

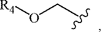

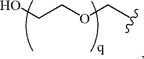

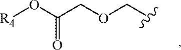

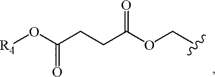

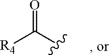

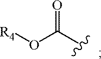

wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20 (e.g., 1, 2, 3, 4, 5, 6, 7, 8, 9, 10, 11, 12, 13, 14, 15, 16, 17, 18, 19, or 20).

In some embodiments of any one of compounds (XXIV), (XXV), or (XXVI), each R₁₇ is, independently, hydrogen,

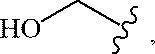

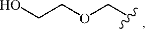

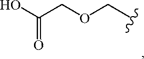

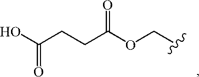

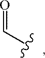

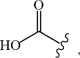

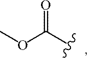

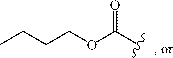

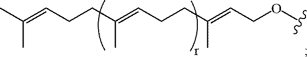

and wherein each r is an integer from 0 to 3 (e.g., 0, 1, 2, or 3).

In some embodiments of any one of compounds (XXIV), (XXV), or (XXVI), each R₁₇ is, independently, hydrogen,

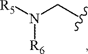

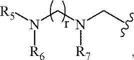

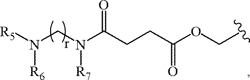

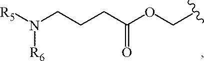

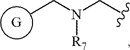

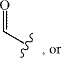

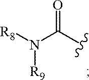

wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6 (e.g., 1, 2, 3, 4, 5, or 6).

In some embodiments, G is optionally substituted piperidinyl, optionally substituted morpholinyl, optionally substituted piperazinyl, optionally substituted thiomorpholinyl, optionally substituted furyl, optionally substituted pyranyl, optionally substituted pyrrolidinyl, optionally substituted tetrahydropyranyl, optionally substituted tetrahydrofuranyl, or optionally substituted 1,3-dioxanyl.

In some embodiments, G is optionally substituted piperidinyl, optionally substituted morpholinyl, or optionally substituted piperazinyl.

In some embodiments, each of R₈ and R₉ is, independently, alkylaminoalkyl or heterocyclyl alkyl.

In some embodiments of any one of compounds (XXIV), (XXV), or (XXVI), n is 1 or 2.

In some embodiments of any one of compounds (XXIV), (XXV), or (XXVI), s is 1.

In some embodiments of any one of compounds (XXIV), (XXV), or (XXVI), t is 1.

Exemplary vaccine adjuvants of the disclosure are further provided in the following table.

TABLE 1

Exemplary Vaccine Adjuvants of the Disclosure

| Compound No. | Chemical Structure |
|---|---|
| 1 | 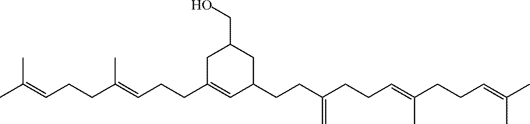 [SMILES: C=C(CC/C=C(\C)CCC=C(C)C)CCC1C=C(CC/C=C(\C)CCC=C(C)C)CC(CO)C1] |
| 2 | 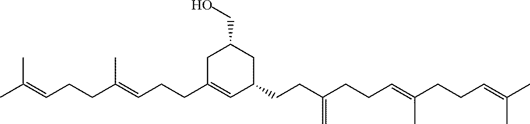 [SMILES: C=C(CC/C=C(\C)CCC=C(C)C)CC[C@@H]1C=C(CC/C=C(\C)CCC=C(C)C)C[C@H](CO)C1] |
| 3 | 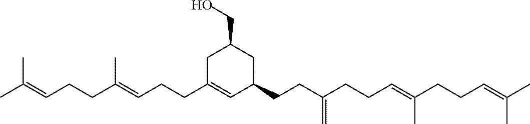 [SMILES: C=C(CC/C=C(\C)CCC=C(C)C)CC[C@H]1C=C(CC/C=C(\C)CCC=C(C)C)C[C@@H](CO)C1] |
| 4 | 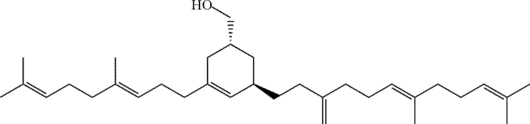 [SMILES: C=C(CC/C=C(\C)CCC=C(C)C)CC[C@H]1C=C(CC/C=C(\C)CCC=C(C)C)C[C@H](CO)C1] |
| 5 | 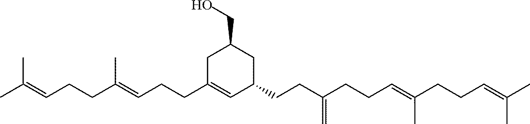 [SMILES: C=C(CC/C=C(\C)CCC=C(C)C)CC[C@@H]1C=C(CC/C=C(\C)CCC=C(C)C)C[C@@H](CO)C1] |
| 6 | 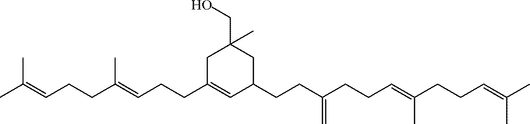 [SMILES: C=C(CC/C=C(\C)CCC=C(C)C)CCC1C=C(CC/C=C(\C)CCC=C(C)C)CC(C)(CO)C1] |
| 7 | 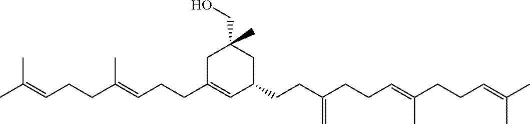 [SMILES: C=C(CC/C=C(\C)CCC=C(C)C)CC[C@@H]1C=C(CC/C=C(\C)CCC=C(C)C)C[C@](C)(CO)C1] |
| 8 | 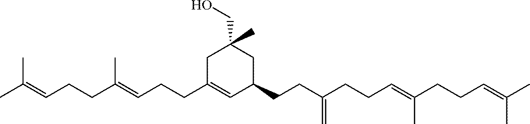 [SMILES: C=C(CC/C=C(\C)CCC=C(C)C)CC[C@H]1C=C(CC/C=C(\C)CCC=C(C)C)C[C@](C)(CO)C1] |
| 9 | 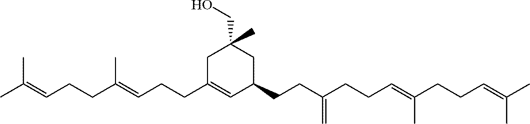 [SMILES: C=C(CC/C=C(\C)CCC=C(C)C)CC[C@H]1C=C(CC/C=C(\C)CCC=C(C)C)C[C@](C)(CO)C1] |
| 10 | 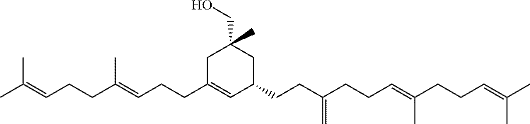 [SMILES: C=C(CC/C=C(\C)CCC=C(C)C)CC[C@@H]1C=C(CC/C=C(\C)CCC=C(C)C)C[C@](C)(CO)C1] |
| 11 | 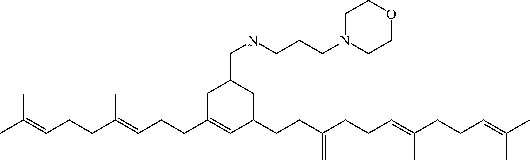 [SMILES: C=C(CC/C=C(\C)CCC=C(C)C)CCC1C=C(CC/C=C(\C)CCC=C(C)C)CC(CNCCCN2CCOCC2)C1] |
| 12 | 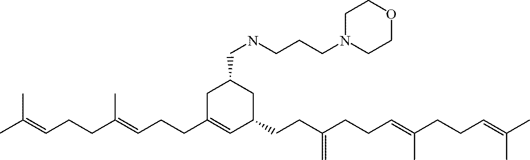 [SMILES: C=C(CC/C=C(\C)CCC=C(C)C)CC[C@@H]1C=C(CC/C=C(\C)CCC=C(C)C)C[C@H](CNCCCN2CCOCC2)C1] |
| 13 | 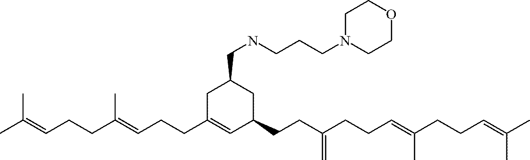 [SMILES: C=C(CC/C=C(\C)CCC=C(C)C)CC[C@H]1C=C(CC/C=C(\C)CCC=C(C)C)C[C@@H](CNCCCN2CCOCC2)C1] |
| 14 | 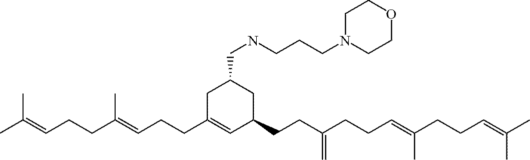 [SMILES: C=C(CC/C=C(\C)CCC=C(C)C)CC[C@H]1C=C(CC/C=C(\C)CCC=C(C)C)C[C@H](CNCCCN2CCOCC2)C1] |
| 15 | 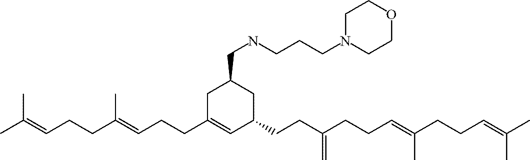 [SMILES: C=C(CC/C=C(\C)CCC=C(C)C)CC[C@@H]1C=C(CC/C=C(\C)CCC=C(C)C)C[C@@H](CNCCCN2CCOCC2)C1] |
| 16 | 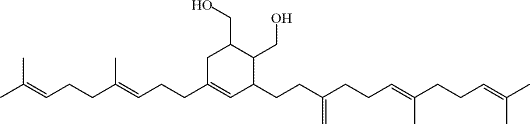 [SMILES: C=C(CC/C=C(\C)CCC=C(C)C)CCC1C=C(CC/C=C(\C)CCC=C(C)C)CC(CO)C1CO] |
| 17 | 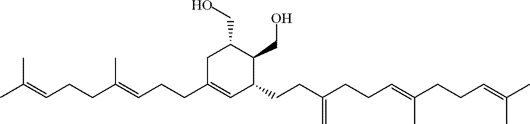 [SMILES: C=C(CC/C=C(\C)CCC=C(C)C)CC[C@@H]1C=C(CC/C=C(\C)CCC=C(C)C)C[C@H](CO)[C@H]1CO] |
| 18 | 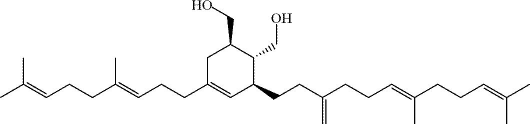 [SMILES: C=C(CC/C=C(\C)CCC=C(C)C)CC[C@H]1C=C(CC/C=C(\C)CCC=C(C)C)C[C@@H](CO)[C@@H]1CO] |
| 19 | 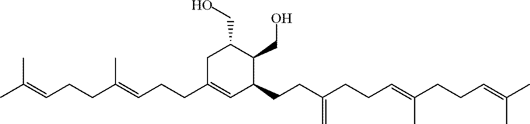 [SMILES: C=C(CC/C=C(\C)CCC=C(C)C)CC[C@H]1C=C(CC/C=C(\C)CCC=C(C)C)C[C@H](CO)[C@H]1CO] |
| 20 | 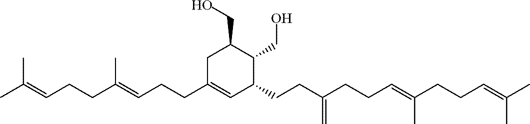 [SMILES: C=C(CC/C=C(\C)CCC=C(C)C)CC[C@@H]1C=C(CC/C=C(\C)CCC=C(C)C)C[C@@H](CO)[C@@H]1CO] |
| 21 | 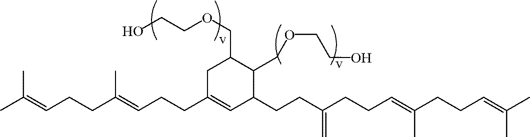 wherein each v is an integer from 1 to 15 (e.g., 0, 1, 2, 3, 4, 5, 6, 7, 8, 9, 10, 11, 12, 13, 14, or 15) |
| 22 | 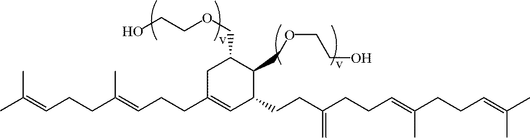 wherein each v is an integer from 1 to 15 (e.g., 0, 1, 2, 3, 4, 5, 6, 7, 8, 9, 10, 11, 12, 13, 14, or 15) |
| 23 | 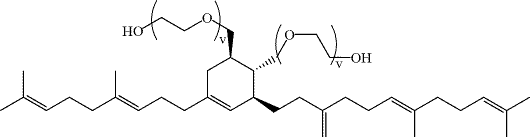 wherein each v is an integer from 1 to 15 (e.g., 0, 1, 2, 3, 4, 5, 6, 7, 8, 9, 10, 11, 12, 13, 14, or 15) |
| 24 | 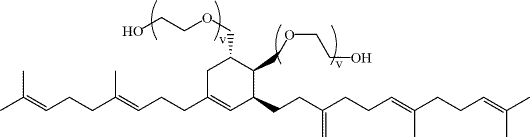 wherein each v is an integer from 1 to 15 (e.g., 0, 1, 2, 3, 4, 5, 6, 7, 8, 9, 10, 11, 12, 13, 14, or 15) |
| 25 | 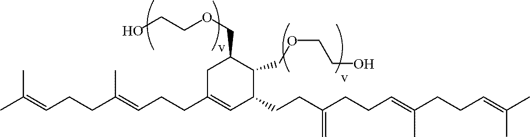 wherein each v is an integer from 1 to 15 (e.g., 0, 1, 2, 3, 4, 5, 6, 7, 8, 9, 10, 11, 12, 13, 14, or 15) |
| 26 | 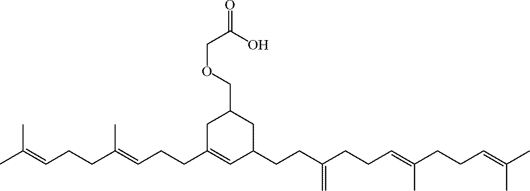 [SMILES: C=C(CC/C=C(\C)CCC=C(C)C)CCC1C=C(CC/C=C(\C)CCC=C(C)C)CC(COCC(=O)O)C1] |
| 27 | 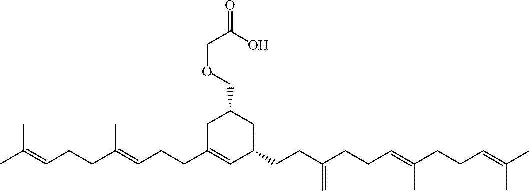 [SMILES: C=C(CC/C=C(\C)CCC=C(C)C)CC[C@@H]1C=C(CC/C=C(\C)CCC=C(C)C)C[C@H](COCC(=O)O)C1] |
| 28 | 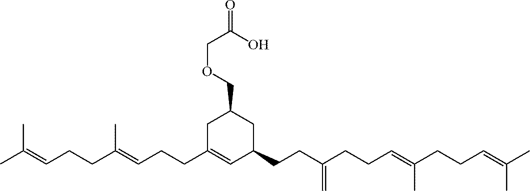 [SMILES: C=C(CC/C=C(\C)CCC=C(C)C)CC[C@H]1C=C(CC/C=C(\C)CCC=C(C)C)C[C@@H](COCC(=O)O)C1] |
| 29 | 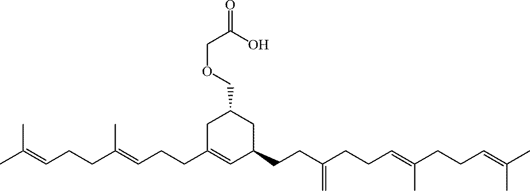 [SMILES: C=C(CC/C=C(\C)CCC=C(C)C)CC[C@H]1C=C(CC/C=C(\C)CCC=C(C)C)C[C@H](COCC(=O)O)C1] |
| 30 | 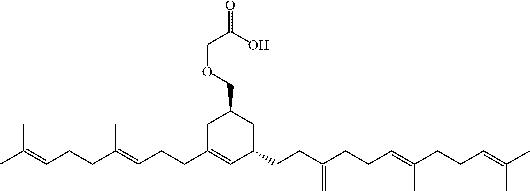 [SMILES: C=C(CC/C=C(\C)CCC=C(C)C)CC[C@@H]1C=C(CC/C=C(\C)CCC=C(C)C)C[C@@H](COCC(=O)O)C1] |
| 31 | 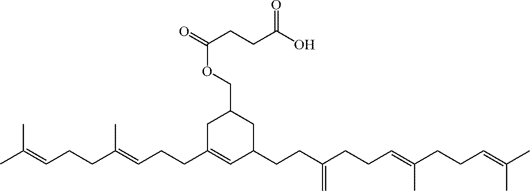 [SMILES: C=C(CC/C=C(\C)CCC=C(C)C)CCC1C=C(CC/C=C(\C)CCC=C(C)C)CC(COC(=O)CCC(=O)O)C1] |
| 32 | 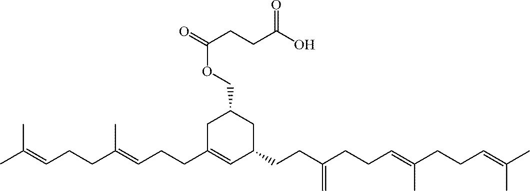 [SMILES: C=C(CC/C=C(\C)CCC=C(C)C)CC[C@@H]1C=C(CC/C=C(\C)CCC=C(C)C)C[C@H](COC(=O)CCC(=O)O)C1] |
| 33 | 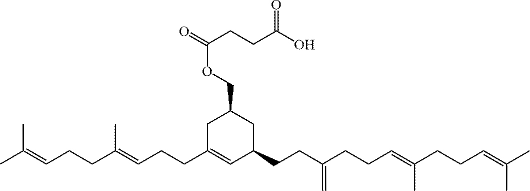 [SMILES: C=C(CC/C=C(\C)CCC=C(C)C)CC[C@H]1C=C(CC/C=C(\C)CCC=C(C)C)C[C@@H](COC(=O)CCC(=O)O)C1] |
| 34 | 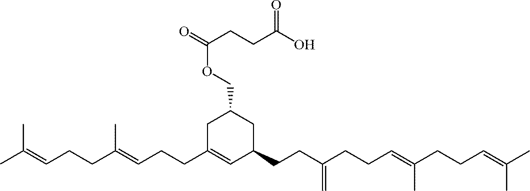 [SMILES: C=C(CC/C=C(\C)CCC=C(C)C)CC[C@H]1C=C(CC/C=C(\C)CCC=C(C)C)C[C@H](COC(=O)CCC(=O)O)C1] |
| 35 | 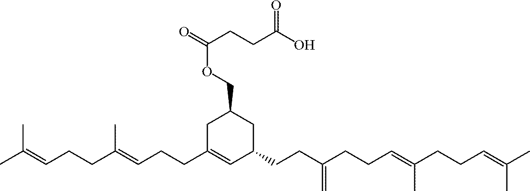 [SMILES: C=C(CC/C=C(\C)CCC=C(C)C)CC[C@@H]1C=C(CC/C=C(\C)CCC=C(C)C)C[C@@H](COC(=O)CCC(=O)O)C1] |
| 36 | 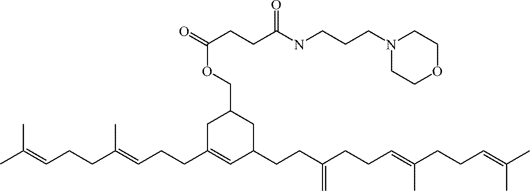 [SMILES: C=C(CC/C=C(\C)CCC=C(C)C)CCC1C=C(CC/C=C(\C)CCC=C(C)C)CC(COC(=O)CCC(=O)NCCCN2CCOCC2)C1] |
| 37 | 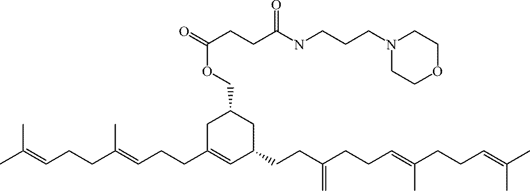 [SMILES: C=C(CC/C=C(\C)CCC=C(C)C)CC[C@@H]1C=C(CC/C=C(\C)CCC=C(C)C)C[C@H](COC(=O)CCC(=O)NCCCN2CCOCC2)C1] |
| 38 | 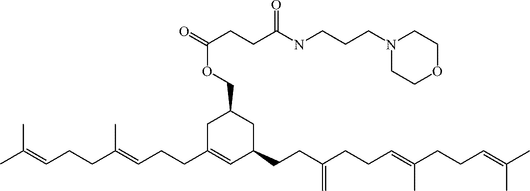 [SMILES: C=C(CC/C=C(\C)CCC=C(C)C)CC[C@H]1C=C(CC/C=C(\C)CCC=C(C)C)C[C@@H](COC(=O)CCC(=O)NCCCN2CCOCC2)C1] |
| 39 | 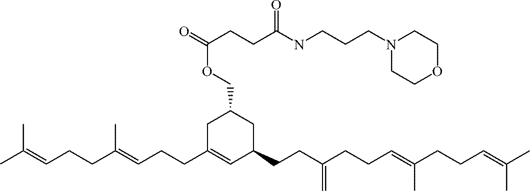 [SMILES: C=C(CC/C=C(\C)CCC=C(C)C)CC[C@H]1C=C(CC/C=C(\C)CCC=C(C)C)C[C@H](COC(=O)CCC(=O)NCCCN2CCOCC2)C1] |
| 40 | 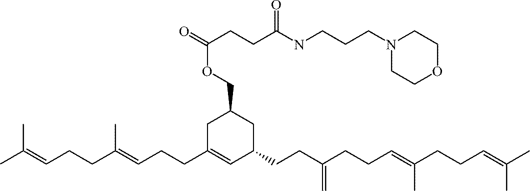 [SMILES: C=C(CC/C=C(\C)CCC=C(C)C)CC[C@@H]1C=C(CC/C=C(\C)CCC=C(C)C)C[C@@H](COC(=O)CCC(=O)NCCCN2CCOCC2)C1] |
| 41 | 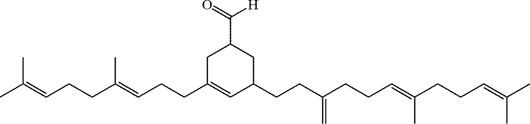 [SMILES: C=C(CC/C=C(\C)CCC=C(C)C)CCC1C=C(CC/C=C(\C)CCC=C(C)C)CC(C=O)C1] |
| 42 | 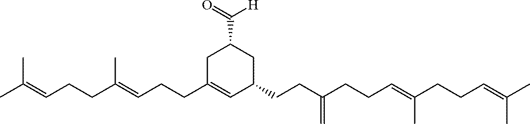 [SMILES: C=C(CC/C=C(\C)CCC=C(C)C)CC[C@@H]1C=C(CC/C=C(\C)CCC=C(C)C)C[C@H](C=O)C1] |
| 43 | 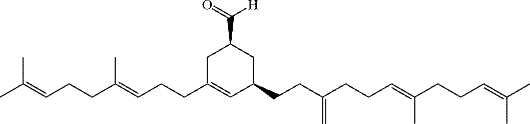 [SMILES: C=C(CC/C=C(\C)CCC=C(C)C)CC[C@H]1C=C(CC/C=C(\C)CCC=C(C)C)C[C@@H](C=O)C1] |
| 44 | 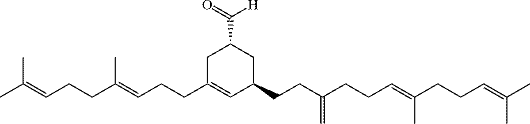 [SMILES: C=C(CC/C=C(\C)CCC=C(C)C)CC[C@H]1C=C(CC/C=C(\C)CCC=C(C)C)C[C@H](C=O)C1] |
| 45 | 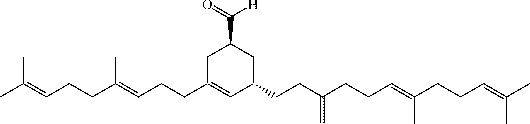 [SMILES: C=C(CC/C=C(\C)CCC=C(C)C)CC[C@@H]1C=C(CC/C=C(\C)CCC=C(C)C)C[C@@H](C=O)C1] |
| 46 | 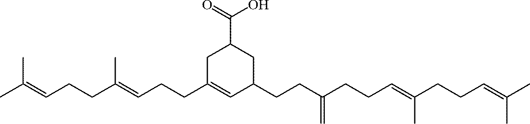 [SMILES: C=C(CC/C=C(\C)CCC=C(C)C)CCC1C=C(CC/C=C(\C)CCC=C(C)C)CC(C(=O)O)C1] |
| 47 | 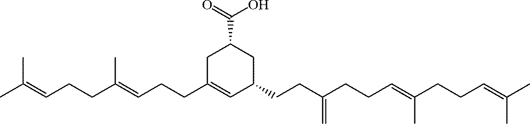 [SMILES: C=C(CC/C=C(\C)CCC=C(C)C)CC[C@@H]1C=C(CC/C=C(\C)CCC=C(C)C)C[C@H](C(=O)O)C1] |
| 48 | 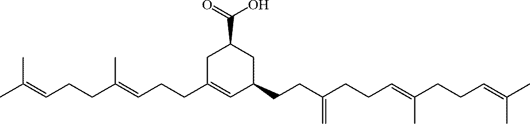 [SMILES: C=C(CC/C=C(\C)CCC=C(C)C)CC[C@H]1C=C(CC/C=C(\C)CCC=C(C)C)C[C@@H](C(=O)O)C1] |
| 49 | 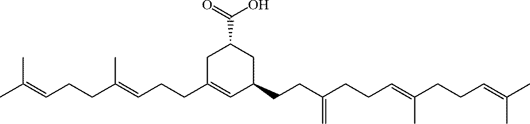 [SMILES: C=C(CC/C=C(\C)CCC=C(C)C)CC[C@H]1C=C(CC/C=C(\C)CCC=C(C)C)C[C@H](C(=O)O)C1] |
| 50 | 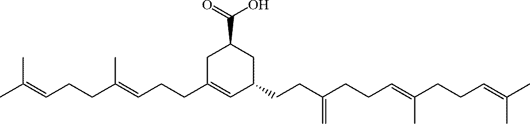 [SMILES: C=C(CC/C=C(\C)CCC=C(C)C)CC[C@@H]1C=C(CC/C=C(\C)CCC=C(C)C)C[C@@H](C(=O)O)C1] |
| 51 | 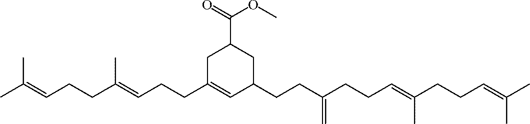 [SMILES: C=C(CC/C=C(\C)CCC=C(C)C)CCC1C=C(CC/C=C(\C)CCC=C(C)C)CC(C(=O)OC)C1] |
| 52 | 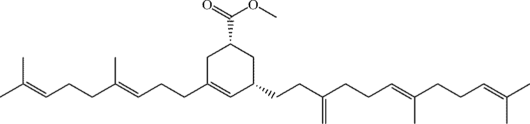 [SMILES: C=C(CC/C=C(\C)CCC=C(C)C)CC[C@@H]1C=C(CC/C=C(\C)CCC=C(C)C)C[C@H](C(=O)OC)C1] |
| 53 | 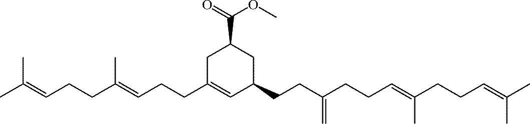 [SMILES: C=C(CC/C=C(\C)CCC=C(C)C)CC[C@H]1C=C(CC/C=C(\C)CCC=C(C)C)C[C@@H](C(=O)OC)C1] |
| 54 | 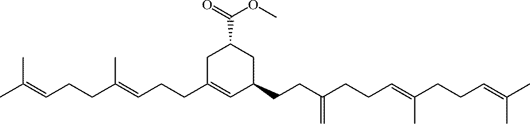 [SMILES: C=C(CC/C=C(\C)CCC=C(C)C)CC[C@H]1C=C(CC/C=C(\C)CCC=C(C)C)C[C@H](C(=O)OC)C1] |
| 55 | 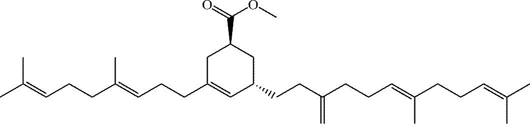 [SMILES: C=C(CC/C=C(\C)CCC=C(C)C)CC[C@@H]1C=C(CC/C=C(\C)CCC=C(C)C)C[C@@H](C(=O)OC)C1] |
| 56 | 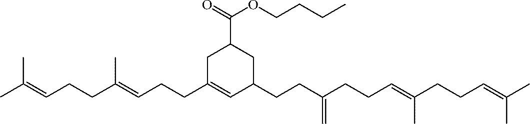 [SMILES: C=C(CC/C=C(\C)CCC=C(C)C)CCC1C=C(CC/C=C(\C)CCC=C(C)C)CC(C(=O)OCCCC)C1] |
| 57 | 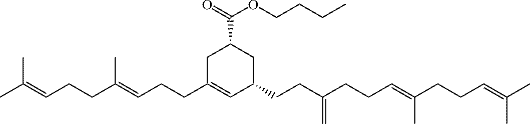 [SMILES: C=C(CC/C=C(\C)CCC=C(C)C)CC[C@@H]1C=C(CC/C=C(\C)CCC=C(C)C)C[C@H](C(=O)OCCCC)C1] |
| 58 | 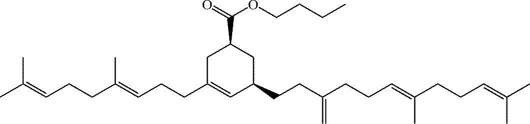 [SMILES: C=C(CC/C=C(\C)CCC=C(C)C)CC[C@H]1C=C(CC/C=C(\C)CCC=C(C)C)C[C@@H](C(=O)OCCCC)C1] |
| 59 | 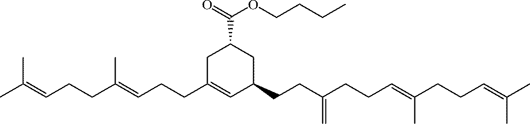 [SMILES: C=C(CC/C=C(\C)CCC=C(C)C)CC[C@H]1C=C(CC/C=C(\C)CCC=C(C)C)C[C@H](C(=O)OCCCC)C1] |
| 60 | 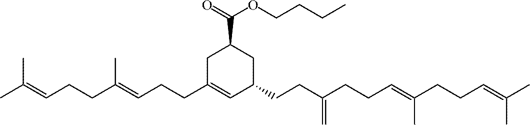 [SMILES: C=C(CC/C=C(\C)CCC=C(C)C)CC[C@@H]1C=C(CC/C=C(\C)CCC=C(C)C)C[C@@H](C(=O)OCCCC)C1] |
| 61 | 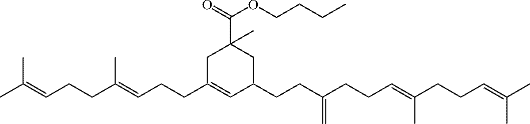 [SMILES: C=C(CC/C=C(\C)CCC=C(C)C)CCC1C=C(CC/C=C(\C)CCC=C(C)C)CC(C)(C(=O)OCCCC)C1] |
| 62 | 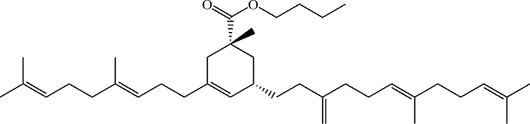 [SMILES: C=C(CC/C=C(\C)CCC=C(C)C)CC[C@@H]1C=C(CC/C=C(\C)CCC=C(C)C)C[C@](C)(C(=O)OCCCC)C1] |
| 63 | 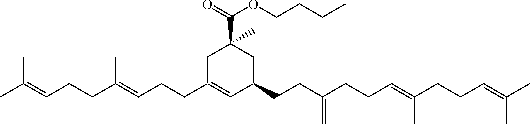 [SMILES: C=C(CC/C=C(\C)CCC=C(C)C)CC[C@H]1C=C(CC/C=C(\C)CCC=C(C)C)C[C@@](C)(C(=O)OCCCC)C1] |
| 64 | 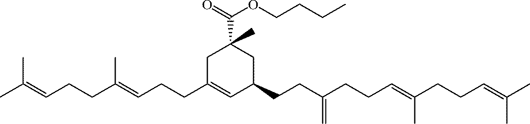 [SMILES: C=C(CC/C=C(\C)CCC=C(C)C)CC[C@H]1C=C(CC/C=C(\C)CCC=C(C)C)C[C@](C)(C(=O)OCCCC)C1] |
| 65 | 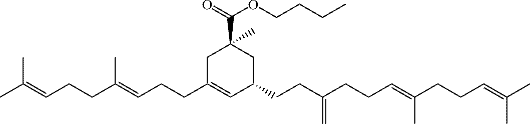 [SMILES: C=C(CC/C=C(\C)CCC=C(C)C)CC[C@@H]1C=C(CC/C=C(\C)CCC=C(C)C)C[C@@](C)(C(=O)OCCCC)C1] |
| 66 | 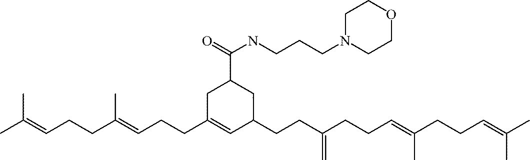 [SMILES: C=C(CC/C=C(\C)CCC=C(C)C)CCC1C=C(CC/C=C(\C)CCC=C(C)C)CC(C(=O)NCCCN2CCOCC2)C1] |
| 67 | 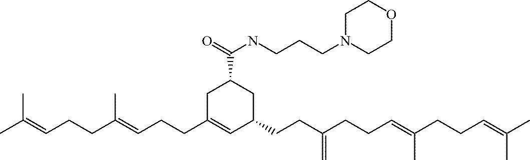 [SMILES: C=C(CC/C=C(\C)CCC=C(C)C)CC[C@@H]1C=C(CC/C=C(\C)CCC=C(C)C)C[C@H](C(=O)NCCCN2CCOCC2)C1] |
| 68 | 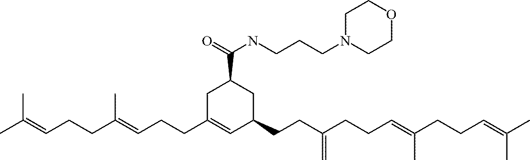 [SMILES: C=C(CC/C=C(\C)CCC=C(C)C)CC[C@H]1C=C(CC/C=C(\C)CCC=C(C)C)C[C@@H](C(=O)NCCCN2CCOCC2)C1] |
| 69 | 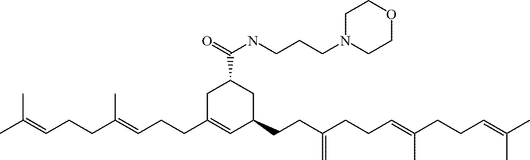 [SMILES: C=C(CC/C=C(\C)CCC=C(C)C)CC[C@H]1C=C(CC/C=C(\C)CCC=C(C)C)C[C@H](C(=O)NCCCN2CCOCC2)C1] |
| 70 | 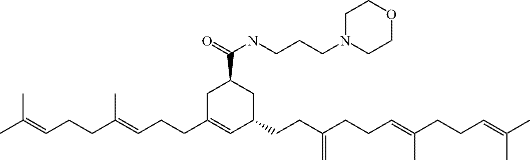 [SMILES: C=C(CC/C=C(\C)CCC=C(C)C)CC[C@@H]1C=C(CC/C=C(\C)CCC=C(C)C)C[C@@H](C(=O)NCCCN2CCOCC2)C1] |
| 71 | 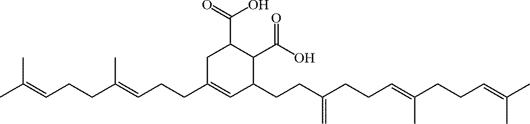 [SMILES: C=C(CC/C=C(\C)CCC=C(C)C)CCC1C=C(CC/C=C(\C)CCC=C(C)C)CC(C(=O)O)C1C(=O)O] |
| 72 | 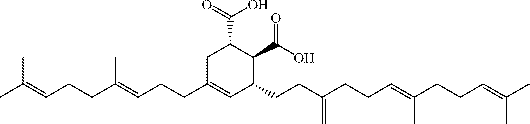 [SMILES: C=C(CC/C=C(\C)CCC=C(C)C)CC[C@@H]1C=C(CC/C=C(\C)CCC=C(C)C)C[C@H](C(=O)O)[C@H]1C(=O)O] |
| 73 | 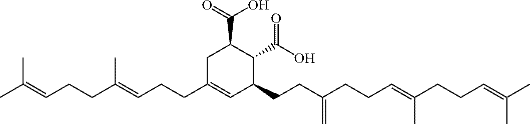 [SMILES: C=C(CC/C=C(\C)CCC=C(C)C)CC[C@H]1C=C(CC/C=C(\C)CCC=C(C)C)C[C@@H](C(=O)O)[C@@H]1C(=O)O] |
| 74 | 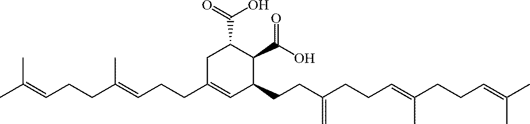 [SMILES: C=C(CC/C=C(\C)CCC=C(C)C)CC[C@H]1C=C(CC/C=C(\C)CCC=C(C)C)C[C@H](C(=O)O)[C@H]1C(=O)O] |
| 75 | 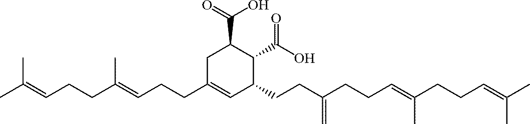 [SMILES: C=C(CC/C=C(\C)CCC=C(C)C)CC[C@@H]1C=C(CC/C=C(\C)CCC=C(C)C)C[C@@H](C(=O)O)[C@@H]1C(=O)O] |
| 76 | 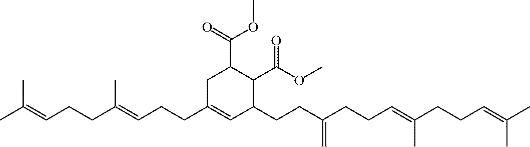 [SMILES: C=C(CC/C=C(\C)CCC=C(C)C)CCC1C=C(CC/C=C(\C)CCC=C(C)C)CC(C(=O)OC)C1C(=O)OC] |
| 77 | 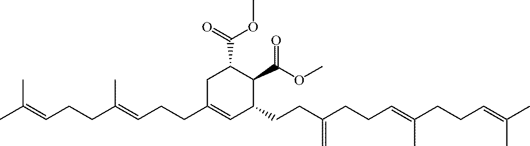 [SMILES: C=C(CC/C=C(\C)CCC=C(C)C)CC[C@@H]1C=C(CC/C=C(\C)CCC=C(C)C)C[C@H](C(=O)OC)[C@H]1C(=O)OC] |
| 78 | 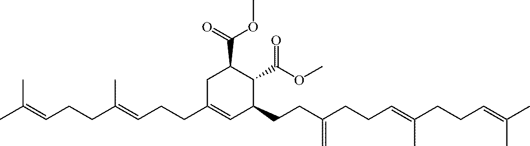 [SMILES: C=C(CC/C=C(\C)CCC=C(C)C)CC[C@H]1C=C(CC/C=C(\C)CCC=C(C)C)C[C@@H](C(=O)OC)[C@@H]1C(=O)OC] |
| 79 | 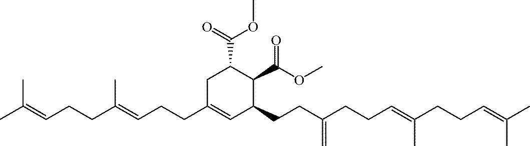 [SMILES: C=C(CC/C=C(\C)CCC=C(C)C)CC[C@H]1C=C(CC/C=C(\C)CCC=C(C)C)C[C@H](C(=O)OC)[C@H]1C(=O)OC] |
| 80 | 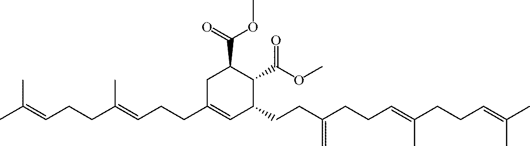 [SMILES: C=C(CC/C=C(\C)CCC=C(C)C)CC[C@@H]1C=C(CC/C=C(\C)CCC=C(C)C)C[C@@H](C(=O)OC)[C@@H]1C(=O)OC] |
| 81 | 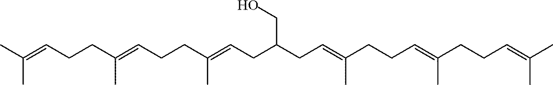 [SMILES: CC(C)=CCC/C(C)=C/CC/C(C)=C/CC(CO)C/C=C(\C)CC/C=C(\C)CCC=C(C)C] |
| 82 | 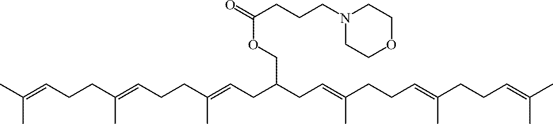 [SMILES: CC(C)=CCC/C(C)=C/CC/C(C)=C/CC(C/C=C(\C)CC/C=C(\C)CCC=C(C)C)COC(=O)CCCN1CCOCC1] |
| 83 | 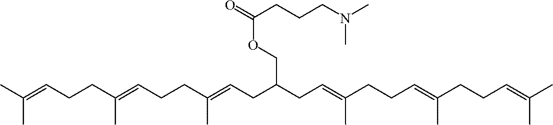 [SMILES: CC(C)=CCC/C(C)=C/CC/C(C)=C/CC(C/C=C(\C)CC/C=C(\C)CCC=C(C)C)COC(=O)CCCN(C)C] |
| 84 | 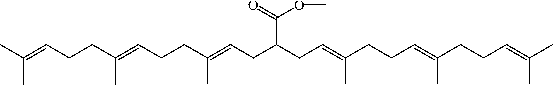 [SMILES: COC(=O)C(C/C=C(\C)CC/C=C(\C)CCC=C(C)C)C/C=C(\C)CC/C=C(\C)CCC=C(C)C] |
| 85 | 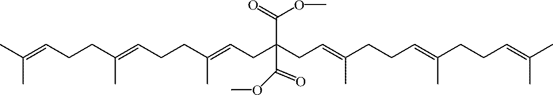 [SMILES: COC(=O)C(C/C=C(\C)CC/C=C(\C)CCC=C(C)C)(C/C=C(\C)CC/C=C(\C)CCC=C(C)C)C(=O)OC] |
| 86 | 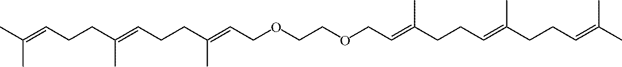 [SMILES: CC(C)=CCC/C(C)=C/CC/C(C)=C/COCCOC/C=C(\C)CC/C=C(\C)CCC=C(C)C] |
| 87 | 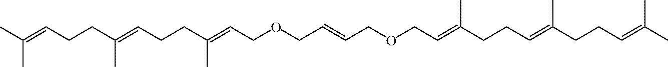 [SMILES: CC(C)=CCC/C(C)=C/CC/C(C)=C/COC/C=C/COC/C=C(\C)CC/C=C(\C)CCC=C(C)C] |
| 88 | 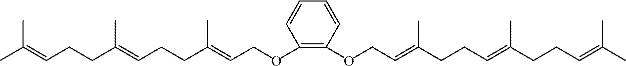 [SMILES: CC(C)=CCC/C(C)=C/CC/C(C)=C/COc1ccccc1OC/C=C(\C)CC/C=C(\C)CCC=C(C)C] |
| 89 | 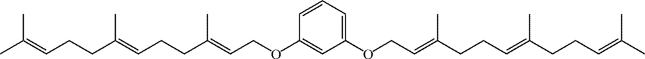 [SMILES: CC(C)=CCC/C(C)=C/CC/C(C)=C/COc1cccc(OC/C=C(\C)CC/C=C(\C)CCC=C(C)C)c1] |
| 90 | 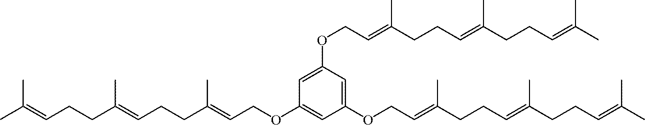 [SMILES: CC(C)=CCC/C(C)=C/CC/C(C)=C/COc1cc(OC/C=C(\C)CC/C=C(\C)CCC=C(C)C)cc(OC/C=C(\C)CC/C=C(\C)CCC=C(C)C)c1] |
| 91 | 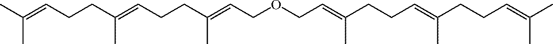 [SMILES: CC(C)=CCC/C(C)=C/CC/C(C)=C/COC/C=C(\C)CC/C=C(\C)CCC=C(C)C] |

### Synthetic Methods

Adjuvant compounds of the disclosure may be prepared by way of various synthetic methods. The synthetic schemes provided in this section are intended to be illustrative of the procedures that may be used to prepare compounds of the disclosure and are not intended to be limiting.

Adjuvant compounds containing a six-membered carbocycle scaffold, such as those represented by formula (II), below, may be produced using a Diels-Alder procedure. Formula (II) is as follows:

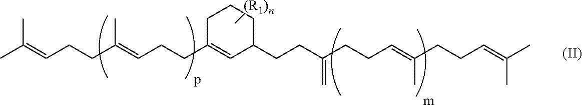

wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, optionally substituted haloalkyl, or halogen;
n is an integer from 0 to 4 (e.g., 0, 1, 2, 3, or 4);
m is an integer from 0 to 3 (e.g., 0, 1, 2, or 3); and
p is an integer from 0 to 3 (e.g., 0, 1, 2, or 3).

This process is outlined in Scheme 1.

### Scheme 1. Diels-Alder Reaction for the Synthesis of Adjuvants Having a Six-Member Carbocycle Core

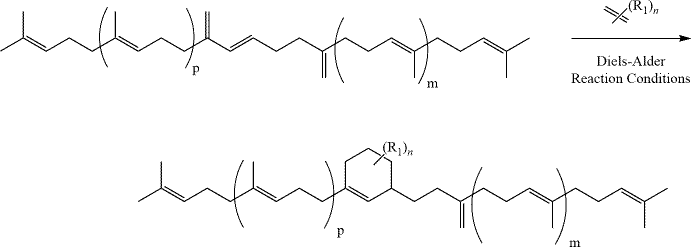

In some embodiments, adjuvant compounds contain one or more carbonyl substituents bound to the six-member carbocycle core. Compounds of this structure can be synthesized using an appropriately substituted dienophile, as shown in Scheme 2.

### Scheme 2. Diels-Alder Reaction for the Synthesis of Carbonyl-Substituted Adjuvants

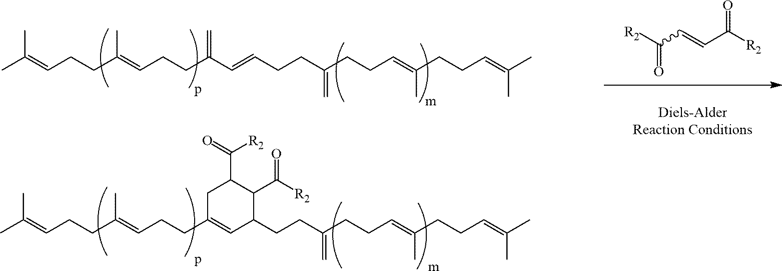

wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted sulfonyloxy, hydroxyl, or halogen;
m is an integer from 0 to 3 (e.g., 0, 1, 2, or 3); and
p is an integer from 0 to 3 (e.g., 0, 1, 2, or 3);
or a pharmaceutically acceptable salt thereof.

In some embodiments, adjuvant compounds of the disclosure contain an optionally substituted hydroxymethyl group bound to the six-member carbocycle scaffold. Compounds of this structure can be synthesized by first forming a corresponding carbonyl-containing compound using, for example, the process shown in Scheme 2, and subsequently reducing the carbonyl compound so as to generate an alcohol. This process is summarized in Scheme 3.

### Scheme 3. Exemplary Procedure for the Synthesis of Alcohol-Containing Adjuvants

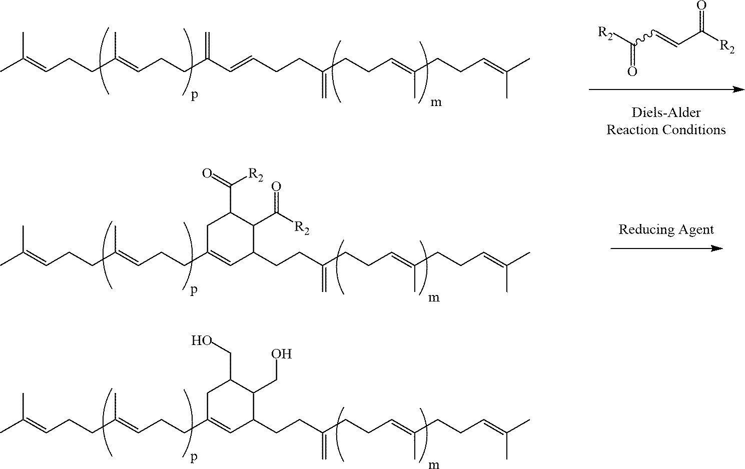

Alcohol-containing adjuvants formed using the procedure shown in Scheme 3 can be further functionalized, for example, by converting the hydroxyl groups joined to the six-member carbocycle core into leaving groups and subsequently displacing one or both leaving groups with an appropriate nucleophile. This process may be used, for example, to produce amine-containing adjuvants by employing an appropriate nitrogen-containing nucleophile. This process is summarized in Scheme 4.

### Scheme 4. Exemplary Procedure for Functional Group Interconversion

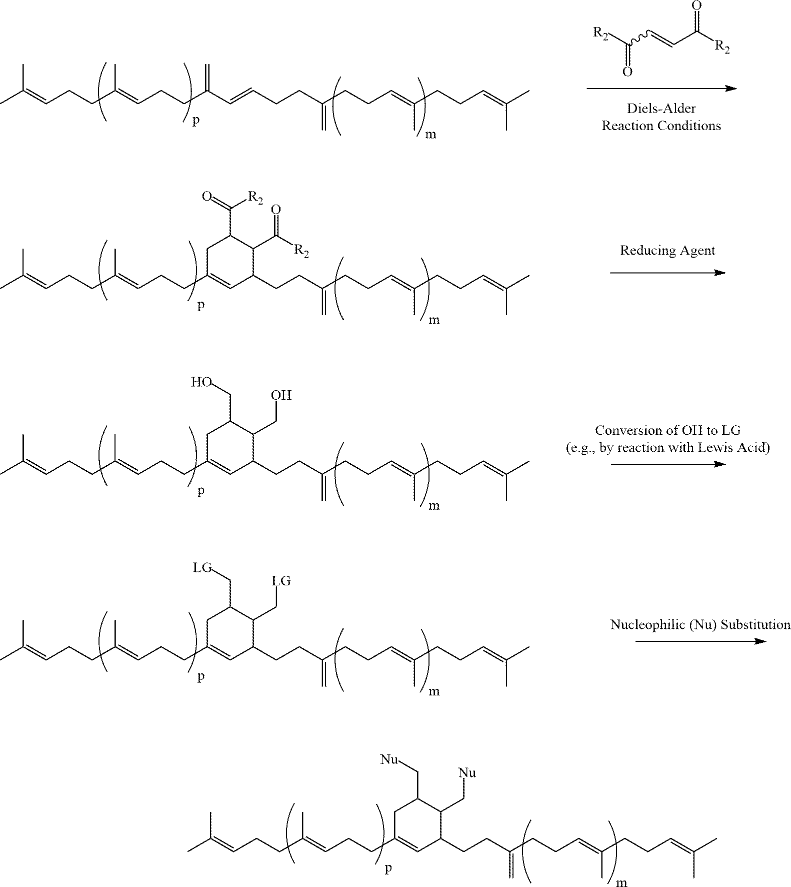

### Vaccine Preparation

The adjuvant compounds of the disclosure may be admixed with an immunogenic antigen (e.g., a protein derived from a pathogen or cancer cell, or a nucleic acid encoding the same) so as to produce a vaccine for therapeutic or prophylactic use. In some embodiments, an adjuvant compound of the disclosure (e.g., a compound of any one of formulas (I) - (XXVI), such as any one of compounds (1) -(91), above) is covalently conjugated to an antigen, thereby forming a self-adjuvanting vaccine. Self-adjuvanting vaccines may exhibit the advantageous effect of being rapidly internalized by antigen-presenting cells of the immune system, such as macrophages and dendritic cells, among others. Moreover, the use of self-adjuvanting vaccines helps to ensure that the antigen-presenting cells activated by the adjuvant are the same cells that are exposed to antigen, thereby promoting an immune response that is highly specific for a desired antigen.

### Self-Adjuvanting Vaccine Synthesis

Self-adjuvanting vaccines of the disclosure may be produced, for example, by reacting an adjuvant of the disclosure with a desired antigen, such as a protein expressed by a virus, bacterium, or protozoan (or a nucleic acid (e.g., a DNA or RNA molecule) encoding the same) or a protein expressed by a cancer cell (or a nucleic acid (e.g., a DNA or RNA molecule) encoding the same). The adjuvant may contain, for example, a reactive chemical substituent, such as a nucleophilic substituent, an electrophilic substituent, or a or dienophilic substituent, among others. The antigen may contain, for example, a chemical substituent that is suitable for reaction with the reactive substituent on the adjuvant, such that reacting the adjuvant and the antigen results in the formation of a stable covalent bond. This process is exemplified in Scheme 5, below.

### Scheme 5. Exemplary Process for the Formation of a Self-Adjuvanting Vaccine by Reaction of A Nucleophilic Substituent On an Adjuvant Compound of the Disclosure with an Electrophilic Substituent On A Desired Antigen

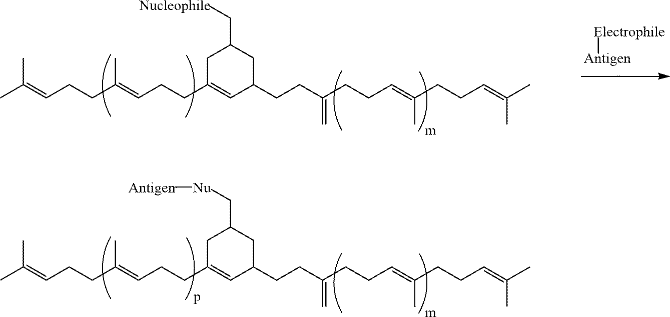

### Viral, Bacterial, and Protozoan Antigens

Exemplary antigens that may be used in conjunction with the vaccines of the disclosure include, without limitation, proteins that are expressed by a virus, bacterium, or protozoan, as well as nucleic acids (e.g., DNA or RNA molecules) encoding the same.

For example, in some embodiments, the antigen is a protein expressed by a virus selected from influenza virus, hepatitis A virus, hepatitis B virus, hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O′nyong′nyong virus, chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi’s sarcoma associated-herpesvirus (KSHV), severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus, rhinovirus, mumps virus, poliovirus, human enterovirus, coxsackievirus, human papilloma virus, adeno-associated virus, astrovirus, JC virus, BK virus, SV40 virus, Norwalk virus, rotavirus, human immunodeficiency virus (HIV), and human T-lymphotropic virus. In some embodiments, the antigen is encoded by a nucleic acid (e.g., a DNA or RNA molecule) encoding the same.

In some embodiments, the antigen is a protein expressed by a coronavirus, such as SARS-CoV-2, MERS-CoV, SARS-CoV, OC43, or HKU1. In some embodiments, the antigen is encoded by a nucleic acid (e.g., a DNA or RNA molecule) encoding the same.

In some embodiments, the antigen is a protein expressed by a bacterium belonging to a genus selected from Mycobacterium (e.g., Mycobacterium tuberculosis), Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasturella, Francisella, Brucella, Bartonella, Clostridium, Vibrio, Campylobacter, and Staphylococcus. In some embodiments, the antigen is encoded by a nucleic acid (e.g., a DNA or RNA molecule) encoding the same.

In some embodiments, the antigen is a protein expressed by a protozoan. The antigen may be a protein expressed by a parasite, such as a parasite selected from the group consisting of Plasmodium malariae, Plasmodium vivax, Plasmodium ovale, Plasmodium falciparum, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Trichomonas vaginalis, and Histomonas meleagridis. The parasite may be a helminthic parasite, such as Richuris trichiura, Ascaris lumbricoides, Enterobius vermicularis, Ancylostoma duodenale, Necator americanus, Strongyloides stercoralis, Wuchereria bancrofti, Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, or Echinococcus granulosus. In some embodiments, the antigen is encoded by a nucleic acid (e.g., a DNA or RNA molecule) encoding a protein expressed by any of the above parasites.

### Cancer Vaccines

The present disclosure also features vaccines useful for the treatment and prevention of cancer. Such vaccines may include, for example, an adjuvant compound described herein (e.g., a compound of any one of formulas (I) - (XXVI), such as any one of compounds (1) - (91)) admixed with, or conjugated to, a cancer antigen. Exemplary cancer antigens useful in conjunction with the compositions and methods of the disclosure include proteins expressed by a cancer cell, as well as nucleic acids (e.g., DNA or RNA molecules) encoding the same. Exemplary cancer cell antigens that may be used in the formation of a vaccine (e.g., a self-adjuvanting vaccine) described herein include, without limitation, gp100, Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-1, MUC-16, L1CAM, HERV-K-MEL, KK-LC-1, KM-HN-1, LAGE-1, Sp17, TAG-1, TAG-2, ENAH (hMena), mammaglobin-A, NY-BR-1, BAGE-1, MAGE-A1, MAGE-A2, MAGE-A4, MAGE-A6, MAGE-A9, MAGE-A10, MAGE-A12, MAGE-C2, mucink, SSX-2, SSX-4, TRAG-3, c-myc, cyclin B1, p62, Survivin, CD45, DKK1, RU2AS, Telomerase, K-ras, G250, Hepsin, Intestinal carboxyl esterase, α-foetoprotein, M-CSF, PSMA, CASP-5, COA-1, OGT, OS-9, TGF-βRII, gp70, CALCA, CD274, mdm-2, α-actinin-4, Elongation factor 2, ME1, NFYC, GAGE-1/2/8, GAGE-3/4/5/6/7, XAGE-1b/GAGED2a, STEAP1, PAP, PSA, FGF5, hsp70-2, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, GnTVf, LY6K, and NA88-A. In some embodiments, the antigen is encoded by a nucleic acid molecule (e.g., a DNA or RNA molecule) encoding any of the above proteins.

Additional examples of tumor-specific antigens are described in Wilkinson et al. Cancer Immunol. Immunother. 61(2):169-79 (2012); Hural et al. J. Immunol. 169(1):557-65 (2002); Tsukahara et al. Cancer Res. 64(15):5442-8 (2004); Kessler et al. J. Exp. Med. 193(1):73-88 (2001); Ikeda et al. Immunity 6(2):199-208 (1997); Asemissen et al. Clin. Cancer Res. 12(24):7476-82 (2006); Ohminami et al. Blood. 95(1):286-93 (2000); Guo et al. Blood. 106(4):1415-8 (2005); Lin et al. J. Immunother. 36(3):159-70 (2013); Fujiki et al. J. Immunother. 30(3):282-93 (2007); Wick et al. Clin. Cancer Res. 20(5):1125-34 (2014); Hassan et al. Appl. Immunohistochem. Mol. Morphol. 13(3):243-7 (2005); Jager et al. J Exp Med. 187(2):265-70 (1998); Jager et al. Proc. Natl. Acad. Sci. U.S.A. 103(39):14453-8 (2006); Chen et al. J Immunol. 165(2):948-55 (2000); and Mandic et al. J Immunol. 174(3):1751-9 (2005), each of which is incorporated herein by reference as it pertains to tumor-specific antigens.

In some embodiments, the cancer antigen is a protein expressed by an ovarian cancer cell. Such proteins include Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-16, L1CAM, Mannan-MUC-1, HERV-K-MEL, KK-LC-1, KM-HN-1, LAGE-1, MAGE-A4, SSX-4, TAG-1, and TAG-2, among others. In some embodiments, the antigen is encoded by a nucleic acid molecule (e.g., a DNA or RNA molecule) encoding any of the foregoing proteins.

In some embodiments, the cancer antigen is a protein expressed by a breast cancer cell. Such proteins include ENAH (hMena), mammaglobin-A, NY-BR-1, EpCAM, NY-ESO-1, BAGE-1, HERV-K-MEL, KK-LC-1, KM-HN-1, LAGE-1, MAGE-A1, MAGE-A2, mucink, Sp17, SSX-2, TAG-1, TAG-2, TRAG-3, Her2/Neu, c-myc, cyclin B1, MUC1, p53, p62, and Survivin, among others. In some embodiments, the antigen is a nucleic acid molecule (e.g., a DNA or RNA molecule) encoding any of the foregoing proteins.

In some embodiments, the cancer antigen is a protein expressed by a pancreatic cancer cell. Such proteins include ENAH (hMena), PBF, K-ras, Mesothelin, and mucink, among others. In some embodiments, the antigen is a nucleic acid molecule (e.g., a DNA or RNA molecule) encoding any of the foregoing proteins.

In some embodiments, the cancer antigen is a protein expressed by a colorectal cancer cell. Such proteins include ENAH (hMena), Intestinal carboxyl esterase, CASP-5, COA-1, OGT, OS-9, TGF-βRII, NY-ESO-1, CEA, HERV-K-MEL, KK-LC-1, KM-HN-1, LAGE-1, MAGE-A2, Sp17, TAG-1, TAG-2, c-myc, cyclin B1, MUC1, p53, p62, Survivin, and gp70, among others. In some embodiments, the antigen is encoded by a nucleic acid molecule (e.g., a DNA or RNA molecule) encoding any of the foregoing proteins.

In some embodiments, the cancer antigen is a protein expressed by a lung cancer cell. Such proteins include CD274, mdm-2, α-actinin-4, Elongation factor 2, ME1, NFYC, NY-ESO-1, GAGE-1/2/8, HERV-K-MEL, KK-LC-1, KM-HN-1, LAGE-1, MAGE-A2, MAGE-A6, Sp17, TAG-1, TAG-2, TRAG-3, XAGE-1b/GAGED2a, c-myc, cyclin B1, Her2/Neu, MUC1, p53, p62, and Survivin, among others. In some embodiments, the antigen is encoded by a nucleic acid molecule (e.g., a DNA or RNA molecule) encoding any of the foregoing proteins.

In some embodiments, the cancer antigen is a protein expressed by a prostate cancer cell. Such proteins include DKK1, ENAH (hMena), Kallikrein 4, PSMA, STEAP1, PAP, PSA, NY-ESO-1, BAGE-1, GAGE-1/2/8, GAGE-3/4/5/6/7, HERV-K-MEL, KK-LC-1, KM-HN-1, LAGE-1, and Sp17, among others. In some embodiments, the antigen is encoded by a nucleic acid molecule (e.g., a DNA or RNA molecule) encoding any of the foregoing proteins.

In some embodiments, the cancer antigen is a protein expressed by a melanoma cell. Such proteins include gp100, Hepsin, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, NY-ESO-1, BAGE-1, GAGE-1/2/8, GAGE-3/4/5/6/7, GnTVf, HERV-K-MEL, KK-LC-1, KM-HN-1, LAGE-1, LY6K, MAGE-A1, MAGE-A6, MAGE-A10, MAGE-A12, MAGE-C2, NA88-A, Sp17, SSX-2, SSX-4, and TRAG-3, among others. In some embodiments, the antigen is encoded by a nucleic acid molecule (e.g., a DNA or RNA molecule) encoding any of the foregoing proteins.

In some embodiments, the cancer antigen is a protein expressed by a squamous cell carcinoma cell. Such proteins include CASP-8, p53, and SAGE, among others. In some embodiments, the antigen is encoded by a nucleic acid molecule (e.g., a DNA or RNA molecule) encoding any of the foregoing proteins.

In some embodiments, the cancer antigen is a protein expressed by a chronic myeloid leukemia cell. Such proteins include BCR-ABL, dek-can, EFTUD2, and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by an acute lymphoblastic leukemia cell. Such proteins include ETV6-AML1, and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by an acute myelogenous leukemia cell. Such proteins include FLT3-ITD, Cyclin-A1, and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by a chronic lymphocytic leukemia cell. Such proteins include FNDC3B and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by a multiple myeloma cell. Such proteins include MAGE-C1, NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

In some embodiments, the cancer antigen is a protein expressed by a bladder cancer cell. Such proteins include BAGE-1, GAGE-1/2/8, GAGE-3/4/5/6/7, MAGE-A4, MAGE-A6, SAGE, NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

In some embodiments, the cancer antigen is a protein expressed by a neuroblastoma cell. Such proteins include NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

Additionally or alternatively, a cancer vaccine of the disclosure may contain an adjuvant compound admixed with, or conjugated to, an agent that activates antigen-presenting cells of the immune system, such as a toll-like receptor 4 (TLR4) agonist. The immune-stimulating agent may be present in addition to, or instead of, the cancer antigen. Exemplary TLR4 agonists useful in conjunction with the compositions and methods of the disclosure include glucopyranosyl lipid A and lipopolysaccharide, among others.

### Protein and Nucleic Acid Antigens

Antigens that may be used in the formation of a vaccine of the disclosure include proteins (e.g., a protein expressed by a virus, bacterium, protozoan, or cancer cell described herein). In some embodiments, the antigen is encoded by a nucleic acid encoding such a protein. The nucleic acid may be, for example a DNA molecule or RNA molecule encoding a protein expressed by a virus, bacterium, protozoan, or cancer cell, such as any of the proteins recited above. Exemplary methods for producing RNA vaccines are described, for example, in Erasmus et al. Molecular Therapy 26:1-16 (2018), the disclosure of which is incorporated herein by reference as it pertains to nucleic acid vaccines.

Nucleic acids encoding a protein of interest (e.g., a protein expressed by a virus, bacterium, protozoan, or cancer cell described herein) may be produced using synthetic chemistry and/or molecular biology techniques known in the art. For example, once a desired protein is identified, an open reading frame (ORF) encoding the protein may be designed using standard codon-amino acid relationships known in the art. The ORF may be a wild-type ORF that occurs naturally for the selected protein, an isoform, or a variant or fragment thereof.

In some embodiments, the nucleotide sequence of the ORF is codon optimized for expression in a desired cell type (e.g., a mammalian cell, such as a human cell). Codon optimization methods are known in the art. Codon optimization may be used, for example, to match codon frequencies in target and host organisms to ensure proper protein folding, bias GC content to increase RNA stability or reduce secondary structures, minimize tandem repeat codons or base runs that may impair gene construction or expression, customize transcriptional and translational control regions, insert or remove protein trafficking sequences, remove or add post-translation modification sites in encoded proteins (e.g. glycosylation sites), add, remove or shuffle protein domains, insert or delete restriction sites, modify ribosomal binding sites and RNA degradation sites, and/or adjust translational rates to allow the various domains of the protein to fold properly.

### Lipid Nanoparticle Formulations

In some embodiments of the disclosure, nucleic acid (e.g., DNA or RNA) vaccines are formulated in a nanoparticle, such as a lipid nanoparticle. The nanoparticle (e.g., lipid nanoparticle) may be constructed such that an adjuvant compound of the disclosure (e.g., an adjuvant compound of any one of formulas (I) - (XXVI), herein, such as any one of compounds (1) - (91)) is located within the core of the nanoparticle, and the nucleic acid (e.g., DNA or RNA) component is located on the nanoparticle’s interior or exterior.

In some embodiments, the nanoparticle (e.g., lipid nanoparticle) includes a polycation. The nanoparticle may be, for example, a lipid-polycation complex. The lipid nanoparticle may be manufactured, for example, using methods described in US 2012/0178702, the disclosure of which is incorporated herein by reference as it pertains to nanoparticle formulations and techniques for producing the same. As a non-limiting example, the polycation may include a cationic peptide or a polypeptide such as polylysine, polyornithine, or polyarginine. In some embodiments, the cationic peptide is one described in WO 2012/013326 or US 2013/0142818, each of which is incorporated herein by reference as it pertains to cationic peptides. In some embodiments, nanoparticle formulations of the disclosure include a non-cationic lipid, such as cholesterol or dioleoyl phosphatidylethanolamine, among others.

### Routes of Administration

Vaccines produced using one or more adjuvants of the disclosure may be administered to a subject (e.g., a mammalian subject, such as a human) for therapeutic or prophylactic treatment. Such vaccines may be administered to a subject by way of any suitable route of administration. Exemplary routes of administration useful in conjunction with the vaccines of the disclosure include, without limitation, injection by way of the intramuscular, intraperitoneal, intradermal, or subcutaneous routes, or by way of transmucosal administration to the oral, respiratory, or genitourinary tract(s).

### Additional Excipients

Vaccine compositions of the present disclosure may contain, in addition to an adjuvant compound describe herein, one or more pharmaceutically acceptable carriers, diluents, excipients, or solvents. Exemplary pharmaceutically acceptable carriers, diluents, excipients, and solvents that may be used in conjunction with the vaccines of the present disclosure include those pharmaceutically acceptable additives described in Adejare, Aldeboye, Remington: The Science and Practice of Pharmacy (Academic Press, 2020).

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Physical Emulsion Stability of Semi-Synthetic Squalene Analogues Compared to Shark Squalene

A series of experiments were conducted to evaluate the physical emulsion stability of the squalene analogs, DHIS and farnesene thermal dimer (shown below), as compared to that of shark squalene.

TABLE 2

Structures of Semi-Synthetic Squalene Analogues

| | |
|---|---|
| Analogue Tested | Chemical Structure |
| DHIS | 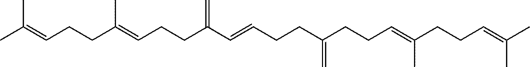 [SMILES: C=C(/C=C/CCC(=C)CC/C=C(\C)CCC=C(C)C)CC/C=C(\C)CCC=C(C)C] |
| Farnesene Thermal Dimer | 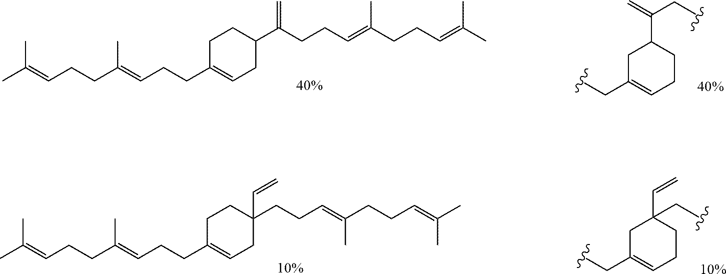 |

Emulsions were manufactured by mixing a buffered aqueous phase and an oil phase with a Silverson Heavy Duty Laboratory Mixer Emulsifier (¾ in. tubular square hole high shear screen attachment; East Longmeadow, MA) at ~7,000 - 10,000 rpm for ~10 minutes, then microfluidizing the mixture using the Microfluidics M110P (Newton, MA) for ~12 passes at 30,000 psi. Particle size was determined using the Malvern Instruments (Worcestershire, UK) Zetasizer Nano-S, -ZS, or -APS via dynamic light scattering (DLS). Five (5) µl of formulation were combined with 500 µl ultrapure water in a 1.5-ml polystyrene disposable cuvette. DLS measurements were then made at least three times on each sample. Emulsions were stored at 2-8° C. and sample aliquots removed for particle sizing at the indicated time points.

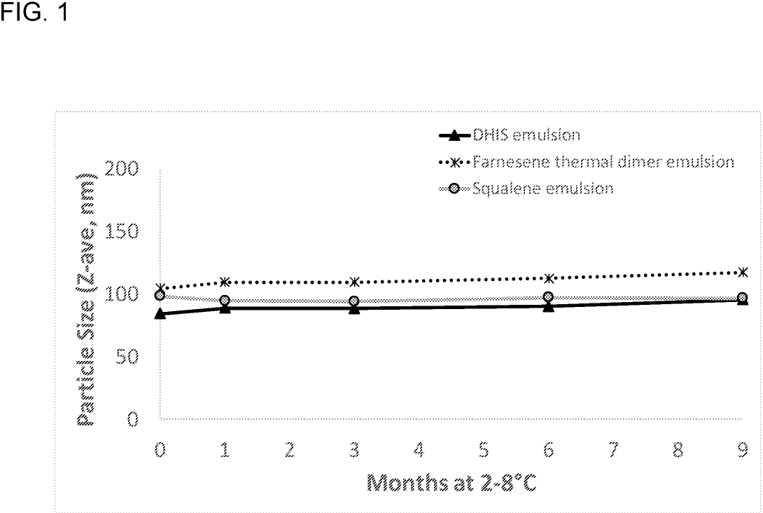

As is shown in FIG. 1, the physical emulsion stability of DHIS emulsion and farnesene thermal dimer emulsion is similar to that of squalene emulsion.

### Example 2. Cytokine (Mip 1β) Stimulation from Human Whole Blood Exposed in Vitro to DHIS or Farnesene Thermal Dimer Emulsions

A series of experiments was conducted to assess the cytokine response to DHIS and farnesene thermal dimer emulsions. Informed consent was obtained from eight human donors and the study was approved by Western IRB, Seattle, WA. Fifty (50) µL formulation was added to 150 µL of heparinized whole blood using 96-well round bottom tissue culture plates, in duplicate. The plates were incubated at 37° C. and 5% CO₂ for 24 h. After incubation, 150 µL extractions of the plasma supernatant from each well were aspirated and assayed for Mip-1β using an ELISA kit.

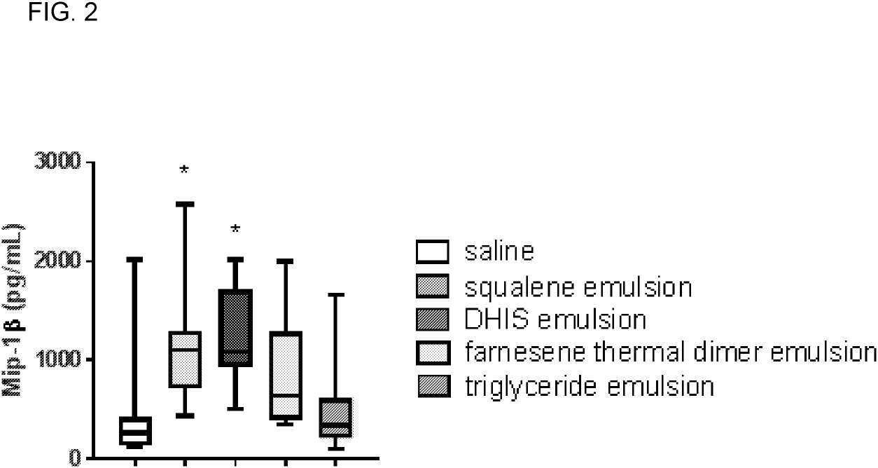

As is shown in FIG. 2, the Mip 1β stimulation response from human blood exposed to DHIS or farnesene thermal dimer emulsions is similar to that of squalene.

### Example 3. Enhancement of Immunological Response to Recombinant Antigens in Mice Injected with Antigens Formulated in Squalene Analogues

### Materials and Methods

C57BL/6 mice were immunized intramuscularly at Day 0 and Day 21 (5 mice/group). Adjuvant formulations consisted of the indicated oil emulsified with phospholipid and poloxamer 188 in a 25 mM ammonium phosphate buffer containing glycerol. Hemagglutination inhibition titers were performed by either Midwest Research Institute (Kansas City, MO, USA) or Tria Bioscience Corp. (Seattle, WA, USA) using horse erythrocytes. Briefly, sera were collected from mice three weeks after a boost immunization. HI antibodies were tested against the vaccine strain (A/Vietnam/1203/04-Clade 1). The HI titer was defined as the reciprocal of the highest dilution of sera, which completely inhibited the agglutination of the RBCs. For IgG midpoint titer assessment, peripheral blood was collected and subsequent centrifugation at 10,000 rpm for 5 minutes was carried out to isolate the serum. Serum titers against ID97 were then evaluated by antibody capture ELISA.

Coming high bind 384 well plates (VWR International) were coated overnight at 4° C. with 2 µg ml-1 ID93 in coating buffer (eBioscience). Next, plates were blocked with 1% BSA-PBS and serum samples serially diluted. Detection antibodies utilized were anti-mouse IgG HRP (Southern Biotech). Plates were analyzed at 450 nm (ELx808, Bio-Tek Instruments Inc.) and midpoint titers were determined as EC50 values from weighted curve fits using the GraphPad Prism software. For the CD4 T cell assay, cells were plated at 2×10⁶ cells/well and either stimulated for two hours at 37° C. with ID97 (10 µg/mL) or left unstimulated. GolgiPlug (BD Biosciences) was added and the cells were incubated for an additional eight hours at 37° C. Cells were washed and surface stained with fluorochrome-labeled antibodies to CD4 (BioLegend and eBioscience) in the presence of anti-CD16/32 (clone 2.4G2) for 20 minutes. Cells were washed and permeabilized with Cytofix/Cytoperm (BD Biosciences) for 20 minutes. Cells were washed with Perm/Wash (BD Biosciences) and stained intracellularly with fluorochrome-labeled antibodies to CD154 (clone MR1) (BioLegend and eBioscience) for 20 minutes. Cells were washed and resuspended in PBS. Up to 10⁶ events were collected on an LSRFortessa flow cytometer (BD Biosciences). Data were analyzed with FlowJo software. Cells were gated as singlets > lymphocytes > CD4+ CD8- > cytokine positive.

ID97-specific response frequencies were determined by subtracting the frequency of response positives of unstimulated cells from ID97-stimulated cells in matched samples.

### Results

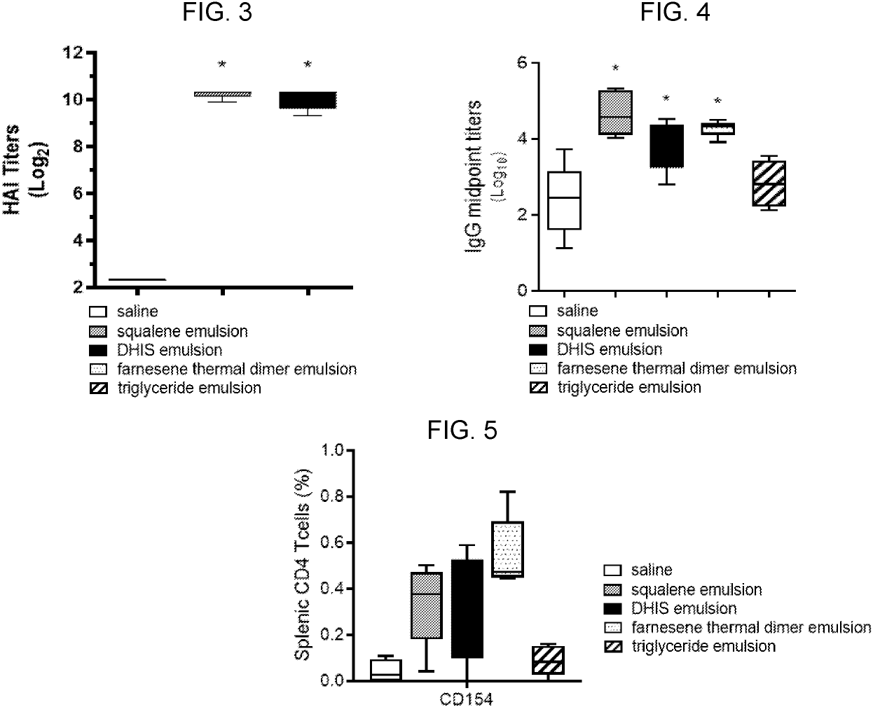

As is shown in FIG. 3, enhancement in HAI titers in C57BL/6 mice immunized twice intramuscularly with recombinant H5N1 and DHIS emulsion (red) is similar to the enhancement that results from the use of the same antigen in combination with shark squalene emulsion.

As is shown in FIG. 4, enhancement in antigen specific IgG response in C57BL/6 mice immunized once intramuscularly with a recombinant tuberculosis antigen (ID97) and DHIS emulsion or farnesene thermal dimer emulsion is similar to the enhancement that results from the use of the same antigen in combination with shark squalene emulsion.

As is shown in FIG. 5, enhancement in antigen specific CD4+ T cell response, as indicated by the CD154 marker, in mice immunized twice with ID97 and DHIS emulsion or farnesene thermal dimer emulsion is similar to the response that results from the use of the same antigen and shark squalene emulsion compared to antigen alone or antigen with triglyceride emulsion. *p<0.05 vs antigen alone (parts 1-3) by one-way ANOVA using Dunnet’s multiple comparison correction.

### Example 4. Synthesis of Compound (91)

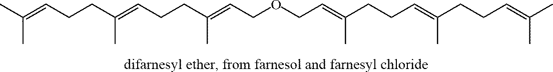

### Procedure

A solution of E,E-farnesol (4.1 g, 18.47 mmol) in 10 mL tetrahydrofuran was added to a solution of potassium t-butoxide (3.96 g, 35.3 mmol) in 38 mL tetrahydrofuran. The solution turned light yellow. After fifteen minutes, a solution of farnesyl chloride was added in 6 mL tetrahydrofuran. The mixture was heated at 56° C. for ninety minutes and allowed to cool to room temperature. Most of the solvent was removed by rotary evaporation. 25 mL of 5% aqueous sodium bicarbonate and 25 mL deionized water was added, and the crude product was evaporated to remove ethyl acetate and purified by silica gel chromatography using 2% ethyl acetate in heptanes to give 0.9 g cleaner fractions (91% by GCMS) and 3.1 g less pure fractions (85% by GCMS). Estimated yield = 47% (unoptimized).

Proton NMR: 5.37{2H, dt, J = 6.83 Hz (triplet), J = 1.2 Hz (doublet)}, 5.10 (4H, m), 3.97 (4H, d, J = 6.6 Hz), 2.16-2.01 (m, 12H), 2.00-1.93 (m, 4H), 1.67 (broad singlet, 12H, 1.59 (broad singlet, 12H).

Carbon 13 NMR: 139.99, 135.22, 131.26, 124.36, 123.93, 121.11, 66.40, 39.70, 39.62, 26.73, 26.34, 25.66, 17.65, 16.46, 15.97.

M/Z = 426.4

### Example 5. Synthesis of C20 Myrcene Linear Dimer

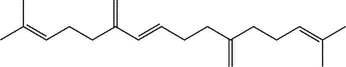 [SMILES: C=C(/C=C/CCC(=C)CCC=C(C)C)CCC=C(C)C]

### Procedure

Myrcene (159.4 g, 1.17 moles) was added to a 1 Liter three neck flask and diluted with 300 mL 2-propanol. Palladium(acac)2 (1.13 g) and triphenylphosphine (1.56 g) were subsequently added. The mixture was heated to 80° C. for 16 hours. The solvent was removed by rotary evaporation, crude weight = 156.9 g. The sample was then diluted with 200 mL hexanes and filtered through a 6-inch tall by 10 cm diameter silica gel column before being eluted with 500 mL n-pentane, followed by 250 mL 10% ethyl acetate in hexanes. The combined filtrates were concentrated by rotary evaporation to give 148.8 g of nearly colorless oil. Additional evaporation and first stage distillation at 160-170° C., 0.35 torr yielded 120.2 g of slightly impure product, which contained some C10 impurities. Upon distillation at 120° C. and 0.5 torr, 5.9 g of light materials (C10 starting material) and 112.0 g desired product resulted. Yield = 70%.

Proton NMR (in ppm): 6.08 (d, J = 15.8 Hz, 1H), 5.77 (dt, J = 15.6, 6.8 Hz, 1H), 5.14 (m, 2H), 4.88 (broad doublet, J = 11.1 Hz, 2H), 4.74 (broad singlet, 2H), 2.26-2.00 (m, 12H), 1.69 (broad singlet, 6H), 1.61 (broad singlet, 6H).

Carbon 13 NMR (in ppm): 148.98, 148.07, 132.26, 131.63, 131.58, 129.55, 124.32, 124.18, 36.16, 35.99, 32.35, 31.24, 26.98, 26.46, 25.69, 17.71.

### Example 6. C25 Farnesene + Myrcene Coupling Product: Preparation of Diels-Alder Diene

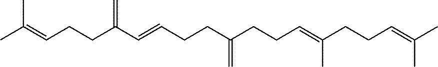 [SMILES: C=C(/C=C/CCC(=C)CC/C=C(\C)CCC=C(C)C)CCC=C(C)C]

### Procedure

Farnesene (79.8 g, 0.39 moles) was added to myrcene (154.0 g, 1.13 moles) and the headspace was subsequently purged with nitrogen. 400 mL of 2-propanol was then added, followed by 1.09 g of Palladium (acac)₂ and 1.43 g of triphenylphosphine. The mixture was heated at 80° C. for 17 hours. Gas chromatography at this stage demonstrated a mixture of C25, C20, and C30 coupling products. Most of the solvent was then removed by rotary evaporation.

The crude product was subsequently diluted with 600 mL 10% ethyl acetate in hexanes and filtered through silica gel. The filter cake was washed with 400 mL 10% ethyl acetate and concentrated to yield 224.2 g of black liquid. 111.3 g of this black oil was diluted with 200 mL hexanes and filtered through silica gel. The silica gel was then washed with 200 mL of hexanes and then 200 mL 10% ethyl acetate in hexanes. This process was repeated with the rest of the batch. At that point, the batch was evaporated to dryness to give 213.1 g nearly colorless liquid. Distillation at 160° C. and 0.35 torr gave a light fraction of mainly C20 compounds and a distillation residue of 167.1 g, which was enriched in C25 compounds and C30 compounds. Further distillation at 160° C. and 0.35 torr gave a light fraction weighing 30.6 g, which was composed of about a 4:1 mixture of C20 to C25 materials. The distillation residue weighed 136.1 g, which was further distilled to give a light fraction which weighed 31.9 g. This light fraction was a mixture of about 70% C20 compounds and 30% of C25 compounds. The nonvolatile residue from the distillation was further distilled at 200° C. and 0.54 torr to give a fraction which weighed 44.0 g and was about 80% C25 products along with some C20 and C30 impurities.

Proton NMR: 6.08 (d, J = 15.8 Hz, 1H), 5.73 (broad doublet of triplets, J = 15.8, 6.6 Hz, 1H), 5.18-5.07 (m, 3H), 4.88 (d, J = 11.5 Hz, 2H), 4.74 (s, 2H), 2.28-1.95 (m, 16H), 1.68 (broad singlet, 6H), 1.61 (broad singlet, 6H.

Carbon 13 NMR: 148.99, 148.98, 146.07, 146.03, 135.26, 135.21, 132.26, 132.24, 131.64, 131.59, 131.28, 129.56, 129.53, 124.40, 124.39, 124.31, 124.17, 124.06, 113.37, 109.26, 109.22, 39.73, 36.15, 35.98, 32.34, 31.24, 26.97, 26.84, 26.75, 26.74, 26.45, 26.36, 25.70, 17.71, 17.70, 16.04.

M/Z = 340.3

### Example 7. Synthesis of Compound (56)

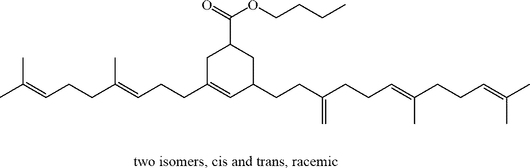

### Batch 1

DHIS (9.2 g, 22.5 mmmol), 6 mL xylenes and butyl acrylate (6 mL, 42.1 mmol) were combined under a nitrogen atmosphere and then heated at 135° C. After 65 minutes, the reaction appeared complete by GC. Xylenes were removed by distillation at 65° C., 0.5 torr and then the desired product was purified by silica gel chromatography using 10% ethyl acetate as eluent. This process yielded 12.6 g slightly impure material containing some residual solvent and hydrocarbon impurities.

### Batch 2

DHIS (69.8 g, 0.171 moles), butyl acrylate (35 mL, 0.245 moles) and 50 mL toluene were combined and heated under a nitrogen atmosphere at 111-120° C. for six hours. GC showed that conversion was very high. Most of the solvent was removed by rotary evaporation. Crude weight = 109.8 g (91.66 g theoretical). 40.2 g of this material was purified by silica gel chromatography using 10% toluene in heptanes to give 7.8 g (fractions 6 to 8, impure product) along with 19.8 g of material (fractions 9 to 26). 1.7 g of the mixed fractions were later purified by silica gel chromatography using 10% toluene in heptanes to give 1.5 g fraction 12 to 18 used for characterization of the intermediate.

Proton NMR: 5.50 (broad singlet, 1H for one isomer), 5.32 (broad singlet, 1H for the other isomer), 5.10 (multiplet, 4H for each isomer), 4.71 (broad singlet, 2H for each isomer), 4.09 (multiplet, 2H for each isomer), 2.62 (dddd, J = 11.4, 10.6, 5.4 and 3.4 Hz, 1H for one isomer), 2.46 (multiplet, 1H for each isomer), 2.24 (ddt, J = 10 for triplet, 3 and 2.3 Hz of the two doublets, 1H for one isomer), 2.2-1.9 (multiplet, 20H for both isomers), 1.67 (broad singlet, 6H for each isomer), 1.60 (broad singlet, 12H for each isomer), 1.44=1.34 (m, 4H for one isomer, 3H for one isomer), 1.32-1.24 (multiplet, 4H for each isomer), 1.14-1.10 (t, J = 14, 1H for one isomer), 0.93 (t, J = 6.9 Hz, 3H for one isomer), 0.88 (t, J = 7.0 Hz, 3H for one isomer).

Carbon NMR:176.22, 174.94, 149.54, 149.45, 137.47, 137.11, 135.10, 131.20, 129.04, 128.24, 124.42, 124.12, 124.07, 123.64, 123.52, 109.09, 108.96, 71.21, 64.09, 63.92, 45.62, 43.78, 39.77, 37.67, 37.65, 37.26, 36.16, 35.96, 35.77, 33.82, 32.93, 32.68, 31.95, 30.83, 30.50, 29.09, 28.14, 27.97, 27.68, 26.83, 26.20, 25.71, 22.76, 20.25, 19.31, 19.25, 19.20, 17.69, 16.06, 16.02, 14.14 and 13.74.

M/Z = 552.5 (isomers not resolved by GCMS)

### Example 8. Synthesis of Compound (1)

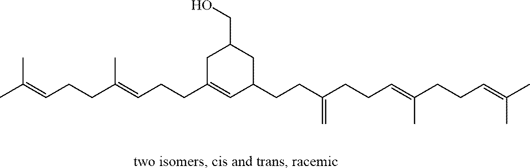

### Batch 1

90 mL dry THF was cooled in an ice water bath and solid lithium aluminum hydride (0.98 g, 25.8 mmoles) was added, followed by dropwise addition of a solution of the esters (19.8 g, 36.9 mmol theoretical from batch 2) in 60 mL THF. After one hour at room temperature, thin layer chromatography showed no starting ester. The mixture was cooled to 0° C. and 5 mL water was added slowly (hydrogen released). Then, 55 mL of 0.50 M hydrochloric acid was added. The phases were separated, and the organic phase was concentrated to give 18.3 g of a nearly colorless oil.

### Batch 2

Lithium aluminum hydride (2.99 g, 79 mmoles) was added to 250 mL dry THF cooled in an ice water bath. A solution of the esters (60.9 g batches 1 and 2, up to 0.113 moles) in 100 mL THF was added dropwise. After the addition was complete, the mixture was stirred at room temperature for two hours. The flask was re-cooled to 0° C. and 20 mL water was added slowly followed by 100 mL 5% aqueous hydrochloric acid. Most of the THF was removed by rotary evaporation and the phases were separated. The aqueous phase was extracted with an additional 100 mL ethyl acetate and the combined organic phases were concentrated to give 61.2 g crude alcohol. The two batches were purified by silica gel chromatography using a 5% ethyl acetate to 30% ethyl acetate step gradient to give 44.9 g desired alcohols as a 55:45 mixture of isomers. Yield = 49.6% over the two steps.

Proton NMR: 5.41 (broad singlet, 1H for one isomer), 5.31 (broad singlet, 1H for one isomer), 5.14-5.08 (multiplet, 4H for each isomer), 4.73 (broad singlet, 2H for each isomer), 3.64 (dt J - 10.5(t), d coupling hard to assign due to incomplete resolution, 2H for one isomer), 3.55-3.45(multiplet, 2H for one isomer), 2.35-1.85 (multiplet, 21H for each isomer), 1.68 (broad singlet, 6H for each isomer), 1.60 (broad singlet, 12H for each isomer), 1.48 (multiplet, 2H for each isomer), 1.27 (multiplet, 2H for each isomer), 0.88 (t, J = 7.0, 1H for each isomer).

Carbon NMR: 149.91, 149.90, 137.52, 137.34, 135.18, 135.00, 131.24, 124.54, 124.41, 124.20, 124.15, 124.11, 108.87, 65.48, 63.54, 39.76, 39.73, 39.13, 37.87, 37.75, 36.16, 36.10, 35.83, 33.91, 33.25,.32.92,.31.92, 29.52, 29.06, 27.22, 22.46, 17.69, 16.05, 16.03 and 14.14.

M/Z = 496.5, 496.5

### Example 9. Synthesis of Compound (76) in Racemic Form (2 Pairs of Diastereomers Produced)

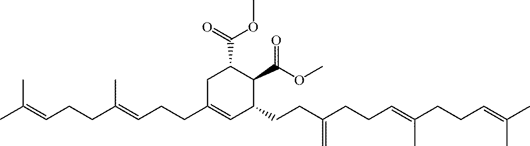 [SMILES: C=C(CC/C=C(\C)CCC=C(C)C)CC[C@@H]1C=C(CC/C=C(\C)CCC=C(C)C)C[C@H](C(=O)OC)[C@H]1C(=O)OC]

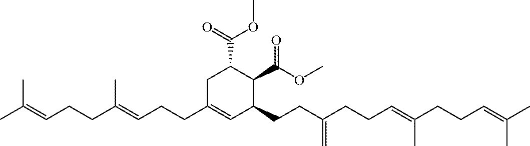 [SMILES: C=C(CC/C=C(\C)CCC=C(C)C)CC[C@H]1C=C(CC/C=C(\C)CCC=C(C)C)C[C@H](C(=O)OC)[C@H]1C(=O)OC]

### Procedure

Briefly, DHIS (81.2 g, 0.199 mol) was diluted in 100 mL toluene and subsequently added to dimethyl fumarate (24.35 g, 0.169 mol). The reaction mixture was heated at 90° C. for 13 hours. The crude product was concentrated by rotary evaporation and vacuum line to give 112.4 g technical diesters. GCMS analysis showed 93% diesters (not resolved) and about 1.5% DHIS. The product was purified by silica gel chromatography in three batches using 10% ethyl acetate in heptanes as eluent. Yield 71.4 g, 65%.

Proton NMR: 5.546 (d, J = 4.7 Hz, 1H for one isomer), 5.313 (broad singlet, 1H for 1 isomer), 5.087 (multiplet, 4H for both isomers), 4.714 (d, J = 8.5 hZ, 2H for both isomers), 3.706 (s, 3H for one isomer), 3.701 (s, 3H for one isomer), 3.682 (s, 3H for one isomer), 3.679 (s, 3H for one isomer), 3.05-2.90 (M, 2H for one isomer, 1H for other isomer), 2.60-2.40 (m, 2H for one isomer, 1H for the other isomer), 2.35-2.20 (M, 1H for each isomer), 2.20-1.9 (m, 19H for each isomer), 1.679 (broad singlet, 6H for each isomer), 1.599 (broad singlet, 12H for each isomer), 1.40-1.2 (m, 2H for each isomer).

Carbon NMR: 176.286, 175.688, 174.944, 174.326, 149.347, 148.980, 135.753, 135.519, 135.421, 135.224, 135.194, 131.347, 131.304, 124.338, 124.324, 124.278, 123.998, 123.950, 123.577, 123.465, 123.212, 109.310, 109.144, 51.92, 51.891, 51.738, 51.607, 47.128, 45.767, 43.137, 39.706, 38.425, 37.894, 37.139, 37.113, 36.044, 35.772, 35.157, 33.501, 32.063, 31.822, 31.692, 31.144, 30.639, 26.744, 26.732, 26.707, 26.265, 26.248, 26.216, 26.178, 25.712, 17.694, 16.088, 16.016.

M/Z = 552.5

### Example 10. Synthesis of Compound (16) in Racemic Form (2 Pairs of Diastereomers Produced)

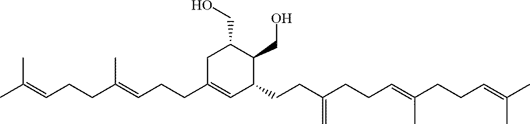 [SMILES: C=C(CC/C=C(\C)CCC=C(C)C)CC[C@@H]1C=C(CC/C=C(\C)CCC=C(C)C)C[C@H](CO)[C@H]1CO]

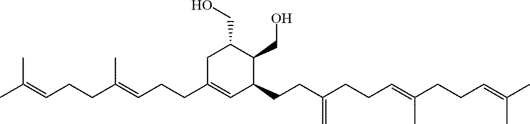 [SMILES: C=C(CC/C=C(\C)CCC=C(C)C)CC[C@H]1C=C(CC/C=C(\C)CCC=C(C)C)C[C@H](CO)[C@H]1CO]

### Procedure

400 mL of tetrahydrofuran was cooled in an ice water bath under nitrogen. After 15 minutes of cooling, solid lithium aluminum hydride (4.9 g, 0.129 mol) was added. After mixing well, a solution of compound (76) (59.3 g, 0.107 mol) in 200 mL tetrahydrofuran was added over 40 minutes. The cooling bath was removed and the mixture was stirred at ambient temperature for 30 minutes and then cooled in an ice water bath. Twenty milliliters of water was added slowly (hydrogen gas is evolved) and the mixture was acidified to pH 2 with 5% aqueous hydrochloric acid and phase separated. The organic phase was concentrated by rotary evaporation to give 163.1 g of water phase and oil. The original aqueous phase was extracted with 200 mL ethyl acetate. The ethyl acetate from this extraction was combined with the water and oil mixture and that new mixture was also phase separated. This organic phase was concentrated to give 54.5 g oil which was purified by silica gel chromatography in three batches using a 20% ethyl acetate/heptanes to 50% ethyl acetate step gradient. Yield = 47.6 g, 89.6%.

Proton NMR: 5.435 ppm (d, J = 4.2, 1H for 1 isomer), 5.297 (broad singlet, 1H for one isomer), 5.15-5.07 (m, 4H for each isomer), 4.724 (s, 2H for each isomer), 3.876 (dd, J = 9.3, 2.6 1H for 1 isomer), 3.805-3.720 (m, 2H for each isomer), 3.368 (dt, t, J = 11.0, d, J = 6.1 1H for each isomer), 3.557 (dd, J = 11.2, 4.9, 1H for 1 isomer), 2.2-1.9 (overlapping multiplets, 21H for each isomer), 1.678 (broad singlet, 6H for each isomer),

1.599 (broad singlet, 12H for each isomer), 1.47-1.35 (multiplet, 2H for each isomer), 1.36-1.25 (multiplet, 2H for each isomer).

Carbon 13 NMR: 150.01, 149.87, 136.85, 136.79, 135.30, 135.29, 135.23, 135.22, 131.19, 124.51, 124.47, 124.24, 124.15, 124.13, 109.04, 108.94, 66.63, 65.69, 64.66, 63.36, 44.34, 41.84, 40.20, 39.84, 39.82, 37.71, 37.63, 37.01, 36.92, 36.32, 36.16, 35.99, 34.13, 32.70, 32.32, 32.15, 31.36, 29.94, 26.89, 26.83, 26.52, 26.46, 26.44, 25.81, 17.80, 16.18, 16.13.

M/Z = 496.5, 496.5

### Example 11. Synthesis of Compound (71) in Racemic Form (2 Pairs of Diastereomers Produced)

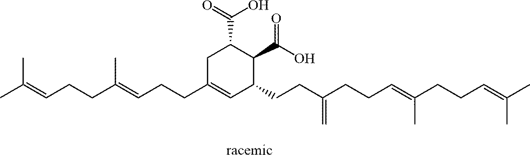

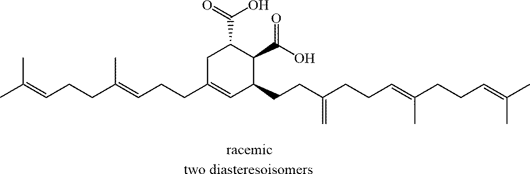

### Procedure

50 mL N,N-dimethyl was added to DHIS (51.9 g, 0.127 mol) followed by fumaric acid (13.95 g, 0.120 mol) under a nitrogen atmosphere. The mixture was heated at 120° C. for 27 hours. The crude NMR result demonstrated conversion with some residual starting material. The solvent was then removed by distillation at from 60° C. to 80° C. and 0.2 torr to give 67.3 g light brown oil. Silica gel chromatography using 5% methanol in methylene chloride + 2% acetic acid yielded 7.2 g of cleaner fractions (11%, 85% pure) along with 41.5 g mixed fractions, which were 70-80% pure. Estimated yield = 59%.

Proton NMR: 5.57 (broad singlet, 1H for 1 compound), 5.33 (broad singlet, 1H for each compound), 5.09 (m, 4H for each compound), 4.73 (broad singlet, 2H for each compound), 3.10-2.85 (m, 2H for 1 compound, 1 H for 1 compound), 2.65-2.55 (m, 1H for 1 compound), 2.52-2.42 (m, 1H for each compound), 2.40-2.33 (m, 1H for each compound), 2.25-1.92 (m, 19H for each compound), 1.676 (broad singlet, 6H for each compound), 1.598 (broad singlet, 12H for each compound), 1.6-1.45 (m, 1H for each compound), 1.42-1.24 (m, 1H for each compound).

Carbon 13 NMR: 182.74, 182.03, 181.62, 181.06, 149.30, 148.97, 135.85, 135.63, 135.34, 135.21, 135.17, 131.32, 131.23, 131.21, 124.39, 124.36, 124.27, 124.02, 123.96, 123.51, 123.47, 123.23, 109.32, 109.18, 46.84, 46.17, 43.39, 39.71, 38.23, 38.19, 37.10, 31.64, 31.26, 31.12, 30.53, 26.73, 26.31, 26.27, 26.24, 26.15, 25.67, 17.67, 16.07, 16.00, 15.99.

M/Z = 523.38. 523.38 (negative ion, M-1)

### Example 12. Synthesis of Compound (61)

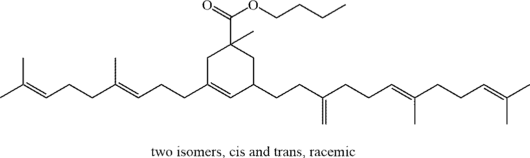

### Procedure

DHIS (14.3 g, 35 mmol) was added to a 250 mL flask followed by n-butylmethacrylate (7 mL, 44 mmol) and 15 mL toluene. The mixture was heated at 120° C. for 8 hours when GC showed an approximate 2:1 ratio of desired product isomers to DHIS. An additional 10 mL of toluene and 2 mL n-butylmethacrylate was added and heating at 120° C. was resumed for 5 hours. GC showed high (but incomplete) conversion. The reaction product was concentrated by rotary evaporation and most of the starting methacrylate was removed by distillation at about 1 torr, 80° C. to give 17.8 g slightly cloudy yellow oil. 1.2 g of this oil was purified by silica gel chromatography using a 10% toluene/heptane to 10% ethyl acetate/heptane step gradient to give 0.6 g colorless oil. Yield was estimated as 46% of a mixture of two isomers.

Proton NMR: 5.447 (4, J = 4.9 Hz, 1H for 1 isomer), 5.30 (broad singlet, 1H for 1 isomer), 5.10-5.06 (m, 4H for each isomer), 4.719 (d, J = 4.3 Hz, 2H for each isomer), 4.16-4.04 (m, 2H for each isomer), 2.3-1.85 (m, 21H for each isomer), 1.687 (singlet, 6H for each isomer), 1.608 (s, 12H for each isomer), 1.70-1.57 (m, 2H for each isomer), 1.50-1.30 (m, 6H for each isomer), 1.18 (3, 3H for each isomer), 1.03 (s, 3H for 1 isomer), 0.937 (t, J = 7.4 Hz, 3H for each isomer).

Carbon 13 NMR: 177.77, 149.57, 135.86, 135.14, 135.12, 131.31, 124.36, 124.06, 122.97, 122.31, 108.98, 64.20, 63.96, 44.46, 42.24, 40.41, 39.74, 39.72, 37.48, 36.06, 35.89, 34.01, 32.74, 31.94, 30.72, 29.77, 26.78, 26.72, 26.39, 26.28, 25.71, 25.45, 22.40, 19.30, 19.25, 17.70, 16.08, 16.07, 16.02, 13.75.

GCMS M/Z = 550.5, 550.5

### Example 13. Synthesis of Compound (6)

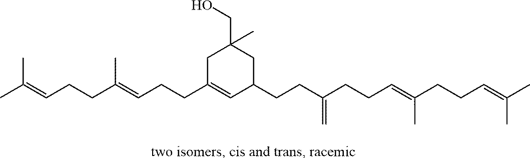

### Procedure

Impure compound (61) (16.6 g, about 50% pure, 18.1 mmol) was dissolved in 20 mL tetrahydrofuran and added to a 0° C. suspension of lithium aluminum hydride (0.545 g, 15.1 mmol) in 20 mL dry tetrahydrofuran over 10 minutes. The suspension was stirred at room temperature for 90 minutes and cooled back to 0° C. 1 mL water (hydrogen liberated) was then carefully added. The mixture was then carefully acidified to pH 4 with 5% aqueous hydrochloric acid, and the product was extracted with 25 mL ethyl acetate. After removal of most of the solvent by rotary evaporation, the alcohols were purified by silica gel chromatography using 10% ethyl acetate as eluent. Yield = 5.5 g colorless oil, 76%.

Proton NMR: 5.359 (broad singlet, 1H for 1 isomer), 5.322 (broad singlet, 1H for 1 isomer), 5.16-5.06 (m, 4H for both isomers), 4.737 (broad singlet, 2H for both isomers), 3.49 (d, J = 18.8 Hz, 1H for 1 isomer), 3.433 (s, 2H for 1 isomer), 3.34 (d, J = 18.8 Hz, 1H for one isomer), 2.26-1.78 (overlapping multiplets, 21H per isomer), 1.679 (broad singlet, 6H for each isomer), 1.600 (broad singlet, 12H for each isomer), 1.35-1.20 (m, 2H for one isomer, 1 H for one isomer), 1.18-1.04 (m, 1 H for one isomer), 0.97 (s, 3H for one isomer), 0.881 (dd (overlapping), J = 6.8 Hz, 2H for each isomer), 0.76 (s, 3H for 1 isomer).

Carbon 13 NMR: 150.03, 149.81,136.75, 136.12, 135.18, 135.15, 135.03, 134.99, 131.25, 124.39, 124.37, 124.19, 124.16, 124.09, 123.36, 123.20, 108.99, 108.96, 70.34, 67.37, 42.72, 39.74, 39.71, 38.68, 37.63, 37.61, 36.47, 36.29, 36.15, 36.08, 34.41, 34.25, 31.88, 30.66, 29.74, 29.02, 28.61, 28.46, 26.78, 26.72, 26.48, 26.46, 26.36, 25.68, 25.58, 22.69, 17.68, 17.27, 16.06, 16.01, 14.10.

GCMS M/Z = 480.4, 480.4

### Example 14. Synthesis of Compound (11)

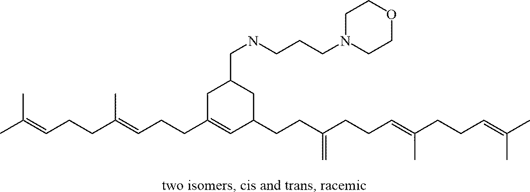

### Procedure

Compound (41) (3.0 g, 6.46 mmol), 3-morpholinopropylamine (1.12 g, 7.76 mmol) and 60 mL 1,2-dichloroethane were added into a 250 mL flask under nitrogen atmosphere at room temperature. The resulting mixture was stirred for 1 hour at room temperature. Sodium triacetoxyborohydride (5.48 g, 7.75 mmol) was slowly added in four portions at 10-minute intervals for 30 minutes, and the mixture was stirred at 20-30° C. for an additional 10 minutes. The reaction was then heated at 40-50° C. for 12 hours. The mixture was cooled to room temperature. Methanol (30 mL) was added slowly over 30 minutes, and then the mixture was stirred for 30 minutes. The solvents were removed by evaporation under vacuum under 40° C. The crude material was purified by CombiFlash column using aminosilica gel as stationary phase and eluted with 40% ethyl acetate in hexanes. The fractions containing pure product were collected and dried under vacuum to give a green liquid (2.00 g, 52% yield). The ratio of diastereoisomers was about 1:1.

Proton NMR: 5.40 (broad singlet, 1H for 1 isomer), 5.30 (broad singlet), 5.20-5.00 (m, 4H for both isomers), 4.80-4.60 (broad singlet, 2H for both isomers), 3.72 (t, J = 4.4 Hz, 4H for both isomers), 2.70-2.60 (m, 2H for one isomer, 3H for one isomer), 2.55-2.30 (m, 8H for both isomers), 2.20-1.88 (m, 21H for both isomers), 1.88-1.75 (m. 2H for both isomers), 1.75-1.65 (m, 2H for one isomer, 3H for one isomer), 1.66 (broad singlet, 6H for both isomers), 1.60 (broad singlet, 12H for both isomers), 1.60-1.34 (m, 2H for both isomers), 1.34-1.22 (m, 1H for both isomers).

Carbon 13 NMR: 150.06, 137.34, 137.16, 135.16, 135.14, 134.96, 131.28, 131.26, 124.71, 124.39, 124.36, 124.27, 124.21, 124.09, 108.76, 67.02, 57.59, 53.85, 53.21, 50.44, 49.04, 48.99, 39.75, 39.72, 38.10, 37.86, 37.72, 37.20, 36.71, 36.55, 36.16, 33.89, 33.35, 33.04, 29.70, 29.43, 27.36, 26.79, 26.74, 26.47, 26.42, 26.39, 25.70, 24.62, 23.77, 17.70, 16.06, 16.03.

Mass analysis: ESI, M/Z = 593.74 [M + H]+, positive ion

HPLC Analysis: 90.68 Area %

### Example 15. Synthesis of Compound (36) in Racemic Form (2 Pairs of Diastereomers Produced)

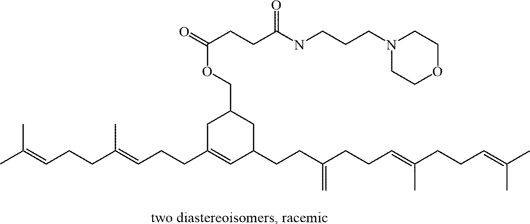

### Procedure

Compound (31) (15.0 g, 0.026 mol) and N-(3-aminopropyl) morpholine (4.77 g, 0.033 mol) were dissolved in 150 mL N,Ndimethylformamide. The solution was cooled to 0-5° C., and diisopropylethyl amine (17.1 g, 0.132 mol) was added over 30 minutes under a nitrogen atmosphere. The reaction mixture was stirred at 0-5° C. for thirty minutes, and then a solution of propyl phosphonic anhydride (25.26 g, 50% in ethyl acetate, 0.040 mol) was added over 30 minutes. The reaction mixture was stirred for 16 hours at 20-25° C. The reaction mixture was cooled to 15-20° C., and water (10 volumes) and 10% aqueous sodium hydroxide (5 volumes) were added. The resulting mixture was stirred for 15 minutes at 20-25° C. The reaction mixture was extracted with ethyl acetate (2 × 10 volumes). The combined organic phases were washed with 10% citric acid solution, saturated sodium bicarbonate solution, water, and then sodium chloride solution. The organic later was dried over anhydrous sodium sulfate and concentrated under vacuum at 40-45° C. to obtain a pale yellow oil (10.0 g, 60.0% yield).

Proton NMR: 7.00-6.90 (m, 1H for both isomers), 5.37 (broad singlet, 1H for one isomer), 5.28 (broad singlet, 1H for one isomer), 5.17-5.07 (m, 4H for both isomers), 4.722 (broad singlet, 2H for both isomers), 4.10-3.89 (m, 6H for both isomers), 3.51 (broad doublet, J = 12.4 Hz, 2H for both isomers), 3.36 (quartet, J =6.0 Hz, 2H for both isomers), 3.15 (triplet, J = 7.6 Hz, 2H for both isomers), 2.89 (broad triplet, J = 8.8 Hz, 2H for both isomers), 2.66 (broad triplet, J = 6 Hz, 2H for both isomers), 2.49 (broad triplet, J = 6.4 Hz, 2H for both isomers), 2.28-1.88 (m, 28 H for both isomers), 1.676 (broad singlet, 6H for each isomer), 1.596 (broad singlet, 12H for both isomers), 1.59-1.27 (m, 4H for both isomers).

Carbon 13 NMR (100 MHz, CDCl3): 172.07, 172.11, 148.68, 148.58, 136.40, 136.35, 134.13, 134.11, 134.02, 130.19, 123.36, 123.10, 123.08, 123.04, 122.95, 107.95, 107.90, 66.10, 66.04, 64.15, 56.62, 52.68, 38.70, 38.19, 36.77, 36.63, 35.34, 35.11, 35.04, 34.92, 34.34, 32.71, 30.17, 30.17, 30.15, 28.67, 28.59, 28.56, 25.75, 25.71, 25.68, 25.42, 24.38, 25.33, 24.68, 24.09, 22.73, 21.79, 16.67, 15.02, 15.01.

Mass Analysis: ESI, M/Z = 693.71 [M + H]+, (positive ion)

HPLC Analysis: 94.8 Area %

### Example 16. Synthesis of Compound (66)

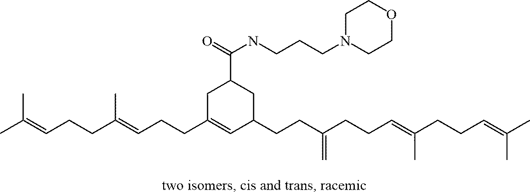

### Procedure

Compound (46) (7.50 g, 0.016 mol), N-(3aminopropyl) morpholine (2.25 g, 0.016 mol) and 150 mL dichloromethane were charged to a 500 mL round bottom flask at room temperature. The reaction mixture was cooled to 0-5° C. Diisopropylethyl amine (10.08 g, 0.078 mol) was added dropwise over 30 minutes through an addition funnel under a nitrogen atmosphere. The reaction was stirred for 30 minutes at 0-5° C. A solution of Propylphosphonic anhydride (14.89 g, 50 wt% in ethyl acetate, 0.023 mol) was added slowly via addition funnel to the reaction mixture at 0-5° C. over 30 minutes under nitrogen atmosphere. The reaction mixture was stirred for 3-4 hours at 20-25° C., and the reaction progress was monitored by TLC. Then, the reaction mixture was cooled to 15-20° C. and quenched with saturated sodium bicarbonate solution. The resulting mixture was stirred for 10 minutes at 20-25° C. The organic layer was washed with 10% citric acid solution, saturated sodium bicarbonate solution, and water followed by sodium chloride solution. The organic solution was dried over anhydrous sodium sulfate and concentrated under vacuum at 35-40° C. to obtain crude compound. The crude compound was subjected to CombiFlash chromatography (amino silica gel cartridge) and eluted with 10% ethyl acetate in petroleum ether to 30% ethyl acetate petroleum ether step gradient. The collected fractions were concentrated to afford a pale green liquid (4.50 g, 46.5% yield). The ratio of the two isomers was approximately 63:37.

Proton NMR (400 MHz, CDCl3): 6.88 (broad triplet, NH for one isomer), 6.76 (broad triplet, NH for one isomer), 5.52 (broad singlet, 1H for one isomer), 5.36 (broad singlet, 1H for one isomer), 5.13-5.80 (m, 4H for both isomers), 4.73-4.71 (singlet, 2H for both isomers), 3.73-3.70 (m, 4H for both isomers), 3.41-3.35 (m, 2H for both isomers), 2.49-2.37 (m, 7H for both isomers), 2.16-2.00 (m, 25H for both isomers), 1.99-1.79 (3H for both isomers), 1.69 (singlet, 6H for both isomers), 1.61 (singlet, 12H for both isomers), 1.60-1.25 (m, 2H for both isomers).

Carbon 13 NMR (100 MHz): 174.68, 173.50, 148.70, 148.45, 136.79, 136.26, 134.19, 130.29, 123.29, 122.93, 122.73, 122.59, 108.02, 107.78,66.01, 65.95, 56.88, 52.76, 52.70, 47.43, 44.10, 38.69, 38.18, 38.04, 36.56, 36.35, 35.41, 35.18, 35.01, 32.62, 28.93, 26.93, 26.10, 25.74, 25.69, 24.06, 20.35. 16.68, 16.63, 15.04, 15.01.

LCMS: ESI, M/Z = 607.73 [M + H]+ and 607.76 [M + H]+ (positive ion)

HPLC Analysis: 85.9 Area % (n = 2), Fortis C18, 150 × 4.6 mm, 3 µm, DAD @ 205 nm

### Example 17. Synthesis of Compound (81)

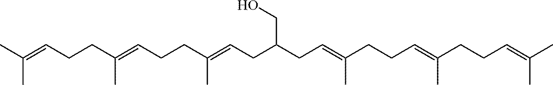 [SMILES: CC(C)=CCC/C(C)=C/CC/C(C)=C/CC(CO)C/C=C(\C)CC/C=C(\C)CCC=C(C)C]

### Procedure

To a solution of 1.0 M LiAlH₄ in THF (43.0 mL, 0.043 mol), a solution of compound (84) (26.0 g, 0.054 mol) was added at 0-5° C. under nitrogen atmosphere. The reaction mixture was maintained for 3-4 hours at 0-5° C. The reaction mixture was carefully quenched with a mixture of THF and water (5 vol) at 0-5° C. pH of the reaction mass was adjusted to 1 with 1 M aqueous HCl (3.0 vol) at 5-10° C. The organic layer was separated, and the aqueous layer was extracted with ethyl acetate (2 × 5.00 vol). The combined organic layer was dried over Na₂SO₄ and evaporated under vacuum at 40° C. to get crude compound. The crude compound was purified by column chromatography on silica gel (100-200 mesh) and eluted with 8-10% ethyl acetate in hexanes. All the fractions containing pure product were collected and dried under vacuum at 40° C. to afford a pale yellow liquid (17.2 g, 70.2 % yield, 99.17 % purity AUC by HPLC (82.0% & 17.17%, two peaks)).

Proton NMR (400 MHz, CDCl3): 5.17 (broad triplet, 2H, J=6.8 Hz), 5.07-5.15 (m, 4H), 3.53 (broad singlet, 2H), 2.14-1.95 (m, 21H), 1.68 (singlet, 6H), 1.65 (singlet, 6H), 1.59 (broad singlet, 12H).

Carbon 13 NMR (100 MHz, CDCl3): 136.52, 135.09, 131.28, 124.38, 124.10, 122.64, 66.10, 42.16, 39.87, 39.73, 29.68, 26.74, 26.59, 25.71, 17.68, 16.13, 16.02.

Mass Analysis: ESI, M/Z = 455 [M+H]+, (positive ion)

HPLC Analysis: 82.0 Area %, Synergi Polar RP, 250 × 4.6 mm, 4 µm, DAD @205 nm

### Example 18. Synthesis of Compound (82)

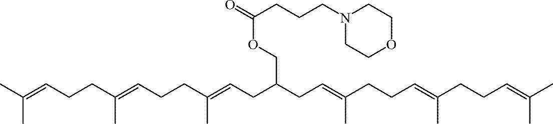 [SMILES: CC(C)=CCC/C(C)=C/CC/C(C)=C/CC(C/C=C(\C)CC/C=C(\C)CCC=C(C)C)COC(=O)CCCN1CCOCC1]

### Procedure

Charged compound (81) (9.50 g 20.9 mmol), 4-Morpholinobutanoic acid (5.07 g, 29.2 mmole), diisopropylethylamine (18.72 mL, 104.4 mmol), 4-4-Dimethylaminopyridine (0.23 g, 2.08 mmol), and dichloromethane (190 mL, 20.0 vol) were added into a 500 mL three neck round bottom flask under nitrogen atmosphere and stirred for 5-10 minutes at 20-25° C. Then, N-Ethyl-N′-(3-dimethylaminopropyl)-carbodiimide hydrochloride (5.59 g, 29.20 mmol) was slowly added into the reaction mixture and stirred for 24 hours at 20-30° C. The reaction mixture was diluted with 190 mL water and stirred for 10 minutes at room temperature. The organic layer was separated, and the aqueous layer was again extracted with dichloromethane (20.0 vol). The combined organic layers were washed with 95 mL saturated sodium bicarbonate solution followed by 48 mL saturated brine solution. The solution was dried over anhydrous sodium sulphate. Then, the organic layer was concentrated under reduced pressure at 35-40° C. to get crude compound. The crude compound was purified by CombiFlash using 4-6% methanol in dichloromethane. All the pure fractions were collected and the solvents completely under vacuum to afford a colorless liquid (7.50 g, 59.7% yield, 82% purity by HPLC/UV).

Proton NMR (400 MHz, CDCl3): 5.18-5.05 (m, 6H), 3.951 (doublet, 2H, J=5.6 Hz), 3.69 (triplet, 4H, J=4.4 Hz,), 2.45-2.40 (m, 4H), 2.37-2.33 (m, 4H), 2.12-1.94 (m, 19H), 1.86- 1.69 (m, 4H), 1.67 (broad singlet, 6H), 1.59 (broad singlet, 18H).

Carbon 13 NMR (100 MHz, CDCl3): 173.58, 136.78, 135.04, 131.22, 124.40, 124.11, 121.90, 67.01, 66.60, 58.08, 53.65, 39.86, 39.71, 39.01, 32.16, 29.26, 26.76, 26.63, 25.70, 21.86, 17.68, 16.10, 16.01.

Mass Analysis: ESI, M/Z = 610.39 [M+H]+, (positive ion)

HPLC Analysis: 82 Area %, DAD @205 nm

### Example 19. Synthesis of Compound (83)

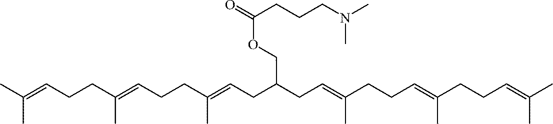 [SMILES: CC(C)=CCC/C(C)=C/CC/C(C)=C/CC(C/C=C(\C)CC/C=C(\C)CCC=C(C)C)COC(=O)CCCN(C)C]

### Procedure

Compound (81) (7.50 g, 0.016 mol), N,N-dimethylamino butanoic acid hydrochloride (4.14 g, 0.025 mol, 1.5 equiv), and 150 mL dichloromethane were added to a 500 mL round bottom flask at room temperature. N, N-Diisopropylethylamine (10.65 g, 0.082 mol) was added dropwise at 20-25° C. over 15 minutes via addition funnel under nitrogen atmosphere. The reaction mixture was stirred for 15 minutes at 20-25° C. 1-Ethyl-3-(3-dimethylaminopropyl) carbodiimide (10.65 g, 0.025 mol) was added to the reaction mixture at 20-25° C. under nitrogen atmosphere. 4-Dimethylaminopyridine (0.18 g, 0.0016 mol) was added to the reaction mixture at 25-30° C. The reaction mixture was stirred for 16 hours at 20-25° C. The reaction mixture was diluted with water (15 vol) and layers were separated. The aqueous layer was extracted with 10 volumes of dichloromethane. The combined organic layers were washed with saturated sodium bicarbonate (10 vol), water (10 vol) and brine solution (5 vol). The organic layer was dried over anhydrous sodium sulphate and concentrated under vacuum at 40° C. to obtain crude product. The crude product was purified by CombiFlash and eluted with 6-8 % methanol in dichloromethane. The pure fractions were collected and evaporated to get pure product (7.0 g, 78.0 % yield, 74.6% pure by LCUV) as a yellow liquid.

Proton NMR (400 MHz, CDCl3): 5.16-5.06 (m, 6H), 3.95 (d, 2H, J=5.6 Hz), 2.334 (triplet, J = 7.6 Hz, 2H), 2.29 (triplet, J = 7.2 Hz, 2H), 2.21 (s, 6H), 2.12-1.94 (m, 19H), 1.84- 1.68 (m, 4H), 1.67 (broad singlet, 6H), 1.59 (broad singlet, 18H).

Carbon 13 NMR (100 MHz, CDCl3): 173.64, 136.78, 135.01, 131.20, 124.40, 124.08, 121.93, 66.51, 58.92, 45.39, 39.86, 39.71, 39.01, 32.14, 29.29, 26.76, 26.63, 25.69, 23.53, 23.00, 17.67, 16.09, 16.00.

Mass Analysis: ESI, M/Z = 568.37 [M+H]+, (positive ion)

HPLC Analysis: 74.6 Area %, DAD @205 nm

### Example 20. Synthesis of Compound (85)

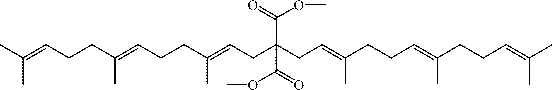 [SMILES: COC(=O)C(C/C=C(\C)CC/C=C(\C)CCC=C(C)C)(C/C=C(\C)CC/C=C(\C)CCC=C(C)C)C(=O)OC]

### Procedure

To a solution of dimethylmalonate (25.0 g, 0.189 mol) in 250 mL methanol, dropwise 30% sodium methoxide in methanol (170.4 g, 0.947 mol) was added at 25-30° C. The reaction mass was heated to 50-55° C. and maintained for 1 hour. The clear homogeneous solution was turned into a white suspension. The reaction mixture was cooled to 25-30° C. Farnesyl chloride (182.4 g, 0.757 mol) was added dropwise to the reaction mass at 25-35° C. The reaction mixture was heated to 40-45° C. and heating continued for 12 hours. The reaction mixture was evaporated under vacuum at 40° C. to get a residue. The residue was diluted with water (10.0 vol) and extracted with ethyl acetate (2 × 10.0 vol). The combined organic layer was washed with water and brine. The organic layer was dried over Na₂SO₄ and evaporated to get crude product. The crude product was purified by column chromatography on silica gel (100-200 mesh) and eluted with 5-6% ethyl acetate in hexanes. All the fractions containing product along with impurity (two spots by TLC) were collected and dried completely under vacuum at 40° C. to afford a yellow liquid (104 g). This crude material was 20-30% pure by LCUV. An analytical sample was prepared by additional chromatography.

Proton NMR (400 MHz, CDCl3): 5.10-5.04 (m, 4H), 4.96 (broad triplet, 2H, J=6.4 Hz), 3.69 (singlet, 6H), 2.60 (doublet, 4H, J=7.2 Hz), 2.08-1.94 (m, 16H), 1.67 (singlet, 6H), 1.59 (broad singlet, 18H).

Carbon 13 NMR (100 MHz, CDCl3): 171.89, 139.17, 135.15, 131.27, 124.38, 123.94, 117.79, 57.88, 52.23, 40.00, 39.71, 30.81, 26.75, 26.61, 25.71, 17.68, 16.15, 16.01.

Mass Analysis: ESI, M/Z = 563 [M+Na]+, (positive ion)

HPLC Analysis: 88.21 Area %, XSelect CSH C18, 150×4.6 mm, 3.5 µm, DAD@205 nm

### Example 21. Synthesis of Compound (84)

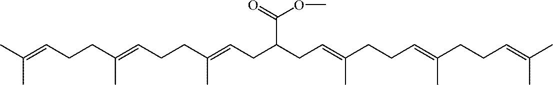 [SMILES: COC(=O)C(C/C=C(\C)CC/C=C(\C)CCC=C(C)C)C/C=C(\C)CC/C=C(\C)CCC=C(C)C]

### Procedure

Lithium chloride (81.5 g, 1.92 mol) was added to a solution of compound (85) (104 g, 0.0578 mol, about 30% pure) in 1.04 L dimethylformamide at 25-30° C. under nitrogen atmosphere. The reaction mixture was heated at 110-120° C. for 16 hours. The reaction mixture was cooled to 25-30° C. The reaction mixture was diluted with water (50.0 vol) and extracted with ethyl acetate (2 × 10.0 vol). The combined organic layers were washed with water and brine. The organic layer was dried over Na₂SO₄ and evaporated under vacuum at 40° C. to get crude product. The crude product was purified by column chromatography on silica gel (100-200 mesh) and eluted on 5-6 % ethyl acetate in hexanes. All the fractions containing product along with impurity (two spots by TLC) were collected and dried under vacuum at 40° C. to afford impure product (26.0 g, 18.6 % yield, about 20% pure, 10.8 mmol,) as yellow liquid. Analytical data were obtained after additional purification to give higher quality material (59% by LCUV).

Proton NMR (400 MHz, CDCl3): 5.14-5.03 (m, 6H), 3.64 (singlet, 3H), 2.45-1.93 (m, 21H), 1.679 (broad singlet, 6H), 1.599 (broad singlet, 18H).

Carbon 13 NMR (100 MHz, CDCl3): 176.19, 137.32, 135.03, 131.26, 124.38, 124.03, 121.15, 51.33, 46.21, 39.80, 39.73, 30.13. 26.76, 26.64, 25.71, 17.68, 16.07, 15.99

Mass Analysis: ESI, M/Z = 505 [M+Na]+, (positive ion)

HPLC Analysis: 59.03 Area %, XSelect CSH (150 × 4.6 mm), 5 µm, VWD @205 nm

### Example 22. Synthesis of Compound (86)

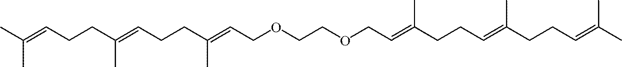 [SMILES: CC(C)=CCC/C(C)=C/CC/C(C)=C/COCCOC/C=C(\C)CC/C=C(\C)CCC=C(C)C]

### Procedure

Ethylene glycol (4.00 g, 64.5 mmol) was added to a stirred solution of sodium hydroxide (15.47 g, 390 mmol) and water (32.0 mL, 8.0 vol) in a 500 mL three neck round bottom flask at room temperature. The resulting mixture was heated to 90-95° C. and stirred for 2 hours. The reaction mixture was cooled to room temperature. Then, farnesyl chloride (77.40 g, 322 mmol) was added slowly into the reaction followed by solid tetrabutylammonium iodide (4.76 g, 12.9 mmol). The mixture was heated at 50-55° C. and maintained overnight at 50-55° C. The reaction mixture was cooled to room temperature and then diluted with 40 mL water and extracted with methyl tertiary butyl ether (2 × 100.0 mL). The combined organic layers were washed with 20 mL saturated brine solution and dried over 4.0 g anhydrous sodium sulphate, filtered and then dried under vacuum below 40° C. to get crude product. This crude product was purified by silica gel (100-200 mesh size) column chromatography and eluted with 4-6% ethyl acetate in petroleum ether. All of the pure fractions were collected and dried under vacuum to afford 7.50 g, 59% yield.

Proton NMR (400 MHz, CDCl3): 5.37 (broad triplet, J = 6.8 Hz, 2H), 5.09 (m, 4H), 4.04 (d, J = 6.8 Hz, 4H), 3.62 (s, 4H), 2.09 (m, 16H), 1.67 (s, 6H), 1.66 (s, 6H) 1.60 (s, 12H).

Carbon 13 NMR (100 MHz, CDCl3): 140.02, 135.25, 131.30, 124.35, 123.90, 120.92, 69.27, 67.71, 39.71, 39.63, 26.73, 26.31, 25.71, 17.70, 16.50, 16.01

Mass Analysis: ESI, M/Z = 488 [M+NH4]⁺

HPLC Analysis: 99.1 Area % at 205 nm

### Example 23. Synthesis of Compound (87)

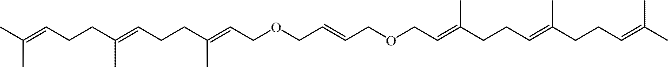 [SMILES: CC(C)=CCC/C(C)=C/CC/C(C)=C/COC/C=C/COC/C=C(\C)CC/C=C(\C)CCC=C(C)C]

### Procedure

Trans 2-butene1,4 diol (4.00 g, 45.4 mmol) was added to a stirred solution of sodium hydroxide (10.9 g, 272 mmol) and water (24.0 mL, 6.0 vol) in a 500 mL three neck round bottom flask at room temperature and then heated at 90-95° C. The resulting reaction mixture was stirred for 2 hours at 90-95° C. The mixture was cooled to room temperature. Then, farnesyl chloride (54.56 g, 227 mmol) was added slowly into the reaction mass. Charged tetrabutylammonium iodide (3.36 g, 9.09 mmol) was then added, and the reaction mass was heated to 50-55° C. The reaction mass was then heated at 50-55° C. overnight. The reaction mass was then cooled to room temperature, diluted with water (40.0 mL, 10.0 vol) and extracted with MTBE (2 × 100 mL). Combined organic layers were washed with 20 mL saturated brine solution. The organic layer was dried over 4.0 g anhydrous sodium sulphate. Then, solvent was removed under vacuum below 40° C. The crude material was purified by column chromatography using silica gel (100-200 mesh), and the compound was eluted with 4-6% ethyl acetate in petroleum ether. All the pure fractions were collected, and the solvents were removed under vacuum to afford pure product as a pale yellow liquid.

Proton NMR (400 MHz, CDCl3): 5.85 (m, 2H), 5.37 (broad triplet, J = 6.8 Hz, 2H), 5.12 (m, 4H), 3.99 (m, 8H), 2.18-1.95 (m, 16H), 1.70 (s, 6H), 1.68 (s, 6H) 1.62 (s, 12H).

Carbon 13 NMR (100 MHz, CDCl3): 140.29, 135.27, 131.30, 124.35, 123.88, 120.75, 69.98, 66.62, 39.71, 39.61, 26.73, 26.32, 25.71, 17.70, 16.53, 16.01.

Mass Analysis: ESI, M/Z = 514, M+!8 [M+NH4]⁺

HPLC Analysis: 98.8 Area %, Phenomenex Synergi Polar RP, 250 × 4.6 mm, 4.0 µm, VWD @205 nm

### Example 24. Synthesis of Compound (88)

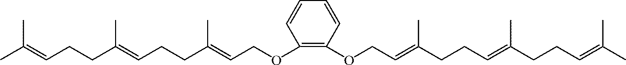 [SMILES: CC(C)=CCC/C(C)=C/CC/C(C)=C/COc1ccccc1OC/C=C(\C)CC/C=C(\C)CCC=C(C)C]

### Procedure

Potassium carbonate (18.82 g, 136.0 mmol, 6.00 equivalents) was added to a solution of catechol (2.50 g, 23.0 mmol, 1.00 equiv) in N,N-dimethyl formamide (50.0 ml, 20 vol.) under nitrogen atmosphere at 25-30° C. The reaction mass heated to 55-60° C. for 1 hour. The reaction mass was cooled to 25° C. Farnesyl chloride (21.8 g, 90.8 mmol, 4.00 equiv) was slowly added to the stirred mixture over a period of 15 minutes. The mixture was heated to 55-60° C. and stirred for 5-6 hours. Reaction conversion was monitored by TLC (Mobile phase: 5% ethyl acetate in hexanes). The reaction mass was cooled and diluted with water (250 mL) at 25-30° C. The product was extracted with methyl t-butyl ether (2 × 100 ml) at 25° C. The combined organic layer was washed with water (5.0 vol) followed by brine (5.0 vol). The organic layer was dried over anhydrous sodium sulphate and evaporated under reduced pressure at below 40° C. to get crude product. The crude product was purified by column chromatography on silica gel (100-200 mesh) and eluted using 2-5% ethyl acetate in hexanes. All the pure fractions were combined and evaporated to get pure product (9.00 g, yield 80.3%, 96.3% purity AUC by HPLC) as yellow liquid.

Proton NMR (400 MHz, CDCl3): 6.87 (m, 4H), 5.52 (t, 2H, J=6.0 Hz), 5.08 (m, 4H), 4.60 (d, 4H, J=6.4 Hz), 2.15-2.09 (m, 4H), 2.07-2.04 (m, 8H), 1.98-1.94 (m, 4H), 1.71 (s, 6H), 1.67 (s, 6H), 1.59 (s, 12H).

Carbon 13 NMR (100 MHz, CDCl3): 148.95, 140.15, 135.32, 131.27, 124.39, 123.84, 121.01, 120.27, 114.23, 66.09, 39.73, 33.59, 26.76, 26.30, 25.74, 17.72, 16.72, 16.04.

Mass Analysis: ESI, M/Z = 519.69 [M+H]⁺ and 541.66 [M+Na]⁺

HPLC Analysis: 96.3 Area % (n=2), XBridge Phenyl, 150 × 4.6 mm, 3.5 µm, VWD @205 nm

### Example 25. Synthesis of Compound (89)

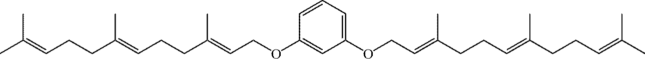 [SMILES: CC(C)=CCC/C(C)=C/CC/C(C)=C/COc1cccc(OC/C=C(\C)CC/C=C(\C)CCC=C(C)C)c1]

### Procedure

N, N-Dimethylformamide (37.5 mL, 25 volumes), Resorcinol (1.50 g, 14.0 mmol) and potassium carbonate (18.82 g, 136.0 mmol, 10.0 equiv) were added into a 250 mL three neck round bottom flask at room temperature under nitrogen atmosphere. The resulting reaction mixture was heated to 75-80° C. and stirred for 2 hours. The reaction mass was cooled to room temperature and farnesyl chloride (13.3 g, 54.0 mmol) was slowly added. The resulting mixture was heated to 55-60° C. and stirred at 55-60° C. overnight. The reaction conversion was monitored by TLC (5% ethyl acetate in hexane). Upon completion of the reaction, the reaction mixture was cooled to room temperature and diluted with water (200 mL). The compound was then extracted with MTBE (2 × 100 mL). The organic layer was dried over anhydrous sodium sulphate followed by concentration at below 40° C. under reduced pressure to get crude product. The crude product was purified by CombiFlash chromatography using ethyl acetate and hexanes. The product was eluted at 3-6% ethyl acetate in hexanes. All the pure fractions were combined and concentrated to get pure product (3.00 g, 43.4% yield, 95.6% purity AUC by HPLC), as a pale yellow liquid.

Proton NMR (400 MHz, CDCl3): 7.14 (m, 1H), 6.50 (m, 3H), 5.49 (t, 2H, J=6.4 Hz), 5.15-5.05 (m, 4H), 4.49 (d, 4H, J=6.4 Hz), 2.18-2.03 (m, 12H), 1.99-1.95 (m, 4H), 1.75 (s, 6H), 1.67 (s, 6H), 1.60 (s, 12H). Carbon 13 NMR (100 MHz, CDCl3): 160.13, 141.15, 135.43, 131.31, 129.73, 124.36, 123.74, 119.52, 106.92, 101.79, 64.85, 39.71, 39.59, 26.74, 26.27, 25.71, 17.70, 16.67, 16.03.

Mass Analysis: ESI, M/Z = 520 [M+H]+, (positive ion)

HPLC Analysis: 95.6 Area %, XBridge Phenyl, 150 × 4.6 mm, 3.5 µm, DAD @205 nm

### Example 26. Synthesis of Compound (90)

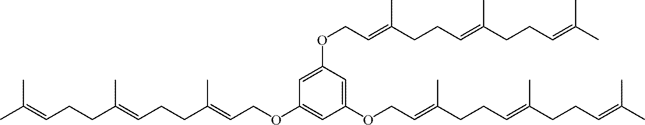 [SMILES: CC(C)=CCC/C(C)=C/CC/C(C)=C/COc1cc(OC/C=C(\C)CC/C=C(\C)CCC=C(C)C)cc(OC/C=C(\C)CC/C=C(\C)CCC=C(C)C)c1]

### Procedure

To a solution of Phloroglucinol (3.00 g, 23.8 mmol, 1.00 equiv) in N,N-dimethyl formamide (60.0 mL, 20 vol.), potassium carbonate (32.8 g, 238 mmol, 10.00 equiv) was added under nitrogen atmosphere at 25-30° C. The reaction mass was heated to 55-60° C. for 1 hour. The reaction mass was cooled to 25° C. Farnesyl chloride (45.8 g, 190 mmol, 8.00 equiv) was slowly added to the stirred mixture over a period of 15-20 minutes. The reaction mass was heated to 55-60° C. and stirred for 16-20 hours. The reaction mass was cooled to 25-30° C. and diluted with 300 mL water at 25-30° C. The product was extracted with MTBE (2 × 150 mL) at 25° C. The combined organic layers were washed with 50 mL water followed by 50 mL brine. The organic layer was dried over anhydrous sodium sulphate, and the solvent was removed under reduced pressure at below 40° C. to get crude product. The crude product was purified by column chromatography on basic alumina and eluted using 0.5-1.0% ethyl acetate in hexanes. All the pure fractions were combined and evaporated to get pure product (8.50 g, 48.3% yield, 79.8% purity AUC by HPLC) as pale yellow liquid. Additional purification by Methods 1 and 2 gave 5.20 g, 29% yield, 96.4% purity by HPLC/UV.

### Purification Method 1

The product (~8.0 g, different batches) was purified by reverse phase CombiFlash and eluted with 95-100% acetonitrile. 1.60 g of pure material was isolated with 94.91% HPLC purity.

### Purification Method 2

The product (~18.0 g, different batches) was purified using SFC purification method. 3.60 g of pure material was isolated with 94.91% HPLC purity.

All pure fractions were dissolved in dichloromethane and mixed and evaporated under vacuum to get 5.20 g with 96.4% HPLC purity.

Proton NMR (400 MHz, CDCl3): 6.13 (s, 3H), 5.50 (t, 3H, J=6.0 Hz), 5.11 (m, 6H), 4.48 (d, 6H, J=6.8 Hz), 2.16-1.97 (m, 24H), 1.73 (s, 9H), 1.69 (s, 9H), 1.61 (s, 18H).

Carbon 13 NMR (100 MHz, CDCl3): 158.67, 139.17, 133.39, 122.37, 121.74, 117.42, 92.12, 62.83, 37.72, 37.59, 24.73, 24.26, 23.72, 15.71, 14.66, 14.03.

Mass Analysis: ESI, M/Z = 740.3 [M+H]⁺

HPLC Analysis: 96.4 Area % (n=2), XBridge Phenyl, 150 × 4.6 mm, 3.5 µm, VWD @210 nm

### Example 27. Production and Administration of a Nucleic Acid Vaccine for the Treatment or Prevention of a Viral, Bacterial, or Protozoan Infection

Using the compositions and methods of the disclosure, a nucleic acid vaccine may be produced for treating or preventing an infection caused by a virus, bacterium, or protozoan. For example, to produce such a vaccine, a protein expressed by the virus, bacterium, or protozoan may be identified. The protein may be, for instance, a protein present in the capsid of a target virus or a protein present in the cell membrane or cell wall of a target bacterium or protozoan. Upon selecting a desired protein, an open reading frame (ORF) encoding the protein may be designed using codon-amino acid relationships known in the art. The ORF may then be synthesized, for example, in the form of a deoxyribonucleic acid (DNA) or ribonucleic acid (RNA) molecule, using nucleic acid synthesis techniques known in the art (see, e.g., U.S. Pat. No. 4,401,796, the disclosure of which is incorporated herein as it pertains to methods for the synthesis of nucleic acid molecules). Alternatively, the ORF may be amplified from a natural source, such as the genome of the target virus, bacterium, or protozoan, by way of polymerase chain reaction (PCR) techniques known in the art. Following the amplification of the desired ORF, a DNA molecule resulting from the PCR process may be converted into an RNA by way of an in vitro transcription (IVT) reaction. Exemplary IVT reaction conditions are described in Pokrovskayal and Gurevich, Analytical Biochemistry 220(2):420-423 (1994), the disclosure of which is incorporated herein by reference.

Upon synthesizing the DNA or RNA molecule encoding the desired viral, bacterial, or protozoan antigen, the DNA or RNA molecule may be admixed with, or covalently conjugated to, an adjuvant compound of the disclosure. The adjuvant may be, for example, a compound of any one of formulas (I) -(XXVI) herein, such as any one of compounds (1) - (91).

The resulting vaccine may then be administered to a subject (e.g., a human patient) having or at risk of developing a viral, bacterial, or protozoan infection. Following administration of the vaccine to the subject, the subject’s responsiveness to the vaccine may be monitored by determining the quantity or concentration of B cells, CD4+ T cells, and/or CD8+ T cells that specifically cross-react with the protein encoded by the DNA or RNA vaccine in a sample (e.g., a blood sample) obtained from the subject. Additionally or alternatively, the subject’s responsiveness to the vaccine may be monitored by determining the quantity or concentration of antibodies that specifically bind to the protein encoded by the DNA or RNA vaccine in a sample (e.g., a blood sample) obtained from the subject. In this context, a finding that the quantity or concentration of such antigen-specific B cells, CD4+ T cells, CD8+ T cells, and/or antibodies has increased following administration of the vaccine to the subject indicates that the subject is responding to administration of the vaccine.

In some embodiments, the subject may be administered one or more additional doses of the vaccine. For example, the subject may be administered a total of from 1-10 doses of the vaccine (e.g., 1, 2, 3, 4, 5, 6, 7, 8, 9, or 10 doses of the vaccine). The doses may be independently administered to the subject in intervals of one or more days, weeks, months, or years. For example, the subject may be administered a first dose of the vaccine, followed by a second dose from 1 day to 1 year thereafter (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 2 months, 3 months, 4 months, 5 months, 6 months, 7 months, 8 months, 9 months, 10 months, 11 months, or 12 months thereafter).

### Example 28. Production and Administration of a Personalized Cancer Vaccine

The compositions and methods of the disclosure may be used to produce and deliver a cancer vaccine to a subject (e.g., a human patient) having or at risk of developing cancer. For example, a subject having a particular cancer may be subjected to a genetic test to determine which antigens are expressed by the subject’s cancer. Examples of known cancer-associated antigens include, without limitation, gp100, Hepsin, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, NY-ESO-1, BAGE-1, GAGE-1/2/8, GAGE-3/4/5/6/7, GnTVf, HERV-K-MEL, KK-LC-1, KM-HN-1, LAGE-1, LY6K, MAGE-A1, MAGE-A6, MAGE-A10, MAGE-A12, MAGE-C2, NA88-A, Sp17, SSX-2, SSX-4, and TRAG-3. The subject may be determined to have a cancer that expresses one or more of the foregoing antigens, or another cancer antigen described herein or known in the art.

Upon identifying one or more antigens that are specifically expressed by the subject’s cancer, the antigen(s) may be recombinantly produced as proteins (for example, using molecular biology techniques known in the art or described herein). Alternatively, a nucleic acid molecule, such as a DNA or RNA molecule, that encodes the antigen(s) may be produced (for example, using synthetic gene manufacturing techniques or molecular biology techniques known in the art or described herein).

The antigenic protein, or the DNA or RNA molecule encoding the same, may then be admixed with, or covalently conjugated to, an adjuvant compound of the disclosure. The adjuvant may be, for example, a compound of any one of formulas (I) - (XXVI) herein, such as any one of compounds (1) -(91).

The resulting vaccine may then be administered to the subject (e.g., human patient). Following administration of the vaccine to the subject, the subject’s responsiveness to the vaccine may be monitored by determining the quantity or concentration of B cells, CD4+ T cells, and/or CD8+ T cells that specifically cross-react with the identified cancer antigen in a sample (e.g., a blood sample) obtained from the subject. Additionally or alternatively, the subject’s responsiveness to the vaccine may be monitored by determining the quantity or concentration of antibodies that specifically bind to the identified cancer antigen in a sample (e.g., a blood sample) obtained from the subject. In this context, a finding that the quantity or concentration of cancer antigen-specific B cells, CD4+ T cells, CD8+ T cells, and/or antibodies has increased following administration of the vaccine to the subject indicates that the subject is responding to administration of the vaccine.

In some embodiments, the subject may be administered one or more additional doses of the vaccine. For example, the subject may be administered a total of from 1-10 doses of the vaccine (e.g., 1, 2, 3, 4, 5, 6, 7, 8, 9, or 10 doses of the vaccine). The doses may be independently administered to the subject in intervals of one or more days, weeks, months, or years. For example, the subject may be administered a first dose of the vaccine, followed by a second dose from 1 day to 1 year thereafter (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 2 months, 3 months, 4 months, 5 months, 6 months, 7 months, 8 months, 9 months, 10 months, 11 months, or 12 months thereafter).

### Example 29. Production and Administration of a Universal Cancer Vaccine

In addition to personalized cancer vaccines generated in accordance with the protocol described in Example 13, the compositions and methods of the disclosure may be used to produce and deliver a cancer vaccine that operates in a manner that is agnostic of the subject’s cancer antigen expression profile. For example, an adjuvant compound of the disclosure, such as a compound of any one of formulas (I) - (XXVI) herein (e.g., any one of compounds (1) - (91)) may be admixed with, or covalently conjugated to, a general immune-stimulating agent, such as a toll-like receptor 4 (TLR4) agonist. The TLR4 agonist may be, for example, glucopyranosyl lipid A or lipopolysaccharide. Such immune-stimulating agents, when combined with an adjuvant compound of the disclosure, may activate the immune system of any cancer patient, regardless of the specific cancer antigens expressed by the patient.

The resulting vaccine, containing an adjuvant compound of the disclosure and an immune-stimulating agent (e.g., a TLR4 agonist) may then be administered to the subject. Following administration of the vaccine to the subject, the subject’s responsiveness to the vaccine may be monitored by determining the quantity or concentration of major histocompatibility complex (MHC) class II molecules, CD40 molecules, CD80 molecules, and/or CD86 molecules expressed in a sample of antigen-presenting cells obtained from the subject. These molecules are indicative of the ability of such antigen-presenting cells to mount an immune response against the target cancer. Accordingly, in this context, a finding that the quantity or concentration of MHC class II molecules, CD40 molecules, CD80 molecules, and/or CD86 molecules in a sample of antigen-presenting cells obtained from the subject has increased following administration of the vaccine to the subject indicates that the subject is responding to administration of the vaccine.

In some embodiments, the subject may be administered one or more additional doses of the vaccine. For example, the subject may be administered a total of from 1-10 doses of the vaccine (e.g., 1, 2, 3, 4, 5, 6, 7, 8, 9, or 10 doses of the vaccine). The doses may be independently administered to the subject in intervals of one or more days, weeks, months, or years. For example, the subject may be administered a first dose of the vaccine, followed by a second dose from 1 day to 1 year thereafter (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 2 months, 3 months, 4 months, 5 months, 6 months, 7 months, 8 months, 9 months, 10 months, 11 months, or 12 months thereafter).

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A compound represented by formula (I-a) 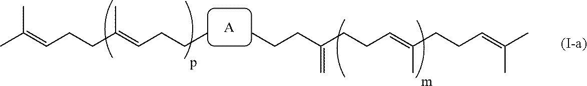
wherein A is 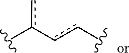 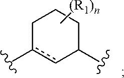
each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
each 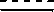
is, independently, a single bond or a double bond;
n is an integer from 0 to 6;
m is an integer from 0 to 3; and
p is an integer from 0 to 3;
or a pharmaceutically acceptable salt thereof;
or a compound represented by formula (I-b) 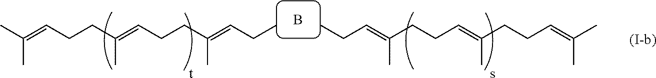
wherein B is —O—, —C(R₁₀)₂—, 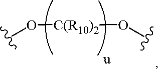 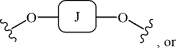 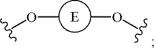
J is optionally substituted alkylene, optionally substituted heteroalkylene, optionally substituted alkenylene, optionally substituted heteroalkenylene, optionally substituted alkynylene, or optionally substituted heteroalkynylene;
E is optionally substituted carbocyclyl or optionally substituted heterocyclyl;
each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
s is an integer from 0 to 3;
t is an integer from 0 to 3; and
u is an integer from 1 to 8;
or a pharmaceutically acceptable salt thereof.

2. The compound of claim 1, wherein the compound is represented by formula (II) 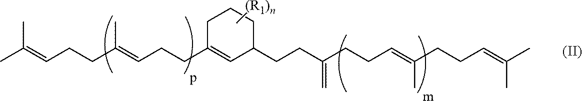
wherein each R₁ is, independently, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6;
m is an integer from 0 to 3; and
p is an integer from 0 to 3;
or a pharmaceutically acceptable salt thereof.

3. The compound of claim 2, wherein the compound is represented by formula (II-a) 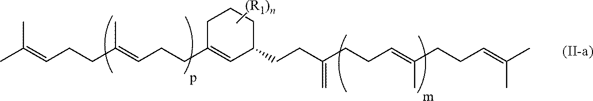
wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6;
m is an integer from 0 to 3; and
p is an integer from 0 to 3;
or a pharmaceutically acceptable salt thereof.

4. The compound of claim 2, wherein the compound is represented by formula (II-b) 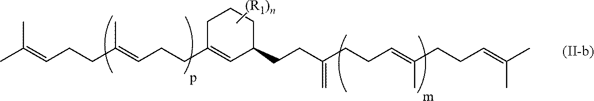
wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 6;
m is an integer from 0 to 3; and
p is an integer from 0 to 3;
or a pharmaceutically acceptable salt thereof.

5. The compound of any one of claims 1-4, wherein each R₁ is, independently, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

6. The compound of claim 5, wherein each R₁ is, independently, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

7. The compound of claim 6, wherein each R₁ is, independently, 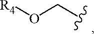 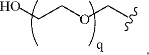 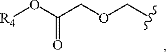 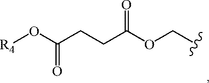 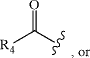 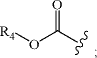
wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20.

8. The compound of claim 7, wherein each R₁ is, independently, 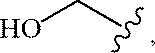 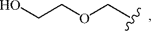 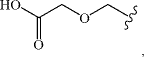 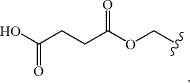 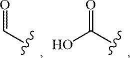 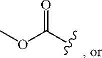 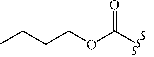
.

9. The compound of claim 6, wherein each R₁ is, independently, 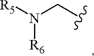 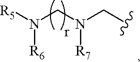 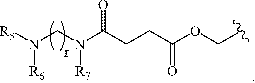 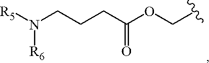 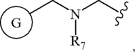 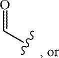 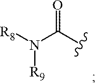
wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6.

10. The compound of claim 9, wherein G is optionally substituted piperidinyl, optionally substituted morpholinyl, or optionally substituted piperazinyl.

11. The compound any of claim 9 or 10, wherein each of R₈ and R₉ is, independently, alkylaminoalkyl or heterocyclyl alkyl.

12. The compound of any one of claims 1-11, wherein n is 1 or 2.

13. The compound of any one of claims 1-12, wherein m is 1.

14. The compound of any one of claims 1-13, wherein p is 1.

15. The compound of claim 1, wherein the compound is represented by formula (III) 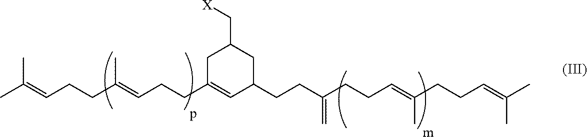
wherein X is optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

16. The compound of claim 15, wherein the compound is represented by formula (III-a) 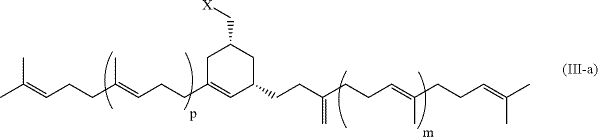
wherein X is optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

17. The compound of claim 15, wherein the compound is represented by formula (III-b) 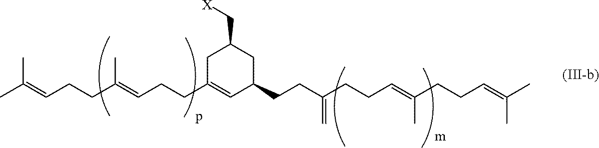
wherein X is optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

18. The compound of claim 15, wherein the compound is represented by formula (III-c) 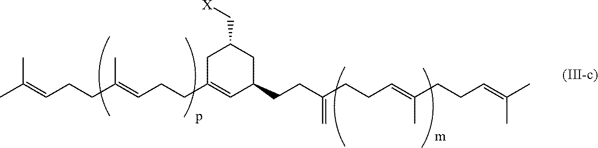
wherein X is optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

19. The compound of claim 15, wherein the compound is represented by formula (III-d) 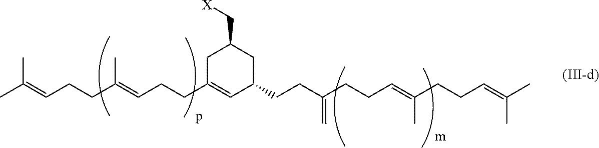
wherein X is optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

20. The compound of claim 1, wherein the compound is represented by formula (IV) 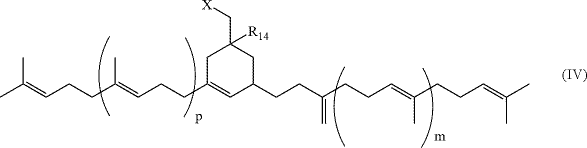
wherein X is optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3; and
p is an integer from 0 to 3;
or a pharmaceutically acceptable salt thereof.

21. The compound of claim 20, wherein the compound is represented by formula (IV-a) 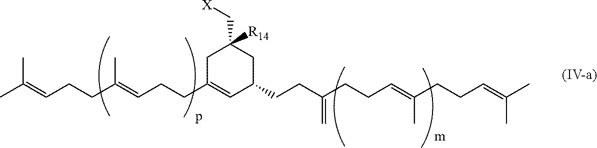
wherein X is optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3; and
p is an integer from 0 to 3;
or a pharmaceutically acceptable salt thereof.

22. The compound of claim 20, wherein the compound is represented by formula (IV-b) 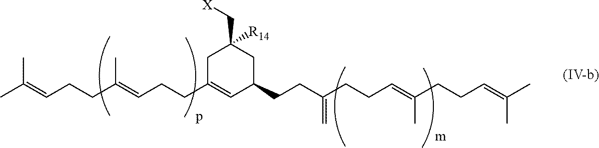
wherein X is optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3; and
p is an integer from 0 to 3;
or a pharmaceutically acceptable salt thereof.

23. The compound of claim 20, wherein the compound is represented by formula (IV-c) 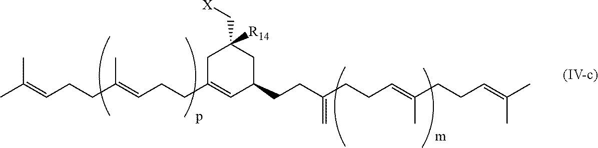
wherein X is optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3; and
p is an integer from 0 to 3;
or a pharmaceutically acceptable salt thereof.

24. The compound of claim 20, wherein the compound is represented by formula (IV-d) 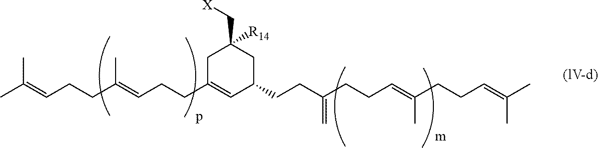
wherein X is optionally substituted alkoxy, optionally substituted acyloxy, hydroxyl, optionally substituted amino, optionally substituted sulfonyloxy, optionally substituted siloxy, carbonyl, or halogen;
R₁₄ is optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
m is an integer from 0 to 3; and
p is an integer from 0 to 3;
or a pharmaceutically acceptable salt thereof.

25. The compound of claim 1, wherein the compound is represented by formula (V) 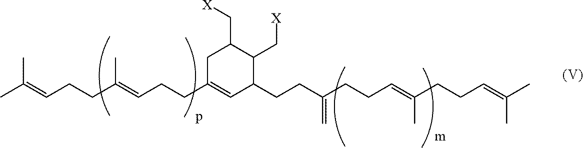
wherein each X is, independently, optionally substituted alkoxy, optionally substituted acyloxy, hydroxyl, optionally substituted amino, optionally substituted aminoalkyl, optionally substituted sulfonyloxy, optionally substituted siloxy, carbonyl, or halogen;
m is an integer from 0 to 3; and
p is an integer from 0 to 3;
or a pharmaceutically acceptable salt thereof.

26. The compound of claim 25, wherein the compound is represented by formula (V-a) 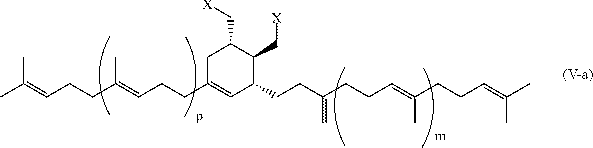
wherein each X is, independently, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

27. The compound of claim 25, wherein the compound is represented by formula (V-b) 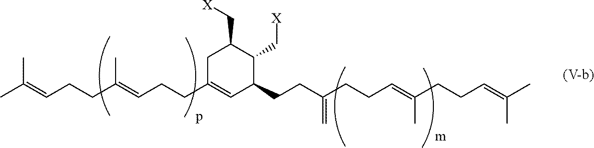
wherein each X is, independently, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

28. The compound of claim 25, wherein the compound is represented by formula (V-c) 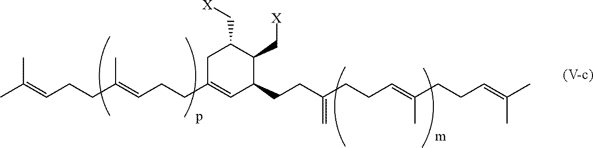
wherein each X is, independently, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

29. The compound of claim 25, wherein the compound is represented by formula (V-d) 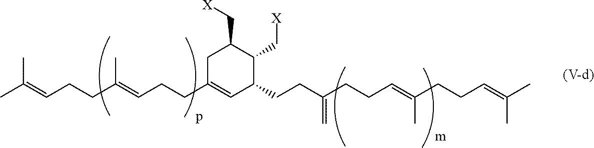
wherein each X is, independently, optionally substituted alkoxy, optionally substituted acyloxy, hydroxyl, optionally substituted amino, optionally substituted sulfonyloxy, optionally substituted siloxy, carbonyl, or halogen;
m is an integer from 0 to 3; and
p is an integer from 0 to 3;
or a pharmaceutically acceptable salt thereof.

30. The compound of any one of claims 15-29, wherein each X is, independently, 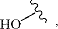 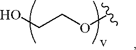 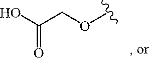 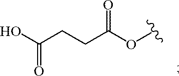
wherein v is an integer from 1 to 15.

31. The compound of any one of claims 15-29, wherein each X is, independently, 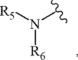 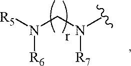 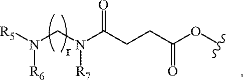 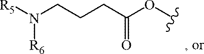 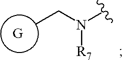
wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 1 to 6.

32. The compound of claim 31, wherein G is optionally substituted piperidinyl, optionally substituted morpholinyl, or optionally substituted piperazinyl.

33. The compound of any one of claims 15-32, wherein m is 1.

34. The compound of any one of claims 15-33, wherein p is 1.

35. The compound of claim 1, wherein the compound is represented by formula (VI) 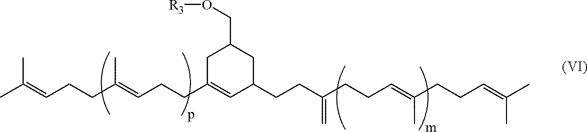
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

36. The compound of claim 35, wherein the compound is represented by formula (VI-a) 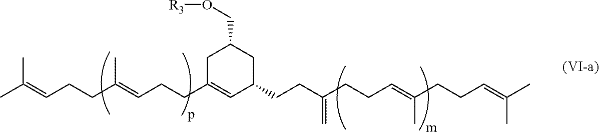
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

37. The compound of claim 35, wherein the compound is represented by formula (VI-b) 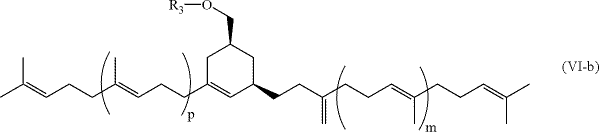
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

38. The compound of claim 35, wherein the compound is represented by formula (VI-c) 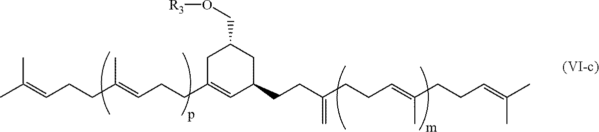
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

39. The compound of claim 35, wherein the compound is represented by formula (VI-d) 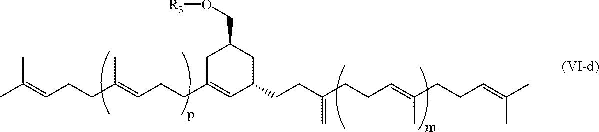
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

40. The compound of claim 1, wherein the compound is represented by formula (VII) 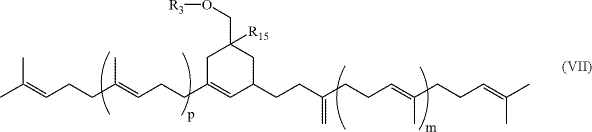
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
R₁₅ is optionally substituted alkyl, optionally substituted alkenyl, or optionally substituted alkynyl;
m is an integer from 0 to 3; and
p is an integer from 0 to 3;
or a pharmaceutically acceptable salt thereof.

41. The compound of claim 40, wherein the compound is represented by formula (VII-a) 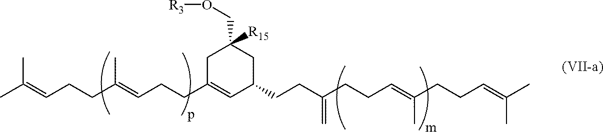
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
R₁₅ is optionally substituted alkyl, optionally substituted alkenyl, or optionally substituted alkynyl;
m is an integer from 0 to 3; and
p is an integer from 0 to 3;
or a pharmaceutically acceptable salt thereof.

42. The compound of claim 40, wherein the compound is represented by formula (VII-b) 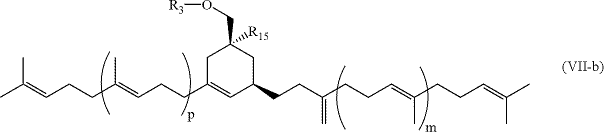
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
R₁₅ is optionally substituted alkyl, optionally substituted alkenyl, or optionally substituted alkynyl;
m is an integer from 0 to 3; and
p is an integer from 0 to 3;
or a pharmaceutically acceptable salt thereof.

43. The compound of claim 40, wherein the compound is represented by formula (VII-c) 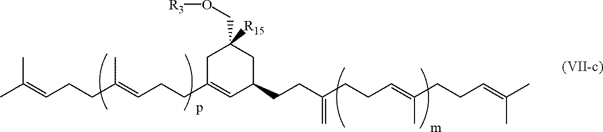
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
R₁₅ is optionally substituted alkyl, optionally substituted alkenyl, or optionally substituted alkynyl;
m is an integer from 0 to 3; and
p is an integer from 0 to 3;
or a pharmaceutically acceptable salt thereof.

44. The compound of claim 40, wherein the compound is represented by formula (VII-d) 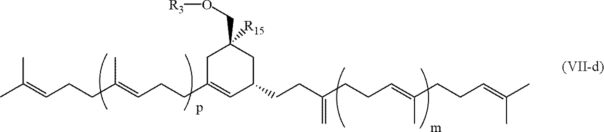
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
R₁₅ is optionally substituted alkyl, optionally substituted alkenyl, or optionally substituted alkynyl;
m is an integer from 0 to 3; and
p is an integer from 0 to 3;
or a pharmaceutically acceptable salt thereof.

45. The compound of claim 1, wherein the compound is represented by formula (VIII) 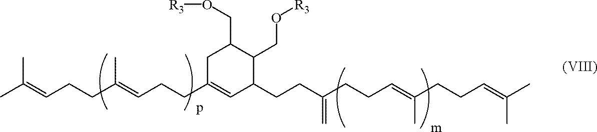
wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

46. The compound of claim 45, wherein the compound is represented by formula (VIII-a) 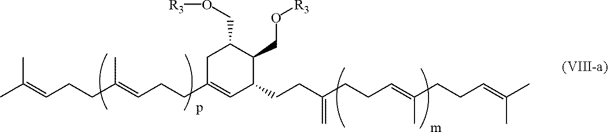
wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

47. The compound of claim 45, wherein the compound is represented by formula (VIII-b) 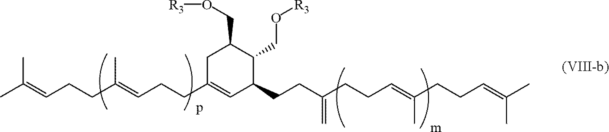
wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

48. The compound of claim 45, wherein the compound is represented by formula (VIII-c) 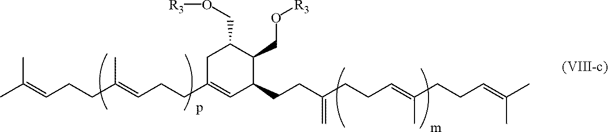
wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

49. The compound of claim 45, wherein the compound is represented by formula (VIII-d) 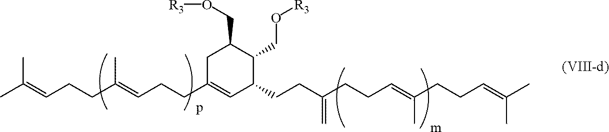
wherein each R₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
m is an integer from 0 to 3; and
p is an integer from 0 to 3;
or a pharmaceutically acceptable salt thereof.

50. The compound of any one of claims 35-49, wherein each R₃ is, independently, hydrogen, 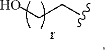 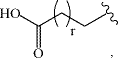 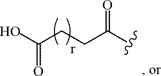 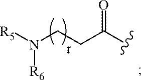
wherein each of R₅ and R₆ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 0 to 6.

51. The compound of any one of claims 35-50, wherein m is 1.

52. The compound of any one of claims 35-51, wherein p is 1.

53. The compound of claim 1, wherein the compound is represented by formula (IX) 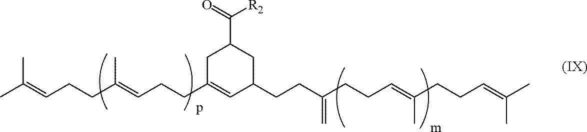
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

54. The compound of claim 53, wherein the compound is represented by formula (IX-a) 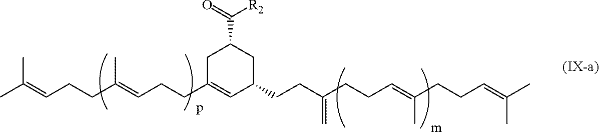
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

55. The compound of claim 53, wherein the compound is represented by formula (IX-b) 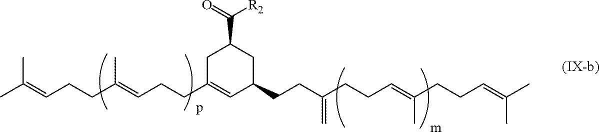
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

56. The compound of claim 53, wherein the compound is represented by formula (IX-c) 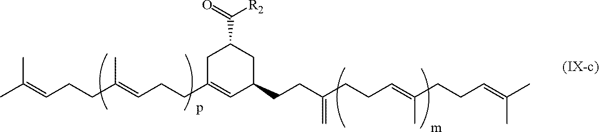
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

57. The compound of claim 53, wherein the compound is represented by formula (IX-d) 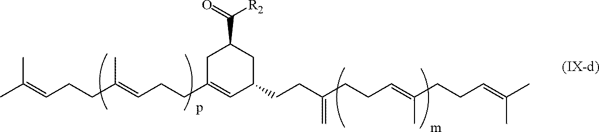
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

58. The compound of claim 1, wherein the compound is represented by formula (X) 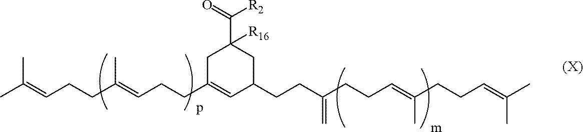
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
R₁₆ is optionally substituted alkyl, optionally substituted alkenyl, or optionally substituted alkynyl;
m is an integer from 0 to 3; and
p is an integer from 0 to 3;
or a pharmaceutically acceptable salt thereof.

59. The compound of claim 58, wherein the compound is represented by formula (X-a) 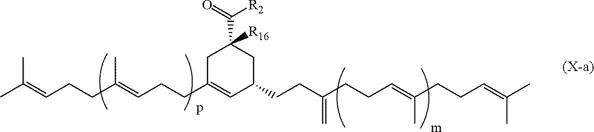
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
R₁₆ is optionally substituted alkyl, optionally substituted alkenyl, or optionally substituted alkynyl;
m is an integer from 0 to 3; and
p is an integer from 0 to 3;
or a pharmaceutically acceptable salt thereof.

60. The compound of claim 58, wherein the compound is represented by formula (X-b) 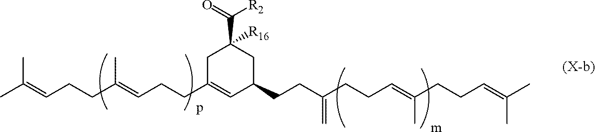
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
R₁₆ is optionally substituted alkyl, optionally substituted alkenyl, or optionally substituted alkynyl;
m is an integer from 0 to 3; and
p is an integer from 0 to 3;
or a pharmaceutically acceptable salt thereof.

61. The compound of claim 58, wherein the compound is represented by formula (X-c) 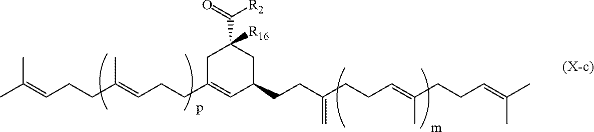
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
R₁₆ is optionally substituted alkyl, optionally substituted alkenyl, or optionally substituted alkynyl;
m is an integer from 0 to 3; and
p is an integer from 0 to 3;
or a pharmaceutically acceptable salt thereof.

62. The compound of claim 58, wherein the compound is represented by formula (X-d) 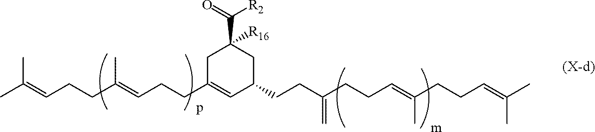
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
R₁₆ is optionally substituted alkyl, optionally substituted alkenyl, or optionally substituted alkynyl;
m is an integer from 0 to 3; and
p is an integer from 0 to 3;
or a pharmaceutically acceptable salt thereof.

63. The compound of claim 1, wherein the compound is represented by formula (XI) 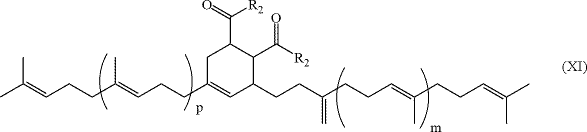
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

64. The compound of claim 63, wherein the compound is represented by formula (XI-a) 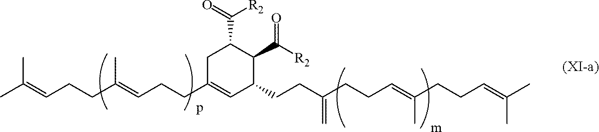
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

65. The compound of claim 63, wherein the compound is represented by formula (XI-b) 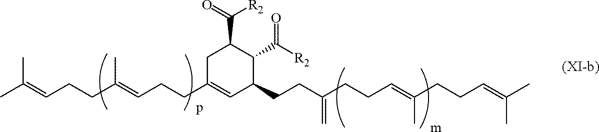
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

66. The compound of claim 63, wherein the compound is represented by formula (XI-c) 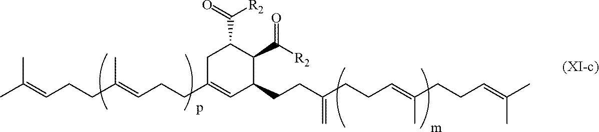
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

67. The compound of claim 63, wherein the compound is represented by formula (XI-d) 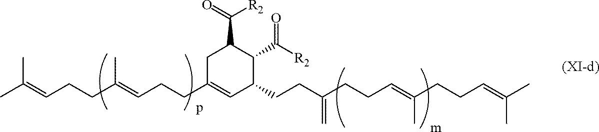
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
m is an integer from 0 to 3; and
p is an integer from 0 to 3;
or a pharmaceutically acceptable salt thereof.

68. The compound of any one of claims 53-67, wherein each R₂ is, independently, hydrogen, hydroxyl, 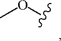 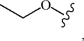 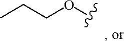 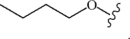
.

69. The compound of any one of claims 53-67, wherein each R₂ is, independently 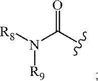
wherein each of R₈ and R₉ is, independently, hydrogen, optionally substituted alkyl, optionally substituted alkenyl, optionally substituted alkynyl, or optionally substituted aminoalkyl.

70. The compound any of claim 54, wherein each of R₈ and R₉ is, independently, alkylaminoalkyl or heterocyclyl alkyl.

71. The compound of any one of claims 53-70, wherein m is 1.

72. The compound of any one of claims 53-71, wherein p is 1.

73. The compound of claim 1, wherein the compound is represented by formula (XII) 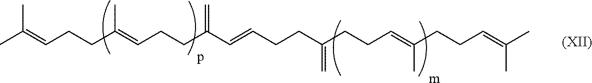
wherein m is an integer from 0 to 3; and
p is an integer from 0 to 3.

74. The compound of claim 73, wherein m is 1.

75. The compound of claim 73 or 74, wherein p is 1.

76. A compound represented by formula (I-b) 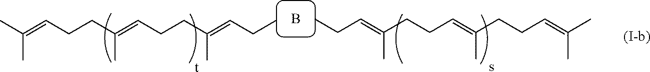
wherein B is 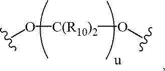 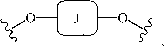 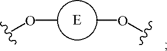 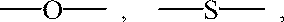
O(C(R
₁₀)₂)ₙO-, -(C(R₁₀)₂)ₙ-, or -N(R₁₁)₂-;
J is optionally substituted alkylene, optionally substituted heteroalkylene, optionally substituted alkenylene, optionally substituted heteroalkenylene, optionally substituted alkynylene, or optionally substituted heteroalkynylene;
E is optionally substituted carbocyclyl or optionally substituted heterocyclyl;
each of R₁₀ and R₁₁ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
each n is, independently, an integer from 1 to 6;
s is an integer from 0 to 3;
t is an integer from 0 to 3; and
u is an integer from 1 to 8;
or a pharmaceutically acceptable salt thereof.

77. The compound of claim 76, wherein the compound is represented by formula (XIII) 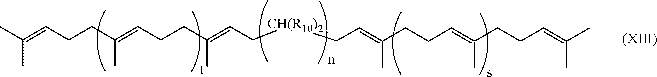
wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
n is an integer from 1 to 6;
s is an integer from 0 to 3; and
t is an integer from 0 to 3;
or a pharmaceutically acceptable salt thereof.

78. The compound of claim 77, wherein each R₁₀ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

79. The compound of claim 78, wherein each R₁₀ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

80. The compound of claim 79, wherein each R₁₀ is, independently, hydrogen, 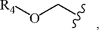 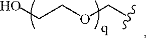 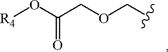 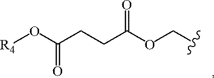 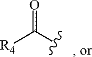 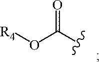
wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20.

81. The compound of claim 80, wherein each R₁₀ is, independently, hydrogen, 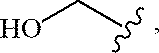 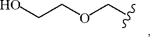 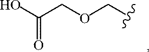 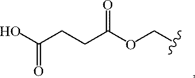 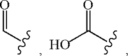 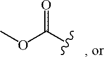 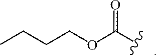
.

82. The compound of claim 79, wherein each R₁₀ is, independently, hydrogen, 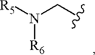 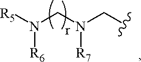 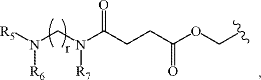 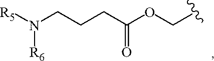 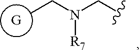 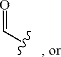 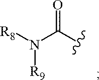
wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6.

83. The compound of claim 82, wherein G is optionally substituted piperidinyl, optionally substituted morpholinyl, or optionally substituted piperazinyl.

84. The compound of claim 82 or 83, wherein each of R₈ and R₉ is, independently, alkylaminoalkyl or heterocyclyl alkyl.

85. The compound of any one of claims 77-84, wherein n is 1 or 2.

86. The compound of any one of claims 77-85, wherein s is 1.

87. The compound of any one of claims 77-86, wherein t is 1.

88. The compound of claim 1, wherein the compound is represented by formula (XIV) 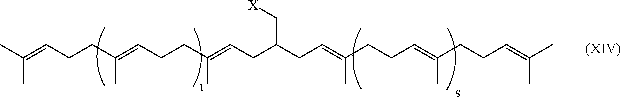
wherein X is optionally substituted alkoxy, optionally substituted acyloxy, hydroxyl, optionally substituted amino, optionally substituted sulfonyloxy, optionally substituted siloxy, carbonyl, or halogen;
s is an integer from 0 to 3; and
t is an integer from 0 to 3;
or a pharmaceutically acceptable salt thereof.

89. The compound of claim 88, wherein X is 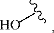 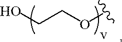 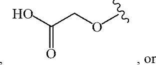 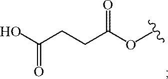
wherein v is an integer from 1 to 15.

90. The compound of claim 88, wherein X is 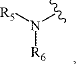 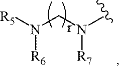 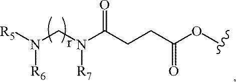 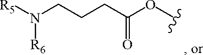 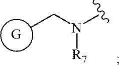
wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 1 to 6.

91. The compound of claim 90, wherein G is optionally substituted piperidinyl, optionally substituted morpholinyl, or optionally substituted piperazinyl.

92. The compound of any one of claims 88-91, wherein s is 1.

93. The compound of any one of claims 88-92, wherein t is 1.

94. The compound of claim 1, wherein the compound is represented by formula (XV) 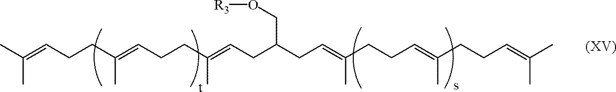
wherein R₃ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted sulfonyl, or optionally substituted silyl;
s is an integer from 0 to 3; and
t is an integer from 0 to 3;
or a pharmaceutically acceptable salt thereof.

95. The compound of claim 94, wherein R₃ is hydrogen, 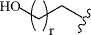 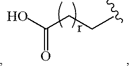 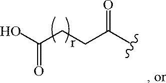 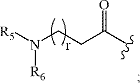
wherein each of R₅ and R₆ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring; and
each r is, independently, an integer from 0 to 6.

96. The compound of claim 94 or 95, wherein s is 1.

97. The compound of any one of claims 94-96, wherein t is 1.

98. The compound of claim 1, wherein the compound is represented by formula (XVI) 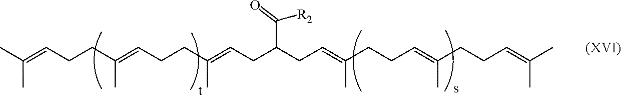
wherein R₂ is hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
s is an integer from 0 to 3; and
t is an integer from 0 to 3;
or a pharmaceutically acceptable salt thereof.

99. The compound of claim 1, wherein the compound is represented by formula (XVII) 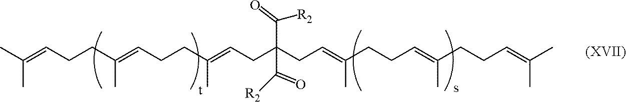
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted amino, optionally substituted sulfonyloxy, hydroxyl, or halogen;
s is an integer from 0 to 3; and
t is an integer from 0 to 3;
or a pharmaceutically acceptable salt thereof.

100. The compound of claim 98 or 99, wherein each R₂ is, independently, hydrogen, hydroxyl, 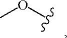 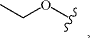 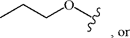 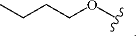
.

101. The compound of any one of claims 98-100, wherein s is 1.

102. The compound of any one of claims 98-101, wherein t is 1.

103. The compound of claim 76, wherein the compound is represented by formula (XVIII) 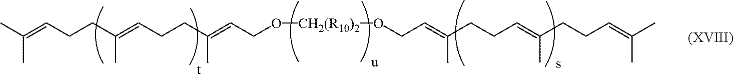
wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
u is an integer from 1 to 8;
s is an integer from 0 to 3; and
t is an integer from 0 to 3;
or a pharmaceutically acceptable salt thereof.

104. The compound of claim 103, wherein each R₁₀ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

105. The compound of claim 104, wherein each R₁₀ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

106. The compound of claim 105, wherein each R₁₀ is, independently, hydrogen, 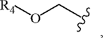 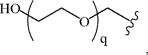 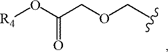 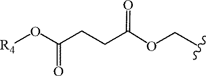 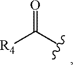 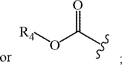
wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20.

107. The compound of claim 106, wherein each R₁₀ is, independently, hydrogen, 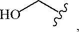 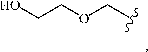 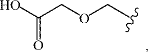 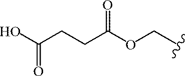 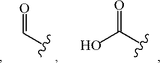 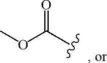 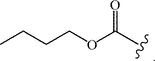
.

108. The compound of claim 105, wherein each R₁₀ is, independently, hydrogen, 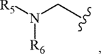 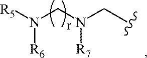 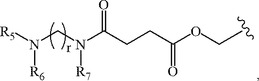 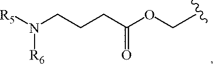 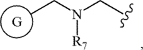 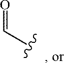 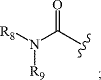
wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6.

109. The compound of claim 108, wherein G is optionally substituted piperidinyl, optionally substituted morpholinyl, or optionally substituted piperazinyl.

110. The compound of claim 108 or 109, wherein each of R₈ and R₉ is, independently, alkylaminoalkyl or heterocyclyl alkyl.

111. The compound of any one of claims 103-110, wherein u is 1 or 2.

112. The compound of any one of claims 103-111, wherein s is 1.

113. The compound of any one of claims 103-112, wherein t is 1.

114. The compound of claim 1, wherein the compound is represented by formula (XIX) 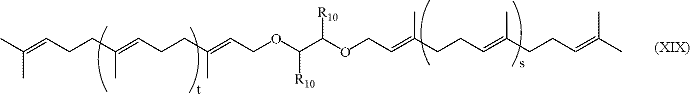
wherein each R₁₀ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, or optionally substituted heteroalkynyl;
s is an integer from 0 to 3; and
t is an integer from 0 to 3.

115. The compound of claim 76, wherein the compound is represented by formula (XX) 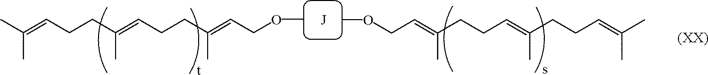
wherein J is optionally substituted alkylene, optionally substituted heteroalkylene, optionally substituted alkenylene, optionally substituted heteroalkenylene, optionally substituted alkynylene, or optionally substituted heteroalkynylene.

116. The compound of claim 115, wherein J is optionally substituted alkenylene or optionally substituted heteroalkenylene.

117. The compound of claim 1, wherein the compound is represented by formula (XXI) 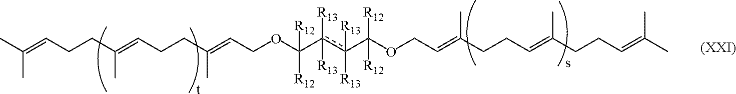
wherein each of R₁₂ and R₁₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
wherein each
$$z o n i n g e r r o r$$
is, independently, a single bond or a double bond;
wherein if
$$z o n i n g e r r o r$$
is a double bond, only one R
₁₃ is present at each carbon of the double bond;
s is an integer from 0 to 3; and
t is an integer from 0 to 3.

118. The compound of claim 1, wherein the compound is represented by formula (XXII) 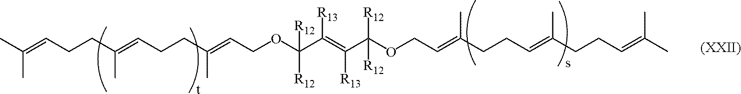
wherein each of R₁₂ and R₁₃ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, carbonyl, optionally substituted alkoxy, optionally substituted acyloxy, hydroxyl, oxo, optionally substituted haloalkyl, or halogen;
wherein s is an integer from 0 to 3; and
t is an integer from 0 to 3.

119. The compound of claim 76, wherein the compound is represented by formula (XXIII) 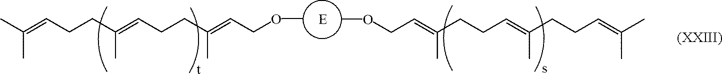
wherein E is optionally substituted cycloalkyl, optionally substituted heterocyclyl, optionally substituted aryl, or optionally substituted heteroaryl.

120. The compound of claim 76 or 119, wherein E is a ring selected from 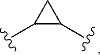 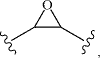 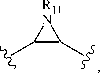 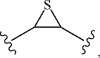 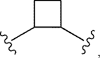 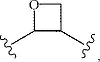 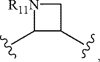 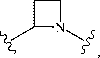 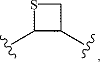 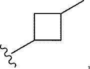 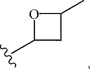 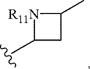 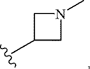 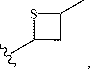 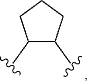 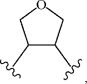 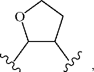 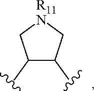 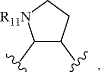 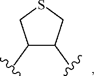 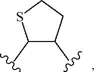 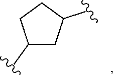 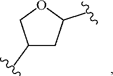 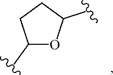 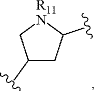 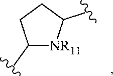 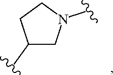 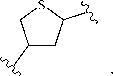 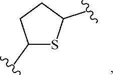 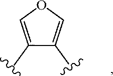 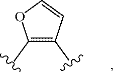 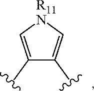 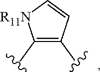 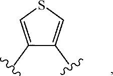 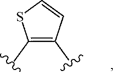 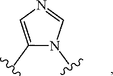 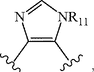 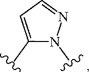 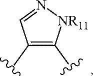 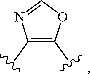 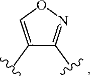 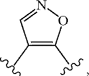 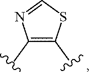 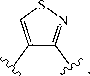 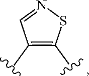 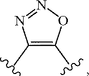 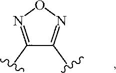 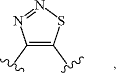 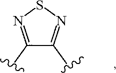 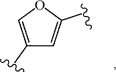 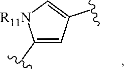 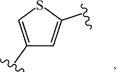 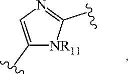 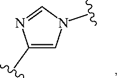 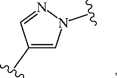 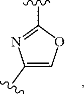 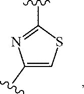 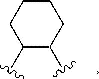 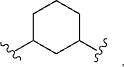 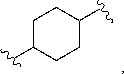 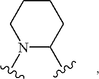 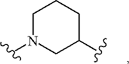 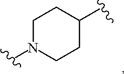 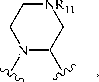 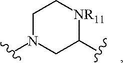 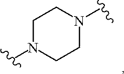 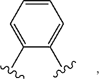 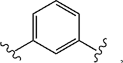 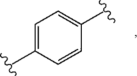 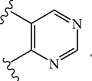 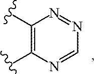 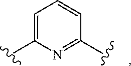 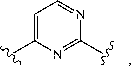 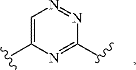 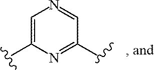 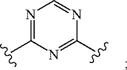
wherein each R₁₁ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted carbocyclyl, optionally substituted heterocyclyl, optionally substituted aryl, optionally substituted heteroaryl, or carbonyl;
and wherein each ring of E may be optionally substituted, as the valency of each ring permits.

121. The compound of claim 120, wherein E is 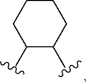 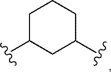 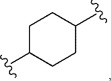 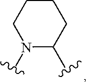 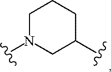 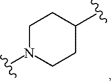 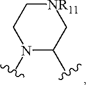 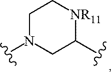 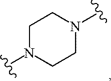 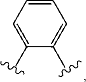 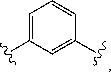 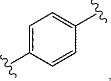 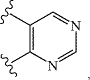 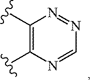 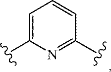 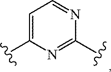 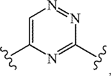 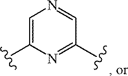 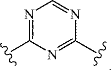
.

122. The compound of any one of claims 76, 119, 120, and 121, wherein s is 1.

123. The compound of any one of claims 76, 119, 120, 121, and 122, wherein t is 1.

124. The compound of claim 1, wherein the compound is represented by formula (XXIV) 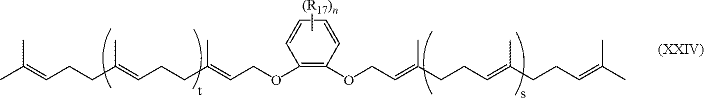
wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4;
s is an integer from 0 to 3; and
t is an integer from 0 to 3;
or a pharmaceutically acceptable salt thereof.

125. The compound of claim 1, wherein the compound is represented by formula (XXV) 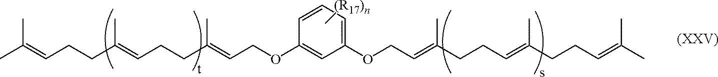
wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4;
s is an integer from 0 to 3; and
t is an integer from 0 to 3;
or a pharmaceutically acceptable salt thereof.

126. The compound of claim 1, wherein the compound is represented by formula (XXVI) 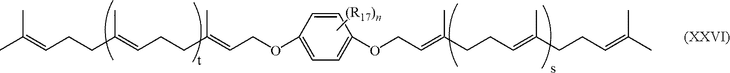
wherein each R₁₇ is, independently, hydrogen, optionally substituted alkyl, optionally substituted aminoalkyl optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, oxo, optionally substituted haloalkyl, or halogen;
n is an integer from 0 to 4;
s is an integer from 0 to 3; and
t is an integer from 0 to 3;
or a pharmaceutically acceptable salt thereof.

127. The compound of any one of claims 124-126, wherein each R₁₇ is, independently, hydrogen, optionally substituted alkoxyalkyl, optionally substituted acyloxyalkyl, optionally substituted aminoalkyl, optionally substituted sulfonyloxyalkyl, optionally substituted siloxyalkyl, optionally substituted hydroxyalkyl, or carbonyl.

128. The compound of claim 127, wherein each R₁₇ is, independently, hydrogen, optionally substituted alkoxymethyl, optionally substituted acyloxymethyl, optionally substituted aminomethyl, optionally substituted sulfonyloxymethyl, optionally substituted siloxymethyl, optionally substituted hydroxymethyl, or carbonyl.

129. The compound of claim 128, wherein each R₁₇ is, independently, hydrogen, 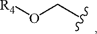 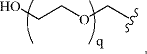 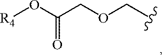 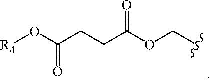 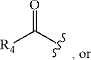 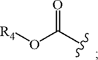
wherein each R₄ is, independently, hydrogen, C₁₋₆ alkyl, C₁₋₆ heteroalkyl, C₂₋₆ alkenyl, C₂₋₆ heteroalkenyl, C₂₋₆ alkynyl, monosaccharide, disaccharide, trisaccharide, an acyl saccharamide, or C₂₋₆ heteroalkynyl; and
each q is, independently, an integer from 1 to 20.

130. The compound of claim 129, wherein each R₁₇ is, independently, hydrogen, 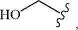 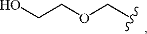 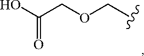 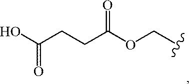 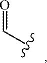 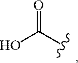 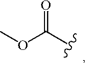 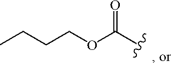 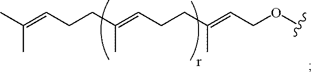
and wherein each r is an integer from 0 to 3.

131. The compound of claim 128, wherein each R₁₇ is, independently, hydrogen, 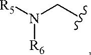 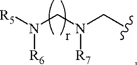 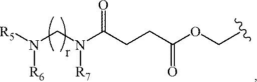 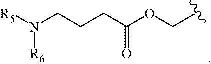 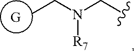 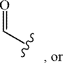 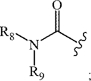
wherein G is optionally substituted carbocyclyl or optionally substituted heterocyclyl;
each of R₅, R₆, and R₇ is, independently, hydrogen, C₁₋₆ alkyl, C₁₋₆ heteroalkyl, C₂₋₆ alkenyl, C₂₋₆ heteroalkenyl, C₂₋₆ alkynyl, or C₂₋₆ heteroalkynyl, or R₅ and R₆, together with the atom to which they are bound, are joined to form an optionally substituted carbocyclyl or optionally substituted heterocyclyl ring;
each of R₈ and R₉ is, independently, hydrogen, optionally substituted alkyl, optionally substituted alkenyl, optionally substituted alkynyl, or optionally substituted aminoalkyl; and
each r is, independently, an integer from 1 to 6.

132. The compound of claim 131, wherein G is optionally substituted piperidinyl, optionally substituted morpholinyl, or optionally substituted piperazinyl.

133. The compound any of claim 131 or 132, wherein each of R₈ and R₉ is, independently, alkylaminoalkyl or heterocyclyl alkyl.

134. The compound of any one of claims 124-133, wherein n is 1 or 2.

135. The compound of any one of claims 124-134, wherein s is 1.

136. The compound of any one of claims 124-135, wherein t is 1.

137. The compound of claim 1, wherein the compound is represented by formula (1) 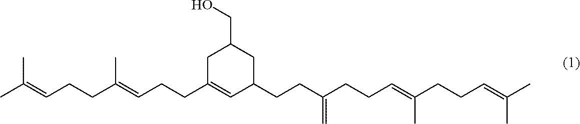 [SMILES: C=C(CC/C=C(\C)CCC=C(C)C)CCC1C=C(CC/C=C(\C)CCC=C(C)C)CC(CO)C1]
.

138. The compound of claim 1, wherein the compound is represented by formula (2) 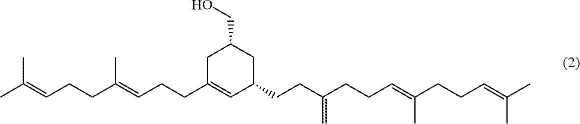 [SMILES: C=C(CC/C=C(\C)CCC=C(C)C)CC[C@@H]1C=C(CC/C=C(\C)CCC=C(C)C)C[C@H](CO)C1]
.

139. The compound of claim 1, wherein the compound is represented by formula (3) 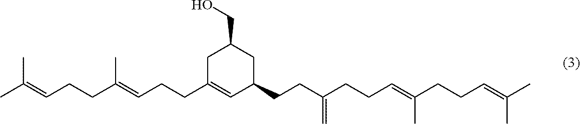 [SMILES: C=C(CC/C=C(\C)CCC=C(C)C)CC[C@H]1C=C(CC/C=C(\C)CCC=C(C)C)C[C@@H](CO)C1]
.

140. The compound of claim 1, wherein the compound is represented by formula (4) 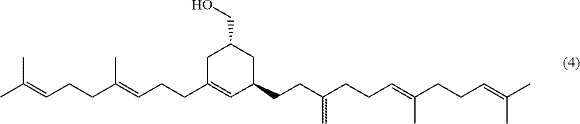 [SMILES: C=C(CC/C=C(\C)CCC=C(C)C)CC[C@H]1C=C(CC/C=C(\C)CCC=C(C)C)C[C@H](CO)C1]
.

141. The compound of claim 1, wherein the compound is represented by formula (5) 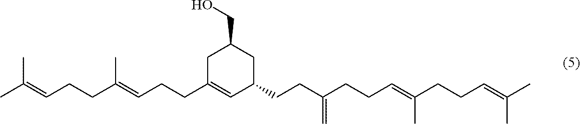 [SMILES: C=C(CC/C=C(\C)CCC=C(C)C)CC[C@@H]1C=C(CC/C=C(\C)CCC=C(C)C)C[C@@H](CO)C1]
.

142. The compound of claim 1, wherein the compound is represented by formula (6) 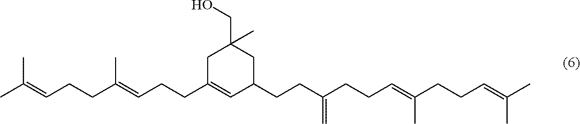 [SMILES: C=C(CC/C=C(\C)CCC=C(C)C)CCC1C=C(CC/C=C(\C)CCC=C(C)C)CC(C)(CO)C1]
.

143. The compound of claim 1, wherein the compound is represented by formula (7) 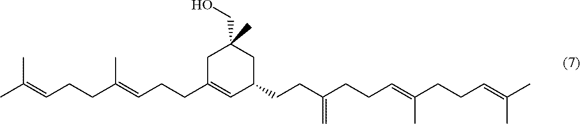 [SMILES: C=C(CC/C=C(\C)CCC=C(C)C)CC[C@@H]1C=C(CC/C=C(\C)CCC=C(C)C)C[C@](C)(CO)C1]
.

144. The compound of claim 1, wherein the compound is represented by formula (8) 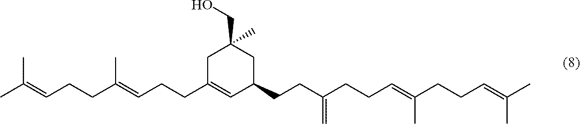 [SMILES: C=C(CC/C=C(\C)CCC=C(C)C)CC[C@H]1C=C(CC/C=C(\C)CCC=C(C)C)C[C@@](C)(CO)C1]
.

145. The compound of claim 1, wherein the compound is represented by formula (9) 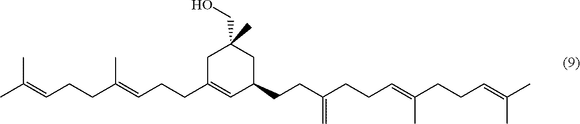 [SMILES: C=C(CC/C=C(\C)CCC=C(C)C)CC[C@H]1C=C(CC/C=C(\C)CCC=C(C)C)C[C@](C)(CO)C1]
.

146. The compound of claim 1, wherein the compound is represented by formula (10) 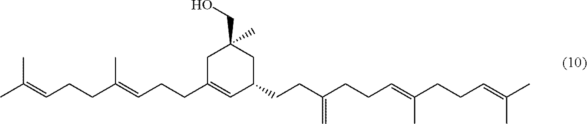 [SMILES: C=C(CC/C=C(\C)CCC=C(C)C)CC[C@@H]1C=C(CC/C=C(\C)CCC=C(C)C)C[C@@](C)(CO)C1]
.

147. The compound of claim 1, wherein the compound is represented by formula (11) 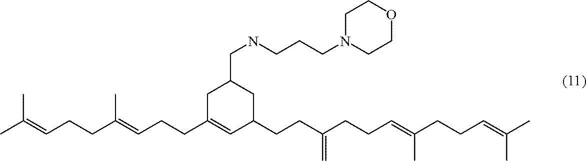 [SMILES: C=C(CC/C=C(\C)CCC=C(C)C)CCC1C=C(CC/C=C(\C)CCC=C(C)C)CC(CNCCCN2CCOCC2)C1]
.

148. The compound of claim 1, wherein the compound is represented by formula (12) 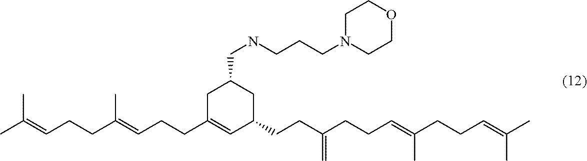 [SMILES: C=C(CC/C=C(\C)CCC=C(C)C)CC[C@@H]1C=C(CC/C=C(\C)CCC=C(C)C)C[C@H](CNCCCN2CCOCC2)C1]
.

149. The compound of claim 1, wherein the compound is represented by formula (13) 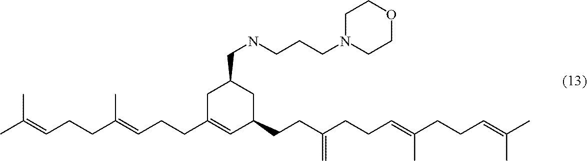 [SMILES: C=C(CC/C=C(\C)CCC=C(C)C)CC[C@H]1C=C(CC/C=C(\C)CCC=C(C)C)C[C@@H](CNCCCN2CCOCC2)C1]
.

150. The compound of claim 1, wherein the compound is represented by formula (14) 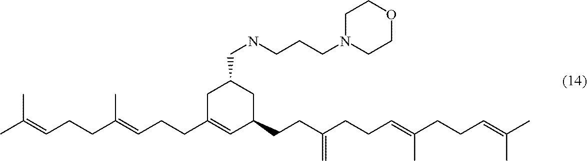 [SMILES: C=C(CC/C=C(\C)CCC=C(C)C)CC[C@H]1C=C(CC/C=C(\C)CCC=C(C)C)C[C@H](CNCCCN2CCOCC2)C1]
.

151. The compound of claim 1, wherein the compound is represented by formula (15) 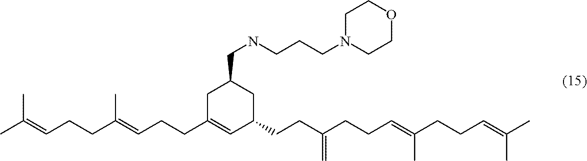 [SMILES: C=C(CC/C=C(\C)CCC=C(C)C)CC[C@@H]1C=C(CC/C=C(\C)CCC=C(C)C)C[C@@H](CNCCCN2CCOCC2)C1]
.

152. The compound of claim 1, wherein the compound is represented by formula (16) 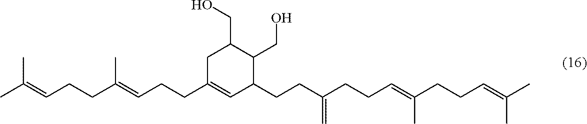 [SMILES: C=C(CC/C=C(\C)CCC=C(C)C)CCC1C=C(CC/C=C(\C)CCC=C(C)C)CC(CO)C1CO]
.

153. The compound of claim 1, wherein the compound is represented by formula (17) 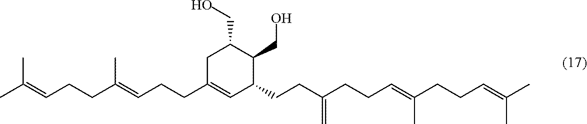 [SMILES: C=C(CC/C=C(\C)CCC=C(C)C)CC[C@@H]1C=C(CC/C=C(\C)CCC=C(C)C)C[C@H](CO)[C@H]1CO]
.

154. The compound of claim 1, wherein the compound is represented by formula (18) 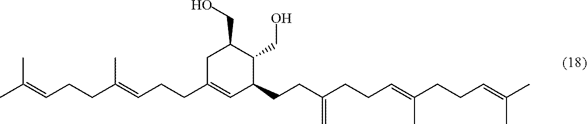 [SMILES: C=C(CC/C=C(\C)CCC=C(C)C)CC[C@H]1C=C(CC/C=C(\C)CCC=C(C)C)C[C@@H](CO)[C@@H]1CO]
.

155. The compound of claim 1, wherein the compound is represented by formula (19) 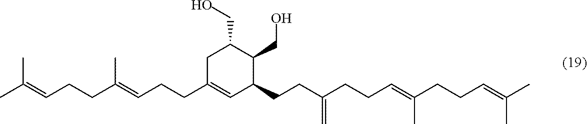 [SMILES: C=C(CC/C=C(\C)CCC=C(C)C)CC[C@H]1C=C(CC/C=C(\C)CCC=C(C)C)C[C@H](CO)[C@H]1CO]
.

156. The compound of claim 1, wherein the compound is represented by formula (20) 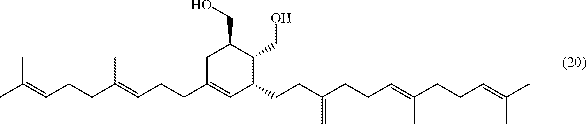 [SMILES: C=C(CC/C=C(\C)CCC=C(C)C)CC[C@@H]1C=C(CC/C=C(\C)CCC=C(C)C)C[C@@H](CO)[C@@H]1CO]
.

157. The compound of claim 1, wherein the compound is represented by formula (21) 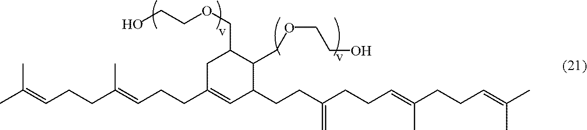
wherein each v is an integer from 1 to 15.

158. The compound of claim 1, wherein the compound is represented by formula (22) 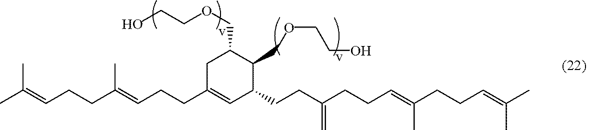
wherein each v is an integer from 1 to 15.

159. The compound of claim 1, wherein the compound is represented by formula (23) 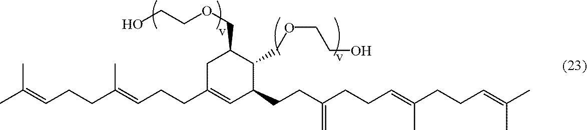
wherein each v is an integer from 1 to 15.

160. The compound of claim 1, wherein the compound is represented by formula (24) 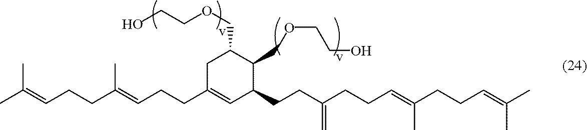
wherein each v is an integer from 1 to 15.

161. The compound of claim 1, wherein the compound is represented by formula (25) 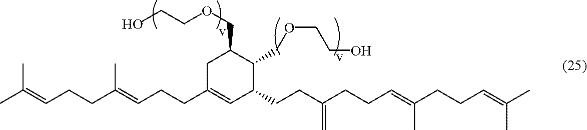
wherein each v is an integer from 1 to 15.

162. The compound of claim 1, wherein the compound is represented by formula (26) 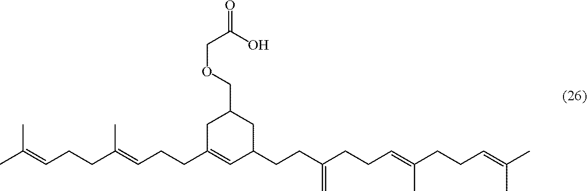 [SMILES: C=C(CC/C=C(\C)CCC=C(C)C)CCC1C=C(CC/C=C(\C)CCC=C(C)C)CC(COCC(=O)O)C1]
or a pharmaceutically acceptable salt thereof.

163. The compound of claim 1, wherein the compound is represented by formula (27) 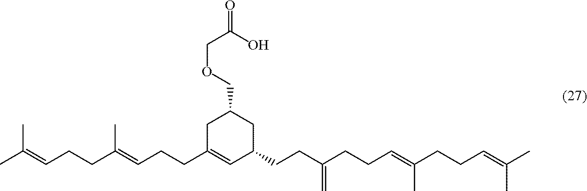 [SMILES: C=C(CC/C=C(\C)CCC=C(C)C)CC[C@@H]1C=C(CC/C=C(\C)CCC=C(C)C)C[C@H](COCC(=O)O)C1]
or a pharmaceutically acceptable salt thereof.

164. The compound of claim 1, wherein the compound is represented by formula (28) 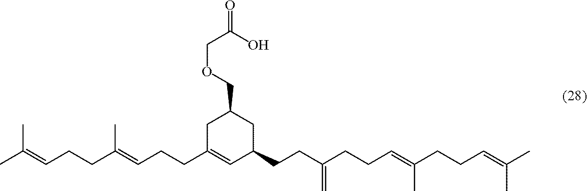 [SMILES: C=C(CC/C=C(\C)CCC=C(C)C)CC[C@H]1C=C(CC/C=C(\C)CCC=C(C)C)C[C@@H](COCC(=O)O)C1]
or a pharmaceutically acceptable salt thereof.

165. The compound of claim 1, wherein the compound is represented by formula (29) 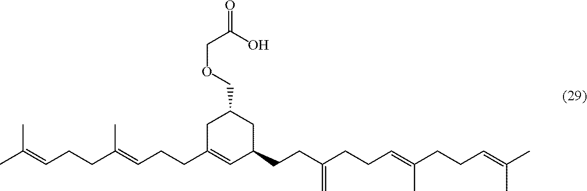 [SMILES: C=C(CC/C=C(\C)CCC=C(C)C)CC[C@H]1C=C(CC/C=C(\C)CCC=C(C)C)C[C@H](COCC(=O)O)C1]
or a pharmaceutically acceptable salt thereof.

166. The compound of claim 1, wherein the compound is represented by formula (30) 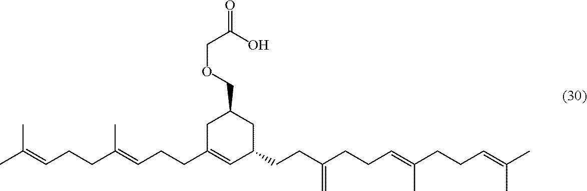 [SMILES: C=C(CC/C=C(\C)CCC=C(C)C)CC[C@@H]1C=C(CC/C=C(\C)CCC=C(C)C)C[C@@H](COCC(=O)O)C1]
or a pharmaceutically acceptable salt thereof.

167. The compound of claim 1, wherein the compound is represented by formula (31) 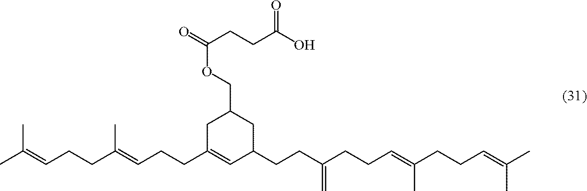 [SMILES: C=C(CC/C=C(\C)CCC=C(C)C)CCC1C=C(CC/C=C(\C)CCC=C(C)C)CC(COC(=O)CCC(=O)O)C1]
or a pharmaceutically acceptable salt thereof.

168. The compound of claim 1, wherein the compound is represented by formula (32) 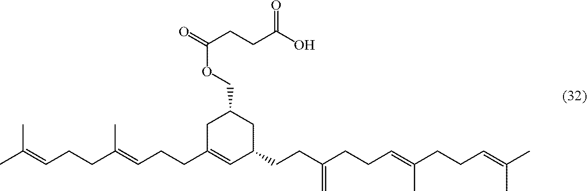 [SMILES: C=C(CC/C=C(\C)CCC=C(C)C)CC[C@@H]1C=C(CC/C=C(\C)CCC=C(C)C)C[C@H](COC(=O)CCC(=O)O)C1]
or a pharmaceutically acceptable salt thereof.

169. The compound of claim 1, wherein the compound is represented by formula (33) 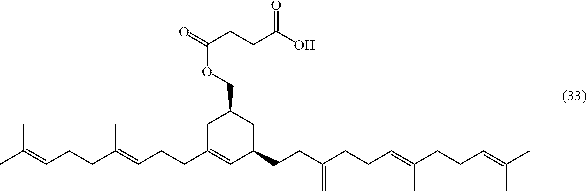 [SMILES: C=C(CC/C=C(\C)CCC=C(C)C)CC[C@H]1C=C(CC/C=C(\C)CCC=C(C)C)C[C@@H](COC(=O)CCC(=O)O)C1]
or a pharmaceutically acceptable salt thereof.

170. The compound of claim 1, wherein the compound is represented by formula (34) 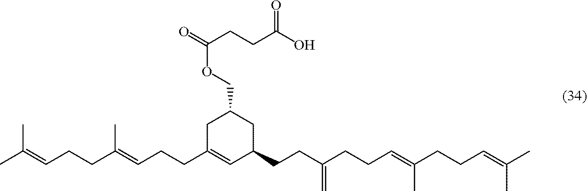 [SMILES: C=C(CC/C=C(\C)CCC=C(C)C)CC[C@H]1C=C(CC/C=C(\C)CCC=C(C)C)C[C@H](COC(=O)CCC(=O)O)C1]
or a pharmaceutically acceptable salt thereof.

171. The compound of claim 1, wherein the compound is represented by formula (35) 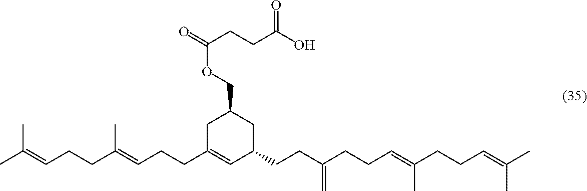 [SMILES: C=C(CC/C=C(\C)CCC=C(C)C)CC[C@@H]1C=C(CC/C=C(\C)CCC=C(C)C)C[C@@H](COC(=O)CCC(=O)O)C1]
or a pharmaceutically acceptable salt thereof.

172. The compound of claim 1, wherein the compound is represented by formula (36) 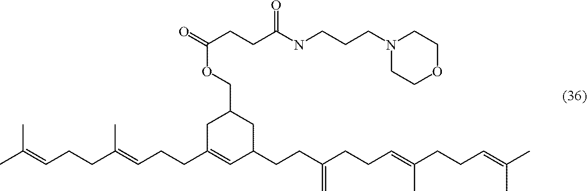 [SMILES: C=C(CC/C=C(\C)CCC=C(C)C)CCC1C=C(CC/C=C(\C)CCC=C(C)C)CC(COC(=O)CCC(=O)NCCCN2CCOCC2)C1]
.

173. The compound of claim 1, wherein the compound is represented by formula (37) 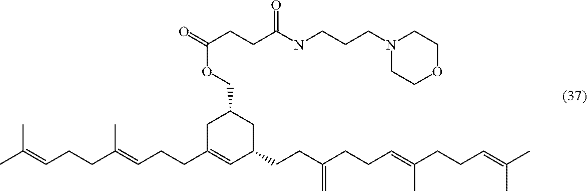 [SMILES: C=C(CC/C=C(\C)CCC=C(C)C)CC[C@@H]1C=C(CC/C=C(\C)CCC=C(C)C)C[C@H](COC(=O)CCC(=O)NCCCN2CCOCC2)C1]
.

174. The compound of claim 1, wherein the compound is represented by formula (38) 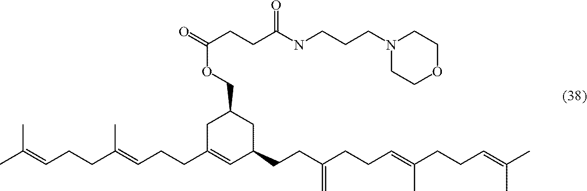 [SMILES: C=C(CC/C=C(\C)CCC=C(C)C)CC[C@H]1C=C(CC/C=C(\C)CCC=C(C)C)C[C@@H](COC(=O)CCC(=O)NCCCN2CCOCC2)C1]
.

175. The compound of claim 1, wherein the compound is represented by formula (39) 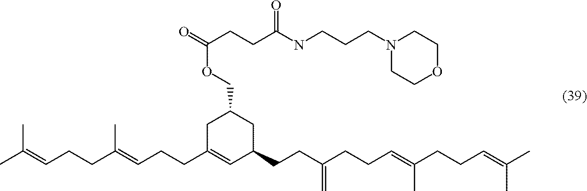 [SMILES: C=C(CC/C=C(\C)CCC=C(C)C)CC[C@H]1C=C(CC/C=C(\C)CCC=C(C)C)C[C@H](COC(=O)CCC(=O)NCCCN2CCOCC2)C1]
.

176. The compound of claim 1, wherein the compound is represented by formula (40) 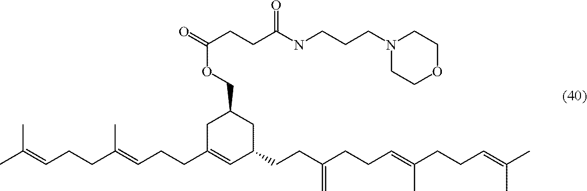 [SMILES: C=C(CC/C=C(\C)CCC=C(C)C)CC[C@@H]1C=C(CC/C=C(\C)CCC=C(C)C)C[C@@H](COC(=O)CCC(=O)NCCCN2CCOCC2)C1]
.

177. The compound of claim 1, wherein the compound is represented by formula (41) 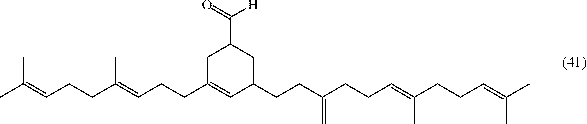 [SMILES: C=C(CC/C=C(\C)CCC=C(C)C)CCC1C=C(CC/C=C(\C)CCC=C(C)C)CC(C=O)C1]
.

178. The compound of claim 1, wherein the compound is represented by formula (42) 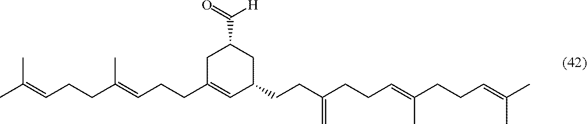 [SMILES: C=C(CC/C=C(\C)CCC=C(C)C)CC[C@@H]1C=C(CC/C=C(\C)CCC=C(C)C)C[C@H](C=O)C1]
.

179. The compound of claim 1, wherein the compound is represented by formula (43) 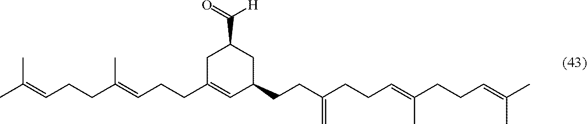 [SMILES: C=C(CC/C=C(\C)CCC=C(C)C)CC[C@H]1C=C(CC/C=C(\C)CCC=C(C)C)C[C@@H](C=O)C1]
.

180. The compound of claim 1, wherein the compound is represented by formula (44) 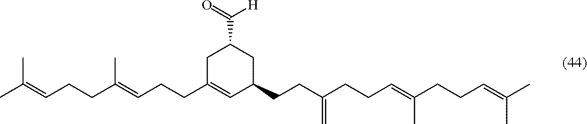 [SMILES: C=C(CC/C=C(\C)CCC=C(C)C)CC[C@H]1C=C(CC/C=C(\C)CCC=C(C)C)C[C@H](C=O)C1]
.

181. The compound of claim 1, wherein the compound is represented by formula (45) 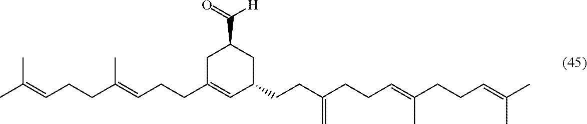 [SMILES: C=C(CC/C=C(\C)CCC=C(C)C)CC[C@@H]1C=C(CC/C=C(\C)CCC=C(C)C)C[C@@H](C=O)C1]
.

182. The compound of claim 1, wherein the compound is represented by formula (46) 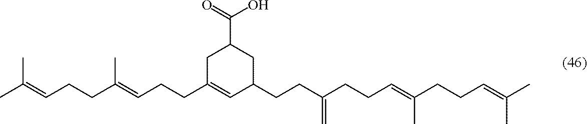 [SMILES: C=C(CC/C=C(\C)CCC=C(C)C)CCC1C=C(CC/C=C(\C)CCC=C(C)C)CC(C(=O)O)C1]
or a pharmaceutically acceptable salt thereof.

183. The compound of claim 1, wherein the compound is represented by formula (47) 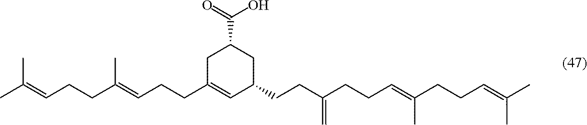 [SMILES: C=C(CC/C=C(\C)CCC=C(C)C)CC[C@@H]1C=C(CC/C=C(\C)CCC=C(C)C)C[C@H](C(=O)O)C1]
or a pharmaceutically acceptable salt thereof.

184. The compound of claim 1, wherein the compound is represented by formula (48) 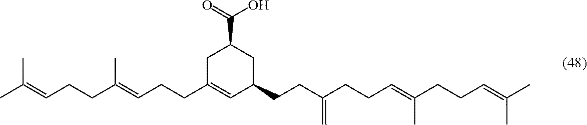 [SMILES: C=C(CC/C=C(\C)CCC=C(C)C)CC[C@H]1C=C(CC/C=C(\C)CCC=C(C)C)C[C@@H](C(=O)O)C1]
or a pharmaceutically acceptable salt thereof.

185. The compound of claim 1, wherein the compound is represented by formula (49) 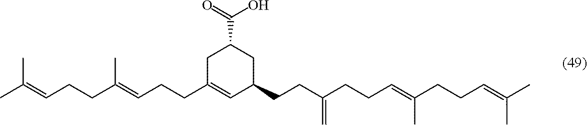 [SMILES: C=C(CC/C=C(\C)CCC=C(C)C)CC[C@H]1C=C(CC/C=C(\C)CCC=C(C)C)C[C@H](C(=O)O)C1]
or a pharmaceutically acceptable salt thereof.

186. The compound of claim 1, wherein the compound is represented by formula (50) 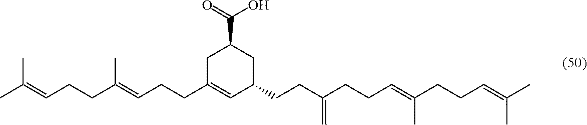 [SMILES: C=C(CC/C=C(\C)CCC=C(C)C)CC[C@@H]1C=C(CC/C=C(\C)CCC=C(C)C)C[C@@H](C(=O)O)C1]
or a pharmaceutically acceptable salt thereof.

187. The compound of claim 1, wherein the compound is represented by formula (51) 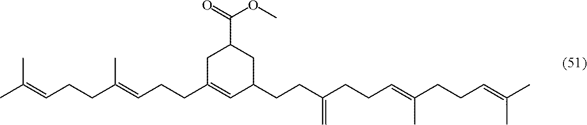 [SMILES: C=C(CC/C=C(\C)CCC=C(C)C)CCC1C=C(CC/C=C(\C)CCC=C(C)C)CC(C(=O)OC)C1]
.

188. The compound of claim 1, wherein the compound is represented by formula (52) 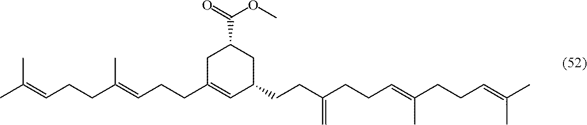 [SMILES: C=C(CC/C=C(\C)CCC=C(C)C)CC[C@@H]1C=C(CC/C=C(\C)CCC=C(C)C)C[C@H](C(=O)OC)C1]
.

189. The compound of claim 1, wherein the compound is represented by formula (53) 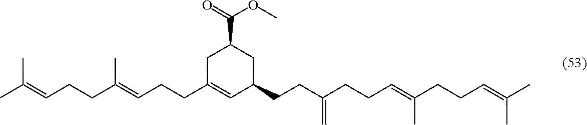 [SMILES: C=C(CC/C=C(\C)CCC=C(C)C)CC[C@H]1C=C(CC/C=C(\C)CCC=C(C)C)C[C@@H](C(=O)OC)C1]
.

190. The compound of claim 1, wherein the compound is represented by formula (54) 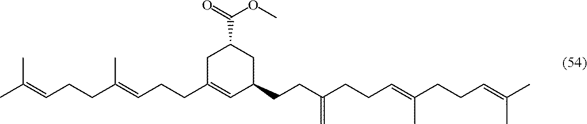 [SMILES: C=C(CC/C=C(\C)CCC=C(C)C)CC[C@H]1C=C(CC/C=C(\C)CCC=C(C)C)C[C@H](C(=O)OC)C1]
.

191. The compound of claim 1, wherein the compound is represented by formula (55) 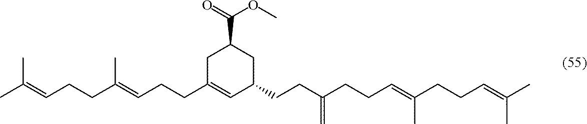 [SMILES: C=C(CC/C=C(\C)CCC=C(C)C)CC[C@@H]1C=C(CC/C=C(\C)CCC=C(C)C)C[C@@H](C(=O)OC)C1]
.

192. The compound of claim 1, wherein the compound is represented by formula (56) 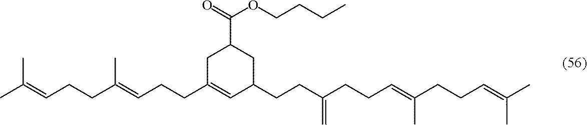 [SMILES: C=C(CC/C=C(\C)CCC=C(C)C)CCC1C=C(CC/C=C(\C)CCC=C(C)C)CC(C(=O)OCCCC)C1]
.

193. The compound of claim 1, wherein the compound is represented by formula (57) 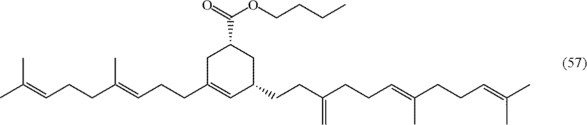 [SMILES: C=C(CC/C=C(\C)CCC=C(C)C)CC[C@@H]1C=C(CC/C=C(\C)CCC=C(C)C)C[C@H](C(=O)OCCCC)C1]
.

194. The compound of claim 1, wherein the compound is represented by formula (58) 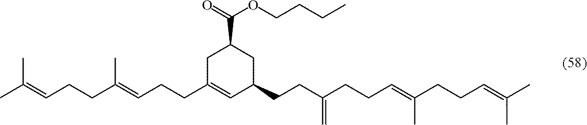 [SMILES: C=C(CC/C=C(\C)CCC=C(C)C)CC[C@H]1C=C(CC/C=C(\C)CCC=C(C)C)C[C@@H](C(=O)OCCCC)C1]
.

195. The compound of claim 1, wherein the compound is represented by formula (59) 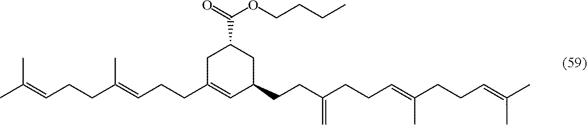 [SMILES: C=C(CC/C=C(\C)CCC=C(C)C)CC[C@H]1C=C(CC/C=C(\C)CCC=C(C)C)C[C@H](C(=O)OCCCC)C1]
.

196. The compound of claim 1, wherein the compound is represented by formula (60) 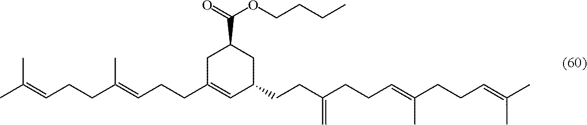 [SMILES: C=C(CC/C=C(\C)CCC=C(C)C)CC[C@@H]1C=C(CC/C=C(\C)CCC=C(C)C)C[C@@H](C(=O)OCCCC)C1]
.

197. The compound of claim 1, wherein the compound is represented by formula (61) 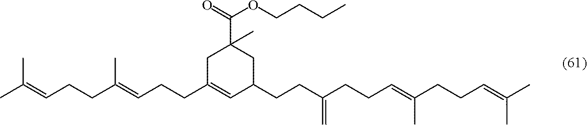 [SMILES: C=C(CC/C=C(\C)CCC=C(C)C)CCC1C=C(CC/C=C(\C)CCC=C(C)C)CC(C)(C(=O)OCCCC)C1]
.

198. The compound of claim 1, wherein the compound is represented by formula (62) 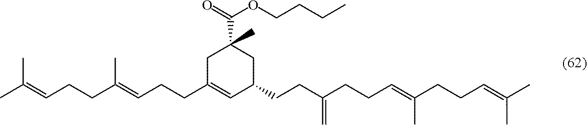 [SMILES: C=C(CC/C=C(\C)CCC=C(C)C)CC[C@@H]1C=C(CC/C=C(\C)CCC=C(C)C)C[C@](C)(C(=O)OCCCC)C1]
.

199. The compound of claim 1, wherein the compound is represented by formula (63) 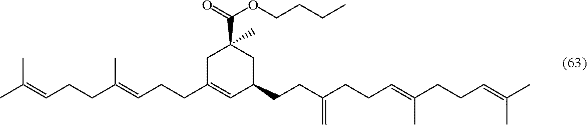 [SMILES: C=C(CC/C=C(\C)CCC=C(C)C)CC[C@H]1C=C(CC/C=C(\C)CCC=C(C)C)C[C@@](C)(C(=O)OCCCC)C1]
.

200. The compound of claim 1, wherein the compound is represented by formula (64) 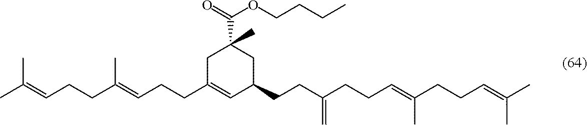 [SMILES: C=C(CC/C=C(\C)CCC=C(C)C)CC[C@H]1C=C(CC/C=C(\C)CCC=C(C)C)C[C@](C)(C(=O)OCCCC)C1]
.

201. The compound of claim 1, wherein the compound is represented by formula (65) 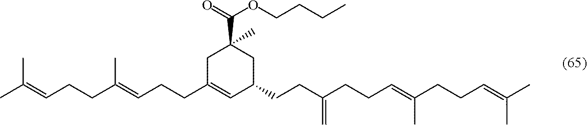 [SMILES: C=C(CC/C=C(\C)CCC=C(C)C)CC[C@@H]1C=C(CC/C=C(\C)CCC=C(C)C)C[C@@](C)(C(=O)OCCCC)C1]
.

202. The compound of claim 1, wherein the compound is represented by formula (66) 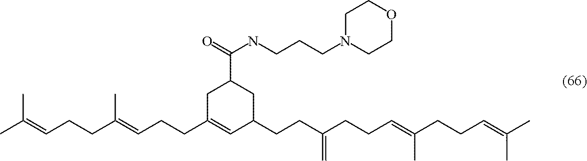 [SMILES: C=C(CC/C=C(\C)CCC=C(C)C)CCC1C=C(CC/C=C(\C)CCC=C(C)C)CC(C(=O)NCCCN2CCOCC2)C1]
.

203. The compound of claim 1, wherein the compound is represented by formula (67) 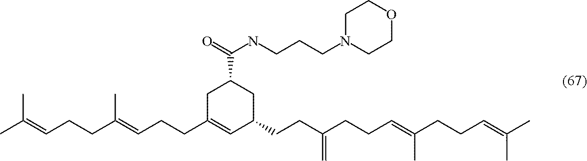 [SMILES: C=C(CC/C=C(\C)CCC=C(C)C)CC[C@@H]1C=C(CC/C=C(\C)CCC=C(C)C)C[C@H](C(=O)NCCCN2CCOCC2)C1]
.

204. The compound of claim 1, wherein the compound is represented by formula (68) 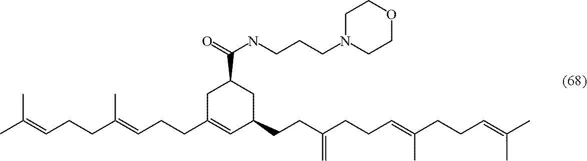 [SMILES: C=C(CC/C=C(\C)CCC=C(C)C)CC[C@H]1C=C(CC/C=C(\C)CCC=C(C)C)C[C@@H](C(=O)NCCCN2CCOCC2)C1]
.

205. The compound of claim 1, wherein the compound is represented by formula (69) 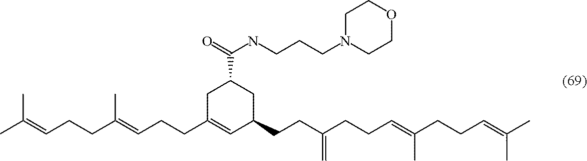 [SMILES: C=C(CC/C=C(\C)CCC=C(C)C)CC[C@H]1C=C(CC/C=C(\C)CCC=C(C)C)C[C@H](C(=O)NCCCN2CCOCC2)C1]
.

206. The compound of claim 1, wherein the compound is represented by formula (70) 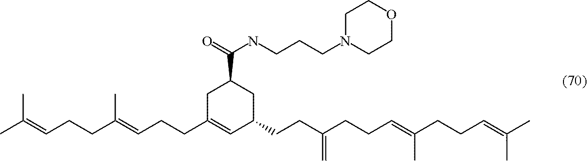 [SMILES: C=C(CC/C=C(\C)CCC=C(C)C)CC[C@@H]1C=C(CC/C=C(\C)CCC=C(C)C)C[C@@H](C(=O)NCCCN2CCOCC2)C1]
.

207. The compound of claim 1, wherein the compound is represented by formula (71) 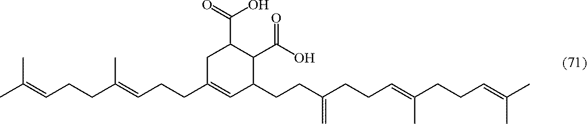 [SMILES: C=C(CC/C=C(\C)CCC=C(C)C)CCC1C=C(CC/C=C(\C)CCC=C(C)C)CC(C(=O)O)C1C(=O)O]
or a pharmaceutically acceptable salt thereof.

208. The compound of claim 1, wherein the compound is represented by formula (72) 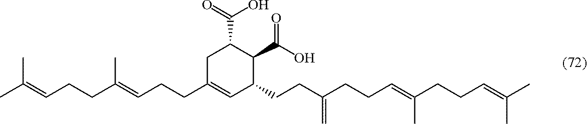 [SMILES: C=C(CC/C=C(\C)CCC=C(C)C)CC[C@@H]1C=C(CC/C=C(\C)CCC=C(C)C)C[C@H](C(=O)O)[C@H]1C(=O)O]
or a pharmaceutically acceptable salt thereof.

209. The compound of claim 1, wherein the compound is represented by formula (73) 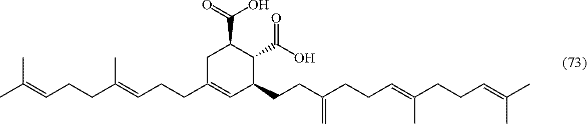 [SMILES: C=C(CC/C=C(\C)CCC=C(C)C)CC[C@H]1C=C(CC/C=C(\C)CCC=C(C)C)C[C@@H](C(=O)O)[C@@H]1C(=O)O]
or a pharmaceutically acceptable salt thereof.

210. The compound of claim 1, wherein the compound is represented by formula (74) 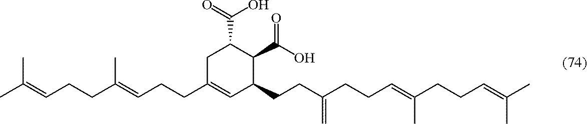 [SMILES: C=C(CC/C=C(\C)CCC=C(C)C)CC[C@H]1C=C(CC/C=C(\C)CCC=C(C)C)C[C@H](C(=O)O)[C@H]1C(=O)O]
or a pharmaceutically acceptable salt thereof.

211. The compound of claim 1, wherein the compound is represented by formula (75) 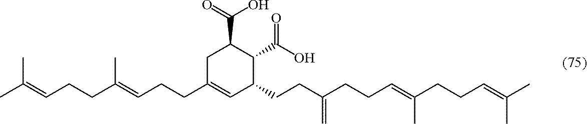 [SMILES: C=C(CC/C=C(\C)CCC=C(C)C)CC[C@@H]1C=C(CC/C=C(\C)CCC=C(C)C)C[C@@H](C(=O)O)[C@@H]1C(=O)O]
or a pharmaceutically acceptable salt thereof.

212. The compound of claim 1, wherein the compound is represented by formula (76) 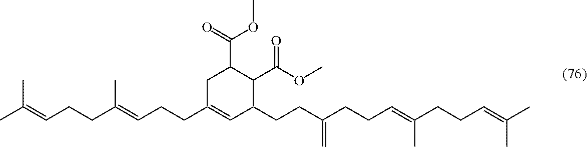 [SMILES: C=C(CC/C=C(\C)CCC=C(C)C)CCC1C=C(CC/C=C(\C)CCC=C(C)C)CC(C(=O)OC)C1C(=O)OC]
.

213. The compound of claim 1, wherein the compound is represented by formula (77) 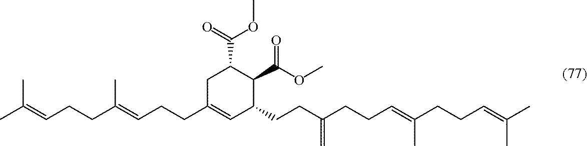 [SMILES: C=C(CC/C=C(\C)CCC=C(C)C)CC[C@@H]1C=C(CC/C=C(\C)CCC=C(C)C)C[C@H](C(=O)OC)[C@H]1C(=O)OC]
.

214. The compound of claim 1, wherein the compound is represented by formula (78) 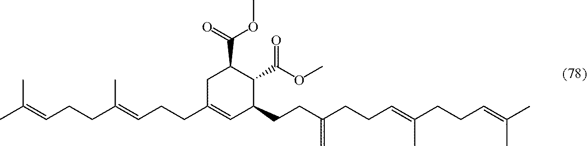 [SMILES: C=C(CC/C=C(\C)CCC=C(C)C)CC[C@H]1C=C(CC/C=C(\C)CCC=C(C)C)C[C@@H](C(=O)OC)[C@@H]1C(=O)OC]
.

215. The compound of claim 1, wherein the compound is represented by formula (79) 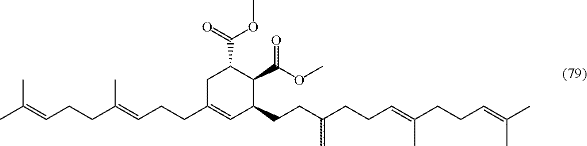 [SMILES: C=C(CC/C=C(\C)CCC=C(C)C)CC[C@H]1C=C(CC/C=C(\C)CCC=C(C)C)C[C@H](C(=O)OC)[C@H]1C(=O)OC]
.

216. The compound of claim 1, wherein the compound is represented by formula (80) 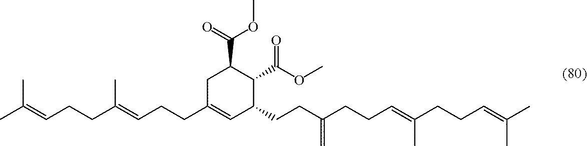 [SMILES: C=C(CC/C=C(\C)CCC=C(C)C)CC[C@@H]1C=C(CC/C=C(\C)CCC=C(C)C)C[C@@H](C(=O)OC)[C@@H]1C(=O)OC]
.

217. The compound of claim 1, wherein the compound is represented by formula (81) 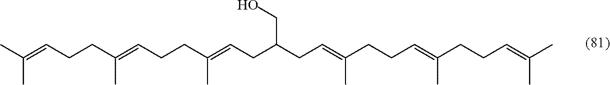 [SMILES: CC(C)=CCC/C(C)=C/CC/C(C)=C/CC(CO)C/C=C(\C)CC/C=C(\C)CCC=C(C)C]
.

218. The compound of claim 1, wherein the compound is represented by formula (82) 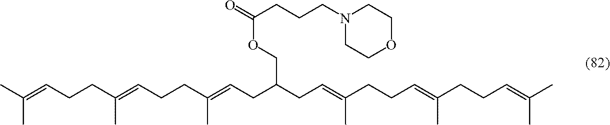 [SMILES: CC(C)=CCC/C(C)=C/CC/C(C)=C/CC(C/C=C(\C)CC/C=C(\C)CCC=C(C)C)COC(=O)CCCN1CCOCC1]
.

219. The compound of claim 1, wherein the compound is represented by formula (83) 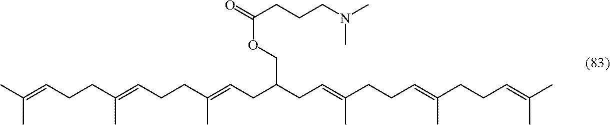 [SMILES: CC(C)=CCC/C(C)=C/CC/C(C)=C/CC(C/C=C(\C)CC/C=C(\C)CCC=C(C)C)COC(=O)CCCN(C)C]
.

220. The compound of claim 1, wherein the compound is represented by formula (84) 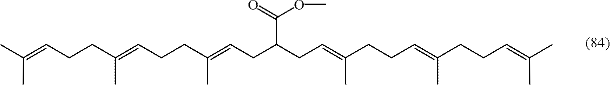 [SMILES: COC(=O)C(C/C=C(\C)CC/C=C(\C)CCC=C(C)C)C/C=C(\C)CC/C=C(\C)CCC=C(C)C]
.

221. The compound of claim 1, wherein the compound is represented by formula (85) 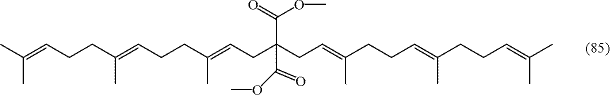 [SMILES: COC(=O)C(C/C=C(\C)CC/C=C(\C)CCC=C(C)C)(C/C=C(\C)CC/C=C(\C)CCC=C(C)C)C(=O)OC]
.

222. The compound of claim 1, wherein the compound is represented by formula (86) 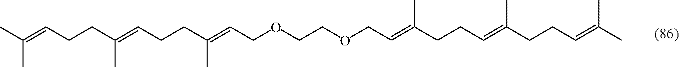 [SMILES: CC(C)=CCC/C(C)=C/CC/C(C)=C/COCCOC/C=C(\C)CC/C=C(\C)CCC=C(C)C]
.

223. The compound of claim 1, wherein the compound is represented by formula (87) 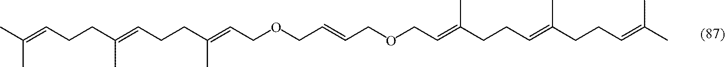 [SMILES: CC(C)=CCC/C(C)=C/CC/C(C)=C/COC/C=C/COC/C=C(\C)CC/C=C(\C)CCC=C(C)C]
.

224. The compound of claim 1, wherein the compound is represented by formula (88) 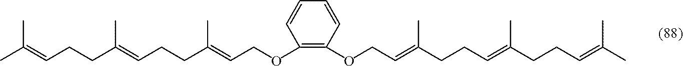 [SMILES: CC(C)=CCC/C(C)=C/CC/C(C)=C/COc1ccccc1OC/C=C(\C)CC/C=C(\C)CCC=C(C)C]
.

225. The compound of claim 1, wherein the compound is represented by formula (89) 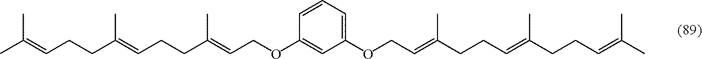 [SMILES: CC(C)=CCC/C(C)=C/CC/C(C)=C/COc1cccc(OC/C=C(\C)CC/C=C(\C)CCC=C(C)C)c1]
.

226. The compound of claim 1, wherein the compound is represented by formula (90) 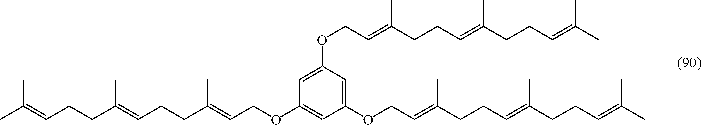 [SMILES: CC(C)=CCC/C(C)=C/CC/C(C)=C/COc1cc(OC/C=C(\C)CC/C=C(\C)CCC=C(C)C)cc(OC/C=C(\C)CC/C=C(\C)CCC=C(C)C)c1]
.

227. The compound of claim 1, wherein the compound is represented by formula (91) 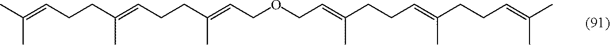 [SMILES: CC(C)=CCC/C(C)=C/CC/C(C)=C/COC/C=C(\C)CC/C=C(\C)CCC=C(C)C]
.

228. A composition comprising the compound of any one of claims 1-227, wherein the compound has a purity of at least 85% by weight.

229. The composition of claim 228, wherein the compound has a purity of from about 85% to about 99.9% by weight.

230. The composition of claim 229, wherein the compound has a purity of from about 90% to about 99.9% by weight.

231. The composition of claim 230, wherein the compound has a purity of from about 95% to about 99.9% by weight.

232. The composition of any one of claims 228-231, wherein the compound is present in the composition in an amount of at least 1 kg.

233. The composition of claim 232, wherein the compound is present in the composition in an amount of from about 1 kg to about 100 kg.

234. An adjuvant formulation comprising the compound of any one of claims 1-227 and a pharmaceutically acceptable carrier, diluent, or excipient.

235. A vaccine comprising therapeutically or prophylactically effective amounts of the adjuvant formulation of claim 234 and an antigen.

236. A vaccine comprising the compound of any one of claims 1-227 covalently bound to an antigen.

237. The vaccine of claim 235 or 236, wherein the antigen is a protein expressed by a virus.

238. The vaccine of claim 235 or 236, wherein the antigen is encoded by a nucleic acid molecule encoding a protein expressed by a virus.

239. The vaccine of claim 238, wherein the nucleic acid molecule is a deoxyribonucleic acid (DNA) or a ribonucleic acid (RNA) molecule.

240. The vaccine of any one of claims 237-239, wherein the virus is selected from influenza virus, hepatitis A virus, hepatitis B virus, hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, llheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O′nyong′nyong virus, chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi’s sarcoma associated-herpesvirus (KSHV), severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus, rhinovirus, mumps virus, poliovirus, human enterovirus, coxsackievirus, human papilloma virus, adeno-associated virus, astrovirus, JC virus, BK virus, SV40 virus, Norwalk virus, rotavirus, human immunodeficiency virus (HIV), human T-lymphotropic virus, SARS-CoV-2, MERS-CoV, SARS-CoV, OC43, and HKU1.

241. The vaccine of claim 235 or 236, wherein the antigen is a protein expressed by a bacterium.

242. The vaccine of claim 235 or 236, wherein the antigen is encoded by a nucleic acid molecule encoding a protein expressed by a bacterium.

243. The vaccine of claim 242, wherein the nucleic acid molecule is a DNA or RNA molecule.

244. The vaccine of any one of claims 241-243, wherein the bacterium belongs to a genus selected from Mycobacterium, Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasturella, Francisella, Brucella, Bartonella, Clostridium, Vibrio, Campylobacter, and Staphylococcus, optionally wherein the bacterium is Mycobacterium tuberculosis.

245. The vaccine of claim 235 or 236, wherein the antigen is a protein expressed by a parasite.

246. The vaccine of claim 235 or 236, wherein the antigen is encoded by a nucleic acid molecule encoding a protein expressed by a parasite.

247. The vaccine of claim 246, wherein the nucleic acid molecule is a DNA or RNA molecule.

248. The vaccine of any one of claims 245-247, wherein the parasite is selected from Plasmodium malariae, Plasmodium vivax, Plasmodium ovale, Plasmodium falciparum, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Trichomonas vaginalis, and Histomonas meleagridis, Richuris trichiura, Ascaris lumbricoides, Enterobius vermicularis, Ancylostoma duodenale, Necator americanus, Strongyloides stercoralis, Wuchereria bancrofti, Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus.

249. The vaccine of claim 235 or 236, wherein the antigen is a protein expressed by a cancer cell.

250. The vaccine of claim 235 or 236, wherein the antigen is encoded by a nucleic acid molecule encoding a protein expressed by a cancer cell.

251. The vaccine of claim 250, wherein the nucleic acid molecule is a DNA or RNA molecule.

252. The vaccine of any one of claims 249-251, wherein the protein is selected from gp100, Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-1, MUC-16, L1CAM, HERV-K-MEL, KK-LC-1, KM-HN-1, LAGE-1, Sp17, TAG-1, TAG-2, ENAH (hMena), mammaglobin-A, NY-BR-1, BAGE-1, MAGE-A1, MAGE-A2, MAGE-A4, MAGE-A6, MAGE-A9, MAGE-A10, MAGE-A12, MAGE-C2, mucink, SSX-2, SSX-4, TRAG-3, c-myc, cyclin B1, p62, Survivin, CD45, DKK1, RU2AS, Telomerase, K-ras, G250, Hepsin, Intestinal carboxyl esterase, α-foetoprotein, M-CSF, PSMA, CASP-5, COA-1, OGT, OS-9, TGF-βRII, gp70, CALCA, CD274, mdm-2, α-actinin-4, Elongation factor 2, ME1, NFYC, GAGE-1/2/8, GAGE-3/4/5/6/7, XAGE-1b/GAGED2a, STEAP1, PAP, PSA, FGF5, hsp70-2, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, GnTVf, LY6K, and NA88-A.

253. A method of inducing an antigen-specific immune response in a subject, the method comprising administering to the subject the vaccine of any one of claims 235-252.

254. A method of treating or preventing a viral, bacterial, or parasitic infection in a subject, the method comprising administering to the subject the vaccine of any one of claims 235-248.

255. A method of treating or preventing a cancer in a subject, the method comprising administering to the subject the vaccine of any one of claims 249-252.

256. The method of any one of claims 253-255, wherein the subject is a mammal.

257. The method of claim 151, wherein the mammal is a human.

258. A method of synthesizing a compound represented by formula (II) 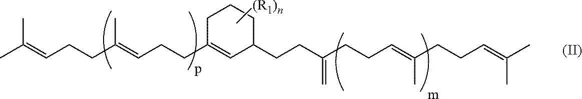
or a pharmaceutically acceptable salt thereof, the method comprising reacting a diene represented by formula (XXVII) 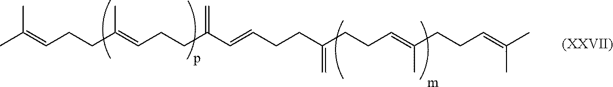
with a dienophile represented by formula (XXVIII) 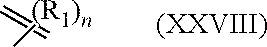
under Diels-Alder reaction conditions,
wherein each R₁ is, independently, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, carbonyl, optionally substituted alkoxy, optionally substituted acyloxy, hydroxyl, optionally substituted haloalkyl, or halogen;
n is an integer from 0 to 4;
m is an integer from 0 to 3; and
p is an integer from 0 to 3.

259. A method of synthesizing a compound represented by formula (XI) 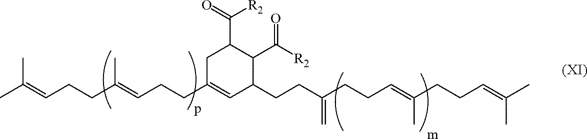
or a pharmaceutically acceptable salt thereof, the method comprising reacting a diene represented by formula (XXVII) 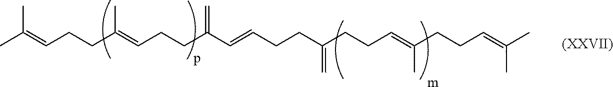
with a dienophile represented by formula (XXIX) 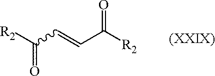
under Diels-Alder reaction conditions,
wherein each R₂ is, independently, hydrogen, optionally substituted alkyl, optionally substituted aminoalkyl, optionally substituted heteroalkyl, optionally substituted alkenyl, optionally substituted heteroalkenyl, optionally substituted alkynyl, optionally substituted heteroalkynyl, optionally substituted alkoxy, optionally substituted acyloxy, optionally substituted sulfonyloxy, hydroxyl, or halogen;
m is an integer from 0 to 3; and
p is an integer from 0 to 3;
or a pharmaceutically acceptable salt thereof.

260. A method of synthesizing a compound represented by formula (XXX) 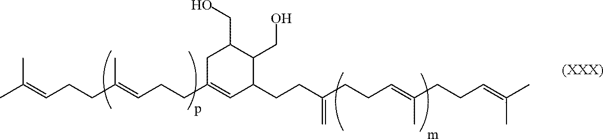
the method comprising reacting a diene represented by formula (XXVII) 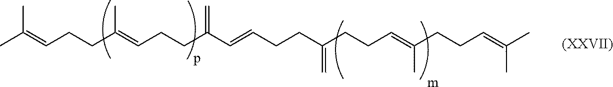
with a dienophile represented by formula (XXIX) 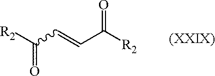
under Diels-Alder reaction conditions, thereby forming an intermediate represented by formula (XXXI) 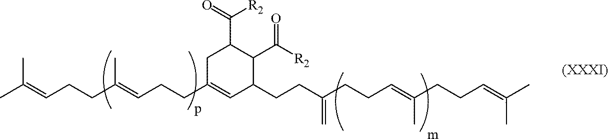
and subsequently reacting the intermediate represented by formula (XXXI) with a reducing agent, thereby producing a compound represented by formula (XXX),
wherein each R₂ is, independently, hydrogen, optionally substituted alkoxy, optionally substituted acyloxy, optionally substituted sulfonyloxy, hydroxyl, or halogen;
m is an integer from 0 to 3; and
p is an integer from 0 to 3;
or a pharmaceutically acceptable salt thereof.
